SEQUENCE LISTING (16 sequences)

SEQ ID NO: 1 - DNA, 25 bp, synthetic construct
  source 1..25: mol_type = other DNA
caccggtctgcttcttgtaaaaacc

SEQ ID NO: 2 - DNA, 25 bp, synthetic construct
  source 1..25: mol_type = other DNA
aaacggtttttacaagaagcagacc

SEQ ID NO: 3 - DNA, 22 bp, synthetic construct
  source 1..22: mol_type = other DNA
attgacactggcaaaacaatgc

SEQ ID NO: 4 - DNA, 20 bp, synthetic construct
  source 1..20: mol_type = other DNA
tccaacacttcgtggggtcc

SEQ ID NO: 5 - DNA, 18 bp, synthetic construct
  source 1..18: mol_type = other DNA
acgctgccacacctacga

SEQ ID NO: 6 - DNA, 21 bp, synthetic construct
  source 1..21: mol_type = other DNA
ccttgaacccaggaatctcag

SEQ ID NO: 7 - DNA, 28 bp, synthetic construct
  source 1..28: mol_type = other DNA
tggagactactggattgaccccaaccaa

SEQ ID NO: 8 - DNA, 24 bp, synthetic construct
  source 1..24: mol_type = other DNA
tctcgccagtctccatgttgcaga

SEQ ID NO: 9 - DNA, 22 bp, synthetic construct
  source 1..22: mol_type = other DNA
attgacactggcaaaacaatgc

SEQ ID NO: 10 - DNA, 20 bp, synthetic construct
  source 1..20: mol_type = other DNA
tccaacacttcgtggggtcc

SEQ ID NO: 11 - DNA, 18 bp, synthetic construct
  source 1..18: mol_type = other DNA
acgctgccacacctacga

SEQ ID NO: 12 - DNA, 21 bp, synthetic construct
  source 1..21: mol_type = other DNA
ccttgaacccaggaatctcag

SEQ ID NO: 13 - DNA, 20 bp, synthetic construct
  source 1..20: mol_type = other DNA
ttgctcctctctgttttcgt

SEQ ID NO: 14 - DNA, 21 bp, synthetic construct
  source 1..21: mol_type = other DNA
atacccttccccacatctccc

SEQ ID NO: 15 - DNA, 17 bp, synthetic construct
  source 1..17: mol_type = other DNA
gtggtggtcggggcctt

SEQ ID NO: 16 - DNA, 17 bp, synthetic construct
  source 1..17: mol_type = other DNA
cggagcaggcaggacac

(19) United States
(12) Patent Application Publication
(10) Pub. No.: US 2025/0241906 A1
(43) Pub. Date: Jul. 31, 2025

(54) ANABOLIC DRUGS STIMULATING TYPE II COLLAGEN PRODUCTION FROM CHONDROCYTES OR THEIR PROGENITORS

(71) Applicant: UNIVERSITY OF CENTRAL FLORIDA RESEARCH FOUNDATION, INC., Orlando, FL (US)

(72) Inventors: Thomas KEAN, Orlando, FL (US); Maria CRUZ, Orlando, FL (US)

(21) Appl. No.: 18/838,734

(22) PCT Filed: Feb. 15, 2023
(86) PCT No.: PCT/US2023/062656

§ 371 Date: Aug. 15, 2024

Related U.S. Application Data
(60) Provisional application No. 63/310,299, filed on Feb. 15, 2022.

(51) Int. Cl. A61K 31/4748; A61K 31/473; A61K 31/475; A61K 31/519; A61K 31/522; A61L 27/20; A61L 27/22; A61L 27/38; C12N 5/077; G01N 33/50

(52) U.S. Cl. CPC A61K 31/4748; A61K 31/473; A61K 31/475; A61K 31/519; A61K 31/522; A61L 27/20; A61L 27/222; A61L 27/3817; A61L 27/3852; C12N 5/0655; G01N 33/502; G01N 33/5044; A61L 2430/06; C12N 2500/02; C12N 2501/15; C12N 2501/2301; C12N 2503/02; C12N 2513/00; C12N 2533/30; C12N 2533/74

(57) ABSTRACT

Disclosed herein are three-dimensional culture system for producing cartilage and/or selecting therapeutic agents for promoting cartilage regeneration.

### CROSS-REFERENCE TO RELATED APPLICATIONS

This application claims the priority benefit of U.S. Provisional Application No. 63/310,299, filed Feb. 15, 2022, which is expressly incorporated herein by reference in its entirety.

### BACKGROUND

Osteoarthritis is a major healthcare burden both in terms of financial and quality of life costs amounting to over 10% of the US healthcare burden. Osteoarthritis is characterized by the degeneration of articular cartilage. Adult cartilage has no innate repair mechanism, and there are currently no disease modifying therapies for osteoarthritis. Current drugs treat pain and inflammation before a total joint replacement is required. As such, there is a strong need for novel cartilage regenerative therapies. What is needed are compositions for treating osteoarthritis.

### SUMMARY

Disclosed herein are methods relates a three-dimensional culture system and uses thereof, including for examples, for producing cartilage and/or selecting therapeutic agents for promoting cartilage regeneration.

The details of one or more embodiments of the invention are set forth in the accompanying drawings and the description below. Other features, objects, and advantages of the invention will be apparent from the description and drawings, and from the claims.

In some aspects, disclosed herein is a three-dimensional culture system for producing cartilage, said culture system comprising a chondrocyte that is genetically engineered to express a reporter protein; wherein the expression of a reporter protein is driven by a promoter of an extracellular matrix protein coding gene. In some embodiments, the promoter a type 2 collagen promoter. In some embodiments, the promoter is a promoter of COL2A1, ACAN, or PRG4. Accordingly, in some aspect, disclosed herein is a three-dimensional culture system for producing cartilage, said culture system comprising a chondrocyte that is genetically engineered to express a reporter protein; wherein the expression of a reporter protein is driven by a type 2 collagen promoter. In some aspect, disclosed herein is a three-dimensional culture system for producing cartilage, said culture system comprising a chondrocyte that is genetically engineered to express a reporter protein; wherein the expression of a reporter protein is driven by a PRG4 promoter.

In some embodiments, the culture system is at about 2% to 8% oxygen. In some embodiments, the culture system is at about 5% oxygen.

In some embodiments, the reporter protein is a bioluminescent protein (for examples, Gaussia luciferase or mCherry protein).

Also disclosed herein is a method of producing cartilage comprising using the three-dimensional culture system of any preceding aspect.

In some aspects, disclosed herein is a three-dimensional culture system for selecting an agent (e.g., a therapeutic agent, a natural product, a mineral, or a biomaterial) for promoting cartilage regeneration, said culture system comprising a chondrocyte that is genetically engineered to express a reporter protein; wherein the expression of a reporter protein is driven by a promoter of an extracellular matrix protein coding gene. In some embodiments, the promoter a type 2 collagen promoter. In some embodiments, the promoter is a promoter of COL2A1, ACAN, or PRG4. Accordingly, in some aspect, disclosed herein is a three-dimensional culture system for producing cartilage, said culture system comprising a chondrocyte that is genetically engineered to express a reporter protein; wherein the expression of a reporter protein is driven by a type 2 collagen promoter. In some aspect, disclosed herein is a three-dimensional culture system for producing cartilage, said culture system comprising a chondrocyte that is genetically engineered to express a reporter protein; wherein the expression of a reporter protein is driven by a PRG4 promoter.

In some embodiments, wherein the culture system is at about 2% to 8% oxygen. In some embodiments, the culture system is at about 5% oxygen. In some embodiments, the reporter protein is a bioluminescent protein (for examples, Gaussia luciferase or mCherry protein).

In some embodiments, the culture system further comprises IL-1β and/or TGF-β.

Also disclosed herein is a method of selecting an agent (e.g., a therapeutic agent, a natural product, a mineral, or a biomaterial) for promoting cartilage regeneration, comprising
a. contacting the chondrocyte of the three-dimensional culture system of any preceding aspect with the therapeutic agent;
b. obtaining a sample of the culture medium; and
c. determining a level of the reporter protein in the sample of step b;
   wherein increased level of the reporter protein in the sample relative to a reference control indicates that the therapeutic agent promotes cartilage regeneration.

In some embodiments, the sample of the culture medium is obtained on day 5 or later after contacting the chondrocyte with the therapeutic agent. In some embodiments, the sample of the culture medium is obtained on day 15 or later after contacting the chondrocyte with the therapeutic agent. In some embodiments, the sample of the culture medium is obtained on day 20 or later after contacting the chondrocyte with the therapeutic agent. In some embodiments, step a) further comprises contacting the chondrocyte with IL-1β and/or TGF-β from day 15 after contacting the chondrocyte with the therapeutic agent.

In some embodiments, the therapeutic agent promoting cartilage regeneration increases an expression level of a dopamine receptor in the chondrocyte. In some embodiments, the dopamine receptor comprises dopamine receptor type 4.

Also disclosed herein is a method of treating a disorder of cartilage in a subject in need, comprising administering to the subject a therapeutically effective amount of a therapeutic agent, wherein the therapeutic agent is a dopamine receptor agonist. In some embodiments, the therapeutic agent increases an expression level of a dopamine receptor. In some embodiments, the therapeutic agent comprises 6-Hydroxy-2-methoxyaporphine, (-)-Apoglaziovine, Pentoxifylline, 1,10-Dihydroxy-2-methoxyaporphine, Deserpidine, Vincristine sulfate, Promoline, or Calcium folinate, or a derivative thereof.

Also disclosed herein is a three-dimensional bioprinting implant comprising one or more biomaterials and cells, wherein the biomaterials are selected from gelatin methacrylate (GelMA), hyaluronic acid methacrylate (HAMA), and oxidized methacrylated alginate (OMA).

### DESCRIPTION OF DRAWINGS

FIG. 1: Plasmid map. Col2gLuc plasmid map (HPRM22364-LvPG02; GeneCopoeia).

FIG. 2A and FIG. 2B: Dopamine receptor expression and induction of type II collagen expression. A) Dopamine receptor expression over a 21-day differentiation B) cartilage tissue response to stimulation with drug 204 over the 22-day assay.

FIG. 3A, FIG. 3B, FIG. 3C, FIG. 3D: Drug screen chondrogenic response FIG. 3A) Time-course type II collagen promoter-driven expression of human col2gLuc expressing cells in the presence of selected drugs (84, 186, 204, 418) from natural product library vs. DMSO and untreated controls. FIG. 3B) Volcano plot identifying statistically significant drug effects at day 8.

FIG. 3C) Type II collagen immunohistochemistry (purple) in untreated (C1) and drug 84 treated (C2) spheroids. FIG. 3D) Glycosaminoglycan (safranin-O; red) staining in untreated (D1) and Drug 84 (D2) treated spheroids.

FIG. 4: Proposed mechanism of Dopamine Receptor D4 induction of type II collagen expression The DRD4 is an inhibitor of adenylyl cyclase, thereby inhibiting the production of CAMP. It is proposed that an antagonist would therefore increase cAMP leading to phosphorylation of SOX9 and CREB transcription factors subsequent nuclear translocation, and upregulation of type II collagen.

FIG. 5A and FIG. 5B: Non-invasive knee injury model. Animals are anaesthetized and place in a compression device. FIG. 5A) Displacement vs force readout of compressive loading of the knee. FIG. 5B) Diagram of loading model ensuing correct alignment and loading of limb causing non-invasive rupture of the ACL.

FIG. 6: Scaling of spheroid assay. In a similar rabbit col2gLuc chondrocyte-based screening assay, 50,000 cells in a 96-well plate triangle are scaled to 6250 cells in a 384-well plate square (n≥6±S.D.)

FIG. 7: time dependent increase in expression of luminescence.

FIG. 8: day 8 indication of significant increase in type II collagen expression.

FIG. 9A, FIG. 9B, FIG. 9C, FIG. 9D, and FIG. 9E: Type II collagen promoter-driven luminescence in primary human chondrocytes FIG. 9A) TGFβ1 dose response curve in primary human chondrocytes under physioxic (5%) oxygen tension at day 7 (n≥10±S.D.) FIG. 9B) Overall results of natural product screen (390 compounds) over 22 day chondrogenic assay, with DMSO control in pink (-) and untreated control in yellow (-). FIG. 9C) Statistical analysis (volcano plot) of natural product screen on day 8 (False discovery rate 1%). FIG. 9D) End of assay immunohistochemistry staining for type II collagen in untreated (D1) and Drug 84 samples (D2).

FIG. 9E) End of assay histochemistry for glycosaminoglycan (safranin-O) in untreated (E1) and Drug 84 samples (E2).

FIG. 10A and FIG. 10B: Expression of dopamine receptors in mesenchymal stromal cells during chondrogenesis FIG. 10A) Expression of dopamine receptor D1 during chondrogenesis.

FIG. 10B) Expression of dopamine receptor D4 during chondrogenesis.

FIG. 11: validation of screening data for candidate drug 84 Pellet size and luminescence are shown over a 22 day chondrogenic assay.

FIG. 12: Promoter-driven Gaussia luciferase construct The 1.4 kb type II collagen promoter drives the secreted Gaussia luciferase reporter.

FIG. 13A, FIG. 13B, FIG. 13C, FIG. 13D, FIG. 13E, FIG. 13F: End point assays of human scaffold-free tissue engineered sheets. FIG. 13A) Mechanical tests showing improved compressive moduli at physioxia FIG. 13B) macro-view of 16 cm human sheet at physioxia and atmospheric 02 culture FIG. 13C) Biochemical assays for glycosaminoglycan/DNA FIG. 13D) total collagen/DNA FIG. 13E) Collagen cross-links with cross-link analysis showing crosslinks between type II, IX and XI collagens. FIG. 13F) Histology showing glycosaminoglycan, and type II collagen staining with little type I or X. scale bar 500 μm; data from 8 experiments, 6 human donors.

FIG. 14: compression testing of 3D bioprinted sheets Sheets of rabbit articular cartilage formed in either a self-assembled, scaffold-free format, a direct-write PCL scaffold, bioprinted in collagen or combined direct-write PCL support and collagen bioprinted, showed significantly lower compressive moduli. Native cartilage had an average modulus of 5.2 MPa.

FIG. 15: High throughput microscopy analysis Analysis of GFP positive human articular chondrocytes in 96-well plate format, inset is a higher magnification of a single chondrocyte pellet. Scale bar 200 μm.

FIG. 16: Overview of experiment design.

FIG. 17: Response surface optimization Plot shows aggrecan: versican (ACAN: VCAN) expression ratio in response to dexamethasone and glucose.

FIG. 18A, FIG. 18B, FIG. 18C: Biochamber and cartilage sheet FIG. 18A) Modified Nalgene container with 22 μm syringe filter for gas exchange, assembled biochamber with seeding chamber (left) removed (right). FIG. 18B) Annotated CAD model of biochamber. FIG. 18C) Tissue engineered cartilage after 1 month of culture.

FIG. 19: Experiment design of rabbit chondral defect repair.

FIG. 20A and FIG. 20B: Double crosslinking glue for implant fixation FIG. 20A) Gelatin is modified by ethyl-dimethyl-aminopropylcarbodiimide/N-hydroxy-succinimide (EDC/NHS) coupling. FIG. 20B) quick crosslinks are formed by dopamine-Fe³⁺ complexation followed by slower, genipin mediated crosslinks.

FIG. 21: Gait analysis setup. Rabbit gait will be assessed by high speed video and paw print analyses.

FIG. 22: μCT assessment of cartilage and trabecular bone. The excised humeral head was scanned in air at 36 micron resolution. The articulating cartilage is shown in green and trabecular bone is clearly seen and will be quantified (Dragonfly 3D). Scale bar=4 mm.

39.

FIG. 23A, FIG. 23B, FIG. 23C, and FIG. 23D. Drug Screen of NCI library via HuCol2gLuc system identified hits that increased type II collagen. (FIG. 23A) Overview of Day 22 luminescence for 390 Natural Drug Screen Library normalized to DMSO (vehicle) control. (FIG. 23B) Volcano Plot of the difference of Day 22 Luminescence between DMSO controls and Natural Drug Compounds 390 compounds of Natural Drug Screen. (FIG. 23C) Luminescence signal for aggregates treated with candidate 84 (promoline) and 204 (deserpidine) over 22 days. Results normalized to DMSO control. (FIG. 23D) Immunohistological staining for type II collagen of day 22 aggregates treated with candidate 84 or 204 for type II collagen (10×). Scale Bars, 200 um. 40. FIG. 24A, FIG. 24B, FIG. 24C, and FIG. 24D. Donor 1 cumulative RLU-one way ANOVA significant, multiple comparisons (NS) DMSO vs untreated controls for the rest of the slides. Donor 1 Graphs from drug dose response curves Donor 2 graphs from repeat results C) 10 ng_N_5 μM—two way repeated measures ANOVA, Dunnett's correction D) 10 ng cumulative one way ANOVA Dunnett correction.

FIG. 25A, FIG. 25B, FIG. 25C, FIG. 25D, FIG. 25E, and FIG. 25F. Biochemical assays on HuCol2gLuc aggregates from two donors at day 22. Total DNA content for donor 1 (μg per sample) is shown in (FIG. 25A) and for donor 2 in (FIG. 25D). Glycosaminoglycan content was quantified in (FIG. 25B) for donor 1 and (FIG. 25E) for donor 2. Total of micrograms of collagen content for donor 1 and donor 2 are shown in (FIG. 25C) and (FIG. 25F) respectively. N=4. Individual replicates or mean of replicates are shown with error bars indicating standard deviation and * indicating p<0.01 vs. untreated control, * promoline; * deserpidine.

FIG. 26A, FIG. 26B, FIG. 26C, FIG. 26D, FIG. 26E, FIG. 26F, FIG. 26G, FIG. 26H, and FIG. 26I. Response of HuCol2gLuc aggregates to promoline is dose dependent. HuCol2gLuc aggregates were treated with promoline (0-10 μM). Dose response curves were generated from luminescence data at day 3 (FIG. 26A), day 10 (FIG. 26B) and cumulative luminescence over 22 days (FIG. 26C). Metabolic activity (FIG. 26D) and DNA content (FIG. 26E) are shown at day 22. N=6. Individual replicates or mean of replicates are shown with error bars indicating standard deviation and * indicating p<0.05 vs. untreated control. Type II collagen staining of day 22 aggregates treated with indicated doses of promoline (FIG. 26F-26I). Scale Bars, 200 μm.

FIG. 27A, FIG. 27B, FIG. 27C, and FIG. 27D. Structure and predicted targets for candidate 84. Chemical structure of promoline (FIG. 27A). SwissTargetPrediction predicted targets for promoline presented by target class (FIG. 27B). DRD4 temporal expression in human mesenchymal stromal cells (MSCs) during 21 days of chondrogenesis FIG. 27C, n=3). DRD4 staining (purple) in mouse brain and in human cartilage (FIG. 27D) Scale Bars=200 μm.

FIG. 28A, FIG. 28B, FIG. 28C, FIG. 28D, and FIG. 28E. DRD4 agonist and antagonist treatment increased type II collagen expression in HuCol2gLuc aggregates. Luminescence signal over 22 days after treatment with DRD4 agonist (ABT 724 (FIG. 28A)) and DRD4 antagonist (PNU 96415E (FIG. 28B)). Statistically significant differences vs. untreated control are indicated by the respective letters for the dose (s, t, u, w, y, z). Results also shown as cumulative luminescence for treatment with ABT 724 (FIG. 28C), and PNU96415E (FIG. 28D). N=6. Individual replicates or mean of replicates are shown with error bars indicating standard deviation and * indicating p<0.05, and ** indicating p<0.01 vs. untreated control. Immunohistological staining for DRD4 and type II collagen in HuCol2gLuc aggregates after ABT 724 and PNU 96415E treatment at indicated doses (FIG. 28E). Scale Bars=200 μm.

FIG. 29A, FIG. 29B, FIG. 29C, FIG. 29D, and FIG. 29E. Promoline increases DRD4 expression in primary human chondrocytes Expression of DRD4 in monolayer cultures of primary human chondrocytes via PCR after treatment with promoline for 24 hours. Show for donor 1 (FIG. 29A) and donor 2 (FIG. 29B) Results shown as fold changed vs. untreated control and normalized to reference gene HPRT. N=3. Immunohistological staining for DRD4 n in HuCol2gLuc aggregates after treatment with promoline (FIG. 29C). Scale Bars=200 μm. Dot blot of kinase phosphorylation after exposure to lysate of primary human chondrocytes after treatment with promoline or DMSO as a vehicle control (FIG. 29D). Data from the dot blots is also shown as normalized integrated density (FIG. 29E). Error bars indicate standard deviation and ** indicate p<0.01.

FIG. 30A and FIG. 30B: Dose effect of TGF-β1 on COL2A1-GLuc reporter rabbit chondrocytes. FIG. 30A and FIG. 30B Primary COL2A1-GLuc rabbit chondrocytes were grown in aggregate culture in the presence of different concentrations of TGF-β1 (0-10 ng/ml). Dose response curves were generated from transformed luminescence data at day 7 (FIG. 30A) and day 21 (FIG. 30B) after seeding. EC50s for each day are shown within each curve. Values are the mean±S.D. n=4.

FIG. 31A, FIG. 31B, FIG. 31C and FIG. 31D: Dose effect of basal chondrogenic media supplemented with DoE micronutrient combinations on COL2A1-GLuc reporter Rabbit chondrocytes. Normal probability plots of the residuals for Gaussia Luciferase signal at weeks 2 (FIG. 31A) and 3 (FIG. 31B) after seeding. 3D surface response plots for interactive effects between vitamin A and linolenic acid at indicated weeks after seeding. End of week 2 (FIG. 31C), and week 3 (FIG. 31D).

FIGS. 32A-32K: Validation of DoE predicted optimal conditions. a-d Conditions predicted by DoE analysis were tested in aggregate culture of COL2A1-GLuc reporter rabbit chondrocytes over 22 days. Results are shown as luminescence over 22 days sampled (FIG. 32A), as well as cumulative luminescence signal (FIG. 32B). To explore temporal effects, data was also analyzed at single day luminescence shown here for Day 10 (FIG. 32C) and Day 22 (FIG. 32D). At day 22, aggregate cultures were assessed for total DNA (FIG. 32E), glycosaminoglycan (FIG. 32F) and collagen (FIG. 32G) content. Results are also shown as total glycosaminoglycan and collagen normalized to DNA content (FIG. 32H, 32I) and to each other (FIG. 32J). Alternatively, aggregates were fixed, embedded in paraffin and sectioned. k Sections were analyzed for type II collagen. Scale Bars, 200 μm. FIG. 32A-32D N=6. FIG. 32E-32J N=5. Replicates or means+S.D. and ** p<0.01 and ***p<0.001 vs. Basal Media control.

FIG. 33A and FIG. 33B: Effect of a single micronutrient removal from DoE predicted condition 25 on type II collagen driven expression of Gaussia Luciferase. Aggregates of COL2A1-GLuc primary rabbit chondrocytes were cultured with condition 25 or condition 25 with a single micronutrient removed as indicated. Results are shown as luminescence over 22 days (FIG. 33a). Luminescence is shown for a single timepoint, Day 10 (FIG. 33b). Ø indicates p<0.05 vs. Basal Media control. N=7. Mean±S.D.

FIG. 34A and FIG. 34B: Effect of absolute versus relative concentration of micronutrients on Type II collagen driven expression of Gaussia Luciferase. Aggregates of COL2A1-GLuc primary rabbit chondrocytes were cultured with predicted DoE conditions (1×) or the same ratios of these conditions at 1/15th the optimal predicted concentration (1/15×) over 22 days. Luminescence results are shown for day 10 (FIG. 34A) and day 17 (FIG. 34B). Ø indicates p<0.05 vs. Basal Media control. N=5 for DoE at 1/15× and N=6 for DoE at 1×. Individual replicates and the mean±S.D. are shown.

FIG. 35A and FIG. 35B: Biochamber for the generation of tissue engineered cartilage sheets. Custom 3D printed ABS biochambers were designed to generate tissue engineered articular cartilage sheets. FIG. 35A Photo of printed biochamber and Nalgene container fitted with sterile filter top. FIG. 35B Model of the biochamber assembly.

FIGS. 36A-36J: Tissue engineered cartilage sheet response to supplementation with condition 25. COL2A1-GLuc primary rabbit chondrocytes were cultured in bioreactors with condition 25 (1×) or condition 25 at 1/15th the concentration (1/15×) over 22 days. FIG. 36A On day 22, engineered sheets were collected. FIG. 36B Luminescence signal over 22 days in culture. Max tensile force (FIG. 36C) and tensile modulus (FIG. 36D) are shown. DNA (FIG. 36E), glycosaminoglycan (FIG. 36F) and collagen content (FIG. 36G) of the tissue were assessed. Data is also calculated as GAG/DNA (FIG. 36H), collagen/DNA (FIG. 36I) and GAG/collagen (FIG. 36J).

FIGS. 37A-37E: Analysis of Type II collagen and heteropolymer formation with Type IX collagen in tissue engineered cartilage. FIG. 37A Engineered cartilage was analyzed for type II collagen. Scale Bars, 300 μm. FIG. 37B Coomassie blue-stained SDS-PAGE gel of pepsin solubilized collagen showing β1(II), α1(XI), α2(XI) and α1(II) chains. Equivalent dry weight (25 μg) was loaded. FIG. 37C Western blot of samples equivalent to those in (FIG. 37B) and probed with anti-type II collagen antibody (1C10). FIG. 37D Western blot of samples equivalent to those electrophoresed in (FIG. 37B) (above) and probed with mAb 10F2. This antibody specifically recognizes the C-telopeptide domain of type II collagen when it is cross-linked to another α1(II) collagen chain. FIG. 37E Western blot of samples identical to those in (FIG. 37B) probed with antibody 5890. This antibody specifically recognizes N-telopeptide domain of α1(XI) collagen when cross-linked to chains of α1(II) and β1(II). X denotes crosslinks.

FIG. 38A and FIG. 38B. Primary COL2A1-GLuc rabbit chondrocytes were seeded in aggregate culture in basal chondrogenic media supplemented with different concentrations of a single vitamin or mineral (FIG. 38A) or combinations (FIG. 38B). Media was assessed for luminescence and results are shown for day 21. Individual values for 4 replicates are shown with green dashed line indicating basal media mean. Error bars (FIG. 38B) indicate standard deviation and *** indicate p<0.001 vs. basal media control.

FIG. 39. COL2A1-GLuc primary rabbit chondrocytes were cultured in custom in house bioreactors. At day 22, biopsy punches of engineered sheet were assessed via compression testing and young's modulus calculated.

FIG. 40. Experimental set up for chondrocyte mobility assessment. Experimental groups had 3 sections 3D bioprinted (2 mm×6 mm each). The chemoattractant was added into the 2:1 GelMA:HAMA ratio, while the cells were mixed into either 1:1 or 2:1. The spacer material ratio was the same as the material ratio used for the cell section. The directional mobility control had no chemoattractant (the extra spacer section represents this). For the 2D positive control, cells were seeded directly onto the culture dish, with the other 2 sections 3D bioprinted.

FIGS. 41A-41K. Stimulation of HuCol2gLuc chondrocytes by TGFβ1. Primary HuCol2gLuc chondrocytes were grown in aggregate culture with TGFβ1 (0-100 ng/ml). A semi-log plot of TGFβ1 dose vs. luminescence data from day 8 (FIG. 41A) and day 17 (FIG. 41B) are shown. Respective EC50 were determined for each day. Cell aggregates were assessed for GAG (FIG. 41C) and hydroxyproline content (FIG. 41D). Symbols show the mean of 6 replicates±S.D. (FIG. 41E) Gene expression analysis correlated COL2A1 vs gLuc, 95% confidence bands indicated by dashed lines. Histology of cell aggregate sections with Safranin-O (FIG. 41F-41H) staining or type II collagen (FIG. 41I-41K) immunohistochemistry for TGFβ1 dose 1.5625 ng/ml (FIG. 41F, 41I), 6.25 ng/ml (FIG. 41G, 41J) and 50 ng/ml (FIG. 41H, 41K). Magnification at 20× and scale bars, 200 μm.

FIGS. 42A-42B. Storage moduli increases as GelMA content and crosslinking time increase. Day 0 storage moduli of DoE generated GelMA:HAMA mixtures. 3D surface plot (FIG. 42A) of storage moduli vs crosslinking and material mixtures. Corresponding normal probability plot of the residuals (FIG. 42B).

FIGS. 43A-43D. Biomaterial composition and storage moduli impact type II collagen production. Day 8 (FIGS. 43A, 43C) and day 22 (FIG. 43B) luminescence for GelMA:HAMA mixtures at different crosslinking times. 3D surface plot (FIG. 43C) of day 8 luminescence vs crosslinking and biomaterial mixtures and the corresponding normal probability plot of the residuals (FIG. 43D). *p<0.05, **p<0.005, ***p=0.0001, or ****p<0.0001.

FIG. 44A and FIG. 44B. Type II collagen expression increases in biomaterials as compared to cell aggregates and storage modulus threshold for chondrogenesis. HuCol2gLuc luminescence over 3-weeks after 60s crosslinking compared to a cell aggregate control (FIG. 44A). Statistical significance, p<0.05, denoted by A (1:1 vs cell aggregate control), B (3:1 vs cell aggregate control) or C (1:0 vs cell aggregate control). (FIG. 44B) Non-linear fit curve of day 0 storage moduli vs. cumulative luminescence (R2=0.7328).

FIGS. 45A-45C. Primary human chondrocytes show high viability in 3D bioprinted constructs. 3D bioprinted constructs vs. pipetted controls on days 0, 1 and 7 (FIG. 45A). Comparison of 3D bioprinted GelMA:HAMA (1:1, 2:1 and 3:1) constructs over days 0, 1 and 7 (FIG. 45B). Viability staining of 3D bioprinted constructs on days 0 and 7 (FIG. 45C, 4× magnification 1 mm scale bar). *p<0.05.

FIGS. 46A-46F. Human chondrocytes are less mobile in stiffer biomaterial. Number of mobile cells (FIG. 46A), average distance (FIG. 46B) and average velocity (FIG. 46C) in the 2D positive control, and in 3D in either 1:1 or 2:1 GelMA:HAMA. Directionality of mobile cells towards basic-FGF chemoattractant (FIG. 46D). Microscopy images of HuChon-GFP cells in culture (FIG. 46E, 100 μm scale bar) and encapsulated in 1:1 GelMA:HAMA (FIG. 46F, 4× magnification 100 μm scale bar). *p<0.05.

FIGS. 47A-47B. Luminescence is greater in GelMA:HAMA 2:1 bioprinted constructs. Temporal luminescence of 3D bioprinted constructs over 3-weeks normalized to day 1 (FIG. 47A), day 8 (FIG. 47B) and day 22 comparisons. *p<0.05, **p<0.005.

FIGS. 48A-48F. ECM deposition is greater in GelMA:HAMA 2:1 bioprinted constructs. Quantification of DNA (FIG. 48A), GAG (FIG. 48B), GAG/DNA (FIG. 48C), and normalized hydroxyproline (FIG. 48D) of 3D bioprinted constructs from day 0 and day 22. Safranin-O (FIG. 48E) staining and type II collagen immunohistochemistry (FIG. 48F) of day 0 and day 22 bioprinted constructs at 40× magnification (scale bar shows 200 μm). *p<0.05, **p<0.005.

FIGS. 49A-49C. Storage moduli decrease from day 0 to day 22. Initial storage moduli (day 0) as compared to final storage moduli (day 22) (FIG. 49A). Storage moduli on day 0 increased with increased frequency, but on day 22 remained constant. *p<0.05, **p<0.005.

FIGS. 50A-50E. HuCol2gLuc reporter cells dose response to TGFβ1. Semi-log dose response curves for TGFβ1 dose vs. luminescence from day 8 (FIG. 50A) and day 22 (FIG. 50B), for reporter cells generated for donor used in all experiments. EC50 values were determined for each day. Cell aggregates DNA quantification (FIG. 50C), GAG/DNA (FIG. 50D) and hydroxyproline/DNA (FIG. 50E) for chondrocytes from donor shown in FIG. 41.

FIGS. 51A-51D. Tan delta increases as HAMA content increases and crosslinking time decreases. 3D surface plots for loss moduli results (FIG. 51A) and tan delta (FIG. 51C) for Day O and the corresponding normal probability plot of residuals (FIG. 51B and FIG. 51D).

FIGS. 52A-52B. HuCol2gluc luminescence over 3-week culture. GelMA:HAMA combinations after 15s (FIG. 51A) or 38s (FIG. 51B) crosslinking, including a cell aggregate control.

FIGS. 53A-53C. Normalized HuCol2gLuc luminescence over 3-week culture. GelMA:HAMA combinations with luminescence normalized to day 1 after 15s (FIG. 53A), 38s (FIG. 53B) or 60s (FIG. 53C) crosslinking, including a cell aggregate control.

FIG. 54. HuCol2gLuc luminescence over 3-weeks for 3D bioprinted GelMA:HAMA. Raw data are shown for 3D bioprinted chondrocytes printed in Gel MA:HAMA with ratios 2:1 and 3:1.

FIGS. 55A-55F. Dynamic mechanical analysis of 3D bioprinted GelMa:HAMA. Complex modulus (FIG. 55A, FIG. 55B), Loss modulus (FIG. 55C, FIG. 55D) and Tan Delta (FIG. 55E, FIG. 55F) are shown for GelMA:HAMA 3D bioprints with ratios 2:1 (FIG. 55A, 55C, 55E) and 3:1 (FIGS. 55B, 55D, 55F) on day 0 and day 22.

FIG. 56. Plasmid map for PRG4gLuc Lubricin promoter-driven Gaussia luciferase (Gluc) plasmid map (9,394 Bp, Genecopoeia). Contains puromycin (Puro) and ampicillin (Amp) selection cassettes.

FIGS. 57A-57F. HuPRG4gLuc characterization Primary human HuPRG4gLuc chondrocytes were grown in aggregate culture with TGFβ1 (0-40 ng/mL). FIG. 57A) Luminescence dose response curves for days 10 and 22. FIG. 57B) Secreted lubricin concentration quantified by ELISA. FIG. 57C) Relative gLuc gene expression correlates with relative PRG4 gene expression, with 95% confidence bands shown. FIG. 57D) DNA (μg/sample) and FIG. 57E) GAG/DNA (μg/μg) dose response curves. FIG. 57F) HDP/DNA (μg/μg) at all concentrations of TGFβ1. n=4-6, +/−SD.

FIG. 58. Histological analysis of cartilage aggregate response to TGFβ1 End of culture (day 22) histology staining of HuPRG4gLuc chondrocytes grown in aggregate culture with TGFβ1. Columns are 1, 10 and 20 ng/mL doses of TGFβ1. The top row is Safranin-O (red) staining for GAG content. The middle row is immunohistological staining (purple) for lubricin and the bottom row is for type II collagen. Scale bar shows 100 μm.

FIG. 59. Biodegradable and photocrosslinkable OMA preparation and characterization. Sodium alginate was oxidized with sodium periodate in aqueous solution, and then methacrylates were introduced onto the oxidized alginate. ¹H-NMR spectrum is shown with the top being the new proton on carbons 1 formed (a′), and the bottom for methacryl groups (b and c).

FIGS. 60A-60D. DoE identification of factors impacting lubricin expression. Design of experiment generated combinations of GelMA and OMA at different crosslinking times were mixed with HuPRG4gLuc cells and luminescence assessed over 22 days. FIG. 60A) Groups with 0% GelMA, FIG. 60B) 6% GelMA, and FIG. 60C) 12% GelMA. FIG. 60D) Corresponding normal probability plot of the residuals.

FIGS. 61A-61E. Validation of bioink combinations. FIG. 61A) Biomaterial groups and cell aggregate control luminescence over 22 days. FIG. 61B) 12% GelMA, 2% OMA luminescence with changing crosslinking time. FIG. 61C) 12% GelMA with either 2% or 4% OMA after either 15s or 38s crosslinking. FIG. 61D) 14% GelMA/2% OMA compared to 12% GelMA/2% OMA after 15s or 38s crosslinking. FIG. 61E) 14% GelMA alone compared to 14% GelMA/2% OMA after 15s or 38s crosslinking. **p value<0.0001, n=6+/−SD. (% GelMA: % OMA_crosslinking time(s)).

FIG. 62. Cell viability of 3D bioprinted groups Bioprinted 14% GelMA, 14% GelMA/2% OMA, and 16% GelMA on days 0, 1 and 7. *p value<0.05, **p value<0.001, n=3+/−SD.

FIGS. 63A-63B. Bioprinting assessment of optimal bioink. Luminescence for 3D bioprinted groups 14% GelMA, 14% GelMA/2% OMA, and 16% GelMA. FIG. 63A) Luminescence over 22 days in culture, 16% GelMA significance compared to the other groups (*p value<0.001). FIG. 63B) Day 10 and day 22 luminescence. ****p value<0.0001, ***p value=0.0001, **p value<0.005. n=7-9+/−SD.

FIGS. 64A-64E. Biochemical and immunohistological characterization of 3D bioprinted constructs. FIG. 64A) DNA content (μg/sample), FIG. 64B) GAG content (μg/sample) and FIG. 64C) GAG/DNA (μg/μg). FIG. 64D) Secreted lubricin concentration (pg/mL) quantified by ELISA on days 1, 10 and 22. FIG. 64E) Immunohistological staining of (top row) lubricin and (bottom row) type II collagen. 20× magnification with 100 μm scale bar. ****p value<0.0001, ***p value=0.0001, **p value<0.005, and *p value<0.05. n=3-9+/−SD.

FIGS. 65A-65C. Mechanical characterization of 3D bioprinted groups. FIG. 65A) Storage modulus determined by DMA. FIG. 65B) Kinetic coefficient of friction and FIG. 65C) static coefficient of friction determined by lap-shear testing. **p value<0.005, and *p value<0.05. ND=not determined. n=3, +/−SD.

FIGS. 66A-66D. Shape fidelity and degradation. FIG. 66A) Day 22 images of 14% GelMA, 14% GelMA/2% OMA and 16% GelMA. 4× images with 2 mm scale bar. FIG. 66B) Surface area of day 22 constructs. FIG. 66C) Swelling ratio over 22 days and FIG. 66D) Mass loss percentage over 22 days. **p value<0.005, and *p value<0.05. n=3, +/−SD.

FIG. 67A and FIG. 67B. DNA and GAG/DNA for validation data to support moving forward with these groups.

FIG. 68. Viability print vs. pipette.

FIG. 69 shows cumulative luminescence.

FIG. 70 shows value of loss modulus, tan delta, and complex modulus.

FIG. 71 shows the loss modulus, tan delta, and complex modulus.

FIG. 72. Zonal Organization of articular cartilage.

FIG. 73. Aggregate culture and analysis.

FIG. 74A and FIG. 74B. Identification of lubricin stimulating natural products in a human chondrocyte 3D aggregate screen. Primary human chondrocytes expressing the lubricin promoter-driven Gaussia luciferase were used in a 3D aggregate temporal assay to identify compounds stimulating lubricin expression (FIG. 74A). While most compounds had a neutral or negative affect on lubricin, volcano plot analysis identified several which increased lubricin production at day 10 (FIG. 74B).

FIG. 75: Distribution of compounds between stimulators, inhibitors and no effect. Compounds with luminescence values≥DMSO control mean+S.D. were categorized as stimulatory, those with values≤DMSO control mean−S.D. were categorized as inhibitors and those with values within 1 S.D. of the DMSO control mean were categorized as no effect (Days 10 and 22 shown). Of the compounds within 1 S.D. of the resazurin (metabolic) activity, 9 showed stimulation, 51 inhibition and 81 no effect.

FIG. 76A and FIG. 76B. Metabolic activity and aggregate size analysis. Lubricin stimulating compounds (188, 217, 270) decreased the aggregate metabolic activity as measured by resazurin assay (FIG. 76A). Aggregates were significantly larger in the natural product stimulated aggregates, but while there was a significant increase in the DMSO control aggregate size between day 10 and 22, there was a significant decrease in the natural product treated aggregates (FIG. 76B).

FIG. 77: Predicted targets of hit compounds. www.swisstargetprediction.ch was queried with the chemical structures of 188, 217 and 270. Compound 270 had no known analogues nor predicted targets. Compounds 188 and 217 are shown above.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10″ as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. 98. Administration” to a subject or “administering” includes any route of introducing or delivering to a subject an agent. Administration can be carried out by any suitable route, including intravenous, intraperitoneal, and the like. Administration includes self-administration and the administration by another.

The term “agonist” refers to a composition that binds to a receptor and activates the receptor to produce a biological response. The terms also encompass pharmaceutically acceptable, pharmacologically active derivatives of agonists specifically mentioned herein, including, but not limited to, salts, esters, amides, proagents, active metabolites, isomers, fragments, analogs, and the like. When the term “agonist” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “biocompatible” generally refers to a material and any metabolites or degradation products thereof that are generally non-toxic to the recipient and do not cause significant adverse effects to the subject.

Contacting: Placement in direct physical association, for example solid, liquid or gaseous forms. Contacting includes, for example, direct physical association of fully- and partially-solvated molecules.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

A “pharmaceutically acceptable salt” is a derivative of the disclosed compound in which the parent compound is modified by making inorganic and organic, pharmaceutically acceptable, acid or base addition salts thereof. The salts of the present compound can be synthesized from a parent compound that contains a basic or acidic moiety by conventional chemical methods. Generally, such salts can be prepared by reacting free acid forms of the compound with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compound further include solvates of the compound and of the compound salt. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term as used herein “engineered” and other grammatical forms thereof may refer to one or more changes of nucleic acids, such as nucleic acids within the genome of an organism. The term “engineered” may refer to a change, addition and/or deletion of a gene. Engineered cells can also refer to cells that contain added, deleted, and/or changed genes.

The term “genetically engineered cell” as used herein refers to a cell modified by means of genetic engineering. In some embodiments, the cell is a chondrocyte.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “promoter” as used herein is defined as a DNA sequence recognized by the synthetic machinery of the cell, or introduced synthetic machinery, required to initiate the specific transcription of a polynucleotide sequence.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions and Methods

In some aspects, disclosed herein is a three-dimensional culture system for producing cartilage, said culture system comprising a chondrocyte that is genetically engineered to express a reporter protein; wherein the expression of a reporter protein is driven by a promoter of an extracellular matrix protein coding gene. In some embodiments, the promoter a type 2 collagen promoter. In some embodiments, the promoter is a promoter of COL2A1, ACAN, or PRG4. Accordingly, in some aspect, disclosed herein is a three-dimensional culture system for producing cartilage, said culture system comprising a chondrocyte that is genetically engineered to express a reporter protein; wherein the expression of a reporter protein is driven by a type 2 collagen promoter and or a PRG4 promoter.

In some embodiments, wherein the culture system is at about 2% to 8% (e.g., about 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, or 8%) oxygen. In some embodiments, wherein the culture system is at about 1% to 10%, about 2% to 9%, about 3% to 8%, about 4% to 6%, about 2% to 7%, about 2.5% to 6.5%, about 3% to 6%, about 3.5% to 5.5%, or about 4.5% to 5.5% oxygen. In some embodiments, the culture system is at about 5% oxygen. In some embodiments, the reporter protein is a bioluminescent protein (for examples, Gaussia luciferase or mCherry protein). In some embodiments, the three-dimensional culture system further comprises IL-1β and/or TGF-β.

The three-dimensional culture system disclosed herein can further comprise a bioprinting material selected from gelatin methacrylate (GelMA), hyaluronic acid methacrylate (HAMA), and oxidized methacrylated alginate (OMA). In some embodiments, the implant comprises GelMA and HAMA or GelMA and oxidized methacrylated alginate (OMA). In some embodiments, the ratio of GelMA (e.g., concentration of 15% v/v) and HAMA (e.g., concentration of 2% v/v) is about 3:1, 2.5:1, 2:1, 1.5:1, 1:1, 1:1.5, 1:2, 1:2.5, or 1:3.

Also disclosed herein is a method of generating cartilage comprising using the three-dimensional culture system disclosed herein.

In some aspects, disclosed herein is a three-dimensional culture system for selecting an agent (e.g., a therapeutic agent, a natural product, a mineral, or a biomaterial) for promoting cartilage regeneration, said culture system comprising a chondrocyte that is genetically engineered to express a reporter protein; wherein the expression of a reporter protein is driven by a promoter of an extracellular matrix protein coding gene. In some embodiments, the promoter a type 2 collagen promoter. In some embodiments, the promoter is a promoter of COL2A1, ACAN, or PRG4. Accordingly, in some aspect, disclosed herein is a three-dimensional culture system for producing cartilage, said culture system comprising a chondrocyte that is genetically engineered to express a reporter protein; wherein the expression of a reporter protein is driven by a type 2 collagen promoter. In some aspect, disclosed herein is a three-dimensional culture system for producing cartilage, said culture system comprising a chondrocyte that is genetically engineered to express a reporter protein; wherein the expression of a reporter protein is driven by a PRG4 promoter. In some embodiments, wherein the culture system is at about 2% to 8% (e.g., about 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, or 8%) oxygen. In some embodiments, wherein the culture system is at about 1% to 10%, about 2% to 9%, about 3% to 8%, about 4% to 6%, about 2% to 7%, about 2.5% to 6.5%, about 3% to 6%, about 3.5% to 5.5%, or about 4.5% to 5.5% oxygen. In some embodiments, the culture system is at about 5% oxygen. In some embodiments, the reporter protein is a bioluminescent protein (for examples, Gaussia luciferase or mCherry protein).

Also disclosed herein is a method of selecting an agent (e.g., a therapeutic agent, a natural product, a mineral, or a biomaterial) for promoting cartilage regeneration, comprising
a. contacting the chondrocyte of a three-dimensional culture system with the agent (e.g., a therapeutic agent, a natural product, a mineral, or a biomaterial), wherein said system comprising a chondrocyte that is genetically engineered to express a reporter protein; wherein the expression of a reporter protein is driven by a promoter of an extracellular matrix protein coding gene;
b. obtaining a sample of the culture medium; and
c. determining a level of the reporter protein in the sample of step b;
   wherein increased level (for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level) of the reporter protein in the sample relative to a reference control indicates that the agent (e.g., a therapeutic agent, a natural product, a mineral, or a biomaterial) promotes cartilage regeneration.

In some embodiments, the extracellular matrix protein coding gene comprise COL2A1, ACAN, and/or PRG4. In some embodiments, the expression of a reporter protein is driven by a type 2 collagen promoter. Accordingly, in some embodiments, the promoter is a promoter of type 2 collagen coding gene and/or lubricin coding gene. In some embodiments, wherein the culture system is at about 2% to 8% (e.g., about 2%, 2.5%, 3%, 3.5%, 4%, 4.5%, 5%, 5.5%, 6%, 6.5%, 7%, 7.5% or 8%) oxygen. In some embodiments, the culture system is at about 5% oxygen. In some embodiments, the reporter protein is a bioluminescent protein (for examples, Gaussia luciferase or mCherry protein).

In some embodiments, the culture system further comprises IL-1β and/or TGF-β. In some embodiments, step a) further comprises contacting the chondrocyte with IL-1β and/or TGF-β from day 5 or later (e.g., day 5, 6, 7, 8, 9, 10, 11, 12, 13, 14, 15, 16, 18, 20, 22, 24, 26, 28, 30, or later) after contacting the chondrocyte with the agent (e.g., a therapeutic agent, a natural product, a mineral, or a biomaterial). In some embodiments, the agent is a therapeutic agent.

In some embodiments, the therapeutic agent promoting cartilage regeneration increases an expression level of a dopamine receptor in the chondrocyte (for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level). In some embodiments, the dopamine receptor comprises dopamine receptor type 4.

“Dopamine receptor type 4” or “DR4” refers herein to a polypeptide that, in humans, is encoded by the DRD4 gene. In some embodiments, the dopamine receptor type 4 polypeptide is that identified in one or more publicly available databases as follows: HGNC: 3025, NCBI Entrez Gene: 1815, Ensembl: ENSG00000069696, OMIM®: 126452, UniProtKB/Swiss-Prot: P21917.

In some embodiments, the sample of the culture medium is obtained on day 5 or later after (e.g., day 5, 6, 7, 8, 9, 10, 11, 12, 13, 14, 15, 16, 18, 20, 22, 24, 26, 28, 30, or later) contacting the chondrocyte with the therapeutic agent. In some embodiments, the sample of the culture medium is obtained on day 15 or later after contacting the chondrocyte with the therapeutic agent. In some embodiments, the sample of the culture medium is obtained on day 20 or later after contacting the chondrocyte with the therapeutic agent.

Also disclosed herein is a method of treating a disorder of cartilage in a subject in need. In some examples, the method comprises administering to the subject a therapeutically effective amount of a therapeutic agent selected by using the three-dimensional culture system disclosed herein.

Also disclosed herein is a method of treating a disorder of cartilage in a subject in need. In some examples, the method comprises administering to the subject a therapeutically effective amount of a therapeutic agent, wherein the therapeutic agent comprises a dopamine receptor agonist or a dopamine receptor antagonist. In some embodiments, the therapeutic agent comprises a dopamine receptor agonist. In some embodiments, the therapeutic agent increases an expression level of a dopamine receptor. In some embodiments, the dopamine receptor comprises dopamine receptor type 4.

In some embodiments, the disorder is osteoarthritis or chronic arthritis.

In some embodiments, the therapeutic agent comprises 6-Hydroxy-2-methoxyaporphine, (-)-Apoglaziovine, Pentoxifylline, 1,10-Dihydroxy-2-methoxyaporphine, Deserpidine, Vincristine sulfate, Promoline, Calcium folinate, Fastigilin B; Parthenicin; or 5-(6-Aminopurin-9-yl)-3-(hydroxymethyl)cyclopent-3-ene-1,2-diol, or a derivative thereof.

Also disclosed herein is a three-dimensional bioprinting implant comprising one or more biomaterials and/or cells, wherein the biomaterials are selected from gelatin methacrylate (GelMA), hyaluronic acid methacrylate (HAMA), and oxidized methacrylated alginate (OMA). In some embodiments, the implant comprises GelMA and HAMA. In some embodiments, the ratio of GelMA (e.g., concentration of 15% v/v) and HAMA (e.g., concentration of 2% v/v) is about 3:1, 2.5:1, 2:1, 1.5:1, 1:1, 1:1.5, 1:2, 1:2.5, or 1:3. In some embodiments, the implant comprises GelMA and oxidized methacrylated alginate (OMA). The three-dimensional bioprinting implant can be used for creating cartilage tissue.

Accordingly, in some aspects, disclosed herein is a method for providing an bioprinted organ to a subject in need thereof, comprising:
a) obtaining a three-dimensional bioprinted organ comprising one or more biomaterials and/or cells, wherein the biomaterials are selected from gelatin methacrylate (GelMA), hyaluronic acid methacrylate (HAMA), and oxidized methacrylated alginate (OMA); and
b) implanting the 3D bioprinted organ into the subject.

The method and/or the three-dimensional bioprinting implant can be used to treat a disorder of cartilage, such as an articular cartilage defect. In some embodiments, the disorder is osteoarthritis or chronic arthritis.

### Examples

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention is not be limited to the particular implementations disclosed herein, but that the invention will include all implementations falling within the scope of the appended claims.

### Example 1: Novel Screening Platform to Identify Anabolic Treatments for Osteoarthritis

Osteoarthritis is a major healthcare burden both in terms of financial and quality of life costs amounting to over 10% of the US healthcare burden. There are currently no curative therapies for osteoarthritis. Drugs treat pain and inflammation before a total joint replacement is required. Further, adult cartilage has no innate repair mechanism. Part of the challenge is that current osteoarthritis drug discovery methods are severely lacking. To overcome these barriers, I have developed in vitro (in a dish) models for cartilage using cells that contain a bioluminescent (light emitting) reporter tied to the crucial cartilage protein, type II collagen. This project uses those reporter cells to screen for drugs that stimulate type II collagen, and therefore promote regeneration of cartilage. Candidate drugs are then analyzed for their mechanism of action and that mechanism investigated as a way to treat osteoarthritis.

Human chondrocytes, the cells that make cartilage, were genetically modified to secrete a bioluminescent protein when production of type II collagen is stimulated. This bioluminescent protein is secreted into the media the cells are fed with; the media can then be sampled, and it glows when the substrate is added. The intensity of the light emitted is proportional to the amount of type II collagen in the media, the more type II collagen produced, the brighter the light. Cells in the body exist in a 3-dimensional environment while cells in culture are typically grown in a 2-dimensional format. As such, previous 2D studies have resulted in identification of potential compounds with activity in vitro that don't translate to activity at the in vivo (organism) level. Three-dimensional culture methods are better predictors of in vivo activity, and the work herein uses a 3D cartilage culture model. Another innovation that approximates the physiological environment is the use of 5% oxygen in culture. Cartilage has no blood supply and therefore has a physiological oxygen tension of between 2 and 8%; however, most research uses atmospheric oxygen (20%) in cell culture. Engineered cartilage is significantly improved by the use of physiological (5%) oxygen. This model is combined with an automated pipetting robot to enable screening of hundreds of drugs on cartilage spheroids arranged in wells on a single plate.

A natural product library (390 compounds) was screened; drug like molecules that have been isolated from plants, marine invertebrates and micro-organisms. Natural products and their derivatives are a common source of drugs and drug like molecules benefitting from the relative similarity between all life and forming an excellent starting point in discovery. 5 active molecules were found that stimulate type II collagen expression. Interestingly, each of these compounds can target the dopamine receptor. Published whole transcriptome (gene) data demonstrates that the dopamine receptor expression increases during chondrogenesis, and it is understudied as a target in osteoarthritis. These in vitro assessments are followed by a non-invasive knee injury rat model to demonstrate efficacy in vivo.

Active compound screens are continued with several other drug libraries using thisin vitro assay and testing hits with injury simulation. To enable higher throughput with fewer cells and reagents, a 96-well plate up to a 384-well plate can be used, requiring fewer cells and reagents.

The overall goal of this study is to identify drugs that can promote cartilage regeneration. Osteoarthritis is a leading cause of disease and disability worldwide with significant socio-economic costs amounting to over 10% of the US healthcare burden. The causes and etiology of osteoarthritis are widely debated. There are no cures for osteoarthritis; this is partly due to sub-optimal screening platforms and the fact that human adult cartilage has no regenerative capacity in vivo. Repair in articular cartilage is severely limited due to low cell density and poor vascularization. It is significant that few new drugs have been approved for treatment, and no anabolic drugs have been identified, restricting therapy to non-steroidal anti-inflammatory pain management and corticosteroids. This study uses a novel, 3D-cell based phenotypic assay to identify lead compounds stimulating an anabolic chondrogenic response. Using this assay, several compounds were discovered from a natural product library (Natural Products Set V; NCI), all dopamine receptor ligands, as identified through in silico analysis. The study investigates those compounds, the dopamine receptor response and screen for new anabolic drugs from several libraries. To address the overall goal of cartilage regeneration, the following experiments are conducted:

Experiment 1—Investigate anabolic lead compounds for efficacy in chondrogenesis.

Experiment 1A determine overall chondrogenic capacity of identified natural products. Drugs 84, 186, 204, 413 and 418 all had stimulatory effects on type II collagen production in the screen of natural products (NCI Natural Products Set V library). These drugs are further investigated in vitro for their effect on chondrogenesis by assessing cartilage spheroid tissue through qPCR, histology and biochemical assays for fibroblastic, chondrogenic and osteogenic genes. Mechanistic insight is pursued with western blots for kinase activity.

Experiment 1B investigates the common target, dopamine receptor D4, to establish this mechanism. Known agonist (ABT 724) and antagonist (PNU 96415E) of the dopamine receptor D4 are tested in the 3D-cell based assay for efficacy in upregulating type II collagen. The natural products are antagonists, and these antagonist can yield the same induction of type II collagen. This validates this receptor as a target for stimulation of type II collagen. Similar assessments as in 1A are performed for chondrogenesis and mechanistic insights.

Experiment 1C determines drug efficacy in a non-invasive knee injury model for post-traumatic osteoarthritis. A non-invasive knee injury model, compressing the joint and causing an anterior cruciate ligament tear, is used with intra-articular administration of active compounds. Cartilage condition is assessed by μCT and histology. Gait analysis gives a longitudinal assessment of joint health.

Experiment 2—Identify new anabolic lead compounds.

Experiment 2A investigates increase capacity of the screening platform. The current 96-well plate assay is scaled up to a 384-well plate. Assessments of lower cell number per spheroid, luciferase output and reproducibility are made.

Experiment 2B investigates promoting cartilage regeneration in the presence of injury. In addition to the natural product library, several new libraries have been identified for screening: a diversity set (1584 compounds; NCI Diversity Set VI) and a mechanistic set (811 compounds; NCI Mechanistic Set VI). Libraries are screened in the 3D-cell culture model with/without IL1β to simulate injury to identify new lead compounds to test for regenerative effects on chondrocyte culture.

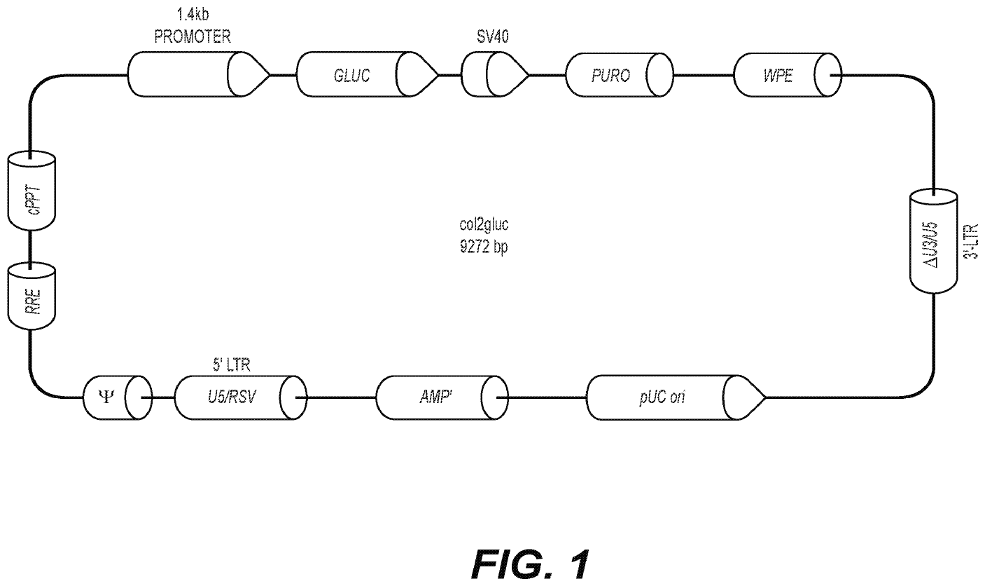

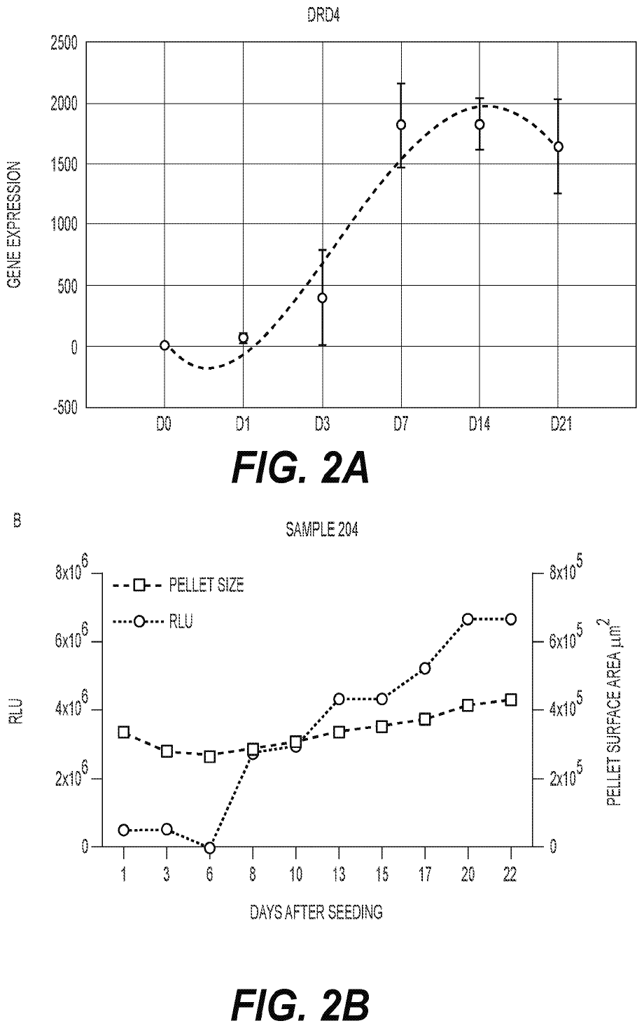

Background: Chondrocytes are the only cell in articular cartilage, responsible for the synthesis and maintenance of the dense extracellular matrix that makes up most of the tissue. The main component of that tissue is type II collagen, a protein that forms a networked mesh that gives cartilage compressive, tensile and shear strength. Degradation of cartilage and lack of a sufficient anabolic response results in osteoarthritis; a common occurrence after cartilage injury. Chondrocytes typically de-differentiate in culture to form fibroblast like cells that are unable to synthesize type II collagen. This obstacle is overcome by growing the cells on a devitalized synoviocyte derived extracellular matrix, achieving twice the population doublings without loss of chondrogenicity. This means that almost half a billion chondrogenic cells can be achieved from an initial isolate of half a million cells. Human chondrocytes were engineered with type II collagen promoter-driven Gaussia luciferase to create a primary human chondrocyte reporter cell for chondrogenesis (Col2gLuc; FIG. 1). This drug screening platform is used to screen a natural product library (NCI Natural Products Set V library) comprised of potential drug like molecules that have been isolated from plants, marine invertebrates and micro-organisms. This screen has identified several drugs which promoted type II collagen expression. A common target between these drug candidates is the dopamine receptor, a novel target in the treatment of osteoarthritis. Looking at receptor expression in chondrocytes and mesenchymal stromal cells differentiated into chondrocytes expression of the dopamine receptor type 4 was observed (DRD4; FIG. 2A). Interestingly, the relative induction of type II collagen coincided with the increase in expression of the dopamine receptor, further strengthening the support for it as a target for the drug (FIG. 2B).

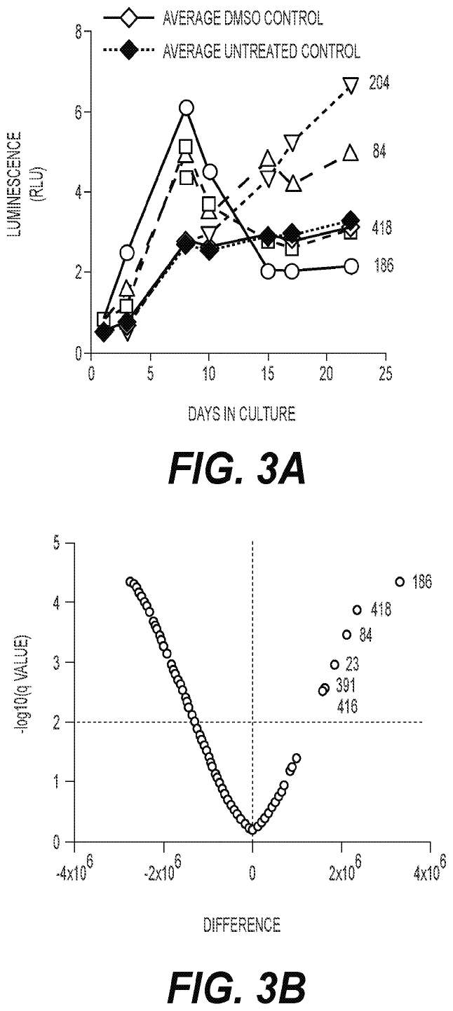

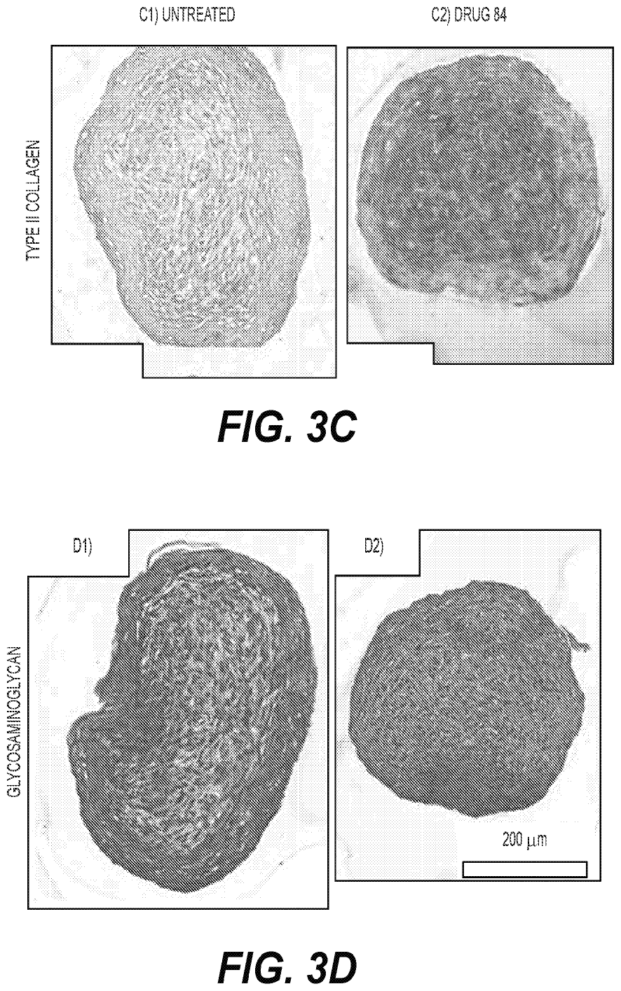

Experiment 1A—Determine overall chondrogenic capacity of identified natural products. Human chondrocytes cultured as 3D cartilage spheroids in non-adherent 96-well plates are treated with the candidate drugs and type II collagen promoter-driven Gaussia luciferase are assessed from the media over time as previously shown (FIGS. 2B and 3A). Concurrent assessment of spheroid size and morphology are performed (FIG. 2B). Spheroid size and morphology give an indication of extracellular matrix accumulation and correlate well with glycosaminoglycan content. At the end of the 22-day chondrogenic induction, cartilage spheroids are fixed, embedded, sectioned and stained for glycosaminoglycan content (safranin-O; FIG. 3D) and collagen (picrosirius red). Immunohistochemistry for collagen type I, II (FIG. 3C), type X is also be performed. Spheroids also undergo papain digestion and biochemical assessment for DNA, glycosaminoglycan and collagen content. Six additional spheroids are lysed and RNA extracted for qPCR of type II collagen, aggrecan, type I collagen, type X collagen, MMP13, SOX9 and lubricin. These analyses give an overall assessment of the nature of the tissue produced, whether fibroblastic (type I collagen), intermediate zone chondrogenic (type II collagen, SOX9 and aggrecan), surface zone chondrogenic (lubricin) or hypertrophic chondrogenesis (MMP13 and type X collagen). Induction of dopamine kinase activity is assessed using western blotting for AKT and phospho-AKT. There is a biphasic response of chondrocytes to chondrogenic induction (FIG. 3A). Some of the drugs were seen to stimulate type II collagen production during the early stages (drugs 186, 418, 84, 23, 291 and 416; FIGS. 3A, 3B). It is unknown if early stimulation can give optimal results. Temporal analyses where drug is added or removed at A) spheroid formation, B) day 6, C) day 13 can elucidate this aspect. Data with drug 84, which was stimulatory throughout the experiment, demonstrated an increase in type II collagen deposition vs. untreated control at the end of culture (FIG. 3C)

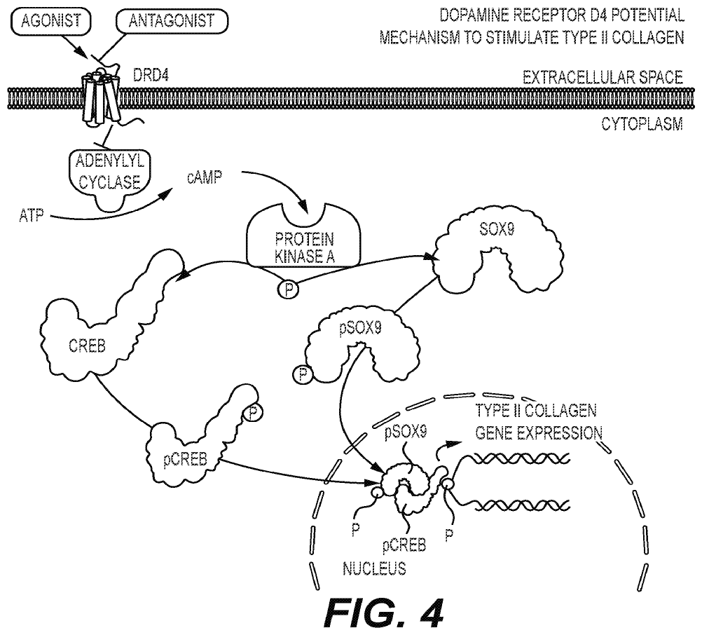

Experiment 1B—Investigate the common target, dopamine receptor D4, to establish this mechanism. Similar approaches to Experiment 1A are used where the known agonist (ABT724) and antagonist (PNU96415E) of the dopamine receptor D4 is tested in type II collagen reporter chondrocytes in the 3D cartilage spheroid model. The dopamine receptor D4 (DRD4) is an inhibitor of adenylyl cyclase (FIG. 4). The experiments herein test whether the natural products are acting as an antagonist at the DRD4 receptor. ABT724 can reduce chondrogenesis and PNU96415E can improve it. The temporal induction of type II collagen expression with the known agonist and antagonist are studied using the human Col2gLuc reporter cells. Combined incubations of the agonist or antagonist with the natural products can establish DRD4 binding as the mechanism of action. Luminescent reporter results in cartilage spheroids will be followed by Establishment of the dopamine receptor as a target for an anabolic response in cartilage opens new avenues for treatment of post-traumatic osteoarthritis, and chronic osteoarthritis. Investigation of post-traumatic osteoarthritis is the focus of Experiment 1C and a component of Experiment 2B.

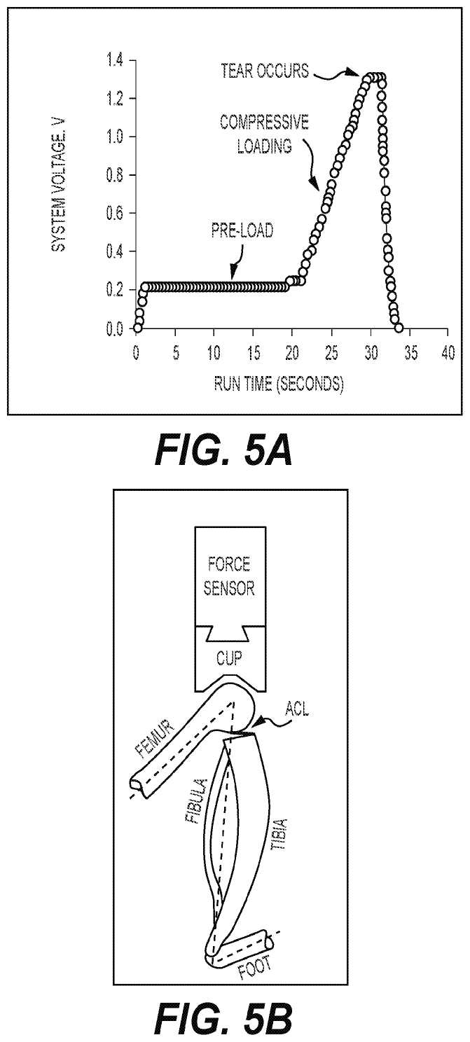

Experiment 1C—Drug efficacy in a non-invasive knee injury model for post-traumatic osteoarthritis. Drug(s) with the highest efficacy in Experiment 1A and/or Experiment 1B can be used in a non-invasive knee injury model. Mice are anaesthetized and restrained in a compression device that loads the joint until the anterior cruciate ligament fails (FIG. 5). This injury replicates common sports injuries well and reliably produces osteoarthritic joints. Animals are be treated with drug candidates with twice weekly intra-articular injections at doses based on the in vitro stimulatory doses established in Experiment 1A and 1B. The dose is based on several studies where an approximate scaling for FGF18 from in vitro efficacy (25-100 ng/ml) to in vivo rat knee efficacy (3-5 μg/knee) was found. Similarly, the small molecule SM04690 had an in vitro efficacy of 15-50 ng/ml and was scaled to 0.3 μg/rat knee in vivo. These studies result in an approximate scale up of 10-50 fold over in vitro levels. Gait assessment would be performed twice weekly for evaluation of limb loading (DigiGate). At 8-weeks post injury, when the majority of animals without treatment can be displaying osteoarthritic deterioration in the joint, animals are sacrificed and joints fixed for μCT imaging then histology.

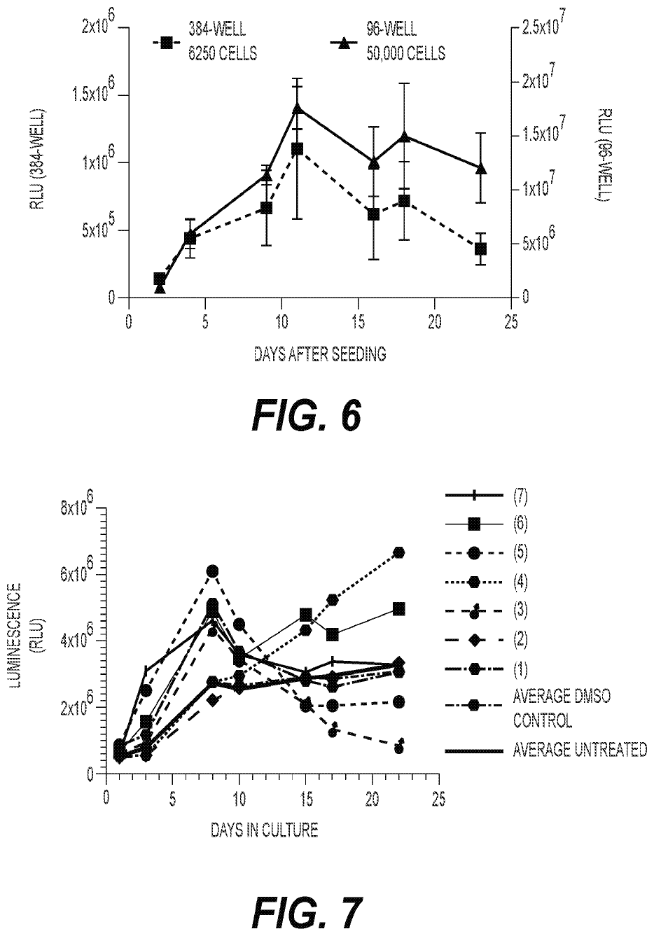

Experiment 2A—Increase capacity of the screening platform. The current 96-well plate assay has significantly improved throughput by both enabling a non-destructive temporal assay of chondrogenesis and utilizing a robotic pipetting device (Opentrons OT-2). The potential to improve output by scale up to a 384-well plate remains. Chondrogenic culture is scaled by both the volume of the well and the reduced chondrocyte number. This has previously been performed with a rabbit chondrogenic cell-based assay (FIG. 6), that approach is translated to the human cells. Further, reduction in cell and well volume reduces assay costs and drug amounts needed for testing.

Experiment 2B—Promote cartilage regeneration in the presence of injury. This novel technique for osteoarthritic drug screening can produce many more lead compounds. The National Cancer Institute has two more libraries that are available for screening, a diversity set which offers pharmacophores selected from almost 140,000 compounds to represent the most diverse pharmacologically active compounds. The mechanistic set represents a diverse array of compounds that have been tested and have mechanistic insights. Each of these libraries is screened, along with other potential libraries, in the phenotypic assay described in Experiment 1. Additionally, injury promotes catalysis of cartilage and tips the scale to catabolism vs. anabolism. In this study, libraries are also screened in the presence of IL1β. IL1 β simulates an injury response and, when cartilage spheroids have been established (day 15), this assay can determine an anabolic response due to the drugs. Temporal analysis of induction of type II collagen expression along with effects on spheroid morphology will be assessed. Lead compounds will be followed up in the same manner as outlined in Experiment 1A.

One or more of the natural products identified can yield significant improvements in overall chondrogenesis. In silico prediction and the correlative gene expression data results in identification of DRD4 as a target for regenerative medicine of the joint. Therefore, in vivo application of the drug(s) results in a significant improvement post-injury. If the DRD4 receptor is not the target, but significant improvement is found with the drug, conjugation approaches with affinity purification and mass spectroscopy is used to identify the target whilst proceeding with efficacy assessment in vivo. If none of the current drug candidates show robust induction of type II collagen while maintaining glycosaminoglycan production, screening of additional libraries (Section 2B) can discover lead candidates. The assay is miniaturized to a 384-well plate format and used to find new anabolic drugs from the other libraries both in the presence and absence of a simulated injury. If miniaturization of the assay is unsuccessful, continued use of the 96-well plate format is made with a resulting decrease in screening output.

Osteoarthritis is a major healthcare burden both in terms of financial and quality of life costs amounting to over 10% of the US healthcare burden. Osteoarthritis is characterized by the degeneration of articular cartilage. Adult cartilage has no innate repair mechanism, and there are currently no disease modifying therapies for osteoarthritis. Current drugs treat pain and inflammation before a total joint replacement is required. As such, there is a strong need for novel cartilage regenerative therapies. I have developed in vitro (in a dish) models for cartilage using cells that contain a bioluminescent reporter tied to the crucial cartilage protein, type II collagen. Human chondrocytes, the cells that make cartilage, were genetically modified to secrete a bioluminescent protein when production of type II collagen is stimulated. This bioluminescent protein is secreted into the media the cells are fed with, and we can then sample the media and assay by adding a substrate. The substrate and bioluminescent protein react to emit light. The intensity of the light emitted is related to the amount of protein in the media, the more type II collagen that is produced, the brighter the light. Cells in the body exist in a 3-dimensional environment, typically cells in culture are grown in a 2-dimensional format. These 2D studies have resulted in identification of potential compounds with activity in vitro that doesn't translate to activity at the in vivo (organism) level. Three-dimensional culture methods are better predictors of in vivo activity, the work shown herein uses a 3D cartilage culture model. On a similar theme, cartilage has no blood supply and therefore has a physiological oxygen tension of between 2 and 8%. Most researchers use atmospheric oxygen (20%) in their cell culture, the current study uses physiological (5%) oxygen for this tissue model culture and have shown that cartilage is significantly improved under these conditions. Altogether, this model, combined with an automated pipetting robot, enables screening of hundreds of drugs on a plate.

This study screened a natural product library (390 compounds), drug like molecules that have been isolated from plants, marine invertebrates and micro-organisms. Natural products and their derivatives are a common source of drugs and drug like molecules benefitting from the relative similarity between all life and form an excellent starting point in discovery. 5 active molecules were found to stimulate type II collagen expression. A common receptor-target for these drugs was found through in silico analysis. Whole transcriptome (gene) data show that the receptor is expressed during chondrogenesis and as of yet, is relatively understudied as a target in osteoarthritis research. These data show that these natural product drugs, and other known drugs targeting the receptor can be used to treat osteoarthritis.

The drugs from the natural product library that were found to stimulate type II collagen are: 1) 6-Hydroxy-2-methoxyaporphine; 2) (-)-Apoglaziovine; 3) Pentoxifylline; 4) 1,10-Dihydroxy-2-methoxyaporphine; 5) Deserpidine; 6) Vincristine sulfate; 7) Promoline; 8) Calcium folinate; 9) Fastigilin B; 10) Parthenicin; 11) 5-(6-Aminopurin-9-yl)-3-(hydroxymethyl)cyclopent-3-ene-1,2-diol. None of which have been used to treat osteoarthritis.

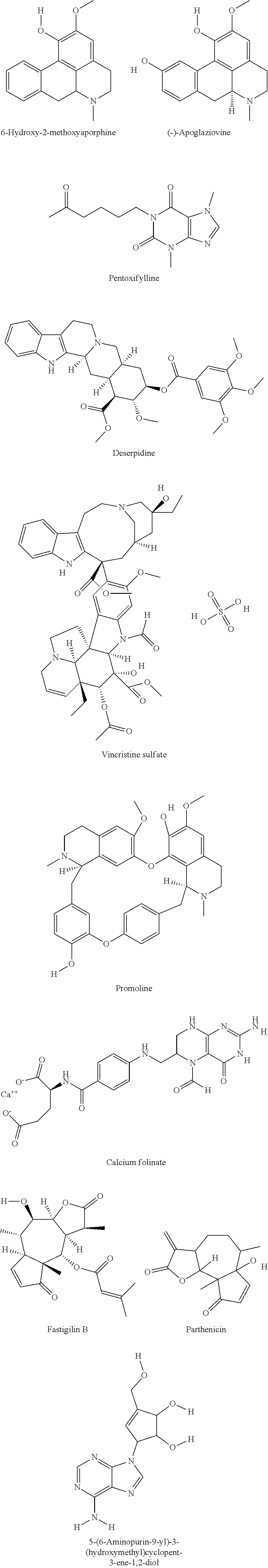

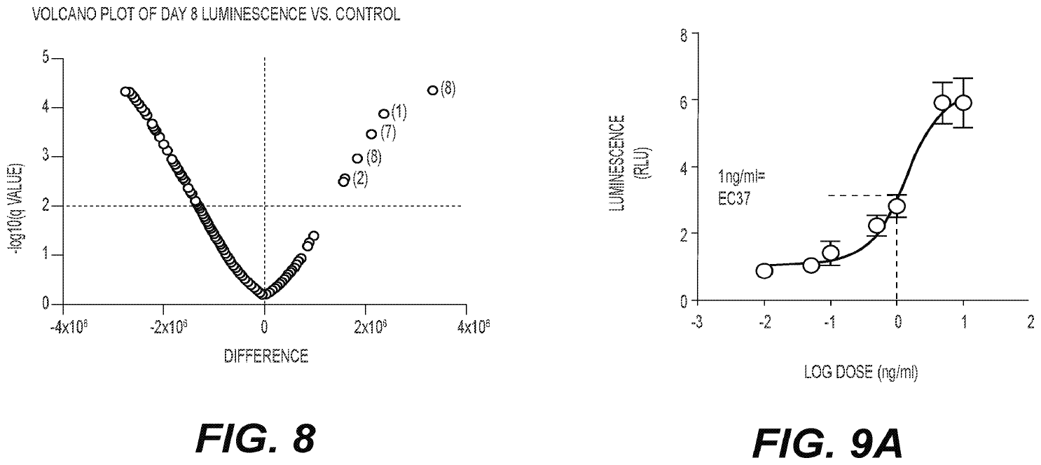

The novel target(s), identified through their commonality using swisstargetprediction.ch and confirmation through transcriptome data (Kean 2019 Cells; Huynh 2018 FASEB) are the Dopamine Receptor D4 (DRD4), Sigma Opioid Receptor (SIGMAR1) and Cholinergic Receptor Nicotinic Beta 1 Subunit (CHRNB1). These drugs and their derivatives and those targeting the receptors identified have the potential to stimulate cartilage regeneration in vitro and in vivo. FIG. 7 shows the temporal increase in type II collagen expression stimulated by each of the drug compounds named. FIG. 8 show the statistical comparison, indicating the drugs that are significantly higher at day 8. Drugs were administered throughout this 22 day chondrogenesis experiment and there is a biphasic process in the generation of extracellular matrix in cartilage.

Osteoarthritis is the most prevalent articular joint disease worldwide. It affects a significant percentage of the population, with socio-economic costs amounting to over 10% of the US healthcare burden. Cartilage degeneration is the primary pathological feature of osteoarthritis.

Cartilage is created by a single cell type, the chondrocyte, which secretes the extracellular matrix that makes up the tissue. After development chondrocytes continue to maintain cartilage tissue homeostasis, however, they are unable to repair tissue damage and thus cartilage has no regenerative capacity. Current therapies for osteoarthritis are primarily palliative and there is a strong need to identify novel therapeutics to promote regenerative chondrogenesis.

This project emerged from the development of this high-throughput in vitro 3D chondrogenic assay which uses a novel secreted reporter to assess chondrogenesis by measuring type II collagen production. This assay has several advantages over traditional cartilage research techniques. It uses 50,000 cells per sample, which allows for maximal use of primary cells. The assay is non-destructive and rapid which allows for temporal analysis, an advantage over traditional cartilage research approaches. In addition, it enables automated feedings and sample collection which allows for high-throughput studies and improves consistency. Findings show the success of this assay, by using it to identify vitamins and minerals that improve chondrogenesis in vitro.

This novel chondrogenic assay is used to identify new lead compounds that can stimulate chondrogenesis in primary human chondrocytes:

Screen several drug libraries to identify potential candidates for chondrogenesis. Preliminary studies have shown that the 3D chondrogenic assay can be used to screen 240 natural products from the Natural Product Set V library (NCI). From this screen 6 potential anabolic drug candidates have been identified. Validation of this study has confirmed that 3 of the 6 candidates improve chondrogenesis in human chondrocytes.

The library screened in the pilot study made use of natural products that were selected due to the presence of multiple functional groups in the compound structures. In this study, additional libraries are screened including the NCI Diversity set VI and the mechanistic set VI. After screening, any drug candidates are validated using cartilage sheets, biochemical assessments, and mechanical tests.

Identify new target receptors for chondrogenesis and characterize the signaling pathways involved. Using in silico analysis a target from the candidates identified have been identified from the drug screen. Similar analyses can be conducted with drug candidates from other screens to identify additional targets. Alternatively, RNA-Seq can be used to identify additional target receptors. This study confirms involvement of target receptors by inhibition/stimulation studies and carry out mechanistic studies to identify signaling pathways involved in improved chondrogenesis.

### Example 2. Targeting the Dopamine Receptor D4 for the Treatment of Osteoarthritis

The prevalence and cost of arthritis is astounding, with total arthritis-attributable medical expenditures and lost earnings surpassing $300 billion in 2013, accounting for more than 10% of healthcare costs. The lack of disease modifying drugs for osteoarthritis is a significant challenge and could be particularly beneficial in post-traumatic osteoarthritis. This project aims to address this challenge through identification and investigation of novel lead compounds using this innovative phenotypic reporter system.

Type II collagen is the quintessential marker of hyaline cartilage and is typically deficient in tissue engineered cartilage constructs. To address this collagen deficiency and the unknown effects of vitamins and minerals on chondrogenesis, we transduced a chondrocyte cell line (ATDC5) with a type II collagen promoter-driven secreted Gaussia luciferase. It was shown that a 2.5-fold increase over TGFβ1 alone was achieved with optimized media. This published work demonstrates the advantages of this phenotypic reporter system which is further established by the data shown herein. For this work, the Gaussia luciferase reporter system was inserted into human primary chondrocytes using a lentiviral vector.

Using this novel, phenotypic, 3D-cell based assay we screened a natural product library (Natural Products Set V; NCI). Data identified several lead compounds stimulating an anabolic chondrogenic response in primary human articular chondrocytes. Also, of significant interest, all lead compounds are identified, via in silico analysis, to target the same dopamine receptor D4. We will pursue those compounds, screen for new anabolic drugs from several libraries, assess the dopamine receptor D4 response and test efficacy in a non-invasive knee injury model.

### Experiment 1—Investigate Lead Compounds for Efficacy in Chondrogenesis

1A—Evaluate drugs that were identified and confirmed in the screen: Several natural products had stimulatory effects in the screen of compounds for type II collagen. These drugs are investigated in vitro for their fibro-, chondro- and osteo-genic effects during chondrogenesis, assessing tissue formed by qPCR, histology, collagen crosslinking, biomechanical and biochemical assays.

1B—Increase capacity of the screening platform and screen new libraries: The current 96-well plate assay is scaled to a 384-well plate format leveraging the use of pipetting robotics, reducing materials and enabling larger library screens, screens following a simulated injury (IL1β) and further temporal assays.

1C—Identify/confirm the targets of lead compounds that stimulate chondrogenesis: Using affinity-based proteomics, knockdown and over-expression experiments targets and signaling pathways targeted by lead compounds are identified.

### To Identify the Role of Dopamine Receptor D4 in Enhanced Chondrogenesis in Human Articular Chondrocytes

Known agonists and antagonists of the dopamine type 4 receptor are tested in the 3D-cell based assay for efficacy in upregulating type II collagen. Similar assessments as in 1A for chondrogenesis are performed. Mechanistic insights are pursued using proteomics for kinase activity and receptor knockdown experiments followed by RNA-Seq analysis.

### Test Drug Efficacy in a Non-Invasive Knee Injury Model for Post-Traumatic Osteoarthritis

A non-invasive knee injury model, compressing the joint and causing an anterior cruciate ligament tear, is used with intra-articular administration of active compounds. Cartilage condition is assessed by in vivo imaging and endpoint μCT and histology. Gait analysis can also give a longitudinal assessment of joint health.

In summary, these experiments provide a platform for drug discovery and investigate targets for anabolic therapy of post-traumatic osteoarthritis.

### Background and Significance

I.I Overall: The ultimate goal of this study is to define a pathway for anabolic drug discovery leading to cartilage regeneration following traumatic injury. Current clinical options for young patients (<60 years old) are limited and mainly consist of palliative care before a total joint replacement is needed. Osteoarthritis is a leading cause of disease and disability worldwide with significant socio-economic costs amounting to over 10% of the US healthcare burden. Post-traumatic osteoarthritis is thought to account for up to 12% of cases, a significant number of patients, ˜12 million. Even patients who undergo anterior cruciate ligament reconstruction have a 50% chance of developing OA within 12-14 years. Cartilage injury and disease is a source of severe debilitation and decreased quality of life. The global burden of musculoskeletal diseases increased by 45% between 1990 and 2010, with osteoarthritis being the fastest growing indication in this area.

The main knowledge gaps addressed by this project are: 1) improving models for cartilage disease modifying drug discovery; 2) the role of dopamine D4 receptor in chondrogenesis, and its potential as a target. Chondrogenesis in 3D pellet culture more faithfully represents native chondrogenesis than 2D culture. Yet many drug discovery efforts have been made in 2D culture with few progressing to the clinic and no disease modifying agents currently available for OA. In addition, it is commonly accepted that oxygen tension is significant in terms of chondrogenesis, yet many efforts are still made under atmospheric oxygen tension.

Natural products represent a diverse array of compounds and still form the basis for many active compounds in the clinic. The NCI natural product library is designed to encompass a diverse range of structures and functional groups. Screening of libraries is an effective way to determine lead compounds for further study. In a traditional cell-based screens in 2D for chondrogenesis from mesenchymal stromal cells, kartogenin was identified based on its ability to induce a rhodamine B staining nodule in 2012. SM04690 was also identified in a cell based screen using intestinal epithelial cells expressing a Wnt reporter. Both compounds are currently in clinical trials for osteoarthritis as potential disease modifying drugs, these studies serve to illustrate both the utility of cell based assays and highlight the potential advantages of a 3D chondrogenic reporter assay.

The role of dopamine in chondrocytes is unclear, one recent study investigated dopamine as a drug that could suppress inflammation in IL-1β stimulated chondrocytes. Much of the study by Lu et al. was conducted on monolayer culture where they found inhibition of NF-κB and JAK2/STAT3 signaling. However, they also found that in a destabilized meniscus model, dopamine reduced cartilage deterioration resulting in a 50% decrease in OARSI score. This study and the expression of dopamine receptor D4 in transcriptomic data from primary human chondrocytes and that of Huynh et al. in mesenchymal stromal cells during chondrogenic differentiation support the investigation of this as a target in osteoarthritis.

One knowledge gap that is partially answered by this study is when to treat chondrocytes to promote extracellular matrix and what pathways are stimulated. A bi- or triphasic chondrogenic response were observed. It is unclear how this relates to the in vivo system in either post-traumatic osteoarthritis or chronic disease. Early, mid and late chondrogenesis is investigated in vitro. These investigations can yield useful information both for tissue engineers and for those seeking to treat osteoarthritis in the patient.

The non-invasive knee injury model is fast becoming a new tool in osteoarthritis research. This study utilizes a device that creates compression of the knee to create an anterior cruciate ligament tear in the rat (FIG. 5).

The establishment of an extracellular matrix, phenotypic 3D cell based reporter system as a mechanism to identify disease modifying osteoarthritis drugs. A novel receptor target, the dopamine receptor D4, as a target for treatment.

### I. Result

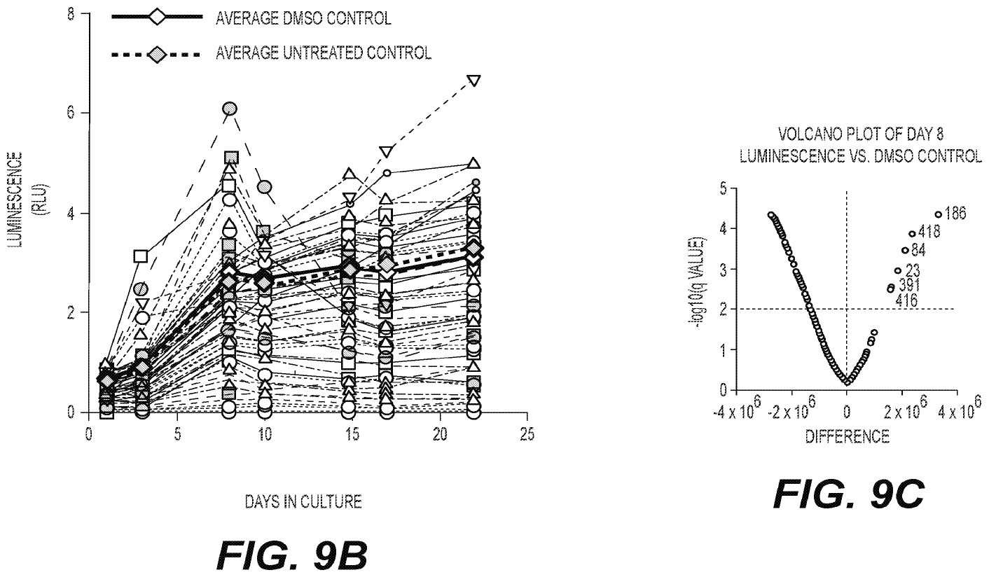

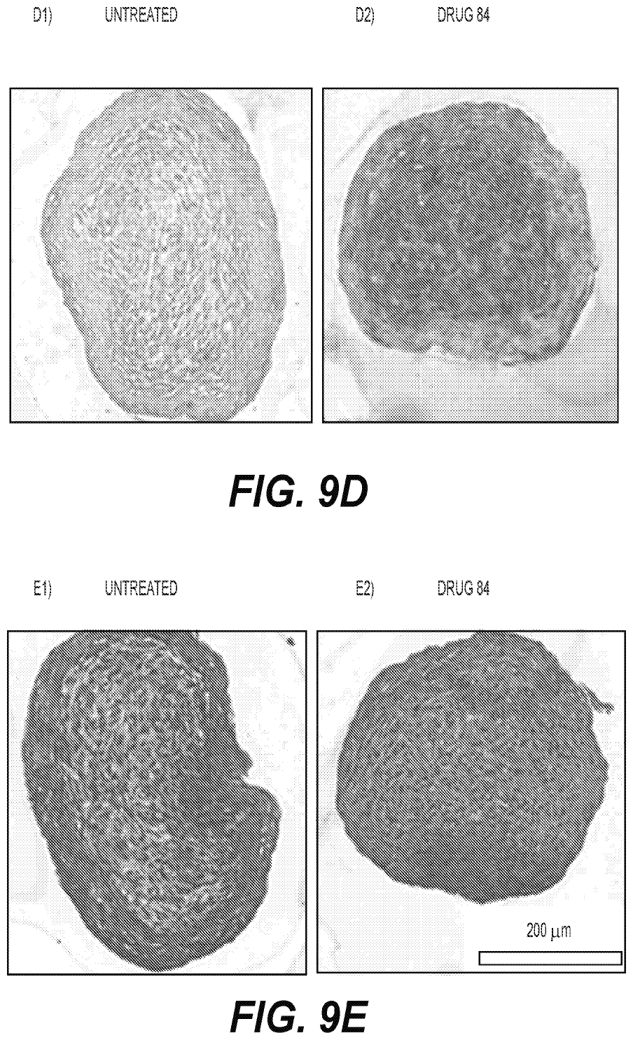

I.II Aim 1—Investigate lead compounds for efficacy in chondrogenesis. Use of the quintessential marker of hyaline cartilage, type II collagen, to drive a secreted luciferase allows for the temporal study of chondrogenesis in 3D culture. ATDC5 cells were demonstrated significant effects across all 14 vitamins and minerals on COL2A1 promoter-driven Gaussia luciferase expression in a high-throughput cartilage spheroid model. The significance of Aim 1 is that a novel platform for anabolic, disease modifying, drug discovery in cartilage is established. The data in primary human chondrocytes first establishes the cells as a non-destructive reporter for type II collagen promoter-driven expression (FIG. 9A). The study then shows that a screen of natural products (FIG. 9B) identified potential candidates stimulating type II collagen expression during early chondrogenesis (FIG. 9C) and that translated into increased type II accumulation within the pellet (FIG. 9D).

It was encouraging to note that all of the initial hits identified in the screen had the dopamine receptor as a potential target identified through in silico analysis using SwissTargetPrediction. That led us to investigate dopamine receptor expression in whole transcriptome data. In this own data on primary human chondrocytes we saw consistent expression of only the dopamine receptor D4 (vs. the other 5 forms). In mesenchymal stromal cells induced to become cartilage in vitro, we see initial expression of dopamine receptor D1 at a low level followed by a strong increase in dopamine receptor D4 expression.

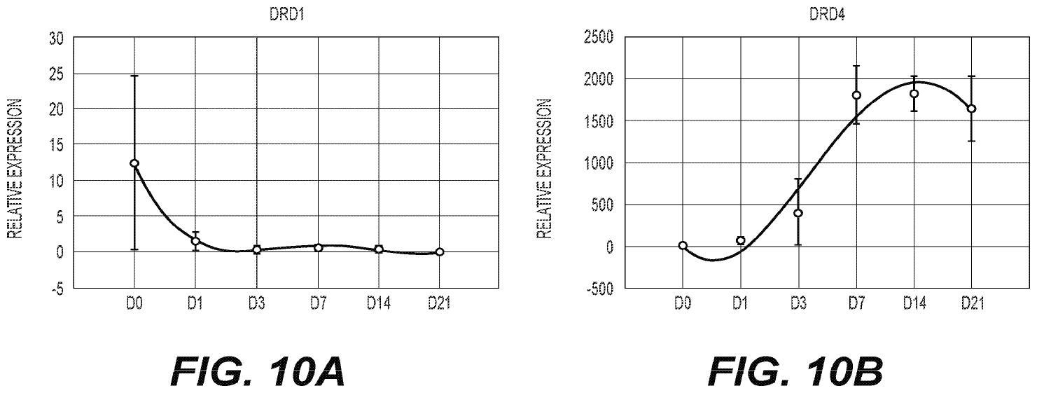

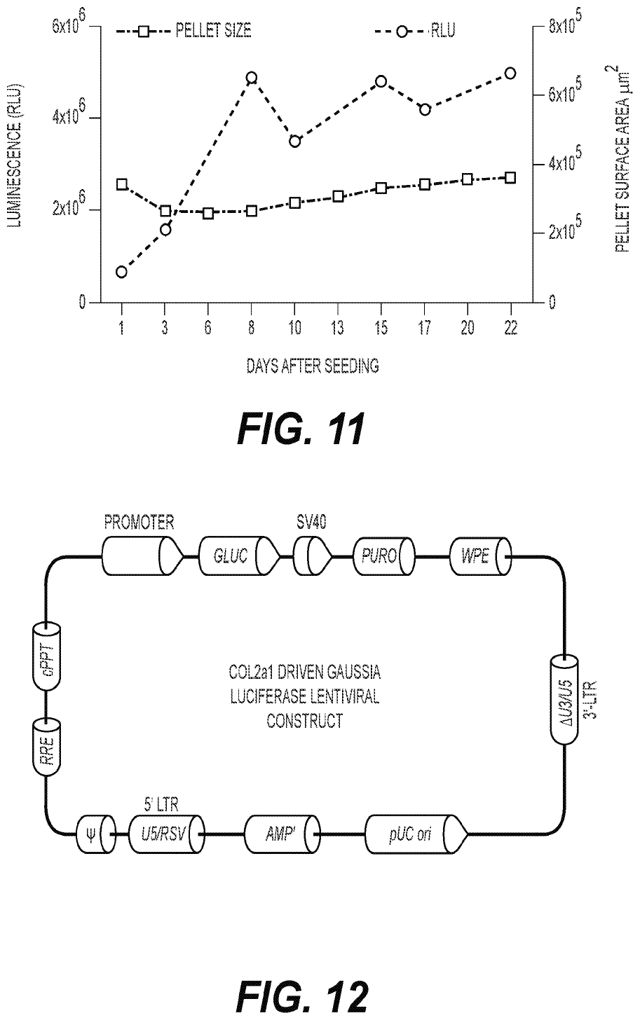

In the iteration of the drug screening assay presented in FIG. 9, all of the drugs were administered at 5 μM continuously throughout the experiment. There appears to be a bi- or tri-phasic pattern of chondrogenesis shown in both articular human chondrocytes (FIG. 9B) and in mesenchymal stromal cells (FIG. 10). A follow up validation of drug candidates (FIG. 11) found similar result to the screen with type II collagen promoter-driven relative luminescence increasing for the expression of the dopamine D4 receptor (FIG. 10).

### II. Innovation

This study determines novel targets and data on un-researched or under-researched areas. Specific areas of innovation are:

II.I Aim 1: There are many areas of innovation in this project, the main innovation being the derivation and use of primary human chondrocytes expressing type II collagen promoter-driven Gaussia luciferase. Study of a phenotypic extracellular matrix component (type II collagen) in a temporal high-throughput human spheroid system for drug discovery is novel. Using the secreted luciferase as a proxy reducing costs, enabling automation and increasing speed is also novel. Identification and confirmation of targets and signaling pathways yields novel data that illuminate the druggable pathways during chondrogenesis, in an established piece of cartilage and in an inflammatory system.

II.II Aim 2: The discovery of a common target between the drugs identified in the screen as the dopamine receptor D4 is a novel insight. Confirmation that this receptor is expressed in primary human chondrocytes and in mesenchymal stromal cells undergoing chondrogenesis from transcriptome data supports a role during chondrogenesis. Investigation of this role during chondrogenesis using agonists, antagonists, knockdown and over-expression with proteomics and RNA-Seq yields critical information on the suitability of this receptor as a target for anabolic stimulation.

II.III Aim 3: Application of the novel compound(s) in a model of post-traumatic osteoarthritis yields data on their suitability for intra-articular administration to reduce or prevent cartilage deterioration.

### III. Approach

Aim 1—Investigate lead compounds for efficacy in chondrogenesis

1A—Evaluate drugs that were identified and confirmed in the screen

1B—Increase capacity of the screening platform and screen new libraries

The development of the primary human chondrocytes expressing the type II collagen promoter-driven Gaussia luciferase followed on from work in ATDC5 cells and rabbit primary chondrocytes. Primary human chondrocytes were isolated from total joint replacement tissue surgical discards by sequential enzymatic digest in hyaluronidase followed by collagenase. Chondrocytes were seeded on tissue culture plastic at ˜30% confluence, allowed to adhere and proliferate for 24 h before infection with lentiviral particles containing the Gaussia luciferase protein under the control of type II collagen promoter (FIG. 12). Variability in the luciferase response using commercial luciferase kits was identified as a potential source of error. Stability of the luminescent signal was optimized by modification of the pH and incorporation of an anti-oxidant, ascorbic acid (0.5M).

Hip and knee revisions are projected to grow by 137% and 601% respectively. Autologous and matrix assisted autologous chondrocyte implantation approaches have been relatively well accepted but predominantly result in fibrocartilage, particularly in large defects, thus limiting their utility. Defined chondrogenic media lacked 14 vitamins and minerals, several of which are known to be involved in skeletogenesis and/or chondrogenesis. Typical chondrogenesis assays such as those for glycosaminoglycan, hydroxyproline (total collagen), DNA and histology are slow, laborious, destructive and difficult to automate. Secreted Gaussia luciferase offers several advantages over intracellular based fluorescent or luminescent reporters: conditioned media can be sampled throughout the experiment at regular feeding intervals thereby not exposing the cells to the stress of fluorescent excitation or lysis for each time point; Gaussia luminescence offers stable, high signal to noise ratios making it readily adaptable to high-throughput analyses; as the spheroids/constructs are 3D extracellular matrix rich, image acquisition/analysis of fluorescent reporters in the full spheroid is difficult if not impossible.

One of the most significant outcomes of this research is a change in the current paradigm where in vitro chondrogenesis is produced in either undefined media (i.e., containing FBS) or media that is lacking in basic vitamins and minerals. It is postulated that the absence of basic vitamins and minerals can skew data to present a false view of what a manipulation can achieve in more physiological conditions. Such a shift in the concept of tissue engineering is translatable to any subfield where defined media have been used. Most basal media were optimized for the growth of cancer cells, and they have since been modified and adapted by addition of growth factors etc. to achieve cell proliferation or differentiation. Whilst the in vitro environment is a model for the in vivo system, it is hard to presume that vitamins and minerals with their myriad and profound effects are unnecessary in these systems. The immediate clinical relevance lies in the improved isolation, expansion and differentiation of cells retaining chondrogenic phenotype. In addition, epigenetic modifications have already been observed as a factor of media composition, which can have significant ramifications on the commonly performed procedure of ACI/MACI™ and applications of the tissue engineered cartilage.

Autologous chondrocytes (Carticel®) were the first FDA approved autologous cell therapy in 1997. Many thought cartilage, due to its avascular and ‘single’ cell type composition, would be one of the first tissues to be engineered; it has yet to be achieved. This is in part due to the view that a chondrocyte is a chondrocyte, where in reality, significant differences were found between articular and auricular chondrocytes. Others have identified that a rare chondroprogenitor cell exists in the surface zone of articular cartilage. It is unclear whether, even with its stem cell like characteristics, enough autologous cells would be achievable from a biopsy given a reasonable amount of time and money.

Focal defects have been treated with autologous chondrocyte implantation (ACI) with variable results: biopsies of 406 ACI patients showed hyaline cartilage in only 14.9% of patients, mixed hyaline and fibrous cartilage in 27.5%, fibrocartilage in 47.7%, and fibrous repair in the remaining 9.9%. ACI has seen significant improvements since it was introduced in the early 1990s, having gone through 3 generations, a characterized chondrocyte implantation (CCI) is now available in Europe. CCI chondrocytes express collagen type II (COL2A1), fibroblast growth factor receptor 3 (FGFR3) and bone morphogenetic protein 2 (BMP2) but not activin receptor-like kinase 1 (ACVRL1); Dell'Accio et al. showed that these genes and the in vivo potential of the chondrocytes were lost after only 4 population doublings. At a recommended 0.8-1 million cells/cm² and a Carticel® biopsy containing 200,000-300,000 cells in 200-300 mg cartilage, a maximum of 2.4 cm² could be filled (3 population doublings). A human femur has an articulating surface area of 64±8.7 cm². In aiming to achieve a biocomposite large defect repair, the improvements in chondrocyte culture methods proposed herein will result in a valuable byproduct of improving both ACI chondrocytes and in vitro methods for cartilage drug discovery; if the chondrocytes that are being tested in vitro are closer to their native counterparts, drug development is significantly improved.

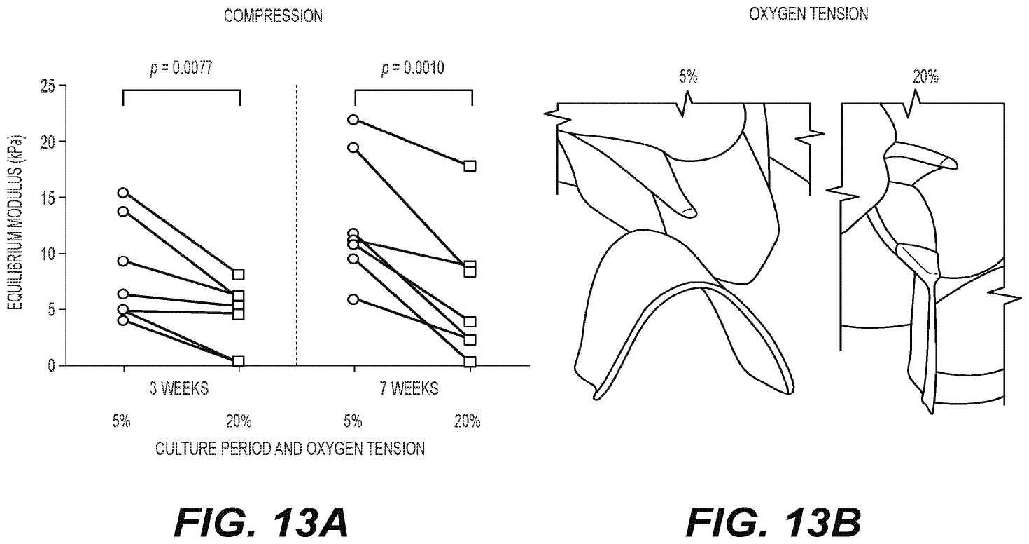

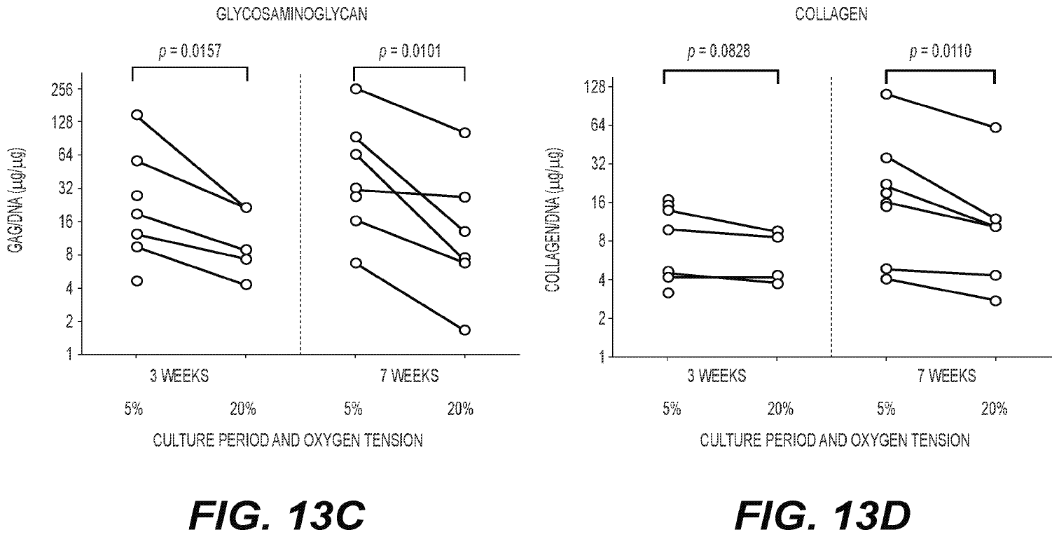

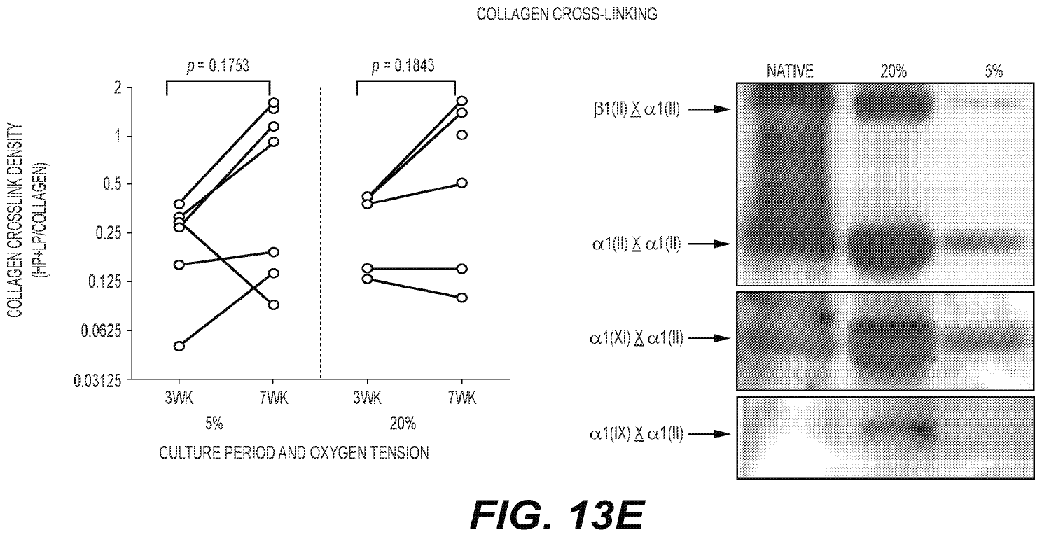

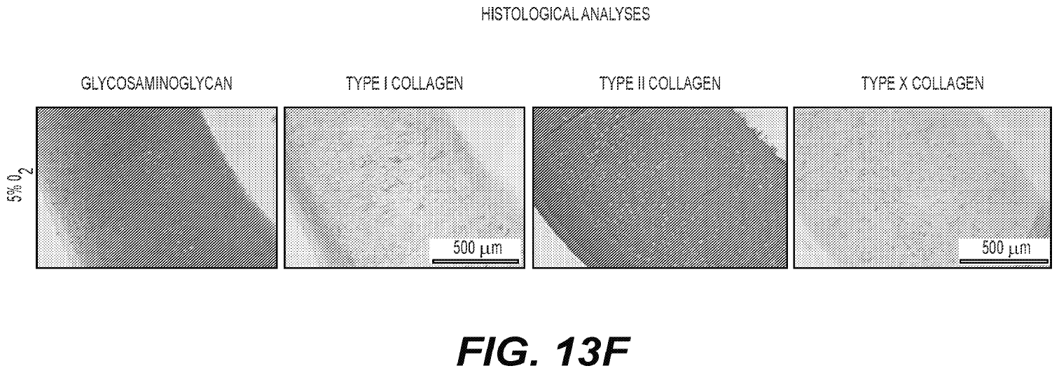

Within the chondrocyte cell source, significant differences were observed in the characteristics of cells isolated from human and rabbit articular, auricular and nasal cartilage with articular chondrocytes producing the most hyaline like cartilage. The improved expansion methodology makes sufficient chondrogenic cell expansion from a small cartilage biopsy attainable. Previous studies making large (16 cm²) human cartilage sheets showed significant variation across donor source in terms or extracellular matrix and biomechanics; however, physioxia was beneficial for all donors (FIG. 13). Media more aligned with physiological conditions can further improve material properties and reduce variability.

Design of Experiment optimization. Design of Experiment optimization is commonly used in engineering and bioprocess manufacturing. It allows the user to optimize across multiple conditions with mathematical and statistical identification of optimal combinations to produce a desired outcome. The value of this approach is in the production of a reliable model from fewer experiments. This technique is under-utilized in cell biology; it has been used in chondrogenesis for articular chondrocytes with some success, though their study was not in physioxic conditions. The optimal concentrations of TGFβ1 and dexamethasone has been included as a factor in the proposed screen. Design of Experiment optimization has not been used on iPSC derived chondroprogenitors.

These factors demonstrate a significant opportunity to apply tissue engineering to the development of a living tissue replacement. This study addresses several issues faced by the tissue engineer and orthopedic surgeon in repair of the cartilage surface, a tissue notorious for its poor intrinsic repair capability. This study improves not only biological defect repair options but also in vitro models of the diarthrodial joint that support drug discovery efforts.

We have successfully used the COL2A1-gLuc construct in ATDC5s, primary rabbit chondrocytes and primary human chondrocytes with detailed analysis of the effect of vitamins and minerals in ATDC5 and rabbit chondrocytes (FIG. 2). As can be seen, more than a two-fold increase in COL2A1-driven luminescence can be achieved over this current ‘gold standard’ of TGFβ1 supplementation alone. In the human cells, a clear dose response to TGFβ1 is shown, with a peak of sensitivity at day 15. The conservation of the effect of several of the combinations from a murine cell line to primary rabbit chondrocytes is encouraging.

Most cartilage tissue engineering strategies incorporate a large proportion of biomaterial or synthetic scaffold to cell derived matrix. These tissue engineered sheets are all cell-derived tissue. It is clear from previous work that even a “simple” change, such as culture in physioxic conditions, significantly modifies tissue properties increasing glycosaminoglycan, collagen, thickness and mechanical properties (FIG. 13). The increase in expression of type II collagen can result in increased biomechanical properties. Glycosaminoglycan, or aggrecan, is typically found at or above physiological levels. The aggrecan promoter-driven assay has been included so that type II collagen expression can be optimized without losing glycosaminoglycan. Lubricin has been included, because loss of lubricin results in OA symptoms and replacement results in protection from OA.

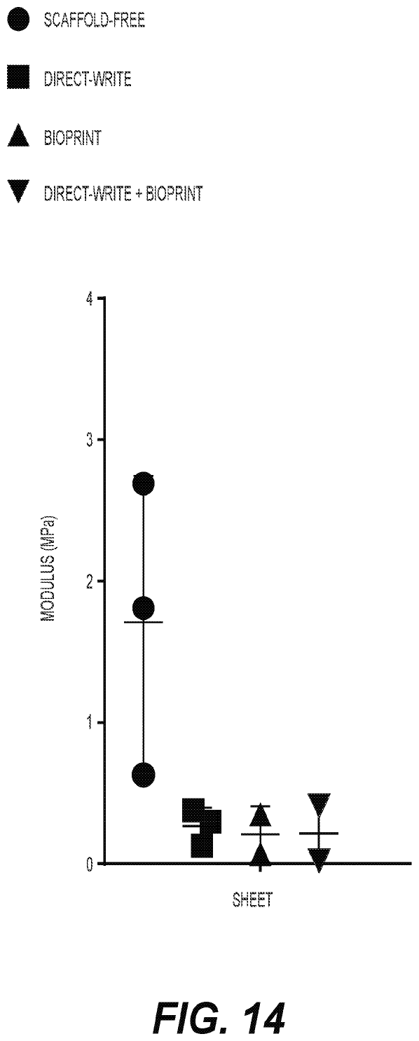

These engineered cells represent an ideal opportunity to monitor and optimize 3D printing and bioprinting efforts, as current techniques resulted in poor shape fidelity over a 1-month culture and lower biomechanical properties in comparison to scaffold-free sheets (FIG. 14). That work highlighted the dire need for a rapid chondrogenic assessment technique in 3D printing and bioprinting.

Aim 2 Human iPSC derivation methods have significantly improved both in terms of forming iPSCs and in the derivation of chondroprogenitors in the past few years. Further, iPSC derived cartilage has limited immunogenicity, making this a source of cartilage for transplantation either from autologous iPSCs or an allogeneic master cell bank. Whether chondroprogenitors derived from iPSCs can respond to vitamin and mineral supplementation in the same way as ACs is unknown. Experiments are performed with already established COL2A1-GFP iPSC derived chondroprogenitors. Current media used in iPSC chondrogenesis uses serum, cartilage development in defined media is studied.

Aim 3 Filling of a chondral defect is an established approach in rabbits. Skeletally and chondrally mature rabbits are used as per ICRS guidelines. A long, 6-month, study is conducted as short term studies often show positive outcomes whilst longer studies with the same approach often show no improvement. Gait analysis is widely used in mice, rats and humans; the same principles are applied in rabbit model. To add to this longitudinal analysis, ultrasound assessment is also made as this has proven to be effective in mice and humans. Whilst earlier studies indicated that cartilage and the joint are an immunologically privileged site due to the chondrocytes being protected from T-cell contact by the dense extracellular matrix more recent work has shown allogeneic cells are well tolerated in this model but xenogeneic cells/constructs are not. Immunosuppression for 6 months is unfeasible, and shorter studies are poorly predictive of long term outcomes. Therefore, allogeneic rabbit chondrocytes are used to test fixation, integration and the effect of construct stiffness at implantation. Current methods using non-optimized defined media to produce articular cartilage sheets from rabbit chondrocytes result in scaffold-free sheets with strong glycosaminoglycan and type II collagen staining throughout the sheet and compressive properties at 25-50% of native tissue (FIG. 14) which can be sufficient for implantation. Mechanical properties at implantation and their effect on integration are unknown. The modifications to media and formation of a zonal construct by 3D printing/bioprinting can result in an improved repair over current scaffold-free sheets. Fixation with a biocompatible glue containing FGF18 can improve integration with the surrounding tissue.

### IV. Innovation

Overall: Study of extracellular matrix components and the effects of vitamin, mineral and growth factor supplementation on them in a temporal high-throughput human spheroid system is novel. Applying those engineered cells to study 3D printing and bioprinting is also novel. The combination of Design of Experiments planning and analysis with the temporal, high-throughput reporter assays allows for rapid optimization across many factors.

Aim 1 Derivation and use of primary human chondrocytes expressing type II collagen promoter-driven Gaussia luciferase (AC-col2-gLuc). Derivation and use of primary human chondrocytes expressing aggrecan promoter-driven Gaussia luciferase (AC-acan-gLuc). Derivation and use of primary human chondrocytes expressing lubricin promoter-driven Gaussia luciferase (AC-prg4-gLuc). Application of AC-col2-gLuc, AC-acan-gLuc and AC-prg4-gLuc cells in a high-throughput temporal spheroid chondrogenic assay. Use of statistical modeling for chondrogenic optimization.

Aim 2 Derivation and use of iPSC derived chondroprogenitors expressing native type II collagen-driven mCherry and Gaussia luciferase (iPSC-col2-mC-gLuc). Derivation and use of iPSC derived chondroprogenitors expressing native aggrecan-driven mCherry and Gaussia luciferase (iPSC-acan-mC-gLuc). Derivation and use of iPSC derived chondroprogenitors expressing native lubricin-driven mCherry and Gaussia luciferase (iPSC-PRG4-mC-gLuc).

Aim 3 Application of the tissue engineered cartilage using mussel inspired gelatin glue in a patellofemoral defect is a novel use of an already developed product, as is the incorporation of FGF18. Gait assessment in rabbits is also under-reported, as is ultrasound monitoring of a defect fill.

### V. Approach

Project overview. Reporter cells are made from articular chondrocytes and iPSCs under the control of extracellular matrix proteins COL2A1, ACAN and PRG4. Vitamins and minerals absent in defined chondrogenic media are screened for stimulation or repression of those extracellular matrix genes and for their effect on metabolic activity. 3D printing and bioprinting are investigated for their effect on extracellular matrix genes, with the goal of creating a biocomposite with a lubricin expressing surface zone, type II collagen and aggrecan expressing middle zone and a bone integrating lower zone. Constructs derived from rabbit articular cartilage are implanted in a critical sized defect in a mature rabbit model.

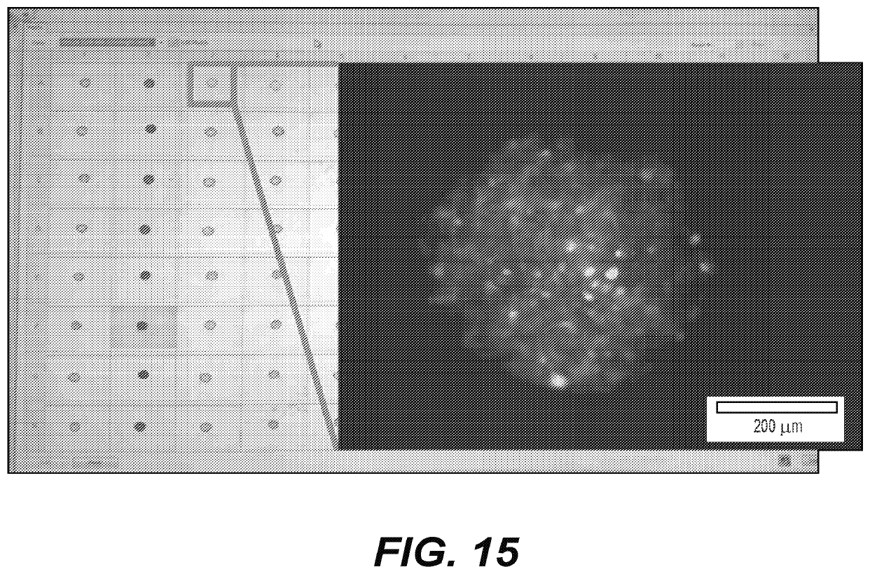

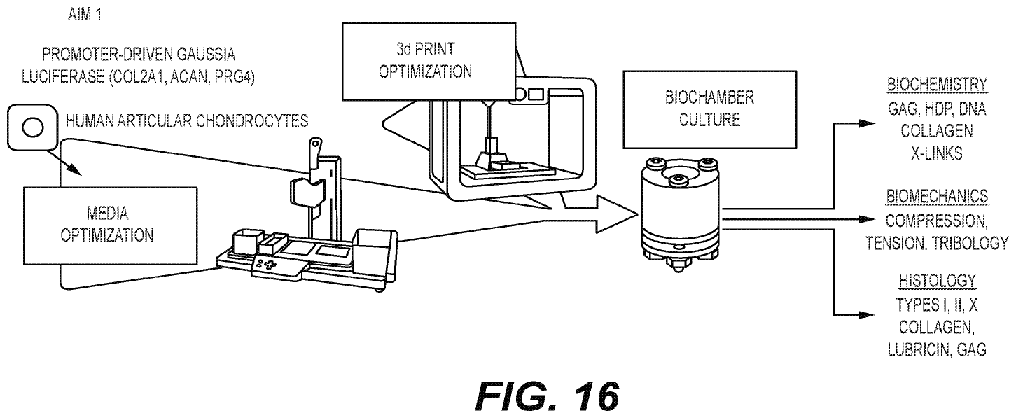

Aim 1: Define optimal supplementation scheme using phenotypic reporter cell analyses. Overview: In Aim 1 (FIG. 16), human articular chondrocytes from 6 donors (3 male, 3 female) are transduced with lentiviral Gaussia luciferase under the control of extracellular matrix promoters. Defined chondrogenic media is supplemented with vitamins, minerals and growth factors and the response analyzed over a 21-day robot assisted aggregate culture. Growth and differentiation of cells on 3D printed architectures and in 3D bioprint inks is also analyzed. Optimal conditions are tested in biochamber cultures producing 1 cm² pieces of cartilage tissue from both transduced cells and frozen primary cell stocks. Tissue engineered cartilage sheets are assessed biochemically, mechanically and by histology.

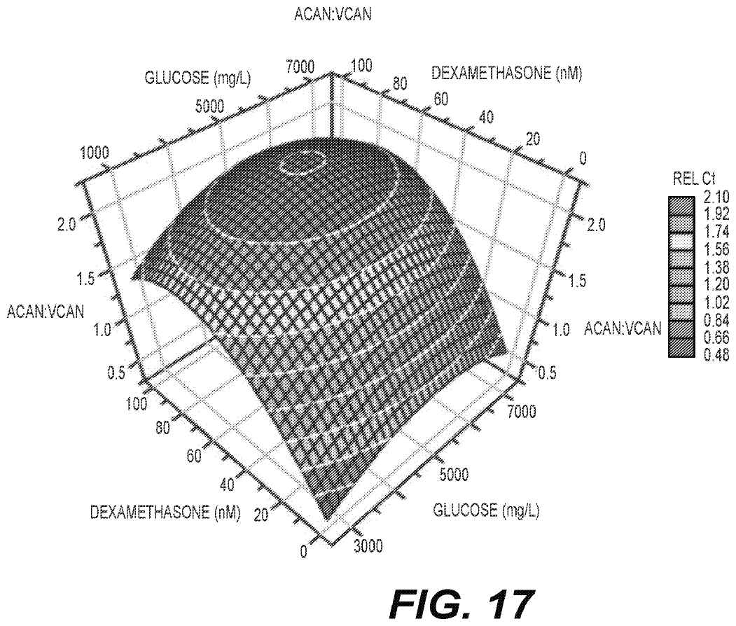

Chondrocyte isolation and infection: Human articular chondrocytes are isolated from a macroscopically normal area in discarded surgical tissue from patients undergoing total joint replacement (6 donors, 3 male, 3 female). Cartilage is minced (1 mm³ pieces) then sequentially digested in hyaluronidase then collagenase. This cell solution is then plated at 6,000 cells/cm² in a humidified physioxic (5% O₂) environment. Typical yields have been >4 million cells which allows for separation between 4 T175 flasks giving an untreated flask and one treated flask for each sub aim (Aim 1A, Aim 1B and Aim 1C). Cells are allowed to adhere for 48 h, then washed with Tyrode's and treated with pseudolentiviral particles (MOI 25) containing promoter-driven-gLuc (FIG. 12) in the presence of polybrene (4 μg/ml). Infectious particles are removed the next day, cells allowed to recover overnight then treated with puromycin (2 μg/ml) to select for transduced cells. When 80-90% confluent, cells are trypsinized and plated onto synoviocyte derived extracellular matrix and grown in FGF containing media. Quantitative PCR and assessment of media for luciferase activity are also performed at this step to confirm transduction. 221. Design of Experiment testing and optimization of media-vitamins, minerals and growth factors: Chondrocytes are trypsinized from the flask at the end of passage 2 and formatted into non-adherent 96-well plates (50,000 cells/well) for chondrogenic spheroid induction using robotic pipetting (Opentrons). TGFβ1, a known stimulator of chondrogenesis, type II collagen, aggrecan and lubricin, is tested (0-50 ng/ml) and media assessed for luciferase activity and metabolic activity (resazurinreduction) at each feeding point over a 21 day chondrogenic assay. This assay establishes the ability of the chondrocytes to produce cartilage matrix. A two-factorial interactive D-optimal response surface screen will be made across 15 vitamins and minerals, 4 base media and two levels of TGFβ1 and dexamethasone supplementation (Table 1). A D-optimal design was chosen, as it produces a design that best estimates the effects of the factors, which is particularly suited for screening studies. Maximal concentrations of vitamins and minerals are based on 2× the optima derived in ATDC5 cells. These designs enable statistical modeling to identify optimal concentrations/mixes (FIG. 17). This design results in 198 conditions tested, significantly less than those tested in the previous work with much more statistical power to determine interactions and synergy between the factors. Optimal media for cartilage spheroids: produces the highest promoter-driven Gaussia luciferase activity, across the whole 3-week assay (Aim 1A). Aims 1B and 1C have the same optima, with their respective aggrecan- or lubricin-promoter driven-Gaussia luciferase expression. In addition, there is no detrimental effect on metabolic activity. An overall optimum is defined.

TABLE 1
Summary of screening optimization factors

| Factor | Min | Max | | Levels |
|---|---|---|---|---|
| Chromium | 0 | 0.56 | μg/L | |
| Cobalt | 0 | 180 | pg/L | |
| Copper | 0 | 13.4 | μg/L | |
| Iodine | 0 | 184 | ng/L | |
| Manganese | 0 | 24 | ng | |
| Molybdenum | 0 | 0.4 | ng/L | |
| a-Linolenic acid | 0 | 1.08 | mg/L | |
| Vitamin A | 0 | 156 | ng/L | |
| Vitamin B7 | 0 | 6 | ng/L | |
| Vitamin B12 | 0 | 1.828 | μg/L | |
| Vitamin D | 0 | 17.2 | pg/L | |
| Vitamin E | 0 | 36.8 | mg/L | |
| Vitamin K | 0 | 22 | μg/L | |
| Thyroxine | 0 | 50 | pg/L | |
| Zinc | 0 | 240 | ng/L | |
| Base Media | DMEM-HG IMDM | | | DMEM-HG/F12 RPMI |
| Base Additives | TGFβ1 10 ng/ml, | | | TGFβ1 13 ng/ml, |
| | Dexamethasone 100 nM | | | Dexamethasone 10 nM [37] |

Optimization of 3D printing and bioprinting for human articular chondrocytes: In order to have a true zonal architecture with lubricin expressing cells on the surface, a mid zone rich in type II collagen and aggrecan and a lower, bone integrating zone, 3D printing/bioprinting is critical. Similar to the screen of vitamins and minerals, cell density, polymer type, geometry, and bioink factors are assessed (Table 2). All of these factors are known to have an effect on cell phenotype, but it is not clear what is optimal for cartilage regeneration. A linear D-optimal response surface screen is used in this screen as it is both time and labor intensive to assess these printing and bioprinting factors. The linear D-optimal design results in 24 experimental conditions. All conditions are tested in an overall optimized media giving the highest expression of promoter-driven luminescence across all 3 genes with no loss of metabolic activity. Each scaffold, or the polyester membrane (our current method) is fibronectin coated then tested for their ability to support reporter cell culture in 24 well plates with metabolic and luciferase measurement at days 1, 5, 9, 16 and 23.

TABLE 2
Summary of 3D print optimization factors

| | Levels | | | | | |
|---|---|---|---|---|---|---|
| Factor | Min | | | Max | | |
| Cell concentration | 1 million/ml | | | 10 million/ml | | |
| Polymer | PLA | PLGA | PLA/PCL | Chitosan | Hyperelastic | polyester |
| Geometry | triangle | square | hexagon | | bone | membrane |
| Bioink | none | alginate | collagen | gelatin | hyaluronan | laminink |

Testing and verification of optimized conditions for human primary chondrocytes in sheet culture: Optimized culture conditions will be tested in this current biochamber culture format (FIG. 16). Chondrocytes are seeded onto fibronectin coated polyester membranes and/or optimized 3D printed supports. Cells are seeded at 5×10⁶ cells/cm² and allowed to adhere/self assemble over 24 h; at this point additional media is added to allow exchange between the top and bottom surfaces. At 48 h, slow (10 rpm) shaking is begun; after a further 48 h, the seeding chamber is removed and shaking is increased to 60 rpm. Media is exchanged every 2-3 days. All studies will be conducted at physiological oxygen tension (5%). Sheets will be grown for 1 month, with media samples for luciferase activity assessment, then tested mechanically, biochemically and histologically. Standard mechanical tests for unconfined compression to give equilibrium modulus, rheometric (friction) and dog bone punches used in tensile test to failure giving the elastic tensile modulus. In addition, samples produced as part of this research are tested using custom designed ultrasound apparatus for acoustic anisotropy measurements and custom-designed combined shear-ultrasound device. These new modes of mechanical assessment and characterization have significant potential in developing non-destructive mechanical assessments as a release criterion for tissue engineered cartilage. Samples for mechanical tests will be collected and stored frozen, shipped on dry ice overnight and tested. Biochemical tests are for glycosaminoglycan, DNA and hydroxyproline with further assessment of collagen crosslinking. Frozen samples are also used for collagen crosslinking analysis. Briefly, samples are wicked dry and hydrated weights taken, followed by lyophilization and a dry weight taken. The heteropolymer collagen network is depolymerized in 0.5M acetic acid with pepsin (100 μg/ml). Histologically, stains for glycosaminoglycan (safranin-O) and calcium (von Kossa) are routinely performed, along with assessment of type I, II and X collagens, aggrecan and lubricin. Samples are ranked based on total glycosaminoglycan, collagen, and DNA content with improvement in type II collagen being most desired and maintenance of glycosaminoglycan and DNA. Increased mechanical properties, both in compression and tension are also well scored. Improved crosslinking is expected to have a positive impact on material properties. Finally, distribution throughout the tissue of glycosaminoglycan staining and type II collagen will be scored. In summary, the optimal condition is defined as: the highest compressive and tensile moduli, lowest friction, total collagen levels ≥20 μg/μg DNA, glycosaminoglycan levels ≥50 μg/μg DNA, most intense type II collagen staining, with distribution throughout the sheet, little type I or X collagen staining and lubricin staining on the surface.

Aim 2: Development of induced pluripotent stem cell (iPSC) derived reporters. Overview: Aim 2 follows much the same pathway as Aim 1 (FIG. 13), with two crucial modifications 1) human iPSCs are used with CRISPR/CAS9 technology to 2) knock in a dual reporter in the 3′ region of the endogenous genes for type II collagen, aggrecan and lubricin. Defined chondrogenic media ar supplemented with vitamins, minerals and growth factors and the response analyzed over a 21-day robot assisted aggregate culture. Growth and differentiation of cells on 3D printed architectures and in 3D bioprint inks will also be analyzed. Optimal conditions are tested in biochamber cultures producing 1 cm² pieces of cartilage tissue from transduced cells and their iPSC progenitors. Tissue engineered cartilage sheets are assessed biochemically, mechanically and by histology.

iPSC reporter line development: Dual reporter cells will be developed by the group of Dr. Fitzgerald (see letter of support). For Aim 2A Guide RNAs developed and proven effective to target the 3′ end of the type II collagen gene will be used. “DNA sequence encoding sgRNA targeting the COL2A1 stop codon was ligated using the following complimentary oligonucleotides: 5′-caccGGTCTGCTTCTTGTAAAAACC-3′ (SEQ ID NO: 1) and 5′-aaacGGTTTTTACAAGAAGCAGACC-3′ (SEQ ID NO: 2). Lower case nucleotides allowed cloning into the SpCas9 expression vectors by annealing to overhangs formed by BbsI restriction enzyme.”. These guide RNAs in combination with an R691A-modified Streptococcus pyogenes Cas9 will be used to knock in a bi-functional cassette expressing Gaussia luciferase and mCherry separated by the P2A cleavage peptide. The modified Cas9 protein minimizes off target effects and increases editing to 90-95% of cells. The fluorescent mCherry allows for cell selection and potential high content analyses. The Gaussia luciferase gives a secreted reporter system for 3D construct analyses as previously described. The ribosomal skipping P2A cleavage peptide facilitates cleavage of the native gene and the reporter gene following expression. The reporters are cleaved so that they do not interfere with native protein function. No targeted knock-ins were identified for either aggrecan (Aim 2B) nor lubricin (Aim 2C), so several new guide RNAs are tested for their ability to target the introduced reporter genes to the 3′ end of the native genes. Clonal populations are assessed for gene inserts and driven down the mesenchymal lineage using established methods. Four chondroprogenitor clones per gene are differentiated into cartilage tissue and assessed during differentiation for fluorescence and luminescence. Chondroprogenitors are formatted into 96-well non-adherent round bottom plates (Cellstar, Greiner) and treated with current iPSC chondrogenic media (DMEM/F-12, 1% FBS, 1% ITS+, 55 μM 2-mercaptoethanol, 100 nM dexamethasone, 1% NEAA, 1% P/S, 10 ng/ml human TGFβ3, 50 μg/ml L-ascorbic acid 2-phosphate, and 40 μg/ml L-proline). After 28 days of culture, cartilage spheroids are assessed biochemically (glycosaminoglycan, hydroxyproline and DNA) and histologically (glycosaminoglycan by safranin-O/fast green, calcium by von Kossa, type I collagen, type II collagen, type X collagen, aggrecan and lubricin). Clones which have robust expression of reporters and hyaline cartilage specific staining are selected for further optimization/differentiation analyses.

Design of Experiment optimization of media for iPSCs: The data gained in Aim 1 is indicative of that which might be achieved through the native type II collagen promoter, an approach that can be studied using knock-in reporters in iPSCs. A recent study used CRISPR/Cas9 targeting to insert GFP after the endogenous type II collagen in human iPSCs. GFP^{high} chondroprogenitor cells are used for this study. Chondroprogenitors are expanded. Briefly, cells are grown on TC plastic coated with 0.1% gelatin (standard conditions) in DMEM-F12 (supplemented with 10% FBS, 1% ITS+, 55 μM 2-mercaptoethanol, 1% MEM NEAA, 1% P/S, 40 μg/ml FGF2 and 50 μg/ml ascorbate-2-phosphate). Once expanded, cells are formatted into 96-well non-adherent round bottom plates (Cellstar, Greiner) and treated with various media supplements (Table 1) along with the current, non-defined media (DMEM/F-12, 1% FBS, 1% ITS+, 55 μM 2-mercaptoethanol, 100 nM dexamethasone, 1% NEAA, 1% P/S, 10 ng/ml human TGFβ3, 50 μg/ml L-ascorbic acid 2-phosphate, and 40 μg/ml L-proline). Round bottom non-adherent plates are used, as polypropylene V-bottom plates do not allow for imaging. As in Aim 1, a Design of Experiment approach will be taken, and 17 factors studied (Table 1): vitamins, minerals and growth factors are screened in a two-factor interaction response surface design, along with the current standard chondrogenic culture conditions. Aggregates are assessed for fluorescence intensity as well as individual cell fluorescence. The main concern here is that signal to noise ratios will be insufficient, hence the development of the new iPSC reporter lines expressing both a fluorescent reporter and the secreted Gaussia luciferase (iPSC-col2-mC-gLuc). These new cell lines, reporting on native gene expression for type II collagen, aggrecan (Aim 2B) and lubricin (Aim 2C), can undergo the same assay as described above, studying the 17 factors in a Design of Experiments layout against the current, non-defined media. The goal here is to switch from a non-defined media and to optimize iPSC differentiation to produce type II collagen, aggrecan and lubricin. The overall goal is stimulation of type II collagen and lubricin, as these are commonly deficient in tissue engineered cartilage compared with native tissue, and aggrecan maintenance, as this is at or above native glycosaminoglycan levels.

Optimization of 3D printing and bioprinting for iPSCs: As in Aim 1, it is envisioned that to translate these tissue engineered cartilage constructs to a zonal implant, a biocomposite design is necessary. 3D printed and bioprinted optimization are performed as described previously (Table 2). The optimal media for total chondrogenesis (type II collagen+aggrecan+lubricin promoting) used. Assessment of chondrogenic activity will be made by both fluorescent microscopy (mCherry) and conditioned media analysis for luciferase and resazurin activity at days 1, 5, 9, 16 and 23. Optimal conditions result in the highest luciferase activity for each gene individually, and/or overall without compromising metabolic activity (resazurin). Additionally, due to the incorporation of the fluorescent reporter, we can account for population diversity within a construct e.g., one highly active cell vs. many similarly active cells.

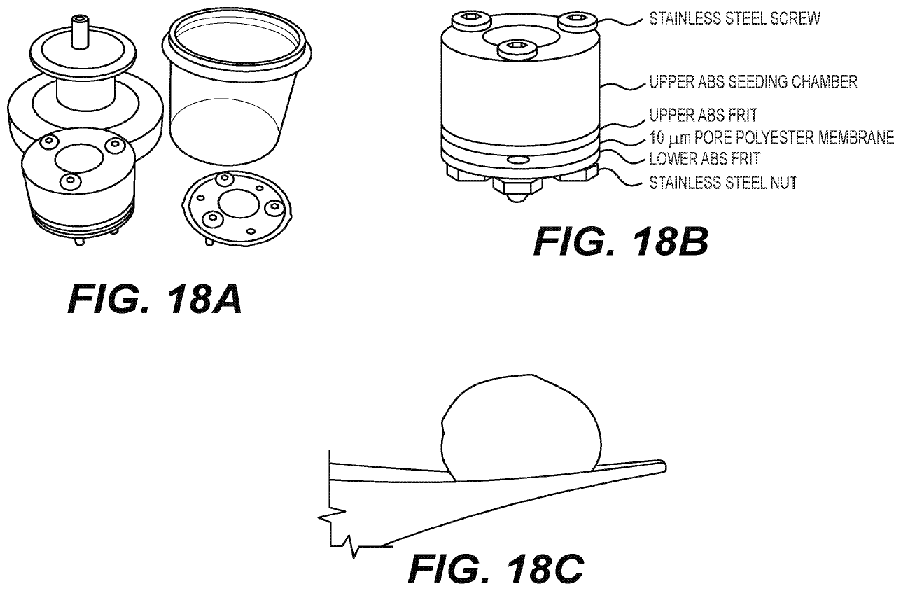

Testing and verification of optimized conditions for human iPSC chondroprogenitors in sheet culture: As in Aim 1, the optimal overall conditions for expression of type II collagen, aggrecan and lubricin can be tested in a scaffold free tissue engineered cartilage biochamber (FIG. 18) and optimized 3D printed/bioprinted conditions. For scaffold free culture, chondroprogenitors are seeded at 5×10⁶ cells/cm² on fibronectin coated polyester membranes as previously described. Chondrocytes are allowed to adhere/self assemble over 24 h; at this point, additional media is added to allow exchange between the top and bottom surfaces. At 48 h, slow (10 rpm) shaking is begun; after a further 48 h, the seeding chamber is removed and shaking is increased to 60 rpm. Media is exchanged every 2-3 days. All studies are conducted at physiological oxygen tension (5%). Sheets are grown for 1 month, with media samples for fluorescence (resazurin) activity assessment, then tested mechanically, biochemically and histologically (FIG. 9). The optimal condition is defined as: the highest compressive and tensile moduli, lowest friction, total collagen levels ≥20 μg/μg DNA, glycosaminoglycan levels ≥50 μg/μg DNA, most intense type II collagen staining, with distribution throughout the sheet, little type I or X collagen staining and lubricin staining on the surface.

Aim 3: Rabbit chondral defect repair. Overview: Aim 3 (FIG. 19) uses current scaffold-free rabbit articular tissue engineered cartilage constructs compared with biocomposite constructs formed using optimal media and 3d printing/bioprinting methods to assess a chondral replacement. These engineered tissues are fixed in the joint with a biocompatible mussel inspired chitosan-based glue containing FGF18. Animals are studied over a 6-month period with assessments at 1-, 2- and 6-months for implant filling and gait analysis. With final assessment at 6-months tissue will be harvested from the implanted and control knee for biomechanics, μCT and histology.

Tissue engineered cartilage implant fabrication: Cartilage sheets are made in this current biochamber format (FIG. 18). Rabbit articular chondrocytes from frozen stocks are expanded on devitalized synoviocyte matrix in growth media (DMEM/F12 supplemented with 10% FBS and 2 ng/ml FGF2) for two passages. Two passages under these conditions typically result in >8 population doublings with no loss of chondrogenicity, i.e. from 1 vial of frozen primary cells containing 1 million cells, more than 256 million can be achieved. Three scaffold-free sheets are formed from 5 million cells, seeded in the top seeding chamber and allowed to adhere/self assemble over 24 h; at this point additional media is added to allow exchange between the top and bottom surfaces. At 48 h, slow (10 rpm) shaking is begun; after a further 48 h, the seeding chamber is removed and shaking is increased to 60 rpm. Media is exchanged every 2-3 days. All studies are conducted at physiological oxygen tension (5%). Sheets are grown for 1 month then taken for implantation. Simultaneously, sheets are grown using optimal conditions determined for zonal culture in Aim 1. Partner sheets not used in implantation are used to assess mechanical, biochemical and histological properties at the time of implant.

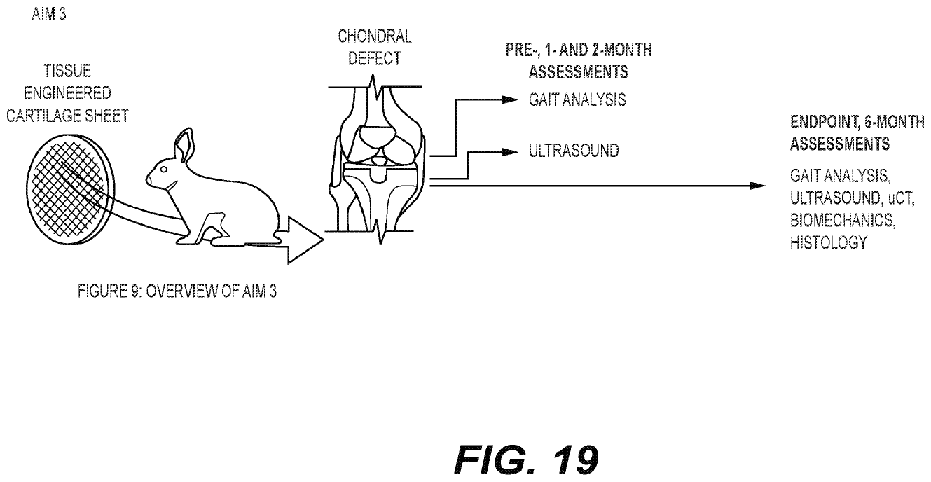

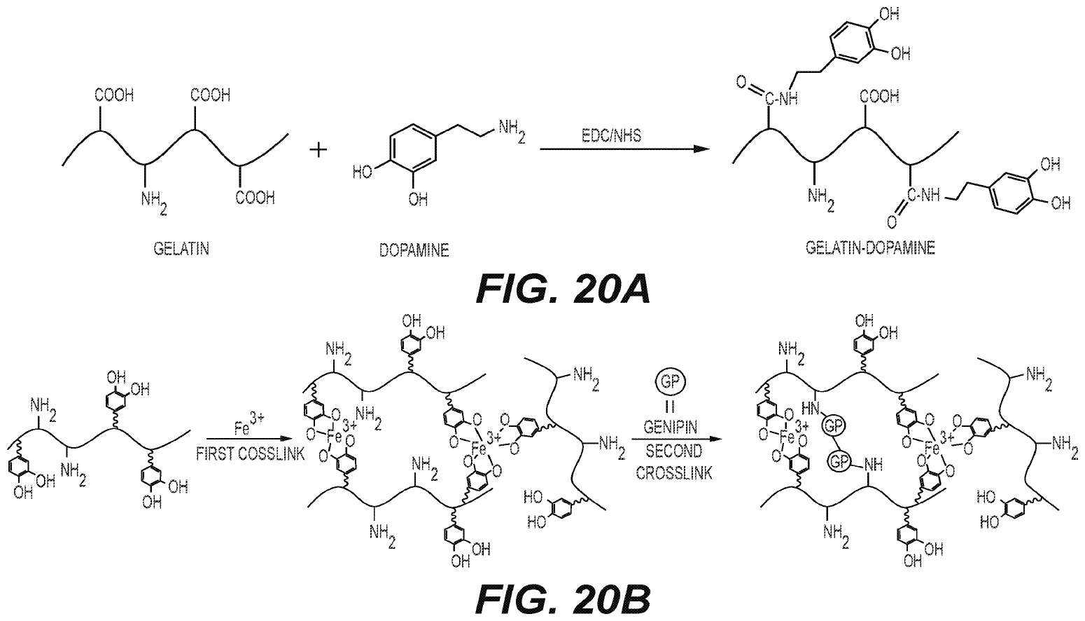

Glue fabrication and testing: The shortcoming of many glues is their inability to stick in wet conditions; mussels have overcome this primarily due to a dopamine linkage. Gelatin is modified with dopamine in a one-step reaction (FIG. 20A). This catechol group allows for the quick formation of crosslinks by iron complexation (FIG. 20B). The tissue are further stabilized in the defect by genipin crosslinking, a biocompatible cross-linker. This dual cross-linked glue resulted in an adhesion strength of 194.4±20.7 KPa for cartilage tissue after 2 h. FGF18 is incorporated into the gelatin-dopamine glue at 100 ng/ml to enhance chondrocyte mediated integration with the surrounding tissue. Glued constructs are tested for delamination and force to failure from debrided defects in cadaver tissue. Release of FGF18 from a fixed defect wis studied in vitro by ELISA assessment of the media, along with integration of the cartilage over a 1-month culture.

Surgical approach: Eighteen skeletally and chondrally mature rabbits (>8-months old) are randomly assigned to 3 groups: 1) Gelatin-dopamine double crosslink glue only; 2) current non-optimized media scaffold-free cartilage construct; 3) optimized zonal cartilage biocomposite construct. Rabbits will be anaesthetized with ketamine/dexmedetomidine (20 mg/kg and 0.15 mg/kg respectively) and maintained with 1-5% isoflurane with monitoring of breathing rate, temperature, pulse and blood oxygenation. The patellofemoral joint surface will be exposed with a medial incision. A single 4-mm critical sized defect is made, guided by a 3D printed guide to aid reproducibility, with a biopsy punch and curette debridement. Grafts are biopsy punched to size match with the defect and glued in place by application of gelatin-dopamine glue with 100 mM FeCl₃, four resorbable vicryl anchoring sutures are placed to aid security. The joint capsule is closed with resorbable vicryl sutures, followed by subcuticular sutures and intradermal sutures to close the wound with lidocaine for localized pain alleviation and slow release buprenorphine for generalized pain relief. Carprofen and baytril are used for inflammation and antibiotic control respectively. Continual monitoring will be carried out after surgery until animal is sternal with fluid and warmth support. Animals are monitored for seven days for grimace, motility and behavior and joint swelling/temperature.

Gait analysis: Whilst there are many published studies on gait analysis in mice, rats and humans those on rabbits, despite their wide use as an osteoarthritis model, are rare with perhaps the best report by Gushue et al. However, the work by Gushue et al. was invasive and hard to translate to other models. A relatively low cost, accessible analysis of rabbit gait before and after critical sized defect surgery could offer insight into pain and repair mechanisms. Using non-toxic paint, stride length, toe out angle and step width can be assessed. Affordable high-speed cameras are also available which, with marked points on the joints, can be used to assess kinematic movements with automated image analysis. Two gait analyses are performed on each rabbit pre-surgery followed by assessments at 1-month, 2-months and 6-months after implantation.

Ultrasound analysis: Ultrasound has clinical and pre-clinical potential in the assessment of cartilage repair. It is an accessible and non-ionizing form of assessment. Ultrasound scans (Sonosite) are made of both knees pre-defect, at 1-month, 2-months and 6-months post defect.

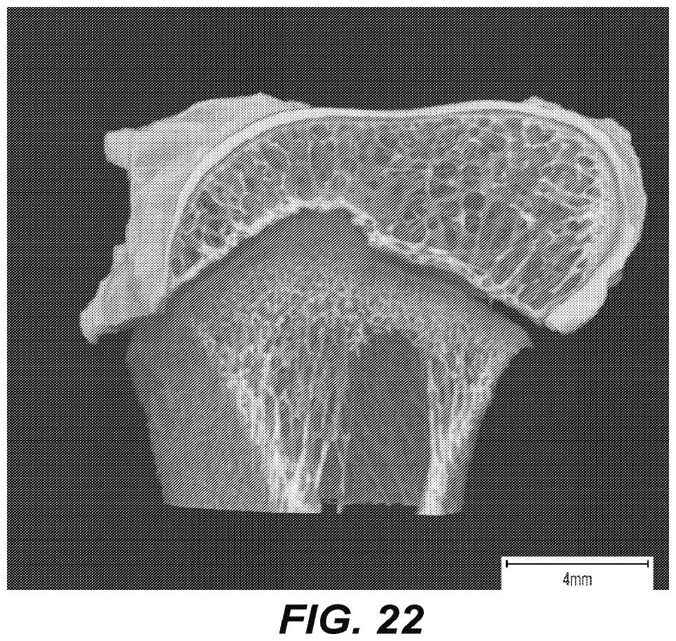

μCT analysis: At sacrifice, 6-months post-implantation, both knees (treated and control) are excised, clamped proximally in a bone mold and cut coronally down the center of the femoral head through the center of the implant with a diamond blade bone saw (IMEB Bone Band Saw, San Marcos, CA). The anterior portion is formalin fixed for μCT assessment (FIG. 22) of the engineered surface and underlying trabecular bone structure. Analysis of epiphyseal μCT includes the following parameters: trabecular bone volume fraction (BV/TV), trabecular number, thickness and separation. Any areas of osteolysis can also be noted. Phase contrast μCT allows the whole joint to be evaluated for cartilage volume and the underlying bone structure for resorption pits. The posterial portion is wrapped in saline soaked gauze and stored frozen for mechanical testing followed by biochemical analyses (GAG/DNA/HDP) of the cartilage surface. Mechanical testing can determine the equilibrium modulus under compressive strain of 1×1 mm plugs at least 1 mm from the cut surface. The tibial plateau and meniscal cartilage are also harvested and analyzed for degradation, fibrillation and changes in GAG/DNA/HDP.

Biomechanical analysis: Mechanical testing determines the equilibrium modulus under compressive strain of 1 mm diameter plugs at least 1 mm from the cut surface, non-operated knees acts as controls. In addition, samples are tested using custom designed ultrasound apparatus for shear and anisotropy.

Histological analysis: Formalin fixed, decalcified, paraffin embedded tissues are sectioned (8 μm) and stained. Stains for glycosaminoglycan, type I collagen, type II collagen, type X collagen, aggrecan and lubricin are performed of both treated and control sections. Histology sections are blinded and scored with an OARSI guided score assessment of thickness, matrix staining, surface regularity, integration and inflammation.

### Example 3. Disease Modifying Osteoarthritis Drug Discovery Using a Temporal Phenotypic Reporter in Human Chondrocytes

### Introduction

Osteoarthritis (OA) is the most prevalent articular joint disease worldwide. It affects approximately 16% of adult in the US, with approximately 32.5 million adults reporting OA between 2008 to 2014 (1). Current pharmaceutical therapies for OA are primarily palliative including non-steroidal anti-inflammatories, opioid analgesics, intra-articular corticosteroids and hyaluronan injections (2). Surgical intervention is the only current treatment that can restore at least partial function to the joint, however results are highly variable (3). There are currently no disease-modifying drugs available to treat OA, as such there is a dire need to identify novel regenerative pharmaceutical alternatives (2).

Cartilage degeneration is the primary pathological feature of osteoarthritis. Cartilage is composed of a dense extracellular matrix interspersed with chondrocytes. Chondrocytes synthesize a combination of glycosaminoglycans (GAG), proteoglycans and collagens that make up native cartilage. Type II collagen makes up 90-95% of all collagens found in articular cartilage, and its degradation is one of the early symptoms of OA (4-6). Furthermore advances in tissue engineering for the treatment of OA and post traumatic osteoarthritis (PTOA) have been hampered by the inability to recapitulate native levels of type II collagen in vitro (7). Previous work by our group has shown that increased type II collagen synthesis correlates with improved mechanical and biochemical properties in engineered tissue in vitro (8-10). In this study we propose using type II collagen expression as a chondrogenic phenotype for drug discovery.

Traditional methods to measure type II collagen, such as biochemical assays, protein assays, and real time qPCR are laborious, costly, time consuming, and destructive to samples (11). We have developed a phenotypic COL2A1-Gaussia luciferase primary human chondrocyte reporter (HuCol2gLuc) that allows for non-destructive temporal analysis of type II collagen production (12). In this work, the modified chondrocytes are used in a 3D aggregate model enabled for high throughput analysis. Published findings by our group have shown the success of this assay, by using it to identify vitamins and minerals that improve chondrogenesis in the murine chondrogenic cell line, ATDC5, and in primary rabbit chondrocytes in vitro (9, 13). This system has also been used to identify biomaterials that improve type II collagen production in human primary chondrocytes in 3D bioprinted constructs (12). In this study we used the phenotypic reporter system to identify new lead compounds, that can stimulate type II collagen production in primary human chondrocytes by screening the NCI natural products Set V library (14).

Natural Products are great source of potential new drugs and lead compounds. This class of compounds have large structural diversity and are enriched in bioactive compounds covering a wider chemical space compared to chemically synthesized compounds (15, 16). They have been a prolific source of leads in drug discovery, with several marketed drugs being or having been modified from natural products (17, 18). Despite their advantages, isolation, and characterization of natural products from their original source followed by lack of high throughput methodologies to screen natural products are primary drawbacks that have hampered natural product advancement in drug discovery (19, 20).

Using our 3D in vitro phenotypic reporter assay we have successfully screened the Natural Product Set V library from NCI. This library consists of 390 compounds selected from the 140,000 products from the DTP Open Repository collection. Compounds for this library were selected based on origin, purity (>90% by ELSD, major peak has correct mass ion), structural diversity and availability of compound. From the screen we have identified a hit for increased type II collagen production. Furthermore, we have identified a target for OA drug discovery.

### Results

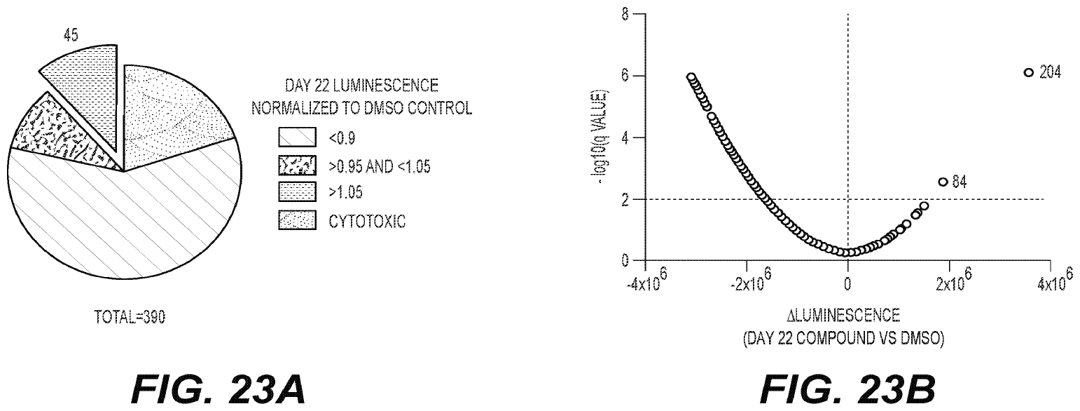

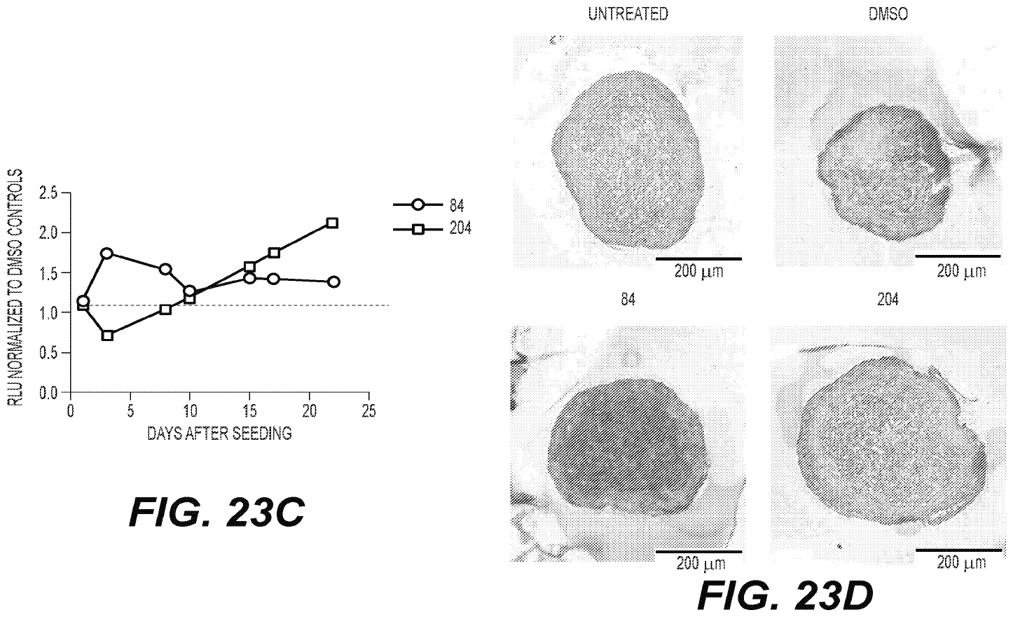

Initial screen of natural products library identified two hits that increased type II collagen synthesis in vitro. To screen 390 natural products from the NCI natural products library, primary human chondrocytes were modified to express a Gaussia luciferase secreted reporter driven by type II collagen promoter. HuCol2gLuc primary human chondrocytes have been previously characterized by our group as a proxy for type II collagen expression (12). Aggregate cultures of HuCol2gLuc cells were seeded in the presence of the 390 compounds from the library over 22 days in chondrogenic differentiation medium with 1 ng/ml of TGF-01. FIG. 23A shows overall results of treatment. Of the 390 compounds, 77 were cytotoxic to cells. These were identified as a failure to form aggregates after initial treatment. Of those that were not cytotoxic 230 compounds were detrimental to chondrogenesis, classified as less than 0.9 luminescence when normalized to the vehicle (DMSO) control aggregates on day 22. We identified 38 compounds had no effect on type II collagen expression and only 45 compounds were considered beneficial or as having over a 1.05 fold increase in luminescence when compared to controls. Of those 45, only two candidates, identified as candidate 84 (promoline) and 204 (deserpidine), had a significant increase in luminescence as compared to the controls by day 22, shown by the volcano plot in FIG. 23B. Luminescence over 22 days normalized to our DMSO control is shown in FIG. 23C. The overall trend of expression for both candidates differs over the 22 days. As seen in FIG. 23C aggregate sample treated with promoline shows an increase over the control as early as day 3 with a sustained increase in expression over 22 days. Alternatively, luminescence of aggregate treated with deserpidine shows an initial decrease in expression with continued increase in luminescence after day 8. Immunohistochemistry of sections collected from aggregates at day 22 shown in FIG. 23D confirm expression of type II collagen in HuCol2gLuc derived aggregates with substantially darker staining in the aggregate treated with promoline.

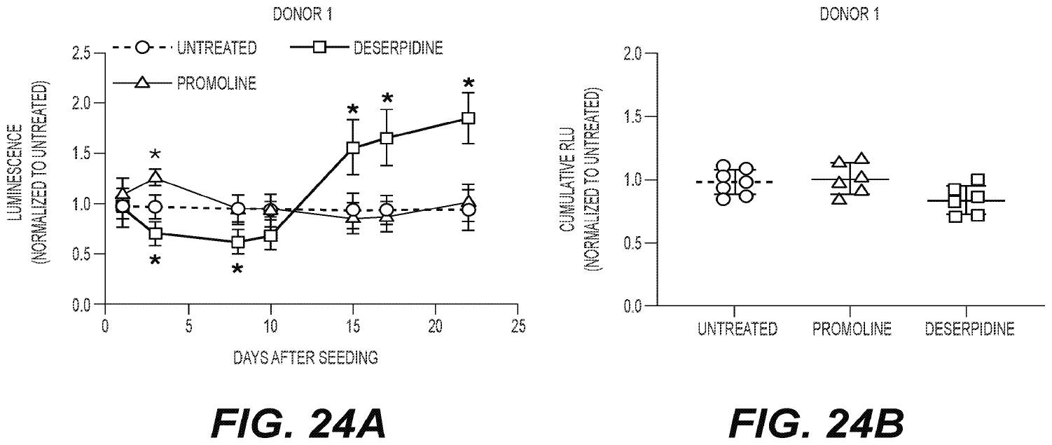

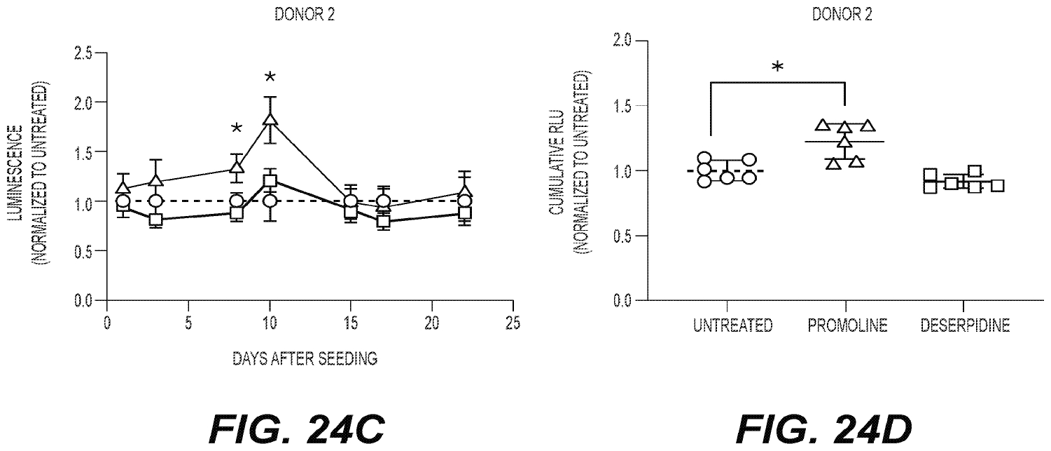

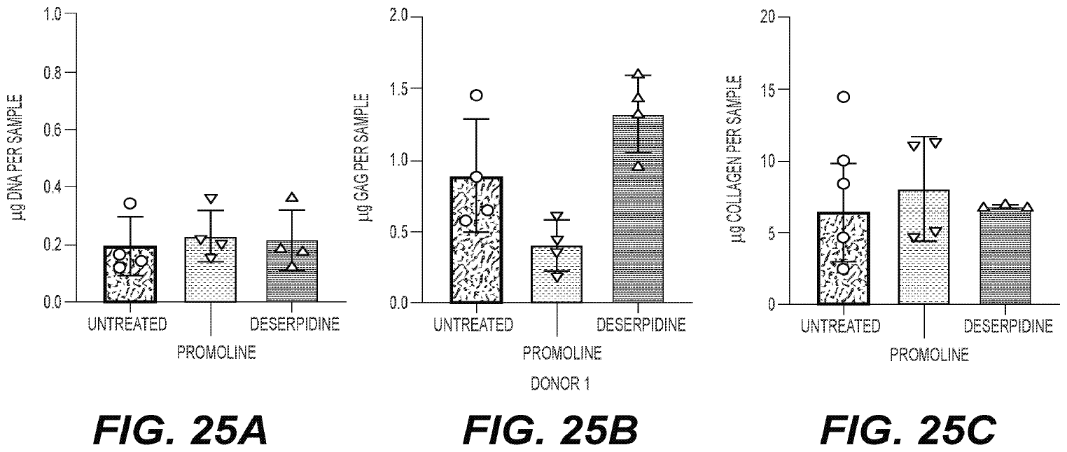

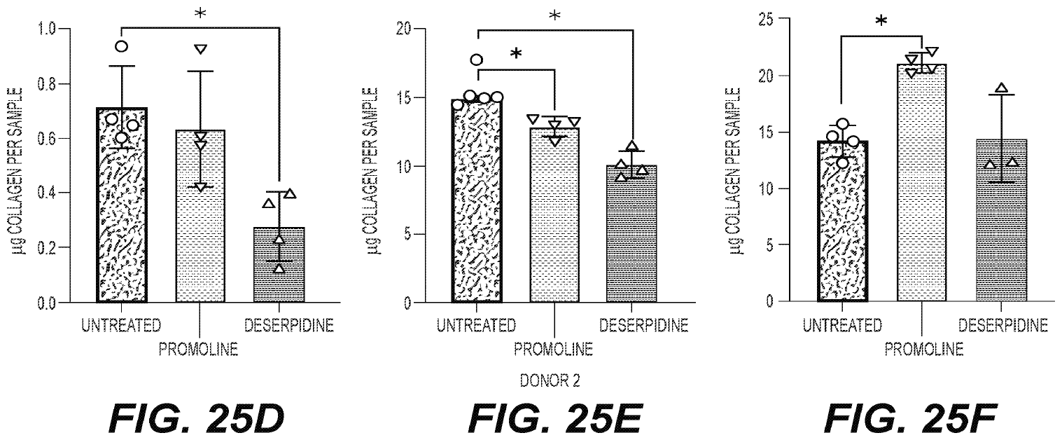

Validation of hits from the screen in two different cell donors verified promoline as a hit. To validate promoline and deserpidine from the drug screen, HuCol2gLuc cells, from two different donors, were treated with either promoline or deserpidine in aggregate culture for 22 days in chondrogenic differentiation medium supplemented with 10 ng/ml of TGF-□1. As seen in FIGS. 24A and 24C, deserpidine treatment resulted in an increase in luminescence in aggregates from donor 1 cells but not donor 2. Luminescence for deserpidine in donor 1 aggregates decreased from day 0 to day 10 with a significant increase from day 15 to day 22, similar to what was observed in the initial drug screen. In contrast, candidate 84 resulted in increased luminescence for donor 2 but not for donor 1, shown in FIG. 24B and FIG. 24D. The increase in luminescence for donor 2 aggregates treated with promoline compared to the untreated control occurred from day 0 to day 10 with no change as compared to the control from day 15 to day 22. This is also similar to the initial screen, where promoline had a peak in luminescence early in chondrogenesis versus late. However, donor 1 aggregates did not show this increase. Further confirmation of promoline effects are seen by cumulative luminescence in donor 2 aggregates. Contrary to FIG. 24A, deserpidine showed no significant increase in donor 1 for cumulative luminescence. As seen in FIG. 24D, promoline showed a significant increase in cumulative luminescence for donor 2 with no significant effect for donor 1. To corroborate luminescence results, biochemical assays to quantitate DNA, glycosaminoglycan (GAG) and total collagen content were performed at day 22 of the experiment. FIGS. 25A and 25D shows an average of ˜0.2 □g of DNA per donor 1 samples and ˜0.7 □g per donor 2 samples. There is no significant difference between drug treatments for donor 1. Treatment with promoline also had no change in DNA content for donor 2, however treatment with deserpidine showed a significant decrease in DNA content compared to the untreated control (FIG. 25D). This indicates that promoline has no effect on cell proliferation or viability over 22 days, while deserpidine seems to have a donor dependent effect on cell viability. Total glycosaminoglycan content is shown in FIGS. 25B and 25E. There seems to be a decrease in GAG content with promoline in donor 1 and 2 aggregates, although donor 1 results were not statistically significant. Deserpidine treatment had no effect on GAG for donor 1 but had a significant decrease in donor 2 to 10 μg, from 15 μg in the untreated controls, corroborating the donor dependent effects of deserpidine. Quantification of total collagen, as seen in FIGS. 25C and 25F shows no significant increase in total collagen with deserpidine treatment in both donor 1 and donor 2 aggregates as compared to the untreated control. Treatment with promoline resulted in a significant increase in donor 2 aggregate collagen, to ˜21 μg over untreated (˜14 μg), and no observable effect in donor 1. Furthermore, a substantial difference in the total DNA, GAG, and collagen content was noted between donor 1 and donor 2 regardless of treatment. Altogether aggregates from donor 1 cells seem to be less chondrogenic than aggregates from donor 2. Based on cumulative luminescence in donor 2 and total collagen content, promoline was identified as a top hit for the expression of type II collagen.

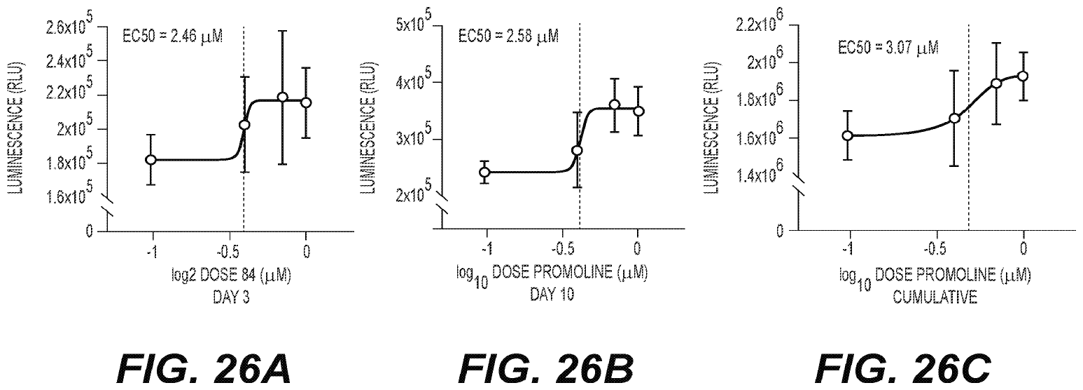

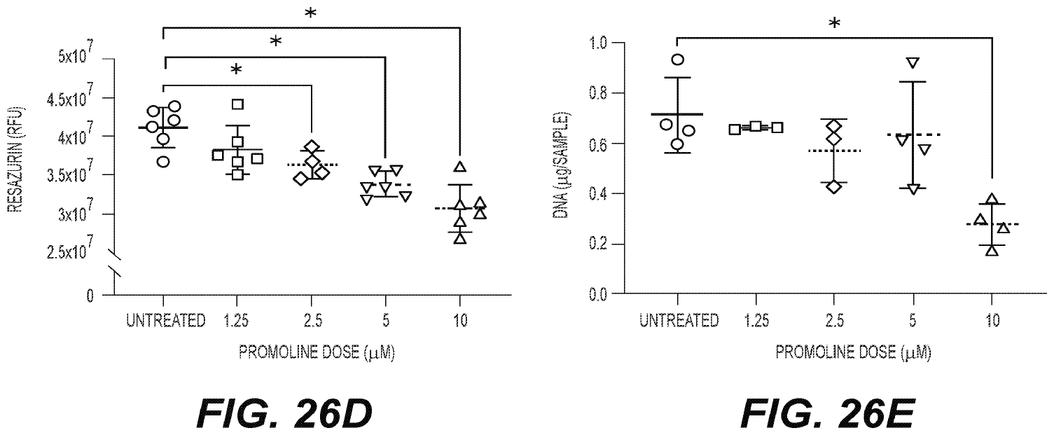

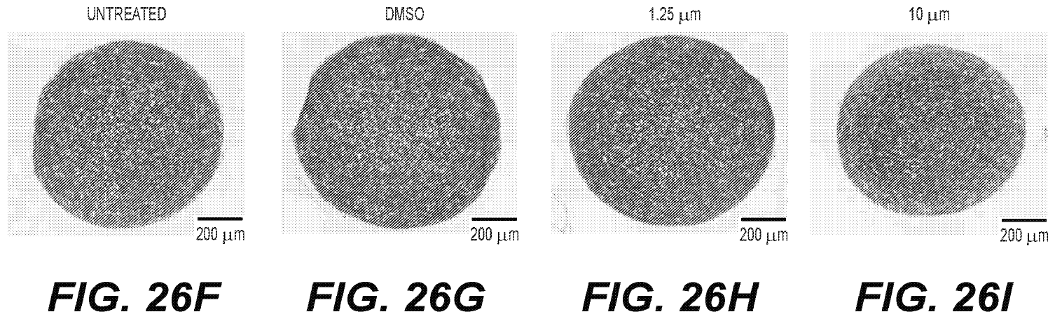

Promoline has a dose dependent effect on HuCol2gLuc aggregate type II collagen expression. To characterize the response of HuCol2gLuc cells, to treatment with promoline, cells were cultured in 3D aggregates in chondrogenic media treated with different concentrations of promoline (0-10 μM). Dose response curves were generated from luminescence data at day 3 (FIG. 26A), day 10 (FIG. 26B) and for cumulative luminescence (FIG. 26C). As seen in FIG. 4, there was a dose dependent increase in type II collagen driven-luminescence with a calculated. 50% effective concentration (EC50) of 2.46 μM, 2.58 μM, and 3.07 μM for day 3, 10 and cumulative luminescence respectively. To determine effects of promoline on viability and cell proliferation, resazurin assay and quantification of DNA content were carried out at day 22. FIG. 26D shows that there is a dose dependent decrease in resazurin fluorescence at day 22 suggestive of decreased metabolic activity due to promoline treatment. DNA quantification shows a similar trend with a significant decrease in aggregates treated with 10 μM promoline (FIG. 26E). This indicates that decreased metabolic activity is due to cytotoxicity at 10 μM promoline. Immunohistochemistry of sections collected from aggregates at day 22 shown in FIG. 26F-26I confirm expression of type II collagen in HuCol2gLuc derived aggregates with no notable differences between aggregates treated with these doses of promoline.

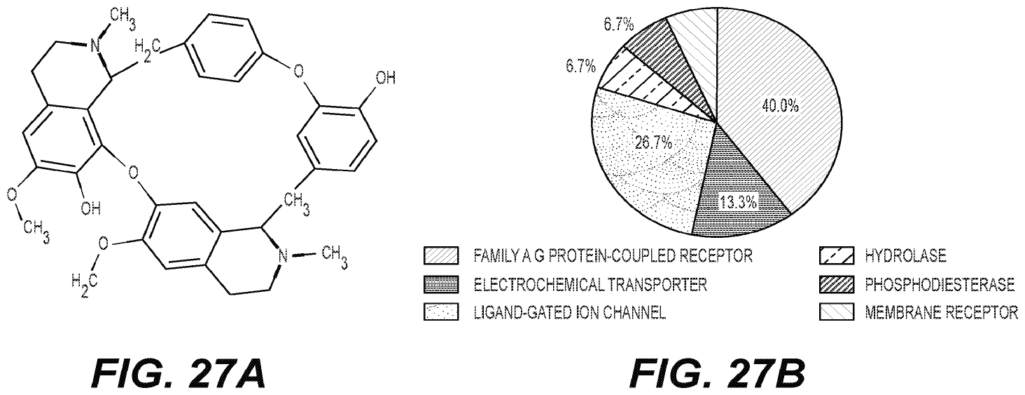

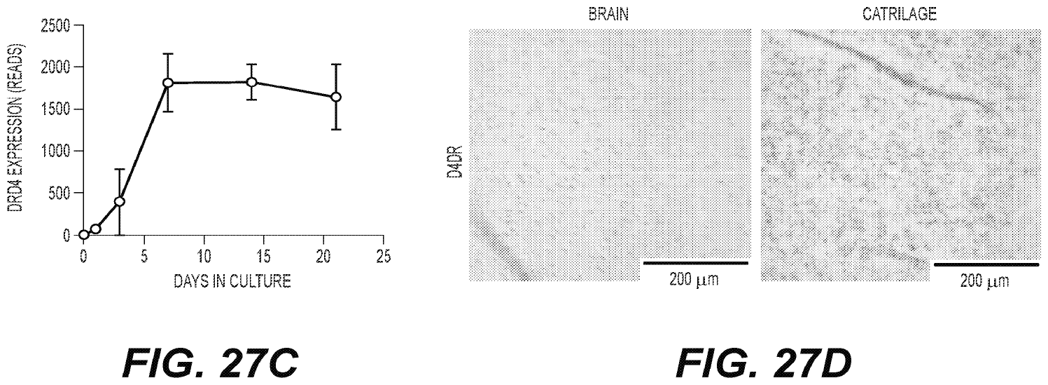

Characterization and target identification of promoline. To identify targets for promoline, this study used its chemical structure (FIG. 27A) for in silico analysis via SwissADME (21). Using the Swiss target prediction tool (22, 23) we determined that about 40% of the predicted targets are family A G-protein coupled receptors (FIG. 27B). By homology, the specific target with the highest probability was the Dopamine 2 receptor (DRD2). Previous studies showed expression of only the dopamine 4 receptor (DRD4), a DRD2-like receptor, in human chondrocytes (24). Another study looking mesenchymal stromal cell chondrogenesis also showed DRD4 as the only dopamine receptor expressed during chondrogenesis (FIG. 27C; (25)). Expression of DRD4, shown in (FIG. 27C) increases during chondrogenesis with a peak at around week 2, similar to the type II collagen pattern observed for aggregates treated with promoline (FIG. 24C). Furthermore, immunohistochemistry confirmed the expression of DRD4 in human cartilage tissue (FIG. 27D), with a different more punctate pattern of staining as compared to DRD4 staining in the mouse brain where dopamine receptors are widely expressed.

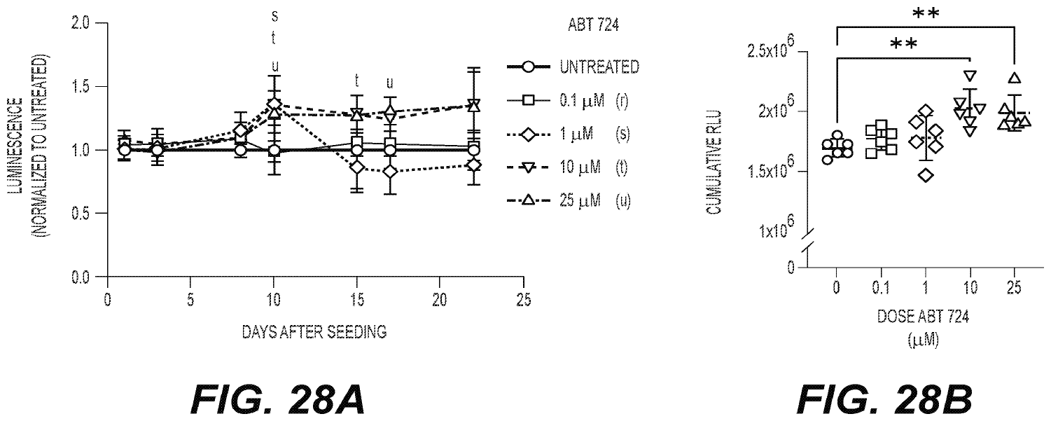

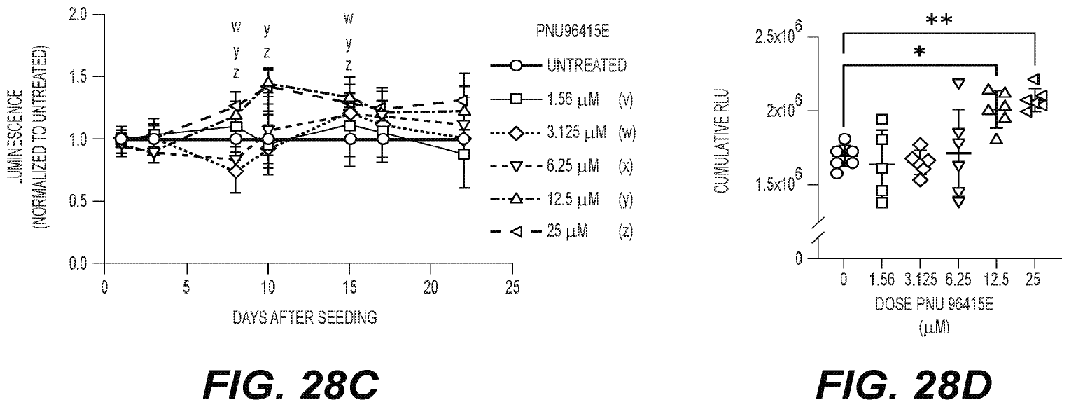

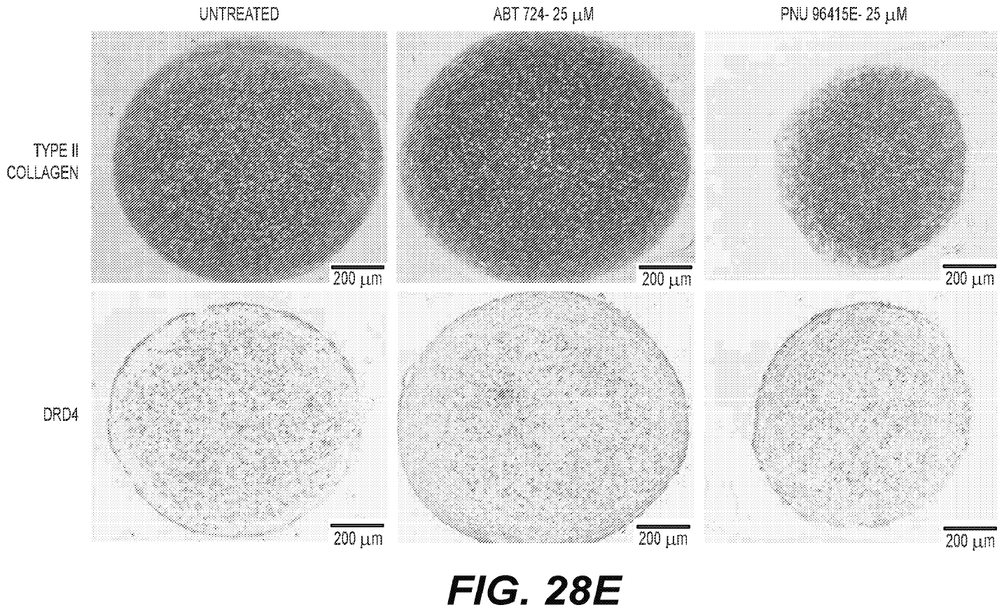

DRD4 selective antagonist and agonists have a dose dependent effect on HuCol2gLuc aggregate type II collagen expression. To determine whether DRD4 signaling affected type II collagen expression in human chondrocytes, HuCol2gLuc cells from donor 2 were cultured in 3D aggregates in the presence of a selective DRD4 agonist (ABT 724) or antagonist (PNU 96415E) at various concentrations (0-25 μM) and luminescence assessed over 22 days. As seen in FIG. 28A, ABT 724 treatment resulted in an increase in luminescence at day 8 at 10 μM and 25 μM concentrations with a sustained signal until day 22. ABT 724 had no effect at 0.1 μM or 1 μM. Cumulative luminescence (FIG. 28B) corroborates the results with concentrations of 10 μM and 25 μM resulting in a significant increase in luminescence as compared to the untreated controls. Unexpectedly, treatment with antagonist, PNU 96415E (FIGS. 28C and 28D) also had a positive effect on type II collagen expression with the higher concentrations, 12.5 μM and 25 μM, resulting in increased luminescence compared to the untreated controls. The pattern of expression over 22 days, seen in FIG. 28C, differed from treatment with ABT 724 (FIG. 28B), with PNU 96415E treatment resulting in peak expression by day 10 followed by a decrease at day 15 and sustained expression until day 22. Cumulative luminescence (FIG. 28D) corroborates the results with concentrations of 12.5 μM and 25 μM resulting in a significant increase in luminescence as compared to the untreated controls. Immunohistochemistry confirmed the expression of type II collagen in aggregates treated with ABT 724 and PNU 96415E. ABT 724 had more intense staining for type II collagen as compared to the untreated control, while PNU 96415E had diminished type II collagen staining as compared to the control. DRD4 immunohistochemistry similarly shows a substantial increase in staining for aggregates treated with ABT 724 as compared to the control (FIG. 28E). Whereas PNU 96415E had similar staining as compared to the untreated control (FIG. 28E) . . .

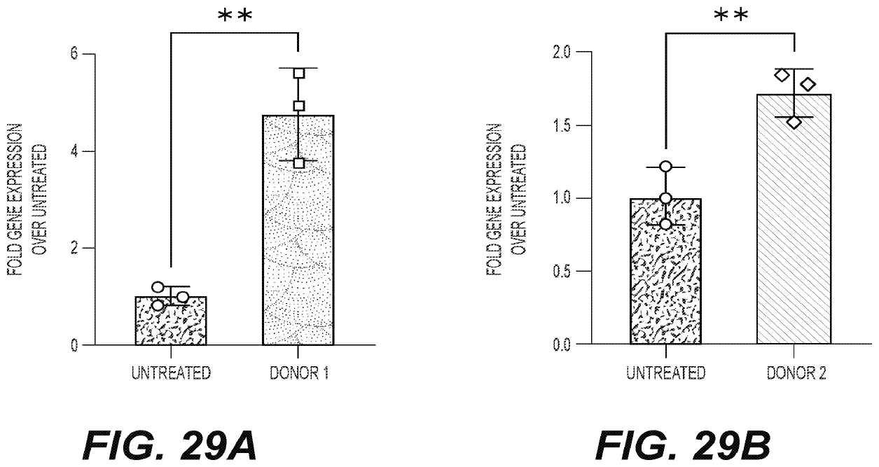

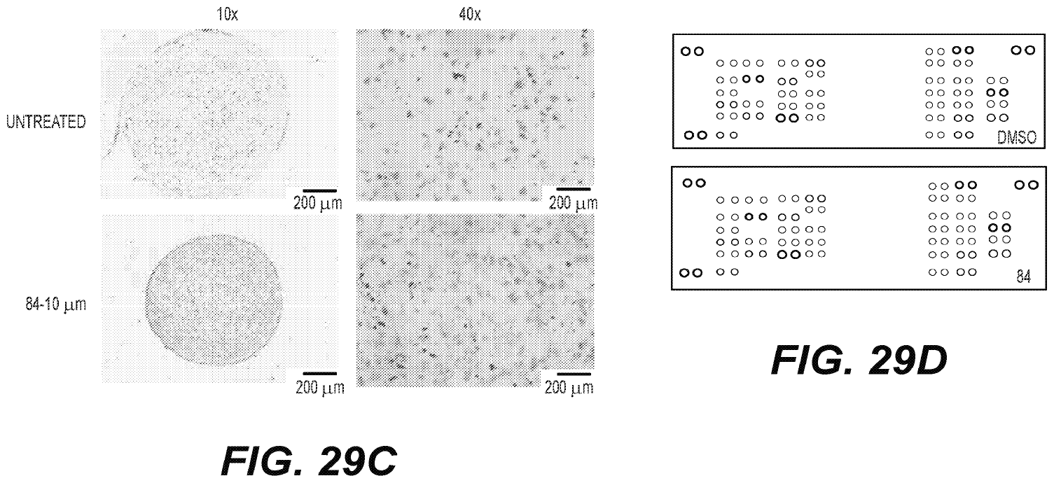

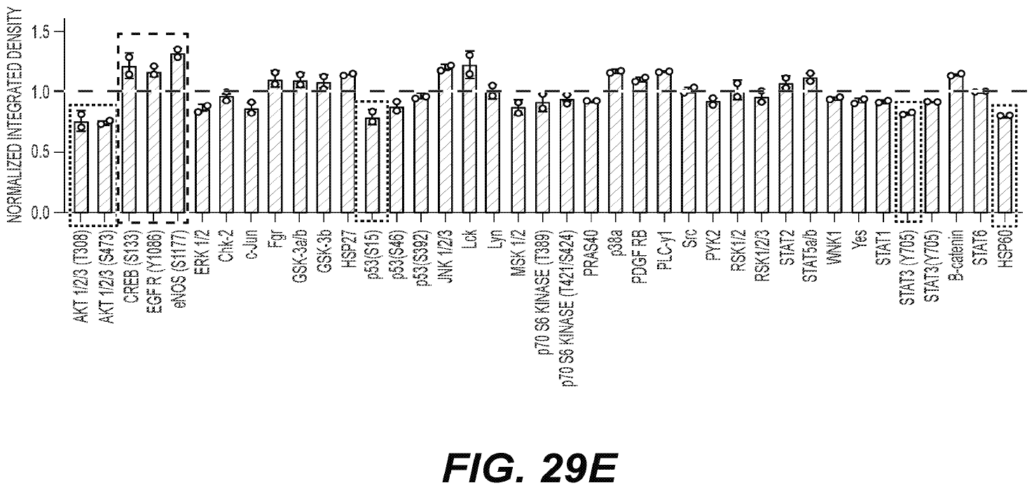

To assess whether promoline had an effect on DRD4 expression in human chondrocytes, monolayer culture of human chondrocytes from donor 1 and donor 2 were treated with drug promoline (5 μM) for 24 h followed by RNA extraction and analysis via qPCR. As seen in FIGS. 29A and 29B, treatment with promoline resulted in significant upregulation of DRD4 in both donor 1 and donor 2. Donor 1 had overall higher DRD4 expression as compared to the untreated control, approximately 5-fold more, while donor 2 had only about 1.8-fold higher expression. Histology of donor 2 HuCol2gLuc aggregates treated with promoline (10 μM) at day 22 shown in FIG. 29C confirm increased expression of DRD4 expression compared to untreated controls. To determine what signaling pathways could be involved in promoline mediated increase of type II collagen, donor 2 primary chondrocytes were treated with promoline in monolayer culture for 24 h, lysate collected, and a phospho-kinase array was used to look at levels of phosphorylation for 17 different kinases (FIG. 29D). Data normalized to reference spots and to DMSO controls is shown in FIG. 29E. There is a substantial decrease in AKT 1/2/3 phosphorylation at both T308 and S473 phosphorylation sites (FIG. 29E). A less considerable decrease in p53 phosphorylation at the S15 site, in STAT3 phosphorylation at Y705, and in HSP 60 was also observed (FIG. 29E). Also observed was a modest increase in phosphorylation of CREB, EGFR, and eNOS (FIG. 29E).

Using our high throughput model of COL2A1-Gaussia luciferase primary human chondrocyte reporter (HuCol2gLuc) in 3D aggregates under physioxia we have successfully screened the NCI Natural product library. Our system has several advantages over other proposed models. It makes use of human primary cells over non-human cells or immortalized cell lines. While chondrocytes are generally limited in availability and could be a barrier for high throughput screening, our use of porcine derived synoviocyte matrix for expansion preserves chondrogenic capacity for more cell divisions and allowed for greater expansion of primary chondrocytes (26, 27). Our system tests compounds in 3D aggregates while other chondrogenic models for drug discovery typically rely on 2D culture (28-30). 2-dimensional culture for many cell types, including chondrocytes, fail to mimic cell-cell, cell to matrix interactions, and paracrine signaling events that are responsible for physiological tissue structure (31, 32). Furthermore, 2D culture cannot correctly model events, such as ECM sequestering of soluble compounds, which can have a large effect on drug-cell interaction (32-34). Overall 3D models better mimic native tissue. The primary advantage of our reporter model is the use of a secreted reporter which not only allows us to quantitate the signal as opposed to other read out methods such as staining and microscopy imaging but also allows for temporal non-destructive assessment of the phenotype during chondrogenesis (9).

From the natural product screen, we identified several candidate drugs that increased type II collagen over controls. Of these, promoline and deserpidine had the most pronounced effect. In a follow up experiment promoline presented as the better candidate while deserpidine had inconclusive results. Promoline, also known as aromoline, is a bisbenzylisoquinoline alkaloid generally derived from members of the Berberis and Stephania genus (35, 36). As their name indicates, and as seen in FIG. 27A, these alkaloids consist of two benzylisoquinoline parts linked through either diphenyl ether, benzyl phenyl ether, or biphenyl bonds (36, 37). Previous screening of alkaloids from Berberis have shown that promoline is a potent inhibitor of butyrylcholinesterase with low blood brain barrier permeability (38). Another study showed weak anti-microbial activity against Plasmodium falciparum with low cytotoxicity in the KB cell line (39). Although there is little published research on promoline, various bisbenzylisoquinoline alkaloids are being studied for their anti-proliferative, anti-inflammatory and anti-microbial properties (40-45). This is the first study to our knowledge that explores the effects of promoline on chondrocytes and chondrogenesis.

With so little known about the signaling pathways activated by promoline, in silico analysis provided an unexpected initial candidate target: the dopamine 2 receptor. Dopamine receptors are G-coupled receptors widely expressed through the central nervous system (46, 47). Expression in the periphery has been observed in immune cells such as neutrophils, basophils, B cells and K cells, as well as the, the heart, kidneys, adrenal glands, blood vessels, and gastrointestinal tract (48-56). Dopamine receptors are classified under two groups the D1-like family that is coupled to Gs and activates adenylyl cyclase, and the D2-like family that is coupled to G□ and inhibits adenyl cyclase (57-59). Based on the in silico analysis, our previous RNA-seq and that of Huynh et al. (FIG. 27C) as well as the fact that dopamine receptors from the same family share significant homology, we hypothesized that Dopamine 4 Receptor (DRD4), one of the members of the D2-like receptors was a likely target for promoline (24, 25, 47). This is the first study to our knowledge that has identified the expression of DRD4, by qPCR and by immunohistochemistry, in primary human chondrocytes and cartilage. Furthermore, we confirmed upregulation in expression of DRD4 and type II collagen after treatment with promoline (FIG. 29A-29D).

Treatment of HuCol2gLuc aggregates with either a selective agonist or antagonist for DRD4 resulted in increased type II collagen expression. These results indicates that DRD4 signaling through Giα and subsequent inhibition of adenylyl cyclase is not the mechanism of action for promoline (60). Moreover, promoline treatment led to a substantial decrease in Akt phosphorylation (FIG. 29E). These observations provide clues as to the mechanism of action for promoline but fail to completely elucidate the pathways involved. Promoline treatment in chondrocytes results in both upregulation of DRD4 expression and decrease in phosphorylation of Akt. A dopamine receptor Giα independent pathway, the β-arrestin 2 pathway, would account for the observed effects of promoline. The β-arrestin 2 pathway is activated as a mechanism for dopamine receptor internalization and desensitization (61). Formation of this complex results in inactivation of Akt by protein phosphatase 2 and activation of the glycogen synthase kinase-3 (GSK3) signaling (62). GSK3 has been linked to maintenance of the chondrocyte phenotype. Inhibition of GSK3 results in cartilage destruction and progression of chondrocytes to terminal differentiation (63-66). Previous studies on dopamine receptors, as well as other GPCRs, have shown that ligand activation of β-arrestin 2 is distinct from its G protein dependent activity (67-71). These findings could offer an explanation for our results with DRD4 agonist and antagonist treatment. Further studies to unravel the signaling pathways involved in promoline mediated type II collagen expression as well as DRD4 involvement in chondrogenesis are needed. Other areas of study include the identification of the ligand for DRD4 in chondrocytes and how this endogenous ligand signals in the cells.

While a similar trend was evident with both donors, there was a significant difference in the extent of their response. This could be an interesting avenue for further, patient specific, tailored care. What is encouraging is the expression of DRD4 in cartilage in three separate studies, this research, the previous RNA-Seq data from our group (24) and in the work by Huynh et al. (25). There are multiple steps that need to be completed before translation to the clinic, confirmation in more donor chondrocytes, with non-modified chondrocytes, in an animal model(s).

This study not only provides novel insights into the complex process of chondrogenesis but identifies a new target receptor and drug candidate for the treatment of osteoarthritis.

To identify novel, chondrogenesis promoting drugs, human primary chondrocytes were genetically modified to express a secreted luciferase reporter under the control of the type II collagen promoter. Those cells were then used in a 3D culture system under physiological oxygen tension to screen a natural product library for promotion of the articular cartilage phenotypic marker, type II collagen. Identified compounds were further screened using in silico reference data to identify drug targets. The dopamine receptor D4 was identified and investigated as a target with significant effects on type II collagen expression.

### Methods

Human primary chondrocyte isolation. Articular cartilage was isolated from visually intact areas of discarded surgical tissue as previously described Tissue was collected from two donors during total joint re-placement surgery with informed consent under IRB approved protocols (Baylor College of Medicine, H-36683, H-36374). Discarded tissue (<3 h post-operation on ice in saline) was briefly stored in defined chondrogenic media (˜4 h, room temperature) before cartilage was dissected under sterile conditions. Isolated cartilage was diced into <1 mm³ pieces before digestion, first in hyaluronidase for 30 min (660 Units/ml Sigma, H3506; in DMEM/F12, 40 ml), then by collagenase type II for ˜16 hours at 37° C. (583 Units/ml Worthington Biochemical Corp.; in DMEM/F12 with 10% FBS, 40 ml). The digest was filtered through a 70 μm cell strainer, washed once with DMEM/F12, and resuspended in growth media (DMEM/F12 supplemented with 10% FBS (mesenchymal stromal cell selected), 1% pen/strep). Cells were subsequently infected as described below or cryopreserved (95% FBS, 5% DMSO).

Lentiviral construct. Lentivirus was generated as previously described. Briefly, custom COL2A1-Gaussia Luciferase plasmid (HPRM22364-LvPG02, GeneCopoeia, Inc.), envelope (pMD2.G) and packaging (psPAX2) plasmids were amplified in Escherichia coli (GCI-L3, GeneCopoeia) and purified via silica column system (Qiagen Maxiprep) before co-transfection into HEK293Ta (GeneCopoeia) cells via calcium chloride precipitation. Newly packaged lentiviral particles were collected in culture medium after 48 h and concentrated via ultracentrifugation (10,000 RCF, 4° C., overnight). Titers for COL2A1-Gluc lentivirus were estimated via real-time PCR and aliquots stored at −80° C.

Lentivirus infection of primary Human Chondrocytes. Isolated primary human chondrocytes, from each donor, were seeded at 6,100 cells/cm² in growth media (DMEM and allowed to adhere overnight (˜20% confluency). Cells were infected with lentivirus (COL2A1-GLuc; MOI 25 in growth media) in the presence of 4 μg/ml polybrene (Opti-mem, Gibco) for 15 minutes at 4° C. followed by overnight incubation at 37° C. Lentiviral medium was replaced with growth medium and cells expanded to ˜70-90% confluency. Cells were trypsinized (trypsin/EDTA 0.25%), then seeded on synoviocyte matrix coated flasks. Primary chondrocytes infection was done in physioxic (37° C., 5% 02, 5% CO₂) conditions. Newly generated COL2A1-GLuc cells were cryopreserved at the end of this first passage (95% FBS, 5% DMSO). These cells were used for all subsequent studies.

Chondrogenic Culture. Human COL2A1-GLuc or uninfected cells were thawed and seeded in growth media on synoviocyte matrix at 6,000 cells/cm² and expanded to 90-100% confluence in physioxia. Cells were trypsinized (0.25% Trypsin/EDTA; Corning), resuspended in growth media (for monolayer culture) or chondrogenic differentiation media for 3D aggregates (93.24% High-Glucose DMEM (Gibco), 1% dexamethasone 10⁻⁵M (Sigma), 1% ITS+premix (Becton-Dickinson), 1% Glutamax (Hyclone), 1% 100 mM Sodium Pyruvate (Hyclone), 1% MEM Non-Essential Amino Acids (Hyclone), 0.26% L-Ascorbic Acid Phosphate 50 mM (Wako), 0.5% Fungizone (Life Technologies) with TGF-β1 (Peprotech) and seeded as described below.

Generation and Maintenance of 3D aggregates. To generate 3D aggregates, cells were seeded at 50,000 cells per well (in 96-well cell repellent u-bottom plates, GreinerBio) and then centrifuged at 500 RCF, 5 min. For the initial drug screen, aggregates were cultured in basal chondrogenic media (1 ng/ml TGF-β1) alone, with DMSO (vehicle control) or 5 μM of the drug compounds from the NCI library (Natural Products V, NCI). For candidate validation and subsequent studies aggregates were cultured in basal chondrogenic media (10 ng/ml TGF-β1) alone, with DMSO (vehicle control) or compounds as indicated in the results. Aggregates were cultured for three weeks in physioxic conditions, culture medium was sampled and replaced three times a week with fresh medium. An OT-2 (Opentrons) python coded robotic pipette, programmed at an aspiration height of 2 mm from the bottom of the wells and aspiration rate of 40 μl/s was utilized for addition of compound, cell feeding, and media sampling for luciferase assay. After three weeks, cell aggregates were either fixed in neutral buffered formalin for histology or medium removed and aggregates frozen dry (−20° C.) for biochemical assays.

Luciferase Assay. Conditioned culture medium sampled from aggregates in 96-wells (20 μL/well) was assessed using a stabilized Gaussia Luciferase buffer mix (50 □l/well) for a final concentration of 0.09 M MES, 0.15M Ascorbic Acid, and 4.2 μM Coelenterazine in white 96-well plates. Luminescence was measured in a plate reader (25° C., relative light units (RLU), En Vision plate reader). An OT-2 (Opentrons) python coded robotic pipette was utilized for luciferase buffer addition to white plates (GreinerBio).

Metabolic Assay (Resazurin). Metabolic activity was assessed on day 22 by adding resazurin to a final concentration of 50 μM to each well and incubating at physioxia for 3 hours. After three hours, media (120 μl) was transferred to a 96-well black plate (Greinerbio) and fluorescence read at an excitation of 535 nm and emission at 588 nm. At OT-2 (Opentrons) python-coded robotic pipette was used to add resazurin to cell plates as well as to transfer medium to black plates.

Immunohistochemistry. At the end of three-week culture, aggregates were fixed in 10% Neutral Buffered Formalin overnight, and subsequently embedded in paraffin wax and sectioned (7 μm sections). Sections were deparaffinized and hydrated, followed by treatment with pronase (1 mg/ml, Sigma P5147, in PBS with 5 mM CaCl₂)) for epitope retrieval. Sections were blocked (BSA 3% w/v, Cohn Fraction V Alfa Aesar) and incubated with mouse primary anti-Collagen Type II (DSHB II-II6B3) or mouse anti-dopamine 4 receptor (MAB125, Millipore) primary for 2 h at room temperature in a humidified chamber. II-II6B3 was deposited to the DSHB by Linsenmayer, T. F. (DSHB Hybridoma Product II-II6B3). Sections were then incubated with a biotinylated secondary followed Streptavidin-HRP (BD Biosciences). Sections were stained with a chromogen-based substrate kit (Vector labs, VIP substrate vector kit). All sections were imaged using a Keyence BZ-X810 microscope at 10× magnification.

Biochemical Assays. Aggregates frozen at day 22, were thawed with PBS, and digested with Papain (25 μg/ml, Sigma, P4762, in 2 mM cysteine; 50 mM sodium phosphate; 2 mM EDTA at a pH 6.5, 100 μl) at 65° C. overnight. Plates were sealed with a qPCR adhesive sealing film (USA Scientific), a silicone sheet, and steel plates clamped to the plate to prevent evaporation during digestion. Half of the digest (50 μl) was transferred to another plate and frozen for hydroxyproline assessment. In the remaining digest, papain was inactivated with 0.1M sodium hydroxide (NaOH, 50 μl) followed by NaOH neutralization (100 mM Na2HPO4, 0.1 N HCL, pH 1.82, 50 μl). To quantitate DNA content, samples of the neutralized digest (20 μl) were combined with buffered Hoechst dye (#33258, 667 ng/ml, phosphate buffer pH 8, 100 μl) and fluorescence measured at an excitation of 365 nm and emission of 460 nm. For GAG assessment, 1,9-Dimethyl-methylene blue solution (195 μl) was added to neutralized digests (5 μl) and absorbance measured at 595 nm and 525 nm. Readings were corrected by subtracting 595 nm reading from 525 nm. Micrograms of DNA and GAG were calculated using a Calf thymus DNA standard (Sigma) or Chondroitin Sulfate standard (Seikagaku Corp.), respectively.

For hydroxyproline (HP) quantification, the frozen digests (50 μl) were thawed at room temperature and incubated overnight at 105° C. with 6M hydrochloric acid (200 μl). Plates were sealed to prevent evaporation. Samples were then dried at 70° C. overnight with a hydroxyproline standard (Sigma). Copper sulfate (0.15 M, 10 μl) and NaOH (2.5 M, 10 μl) were added to each well and incubated at 50° C. for 5 minutes, followed by hydrogen peroxide (6%, 10 μl) for 10 minutes. Sulfuric acid (1.5 M, 40 μl) and Ehrlich's reagent (20 μl) were added and samples incubated at 70° C. for 15 minutes. Absorbance was measured at 505 nm. Micrograms of hydroxyproline were calculated using the standard. Total collagen was calculated by the following formula (μg of HP×7.6=μg Total Collagen).

Reverse Transcription and Real-Time PCR. Primary human chondrocytes were seeded at 280,000 cells/well (12-well adherent plate, Corning) in growth media and allowed to adhere overnight. Growth media was replaced with chondrogenic media supplemented with 1 ng/ml TGFβ1 alone or with drug candidate promoline. Cells were treated for 24 h and then lysed and frozen. Lysate was thawed and RNA extraction and purification done following manufacturer's protocol (Ambion PureLink RNA Mini kit). RNA purity and integrity was assessed by RNA ScreenTape (Agilent Technologies) before use. cDNA was synthesized from 400 ng RNA using a Maxima H Minus reverse transcriptase master mix following manufacturer's protocol. Quantitative real-time PCR for DRD4 and HPRT (endogenous control) gene expression was done (qPCR) using SYBR green master mix (Applied Biosystems) and QuantStudio7 Flex Real-Time PCR system (ThermoFisher Scientific). Cycling parameters: 95° C. for 20 s then 45 cycles of 95° C. 10 s, 60° C. 20 s, 72° C. 19 s, followed by melt curve analysis. Primers: HRPT, forward primer: 5′ ATTGACACTGGCAAAACAATGC 3′, reverse primer: 5′ TCCAACACTTCGTGGGGTCC 3′ (SEQ ID NO: 10). DRD4, forward primer: 5′ GTGGTGGTCGGGGCCTT 3′ (SEQ ID NO: 15), reverse primer: 5′ CGGAGCAGGCAGGACAC 3′ (SEQ ID NO: 16). DRD4 CT values were normalized to HRPT expression and DRD4 relative gene expression vs untreated calculated.

Phospho-kinase Array. Primary human chondrocytes were seeded at 5×10⁶ cells (2×10 cm adherent dish, Corning) in growth media and allowed to adhere overnight. Growth media was replaced with chondrogenic media supplemented with 1 ng/mL TGFβ1 with DMSO or with drug proline. Cells were treated for 24 h before processing according to the manufacturer's protocol (ARY003C, R&D Systems). This phospho-kinase array contains capture antibodies to measure relative levels of phosphorylation of 37 kinases on a nitrocellulose membrane. Signal intensity was quantified using ImageJ.

### Example 4. Micronutrient Optimization Using Design of Experiments Approach in Tissue Engineered Articular Cartilage for Production of Type II Collagen

Tissue Engineering of cartilage has been hampered by the inability of engineered tissue to express native levels of type II collagen in vitro. Inadequate levels of type II collagen are, in part, due to a failure to recapitulate the physiological environment in culture. In this study, primary rabbit chondrocytes were engineered to express a secreted reporter, Gaussia Luciferase, driven by the type II collagen promoter, and applied a Design of Experiments approach to assess chondrogenic differentiation in micronutrient-supplemented medium. Using a Response Surface Model, 240 combinations of micronutrients absent in standard chondrogenic differentiation medium, were screened and assessed for type II collagen expression. Five conditions predicted to produce the greatest Luciferase expression were selected for further study. Validation of these conditions in 3D aggregates identified an optimal condition for type II collagen expression. Engineered cartilage grown in this condition, showed a 170% increase in type II collagen expression (Day 22 Luminescence) and in Young's tensile modulus compared to engineered cartilage in basal media alone. Collagen cross-linking analysis confirmed formation of type II-type: II collagen and type II-type: IX collagen cross-linked heteropolymeric fibrils, characteristic of mature native cartilage. Combining a Design of Experiments approach and secreted reporter cells in 3D aggregate culture enabled a high-throughput platform that can be used to identify more optimal physiological culture parameters for chondrogenesis.

### Introduction

Osteoarthritis (OA) is the most common degenerative musculoskeletal disease and is projected to increase in prevalence. OA is characterized by progressive degeneration of articular cartilage in the joints of the hands, knees, and hip due to an imbalance of cartilage anabolism and catabolism. Articular cartilage is a form of specialized connective tissue, primarily composed of type II collagen, water, and proteoglycans with sparsely distributed chondrocytes. Cartilage has limited healing and regenerative abilities given that its avascular nature limits access to circulating progenitor cells following physical insult. Currently, there are no disease-modifying treatments for OA. Available therapeutics offer short-lived relief of acute symptoms and do not prevent endpoint joint damage, therefore there is a strong need for the development of new disease-modifying therapeutics.

Tissue engineering of cartilage has the potential to revolutionize the field by providing improved in vitro models for drug discovery and/or a biological replacement. Tissue engineering incorporates the use of components such as cells, scaffolds, growth factors, and physical stimulation to generate biomimetic tissue. However, tissue engineering of cartilage has been hampered by an inability to recapitulate the properties of native cartilage tissue, which can be due to insufficient type II collagen production. Whereas 90-95% of collagen in native tissue is type II collagen, several studies have reported much lower type II collagen levels in engineered tissue with values hovering around 20% despite modifications to increase collagen deposition. Deficiency in type II collagen can be due to sub-optimal formulation of the culture medium used for cartilage engineering in vitro.

Differentiation media traditionally used for chondrocyte cell culture was noted to lack several micronutrients which are known to be physiologically essential for a host of biological processes. Although the specific roles that some of these micronutrients have in chondrogenesis remain undefined, there are several findings that point towards these biomolecules having significant effects on cartilage generation and maintenance. This indicates the addition of these vitamins and minerals to basal differentiation medium can promote type II collagen production in vitro and better mimic the physiological environment.

A Design of Experiments (DoE) approach was implemented in this study to screen different combinations of vitamins and minerals. DoE is a statistical technique that facilitates systematic optimization by producing experimental design models to study interactions of multiple factors on a desired outcome or response. DoE allows for a multi-factor, rather than a one-factor approach, that evaluates synergistic effects, and can predict optimal conditions while reducing the burden of conducting repetitive experiments. DoE has provided significant benefits to other fields of engineering and biotechnology but has rarely been used in cartilage tissue engineering and regenerative medicine.

This study for the first time identified an optimal supplementation of physiologically necessary micronutrients to chondrogenic media, using a streamlined platform that includes a type II collagen promoter-driven Gaussia luciferase construct in primary rabbit articular chondrocytes combined with a DoE approach. This optimized chondrogenic media significantly enhances type II collagen expression in primary rabbit chondrocytes cultured in 3D cell aggregates and engineered cartilage sheets.

### Results

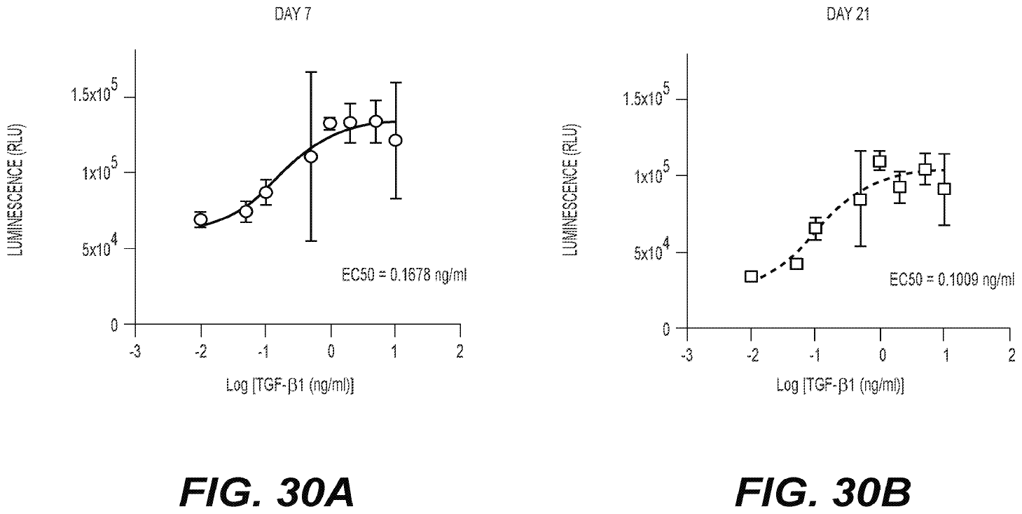

Stimulation of type II collagen by TGF-β1 in primary rabbit chondrocytes. To characterize the TGF-β1 response of engineered type II collagen promoter-driven Gaussia luciferase reporter (COL2A1-Gluc) in primary rabbit chondrocytes, cells were cultured in 3D aggregates in defined chondrogenic media supplemented with 0-10 ng/ml of TGF-β1, a known stimulator of type II collagen. Conditioned media, containing the secreted Gaussia luciferase, was assayed for luminescence over three weeks. Dose response curves were generated from luminescence data at Day 7 (FIG. 30a) and Day 21 (FIG. 30b). As seen in FIG. 30, there was a dose dependent increase in luminescence with a calculated 50% effective concentration (EC50) of 0.17 ng/ml and 0.10 ng/ml for Day 7 and Day 21 respectively.

Response Surface Model and subsequent ANOVA analysis identified interactions between micronutrients that increased type II collagen promoter-driven expression of Gaussia luciferase. To identify potential interactions between factors and their effect on type II collagen expression, COL2-GLuc rabbit chondrocytes were seeded in 3D aggregate culture with DoE generated combinations of vitamins and minerals; media was sampled and replaced over three weeks. Combinations and concentrations are defined by the parameters set in the response surface model (Table 3) and Table 5.

TABLE 3
Concentrations of micronutrients absent in chondrogenic media with input
parameters for DoE screen.

| | | | | Maximal Serum | Minimum | Maximum |
|---|---|---|---|---|---|---|
| Term | Name | Abbreviation | Manufacturer | Concentration¹ | (μg/ml) | |
| A | Linolenic Acid | ALA | Sigma-Aldrich | 54 mg/L | 1.0E−15 | 270.43 |
| B | Chromium | Cr | Sigma-Aldrich | 28 μg/L | 1.0E−15 | 0.14 |
| C | Cobalt | Co | Sigma-Aldrich | 0.9 μg/L | 1.0E−15 | 4.52E−03 |
| D | Copper | Cu | Sigma-Aldrich | 670 μg/L | 1.0E−15 | 3.35 |
| E | lodine | I | Alfa-Aesar | 92 μg/L | 1.0E−15 | 0.46 |
| F | Manganese | Mn | Sigma-Aldrich | 12 μg/L | 1.0E−15 | 0.06 |
| G | Molybdenum | Mo | Sigma-Aldrich | 2.0 μg/L | 1.0E−15 | 0.01 |
| H | Thyroxine | T4 | Sigma-Aldrich | 17 ng/L | 1.0E−15 | 0.12 |
| J | Vitamin A | Vit A | Alfa-Aesar | 780 μg/L | 1.0E−15 | 0.08 |
| K | Vitamin B12 | Vit B12 | Sigma-Aldrich | 914 ng/L | 1.0E−15 | 4.56E−03 |
| L | Biotin | Vit B7 | Alfa-Aesar | 3004 ng/L | 1.0E−15 | 0.01 |
| M | Vitamin D | Vit D | Selleck Chemicals | 86 ng/L | 1.0E−15 | 4.31E−04 |
| N | Vitamin E | Vit E | Sigma-Aldrich | 18.4 mg/L | 1.0E−15 | 91.84 |
| O | Vitamin K | Vit K | Sigma-Aldrich | 2.2 μg/L | 1.0E−15 | 0.01 |
| P | Zinc | Zn | Alfa-Aesar | 1.2 mg/L | 1.0E−15 | 5.99 |

¹Note:
References for serum concentrations: 7, 10-11, 13-15, 40, 41, 43 62-71

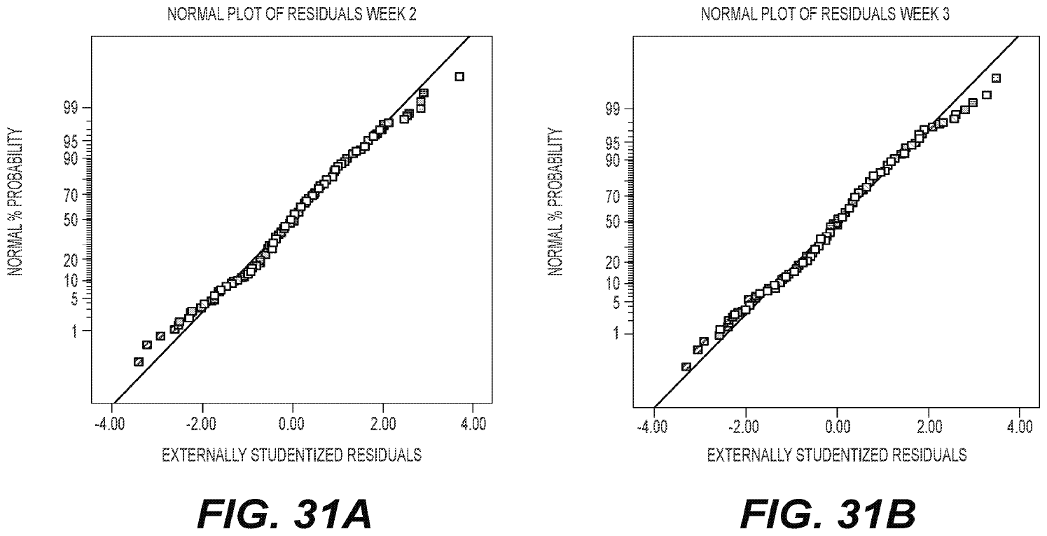

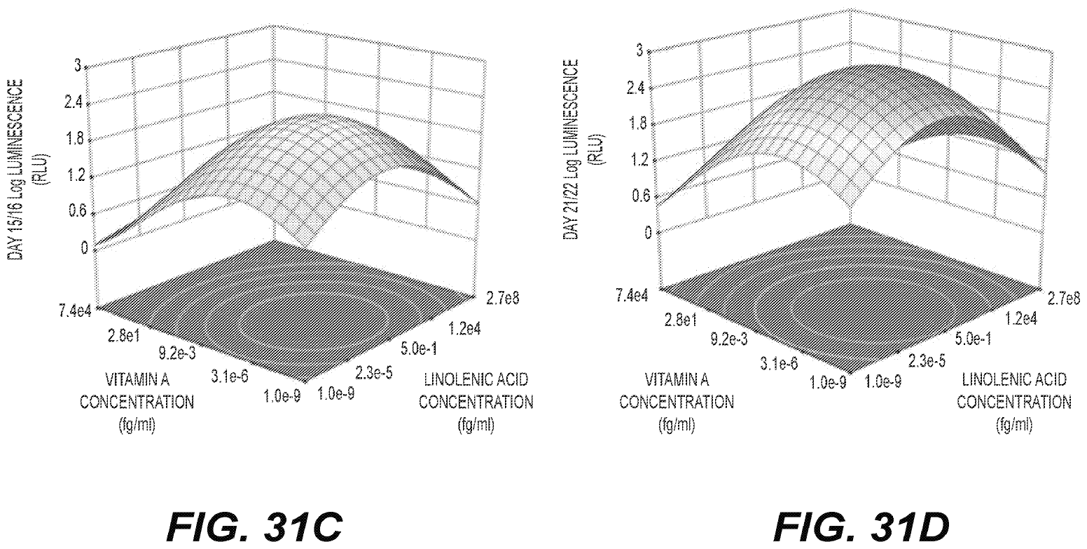

In the surface response model, each vitamin or mineral is introduced as an independent variable and is defined in Design-Expert (V.12, StatEase) as a model term. Luminescence signal over time, cumulative luminescence and resazurin data are defined as responses. The response surface study was designed as a quadratic model. FIG. 31a-31b shows the normal probability plot after the data was transformed to fit the quadratic model for week 2 and week 3. The residuals are the deviation of each sample compared to its predicted value. For the residuals to be normally distributed they must show a linear trend, indicated by the red line, with little variation outside of it. As seen in FIG. 31a-31b the residuals are normally distributed for all timepoints.

ANOVA analysis of luminescence expression for week 2 and week 3 of chondrogenesis identified significant model terms, i.e. factors that have significant effects on the responses (Table 4).

Table 4: ANOVA analysis of surface response model to determine effects of micronutrients in COL2A1-GLuc reporter rabbit chondrocytes.

TABLE 5
predicted combinations of vitamins and minerals as derived by DoE.

| | Conditions (μg/ml) | | | | |
|---|---|---|---|---|---|
| Term | 12 | 25 | 52 | 72 | 89 |
| Lin | 6.6E−09 | 3.3E−08 | 1.1E−08 | 6.8E−09 | 5.6E−09 |
| Cr | 3.9E−04 | 6.8E−04 | 4.9E−05 | 6.6E−04 | 1.1E−03 |
| Co | 5.8E−06 | 1.2E−04 | 1.3E−06 | 1.8E−06 | 5.5E−06 |
| Cu | 2.1E−01 | 2.2E−08 | 6.7E−01 | 4.6E−02 | 5.0E−02 |
| I | 9.0E−02 | 1.5E−02 | 9.2E−02 | 9.2E−02 | 9.0E−02 |
| Mn | 1.2E−02 | 4.5E−05 | 4.7E−04 | 8.6E−03 | 3.8E−03 |
| Mo | 1.9E−03 | 1.5E−03 | 2.0E−03 | 2.0E−03 | 1.1E−03 |
| Thy | 2.5E−02 | 2.6E−02 | 2.4E−02 | 2.4E−02 | 2.4E−02 |
| Vit A | 8.4E−11 | 3.0E−11 | 2.5E−11 | 1.0E−10 | 9.8E−11 |
| Vit B12 | 9.1E−07 | 6.3E−12 | 1.3E−06 | 9.1E−07 | 9.7E−07 |
| Vit B7 | 2.8E−03 | 2.3E−03 | 3.0E−03 | 9.3E−05 | 1.9E−03 |
| Vit D | 7.2E−06 | 9.9E−10 | 1.7E−05 | 8.7E−08 | 8.7E−08 |
| Vit E | 1.5E−08 | 5.9E+01 | 1.6E−08 | 1.5E−08 | 2.0E−08 |
| Vit K | 4.7E−06 | 5.0E−12 | 5.6E−06 | 2.8E−05 | 9.0E−06 |
| Zn | 3.6E−03 | 2.3E+00 | 1.8E−03 | 4.4E−03 | 1.8E−03 |
| Desirability | 0.835 | 0.595 | 0.831 | 0.829 | 0.827 |

Results of this analysis include, F-values, P-values, and lack of fit test, which indicate how well the responses fit the model. As shown in Table 4 the F- and P-values of the model, as well as the lack of fit test, over the three weeks support that the model is significant and thus the analysis for the associated terms is valid. Table 4 displays model terms that were significant for at least one of the timepoints indicated shown by a P-value<0.05. Significant single terms for all timepoints were linolenic acid, copper, and vitamin A. Several interactions were defined as significant for at least one of the timepoints shown (orange highlighted), while others including: chromium and cobalt, manganese and molybdenum, and cobalt and vitamin D were significant for all timepoints.

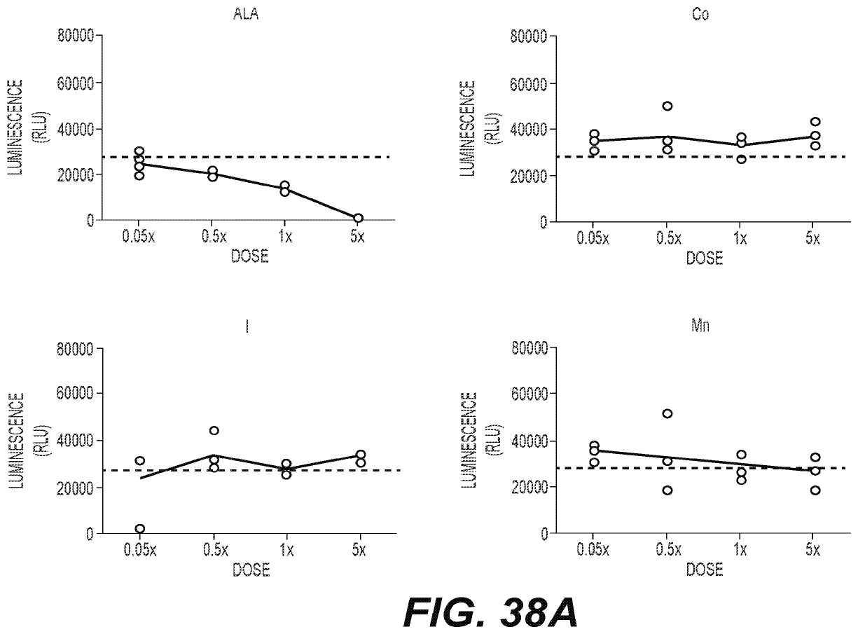

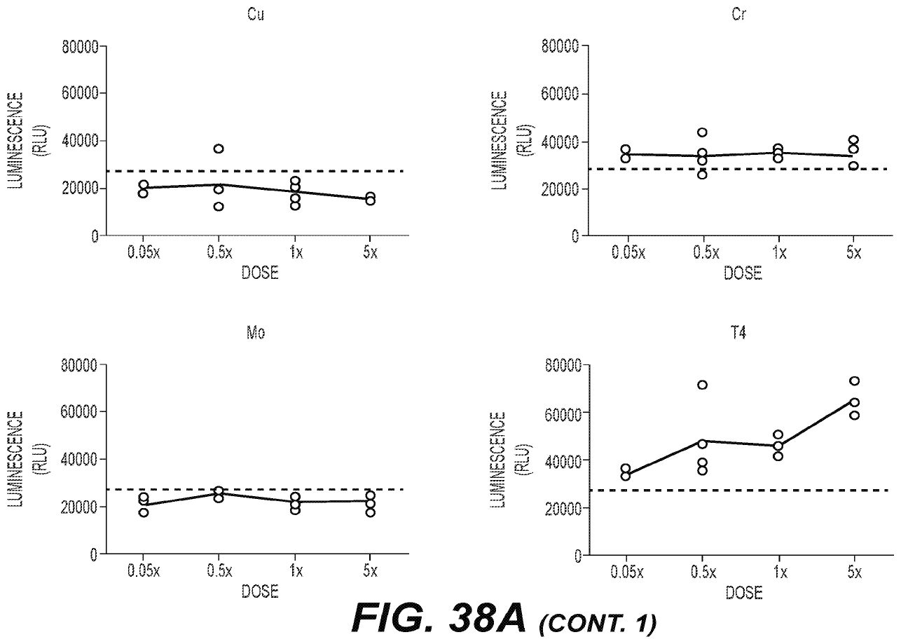

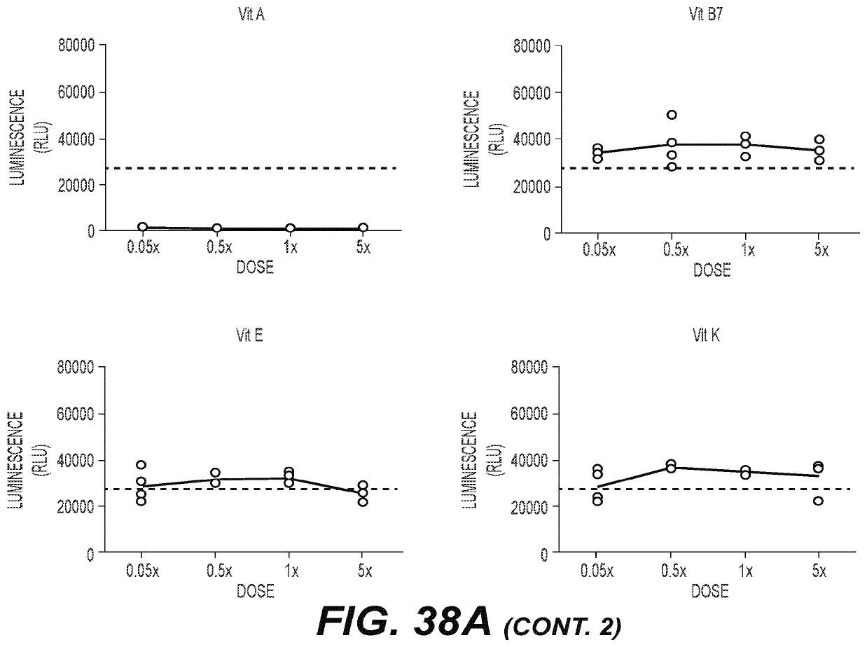

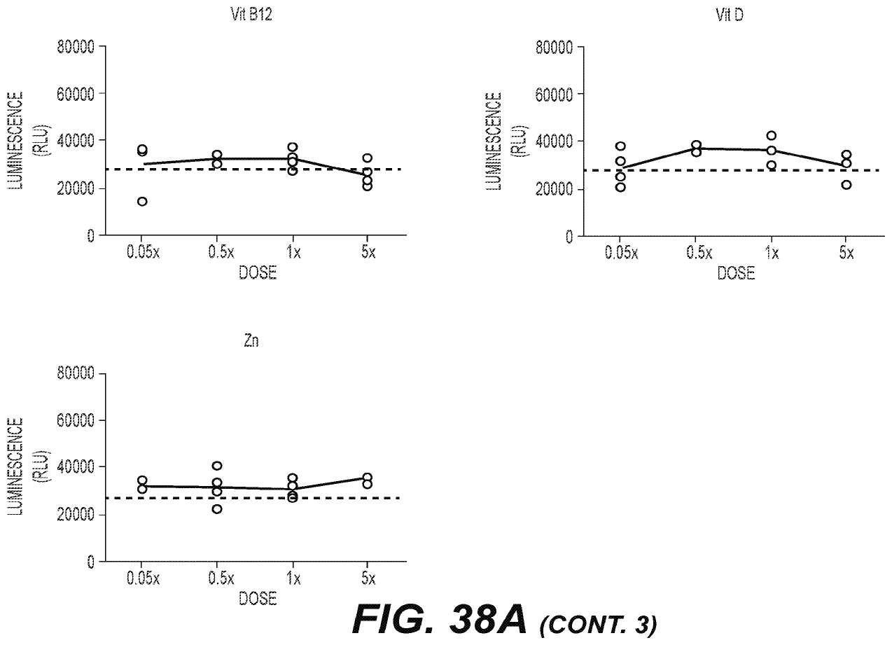

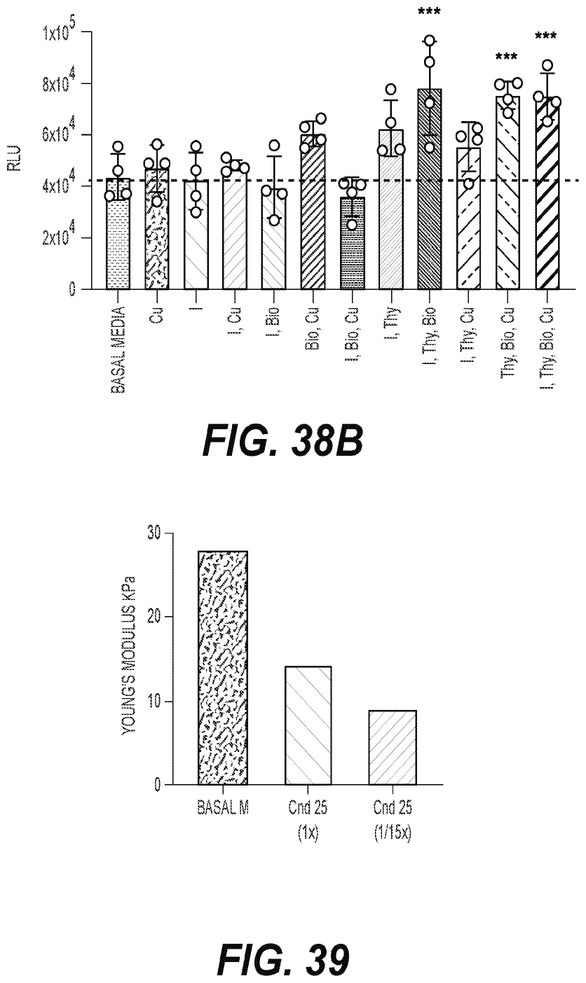

To contrast these results, testing factors in one factor at a time approach previously identified basal media supplemented with cobalt, chromium, thyroxine, or Vitamin B7, as having higher luminescence at multiple concentrations as compared to basal media alone (FIG. 38a). Combinations of these factors, with factors previously shown to have an effect⁶, are shown in FIG. 38b. Copper and Vitamin B7 alone or in combination with each other seemingly had no effect on luminescence, ˜40,000 RLU; however, in combination with thyroxine they significantly increased luminescence ˜70,000 RLU as compared to basal media alone, ˜40,000 RLU. This supports that there a synergistic effect between copper, biotin, and thyroxine. Furthermore, this supports the use of a Design of Experiments approach over a one factor a time approach as the DoE screen also identified factors that were significant in interactions but were not identified as individually significant.

Using a response surface model allows us to determine significant interactions between terms as well as determine and predict optimal concentrations of the terms within the parameters input into the initial model. 3D surface plots in FIG. 31c-31d show the dose effect of two terms (linolenic acid and vitamin A) in relation to each other and to the response (luminescence) at week 2 (FIG. 31c) and week 3 (FIG. 31d) of chondrogenesis. During week 2 (FIG. 31c) there is a predicted optimal concentration for linolenic acid and vitamin A (approximately 3×10⁻⁶ fg/ml and 5×10⁻¹ fg/ml respectively) that results in a maximal response that increases in week 3 (FIG. 31d), from 10^{1.8} RLU to 10^{2.4} RLU, although the optimal concentrations of the terms remain the same. Interestingly, the DoE model predicts that there is an optimal concentration for these in the femtomolar range (FIG. 31c-31d) while each of the two factors, when analyzed as sole additives, were detrimental to chondrogenesis (FIG. 38a).

FIG. 31c-31d is a representation of only two of the terms and a predicted optimal for each individual timepoint. Through Design-Expert, multiple terms and responses can be analyzed together to derive predicted optimal concentrations for all terms. These predicted optima account for individual responses as well interactions between terms. Out of the generated predicted optimal conditions, 5 were selected for validation, designated as conditions 12, 25, 52, 72, and 89 (Table 5).

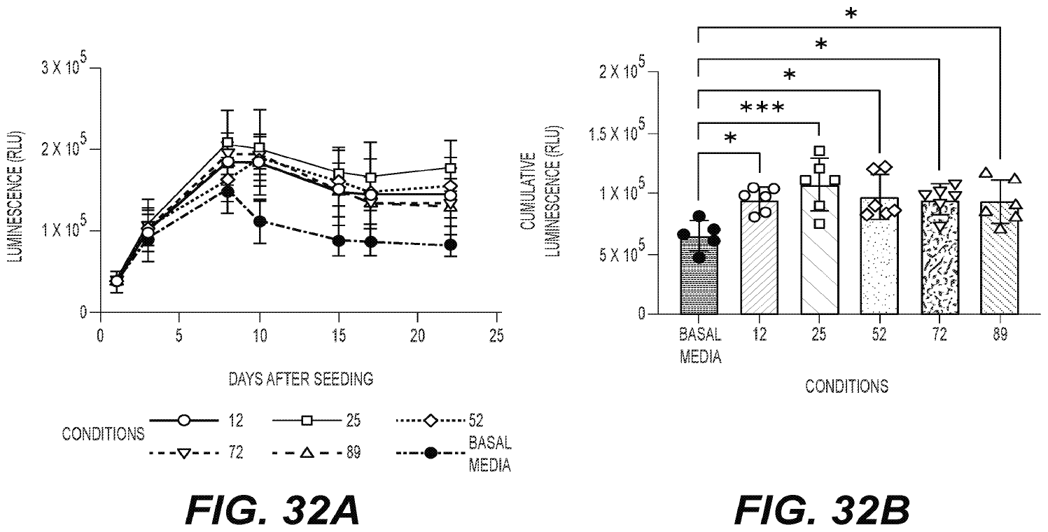

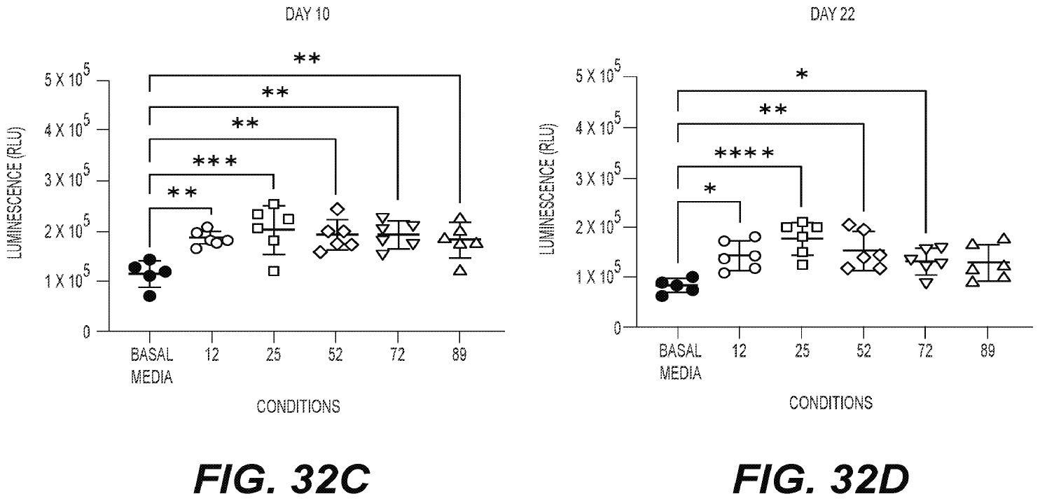

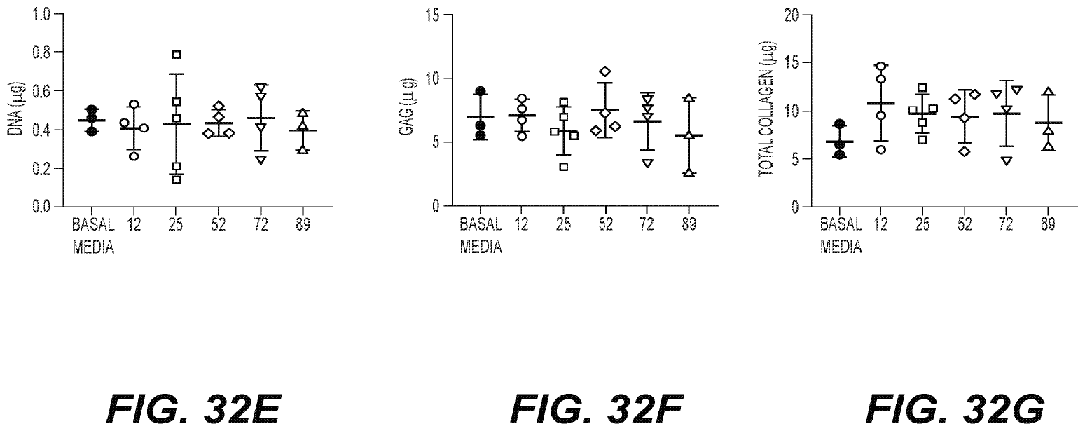

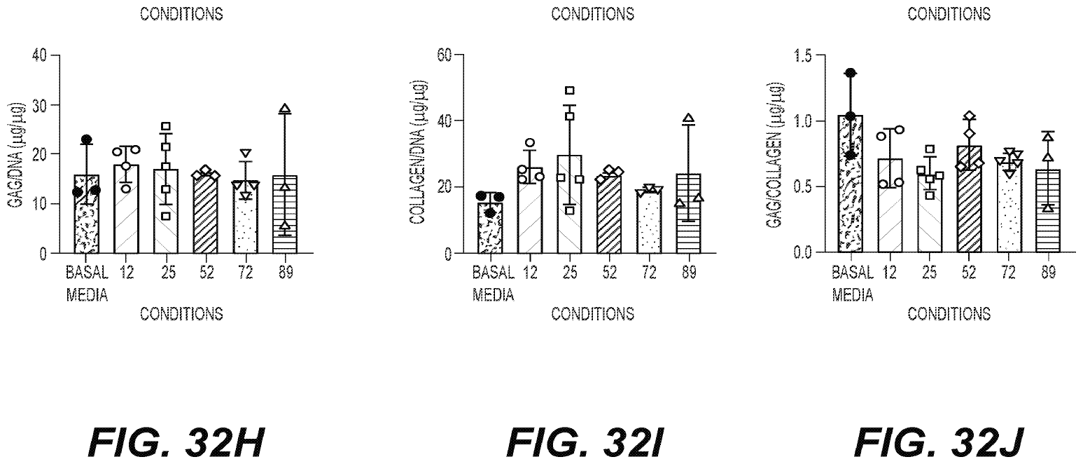

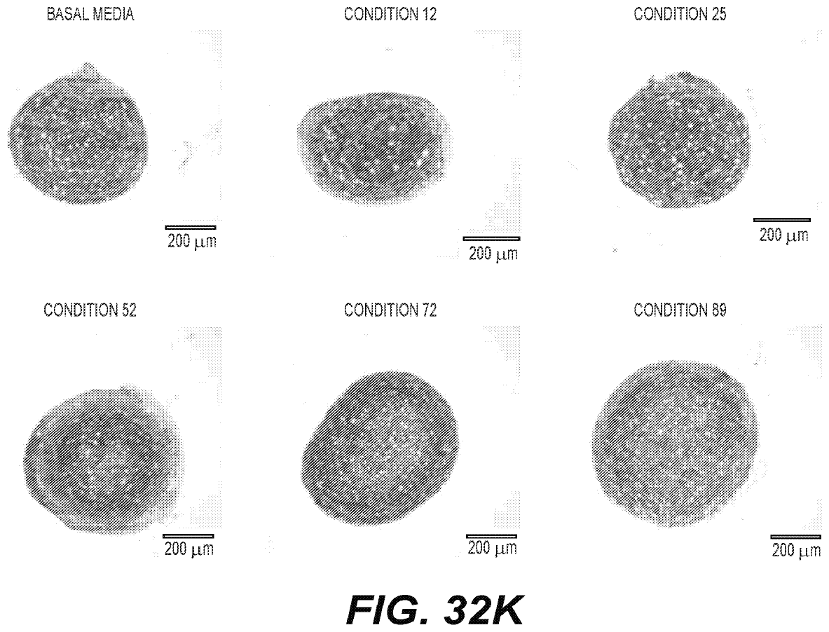

DoE predicted conditions improved Gaussia luciferase expression over basal media. To validate the Design-Expert generated predicted conditions, COL2A1-GLuc cells in aggregate culture were maintained in media supplemented with the predicted conditions (Table 5) for three weeks. As seen in FIG. 32a, all conditions tested had increased luminescence over basal media control for all timepoints after day 8, which indicates that predicted conditions have an anabolic effect early in chondrogenesis. Cumulative luminescence seen in FIG. 32b is the sum of luminescence signal over all days in culture and confirms that increased luminescence at each timepoint results in an overall significant increase in type II collagen promoter-driven activity for all predicted conditions, with condition 25 having a higher cumulative signal of ˜1×10⁶ RLU as compared to basal media, ˜6×10⁵ RLU, and other predicted conditions. Single day luminescence shown for day 10 (FIG. 32c) and day 22 (FIG. 32d) supports an increase in luminescence that is statistically significant for all conditions tested as compared to basal media with condition 25 having an average luminescence signal that is twice of that in basal media, ˜2×10⁵ RLU vs 1×10⁵ RLU respectively, for both timepoints.

To corroborate results seen by luminescence output, endpoint biochemical assays were performed at day 22 of the experiment to quantitate DNA, glycosaminoglycan (GAG) and total collagen content. FIG. 32e shows an average of ˜0.4 μg of DNA per sample with no significant difference between conditions tested, which indicates that predicted conditions have no effect on cell proliferation or viability over 22 days. Total glycosaminoglycan content is shown in FIG. 32f and as amount per microgram of DNA in FIG. 32h. As expected, DoE predicted conditions did not significantly affect glycosaminoglycan production over 22 days, although condition 89 shows large variability between samples as compared to other conditions. Quantification of total collagen, as seen in FIG. 32g shows an increase in aggregates cultured in predicted conditions to ˜10 μg over basal media (˜6 μg). However, when normalized to micrograms of DNA (FIG. 32i) only conditions 12, 25 and 89, show increased collagen as compared to aggregates cultured in basal media with significant variability within each group. Glycosaminoglycan to collagen ratio (FIG. 32j) further support an increase in collagen with no change in GAG content. Immunohistochemistry for type II collagen (FIG. 32k) confirms the presence of type II collagen for cell aggregates in all conditions at day 22 with similar staining pattern across all conditions.

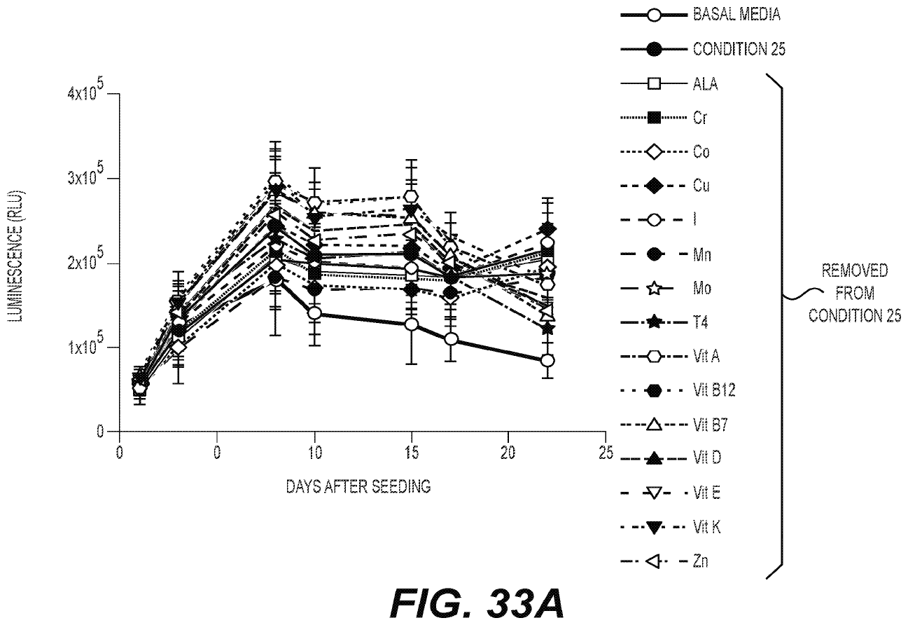

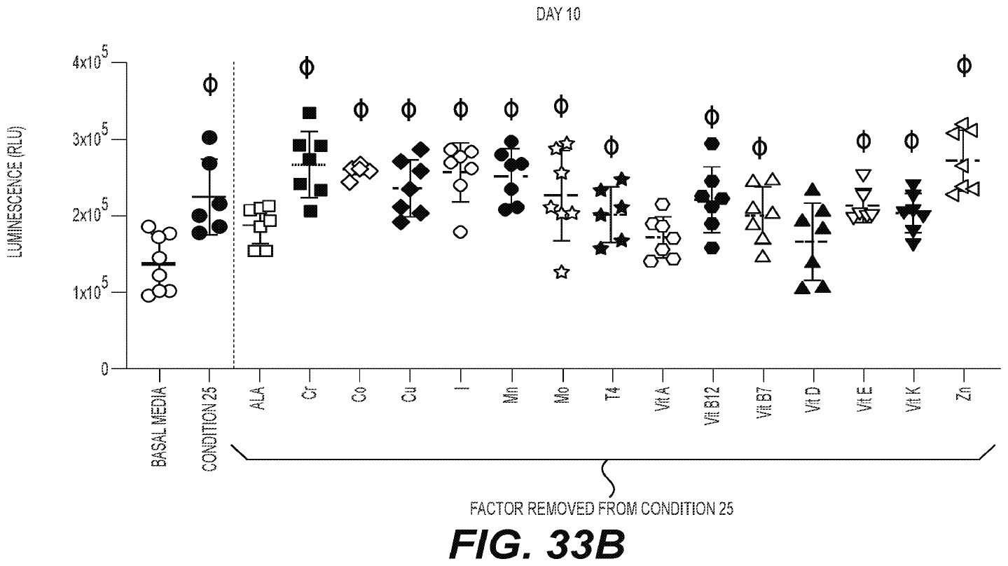

No single factor from optimized condition 25 significantly impacts type II collagen stimulation in primary rabbit chondrocytes. When using a one factor at a time approach, thyroxine (T4) was required in the tested combinations for type II collagen stimulation over basal media (FIG. 38b). To test if one factor was solely responsible for the enhancement in luminescence seen over basal media, aggregates were cultured in the predicted DoE condition 25, or combinations where one factor was removed from condition 25 for 22 days. FIG. 33a represents luminescence data for all days tested with basal media shown by a thicker black line and complete condition 25 shown by the thicker red line. All conditions had increased luminescence as compared to basal media after day 8. For statistical analysis, day 10 luminescence (FIG. 33b) is shown for all conditions tested.

Aggregates cultured in all conditions had increased luminescence, however, effects by condition 25 with Linolenic acid, vitamin A or with vitamin D removed, were not statistically significant as compared to basal media, suggestive of a major role for these biomolecules. Formulations where any of the other factors were removed from Condition 25 had significant increases in luminescence as compared to basal media alone. There was no statistical significance between any of the conditions with a single factor removed as compared to complete condition 25. These results are evidence that no single factor within DoE predicted condition 25 is solely responsible for the higher stimulation of type II collagen promoter activity observed.

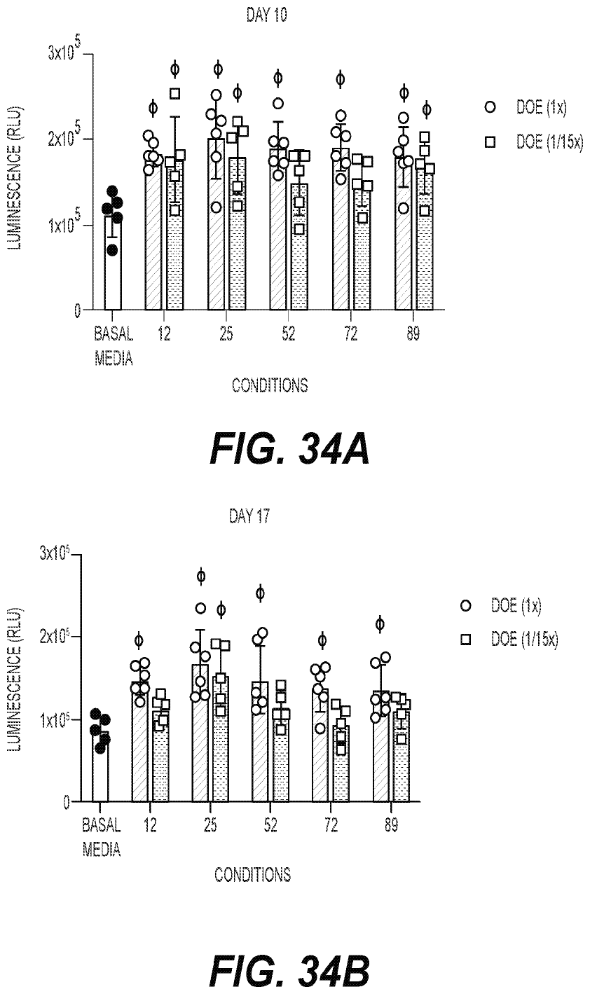

Relative concentrations of vitamins and minerals, regardless of absolute concentrations, in DoE predicted conditions play a significant role in type II collagen stimulation in primary rabbit chondrocytes. To determine if ratios, regardless of the absolute concentration, within the DoE predicted conditions were sufficient for type II collagen stimulation, aggregate cultures were treated with the DoE predicted conditions at the concentrations given or at 1/15^{th} of the predicted concentrations. Statistical analysis of luminescence at Day 10 (FIG. 34a) and Day 17 (FIG. 34b) showed no significant differences between the conditions at 1× or 1/15× of their DoE predicted concentrations.

Interestingly, while aggregates cultured at the DoE predicted conditions had significantly higher luminescence as compared to those in basal media, aggregates cultured in most conditions at 1/15^{th} did not. Only condition 25 at 1/15^{th} the concentration showed a significantly higher level of luminescence at both days vs. Basal Media, ˜1.5 to ˜1.7×10⁵ RLU vs ˜1×10⁵ RLU. This indicates that the combinatorial effect of the vitamins and minerals plays a significant role in type II collagen stimulation in primary rabbit chondrocytes, but concentrations as predicted by the DoE are optimal for type II collagen stimulation.

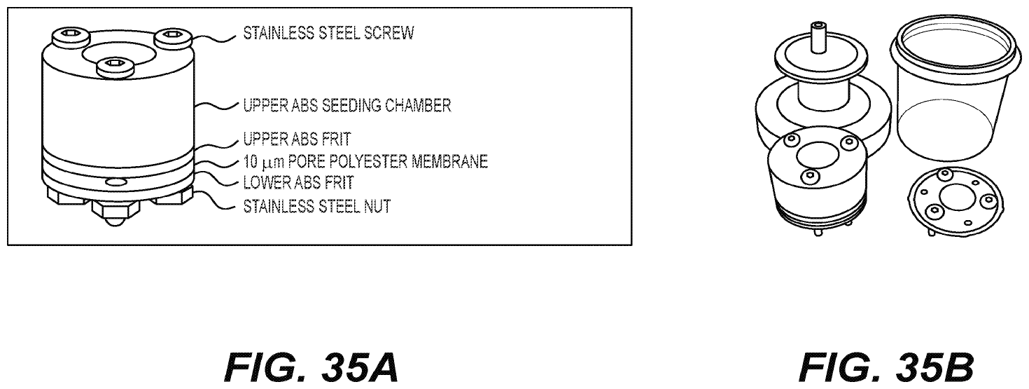

DoE predicted condition 25 stimulates type II collagen in tissue engineered rabbit cartilage. To determine if condition 25, the best performing DoE predicted condition, can have an effect in cartilage tissue engineering, COL2A1-GLuc or Non-transduced (NonTr) primary rabbit chondrocytes were cultured in custom, 3D printed bioreactors adapted from Whitney G A, et al. shown in FIG. 35a-35b over 22 days.

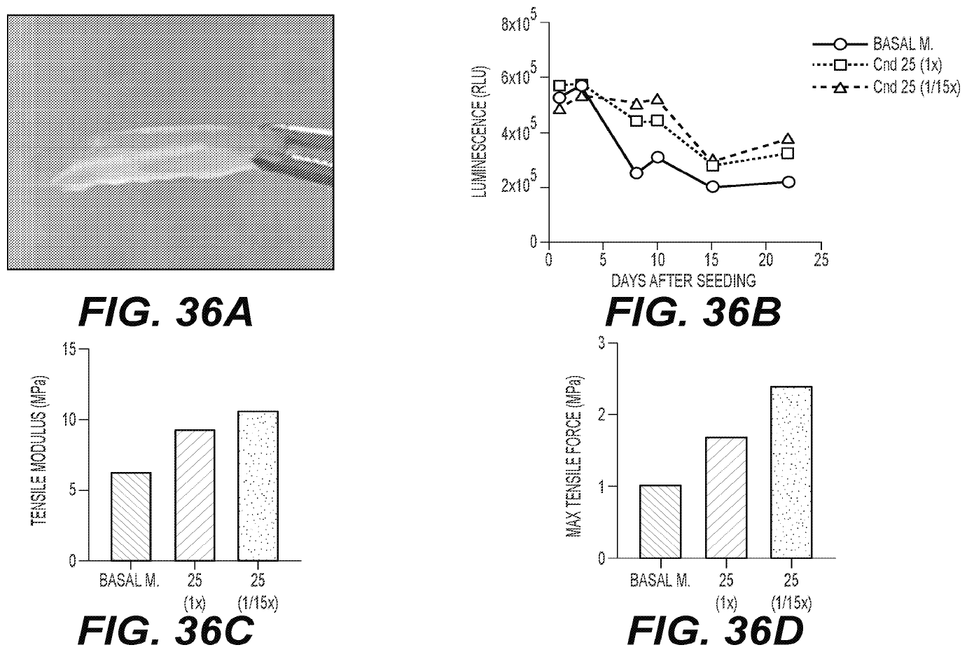

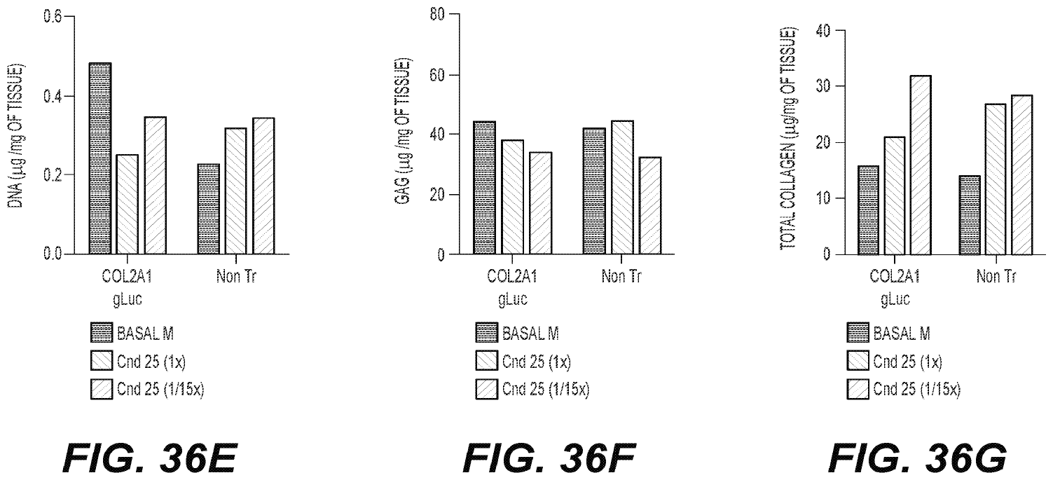

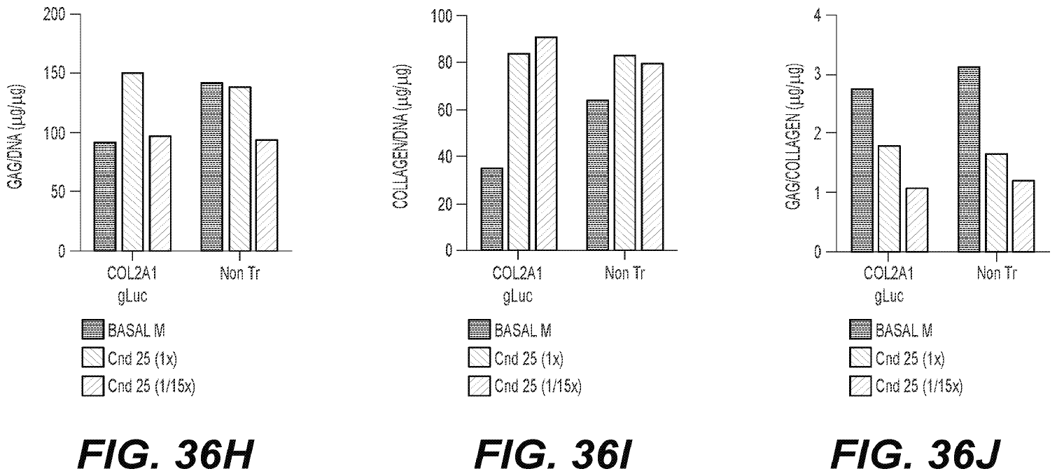

At the end of this culture period, 1.2 cm² cartilage sheets were collected as shown in FIG. 36a. Over the 22 days bioreactors housing COL2A1-Gluc cells were regularly sampled for luminescence. FIG. 36b represents luminescence data for all days tested.

Similar to results seen when conditions were tested in aggregates (FIG. 32), both condition 25 at 1× and at 1/15^{th} concentration showed increased luminescence as compared to basal media after day 8. A different curve in luminescence is observed in FIG. 36b as compared to FIG. 32a because samples were collected from inside of the biochamber for day 1 and day 3. After day 3, medium was added to increase exchange between the inside and outside of the chamber, thus samples collected are from a larger volume resulting in a decrease in luminescence from day 3 to day 8. After sheets were collected, several biopsy punches of the sheets were obtained for endpoint analysis. To determine whether increased COL2A1 reporter activity translates to improved mechanical properties of engineered cartilage, tissue elasticity was assessed via tensile testing. A marked increase was observed in the Tensile modulus (FIG. 36c) as well as maximal tensile force (FIG. 36d), in sheets generated in condition 25 media at 1× and 1/15^{th} of the concentration as compared to basal media, from ˜6 MPa in basal media to ˜8 MPa in condition 25 and ˜10 MPa in condition 25 at 1/15^{th} concentration. In contrast, compression testing resulted in a marked decrease in stiffness for sheets generated in conditions 25 regardless of concentration (FIG. 39).

To evaluate whether the luminescence results reflected matrix accumulation, biochemical assays were performed at day 22 of the experiment to quantitate DNA, glycosaminoglycan (GAG) and total collagen content from bioreactors seeded with COL2A1-Gluc and non-transduced cells. Both COL2A1-gLuc and non-transduced cell generated sheets had similar trends in DNA (FIG. 36e), GAG (FIG. 36f), and total collagen (FIG. 36g) except for the sheet generated with COL2A1-Gluc cells in condition 25 at (1×), which shows substantially lower collagen as compared to its non-transduced counterpart (FIG. 36g). Sheets in supplemented groups had overall higher collagen content per milligram of tissue, as well as per microgram of DNA (FIGS. 36g and 36i). Total glycosaminoglycan content is relatively constant for all groups at ˜40 μg per mg of wet tissue weight (FIG. 36f), when normalized to collagen content (FIG. 36j) there is a noticeable decrease in sheets generated in condition 25 (both 1× and 1/15×) indicating that condition 25 specifically affects collagen content and not glycosaminoglycan content.

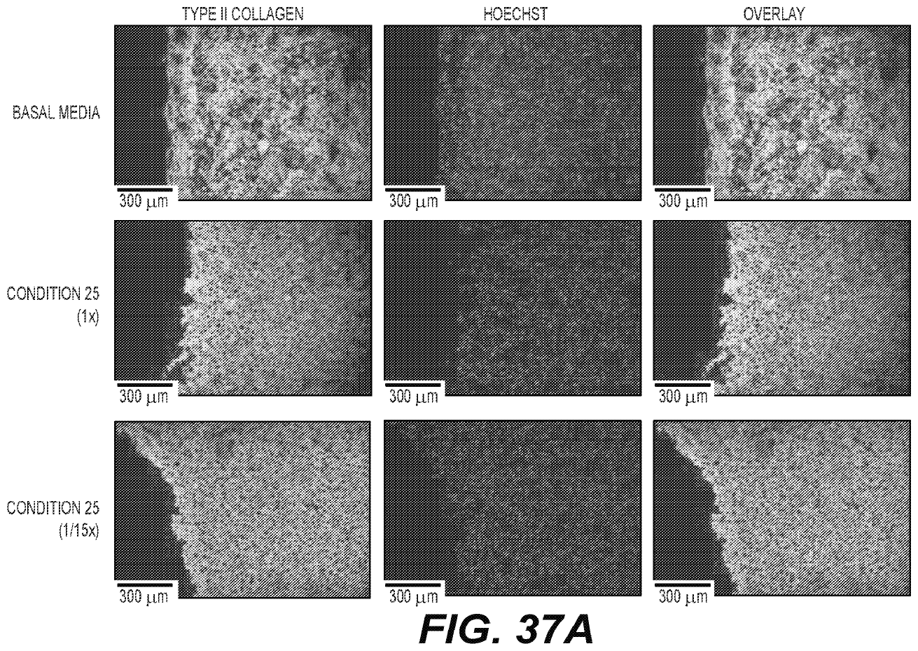

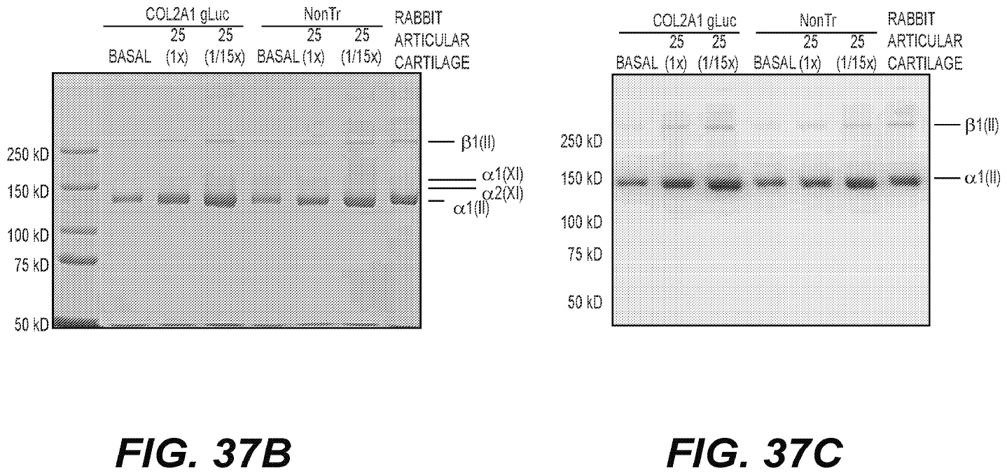

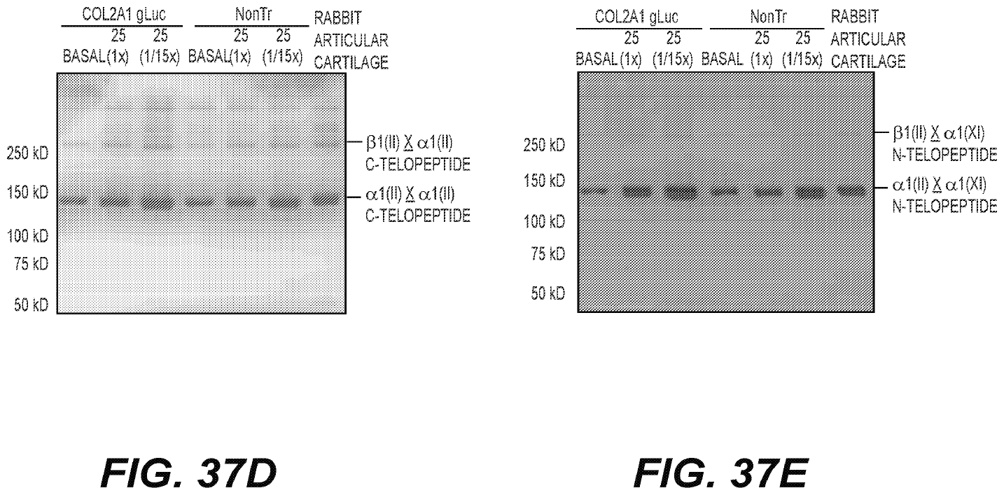

Immunofluorescence of sections of the engineered cartilage confirmed the presence of type II collagen (FIG. 37a). Interestingly, condition 25 (1/15^{th}) had a more extensive distribution of type II collagen signal as compared to basal media alone which showed an uneven pattern of staining. Collagen heteropolymer analysis was also carried out on samples of the engineered cartilage sheets and compared to native rabbit articular cartilage to further analyze collagen content. FIG. 37b shows that the major pepsin-resistant Coomassie blue-stained band in both transfected and non-transfected articular chondrocyte cultures migrated identically to the α1(II) chain of type II collagen in the adult rabbit articular cartilage. β1(II) chains (dimers of α1(II) chains) were also observed in all lanes (FIG. 37b). Two faintly stained bands migrating slightly slower than that of the α1(II) chains, are the α1 (XI) and α 2 (XI) chains of type XI collagen as previously identified by mass spectrometry and described by McAlinden et al. The α1(I) and α2(I) chains of type I collagen were neither detected in engineered cartilages nor in the control articular cartilage (if present the α2 (I) chain migrates slightly faster than the α1(II) chain), indicating that type II collagen and type XI collagen were the major collagens synthesized by the cultured chondrocytes and the cartilage collagen phenotype was maintained (FIG. 37b). The western blot shown in FIG. 37c confirms that the Coomassie blue stained bands were indeed α1(II) chains of type II collagen. 293. Using mAb 1C10, which specifically recognizes type II collagen chains, intense staining of both the α1(II) and β1(II) chains were observed in all the cultures and the adult rabbit cartilage. Since equivalent engineered cartilage dry weights loads were electrophoresed in all the lanes, densitometry of the Western blot revealed increased levels of type II collagen (α1(II)+β1(II)) reactivity under condition 25 in both transfected and non-transfected cultures compared to basal conditions. Condition 25 (1/15×) however showed highest levels of reactivity indicating highest type II collagen retained in the Condition 25 (1/15×) sheet. This is consistent with the analytical results in FIG. 36g showing highest collagen content under this condition on a per mass basis. Using a refined Western blot method precise domains of collagen chains that were cross-linked in these cultures was identified. As seen in FIG. 37d, western blotting using mAb 10F2 recognized the α1(II) and β1(II) chains in all the cultures and the adult rabbit cartilage. This is evidence that the C-telopeptide of the α1(II) chain specifically recognized by this antibody was cross-linked to the helical lysine (K87) residues in a fraction of α1(II) collagen chains and, thus, type II to type II collagen cross-links had formed in these cultures. It must be reiterated that pepsin-extracted α1(II) collagen chains are devoid of telopeptides unless they are cross-linked to the lysine residues in the helical regions of α1(II) chains.

To examine if type II and type XI collagen molecules in these cultures were stabilized by these cross-links, the pAb 5890 was used. As seen in FIG. 37e, this antibody also recognized the α1(II) chains and the β1(II) chains of type II collagen in the tissue engineered sheets and adult rabbit cartilage. This means that the N-telopeptide of the α1(XI) chain to which this antibody was raised was cross-linked to the helical lysine (K930) residue in a fraction of α1(II) chains of type II collagen molecules and thus a hetero-polymer of type II and type XI collagens had formed in all these cultures. A faint reactivity of the α1(XI) chain was observed in some of the engineered cartilage cultures that probably indicates that N-telopeptides of α1(XI) chain are cross-linked to helical lysine of another α1(XI) chain and a homo-polymer in a fraction of type XI collagen had also formed in these cultures. The data confirms that a polymer of type II collagen had formed in tissue engineered cartilage sheets and a mature collagenous heteropolymer of cross-linked type II-XI collagen fibrils had formed.

The previous efforts to optimize media conditions tested the effect of 15 different micronutrients and thyroxine on murine chondrocytes using proposed concentrations based on physiologic levels in a one-factor at a time approach. In that work, copper, vitamin A and linolenic acid were identified as having a positive effect on chondrogenesis. Overall, this study found that combinations of these micronutrients were able to increase the expression of type II collagen when tested temporally and in a dose-dependent manner. While this study showed that vitamins and minerals affect type II collagen production in murine chondrocytes in vitro, this study noted several limitations of using a traditional one factor at a time approach. This approach consists of experimental runs that are executed to hold every factor constant except for the variable of interest. This approach poorly reflects the complexity of in vivo conditions by failing to account for important interactions and largely relies on iterative experiments and trial and error for optimization. In the current study, a non-destructive reporter, primary rabbit chondrocytes, 3D culture in 96-well plates, automated pipetting, and Design of Experiments approach were combined as an efficient high throughput platform. This study was able to not only identify interactions of micronutrients that had an effect on type II collagen expression, but to also derive an optimal combination containing all missing factors as a supplement to traditional basal media, that simultaneously increased type II collagen expression.

There are several advantages to the platform implemented in this study. 1) use of primary rabbit articular chondrocytes as a model for healthy cartilage. Primary cells have the advantage of being more relevant in orthopedic research than cell lines and thus more likely to mimic responses in vivo. 2) using 3D cell aggregates adapted to 96-well plates cultured in physioxic conditions, which we adapted for use in an automated system, allows chondrocytes to maintain their phenotype as compared to 2D culture. 3) use of a secreted Gaussia Luciferase reporter. Traditional biochemical assays to evaluate chondrogenesis typically rely on destructive endpoint analysis, and due to the length of culture of the samples, low cell number in aggregates, and long and laborious processes, can result in high variability as seen in FIG. 32e-32j. Using a secreted reporter allows us to sample the media without lysis of the aggregate and thus provides the ability to examine the temporal effects of the treatment conditions on chondrogenesis. Because media is replaced every 2-3 days Gaussia luciferase readings provide a readout of the activity of the type II collagen promoter at early, mid and late timepoints in chondrogenesis. This was seen and confirmed by the TGF-β1 dose response curve which showed, for the first time, that the effective dose of TGF-β1 on type II collagen stimulation differs throughout the process of chondrogenesis. Furthermore, type II collagen expression levels are 66% higher at early timepoints with a decrease in activity at later timepoints of chondrogenesis (FIGS. 30a and 30b). The Gaussia luciferase assay is simple, sensitive and fast to perform and thus reduces variability between samples.

Using this platform, this study successfully screened 240 combinations of vitamins and minerals for their ability to promote type II collagen. Previous studies have shown that several of the micronutrients can play a role in chondrogenesis. Previously, vitamins D and K were shown to play a role in the development and regulation of chondrogenesis, while vitamin A exhibited inhibitory action on in vitro chondrogenic differentiation. Additionally, vitamin E has exhibited oxidative stress inhibition during in vivo and clinical studies. Other trace minerals such as copper and zinc promote extracellular matrix formation and deficiencies in selenium and iodine have been shown to impair bone and growth formation. Molecules like linoleic acid are known to enhance the metabolic activity of differentiating cells, while thyroxine was shown to increase type II collagen expression and glycosaminoglycan (GAG) deposition in scaffold-free engineered cartilage tissue. This study identified linolenic acid, copper, and vitamin A, as well as interactions between various vitamins and minerals (Table 2) as having significant effects on type II collagen stimulation.

These findings relied on the use of Response Surface Methodology based on Design of Experiments which significantly reduces the number of trials, accounts for errors in the model, and for interactions between factors. Statistical analysis with this approach allowed us to predict an optimal combination of vitamins and minerals, condition 25, that when tested in vitro showed significant increases in type II collagen as compared to the basal media control. Furthermore, one factor was removed at a time from condition 25, which confirmed the importance of linolenic acid, vitamin A and vitamin D and their interactions in type II collagen stimulation, further confirming the validity of the DoE results.

While Response Surface Methodology has significant advantages, its effectiveness does rely on the data fitting a second order polynomial model, thus fit statistics are crucial to ensure that the data fits the model. In addition, the validation of any findings is essential. Other aspects to consider include parameter selection for optimization of factors and response, as well as examining predicted values before validation. In the current study, this is seen by the predicted condition 25, while having a predicted desirability of 0.595, it was selected for validation due to the high predicted individual responses during analysis. When tested in vitro, it showed similar if not better responses than other selected conditions. There are few studies that have used Design of Experiments to look at biological processes, typically investigating fewer factors. To date, this study is the first to apply a response surface model to primary chondrocytes.

After validation of condition 25 in aggregates this supplementation was explored in tissue engineered cartilage. Custom 3D printed bioreactors adapted from Whitney, G A et al. were used to generate cartilage sheets in vitro (FIG. 35 and FIG. 36a). Similar to these findings in cell aggregates, type II collagen promoter-driven expression of Gaussia Luciferase was significantly increased as compared to cells in basal media in engineered cartilage. Biochemical studies supported an increase in total collagen content. Western blots of pepsin extracted samples confirm the increase is type II collagen, specifically, in sheets supplemented with condition 25 (FIG. 37c). Collagen x-link analysis supports the formation of type II collagen to type II collagen and type II collagen to type IX collagen heteropolymers, as in native rabbit cartilage (FIG. 37d and FIG. 37e). These crosslinks are characteristic of mature cartilage. This is significant as cell processes, particularly in tissue engineering, are often context dependent. It is interesting to note that condition 25 at 1/15^{th} was optimal for type II collagen expression as compared to condition 25 at 1×, as seen by luminescence, immunofluorescence and western blot. It is possible that higher concentrations are not needed by chondrocytes and can even be detrimental for chondrogenesis resulting in greater type II collagen expression when the concentrations are decreased. Multiple cell types, like osteoblasts, endothelial cells and vascular smooth muscle cells, have specialized mechanisms to recycle and fully utilize vitamins and minerals, as these cannot be synthesized by humans. Investigation of micronutrient recycling in chondrocytes has not been well studied and was beyond the scope of this work.

Supplementation with condition 25 also altered the mechanical properties of the engineered cartilage. While it increased the tensile modulus of engineered cartilage, unexpectedly, a decrease in Young's modulus was observed in compressive tests as compared to basal media (FIG. 39), indicative of decreased stiffness. While it is thought that type II collagen generally increases the tensile properties of cartilage, there is no clear correlation between type II collagen and stiffness. Furthermore, mechanical testing of live biological tissue is also confounded by the method of testing. Patel J M et al., has explored the inconsistencies present with various modes of mechanical testing which make any comparison of these findings to previous literature extremely difficult. Despite a decrease in the compressive modulus the engineered cartilage generated with condition 25 shows mechanical and biochemical properties closer to that of native cartilage than engineered cartilage generated in basal media alone.

This study demonstrates that the physiologic environment of micronutrients to culture chondrocytes has a far greater impact on chondrogenesis than previously appreciated. Supplementation of culture medium with 15 micronutrients, that are physiologically present in the articular joint, can be tailored to improve in vitro chondrogenesis, and the biochemical and mechanical properties of tissue engineered cartilage. These results show that the presence and concentrations of seemingly minor components of culture medium can have a major impact on chondrogenesis. Furthermore, this study established a streamlined process using Design of Experiments and primary reporter chondrocytes as a way to identify optimal chondrogenic conditions in vitro.

### Methods

Rabbit Primary Chondrocyte Isolation. Rabbits were euthanized under American Veterinary Medical Association guidelines and knees were isolated within 2 hours of euthanasia. The articular knee joints were dissected under sterile conditions, and articular cartilage was isolated from both the femoral condyle and the tibial plateau. Isolated cartilage was diced into <1 mm³ pieces before sequential digest, first in hyaluronidase for 30 min (660 Units/ml Sigma, H3506; in DMEM/F12 with pen/strep/amphotericin B, 30 ml), followed by collagenase type II for ˜16 hours at 37° C. (583 Units/ml Worthington Biochemical Corp.; in DMEM/F12 with 10% FBS, 1% pen/strep/fungizone, 30 ml). The digest was then filtered through a 70 μm cell strainer, washed with DMEM/F12, and resuspended in growth media (DMEM/F12 supplemented with 10% FBS, 1% pen/strep). Cells were subsequently infected as described below or cryopreserved (95% FBS, 5% DMSO).

Lentiviral Construct. Lentivirus was generated as previously described. Briefly, an HIV based lentiviral third generation system from GeneCopoeia was used to generate pseudolentiviral particles. Custom ordered COL2A1-Gaussia Luciferase plasmid (HPRM22364-LvPG02, GeneCopoeia, Inc.), envelope (pMD2.G) and packaging (psPAX2) plasmids were amplified in Escherichia coli (GCI-L3, GeneCopoeia) and silica column purified (Qiagen Maxiprep) before being co-transfected into HEK293Ta (GeneCopoeia) cells via calcium chloride precipitation. Pseudolentiviral particles were harvested from conditioned media after 48 h and concentrated via ultracentrifugation (10,000 RCF, 4° C., overnight). Titers for COL2A1-Gluc lentivirus were estimated via real-time PCR and aliquots stored at −80° C.

Lentivirus Infection of Primary Rabbit Chondrocytes. Isolated rabbit chondrocytes were seeded at 6,200 cells/cm² in growth media and allowed to adhere overnight (˜20% confluency). Cells were infected with lentivirus (COL2A1-GLuc; MOI 25 in growth media) in the presence of 4 μg/ml polybrene (Opti-mem, Gibco) for 12 h. Lentiviral medium was replaced with growth medium and cells expanded to ˜90% confluency. Cells were subsequently plated on flasks coated in porcine synoviocyte matrix and selected with puromycin (2 μg/ml) when 70% confluent for 48 hours. Culturing of rabbit chondrocytes during infection was done in physioxic (37° C., 5% O₂, 5% CO₂) conditions. Newly generated COL2A1-GLuc cells were cryopreserved at the end of this first passage (95% FBS, 5% DMSO). These cells were used for all subsequent studies.

Chondrogenic Culture. Rabbit COL2A1-GLuc were thawed and seeded in growth media at 6000 cells/cm² and expanded to 90-100% confluence in physioxia. Cells were trypsinized (0.25% Trypsin/EDTA; Corning), resuspended in basal chondrogenic media (93.24% High-Glucose DMEM (Gibco), 1% dexamethasone 10⁻⁵ M (Sigma), 1% ITS+premix (Becton-Dickinson), 1% Glutamax (Hyclone), 1% 100 mM Sodium Pyruvate (Hyclone), 1% MEM Non-Essential Amino Acids (Hyclone), 0.26% L-Ascorbic Acid Phosphate 50 mM (Wako), 0.5% Fungizone (Life Technologies) with TGF-β1 (Peprotech) and seeded as described below.

Generation and Maintenance of 3D Aggregates. To generate 3D aggregates, cells were seeded at 50,000 cells per well (in 96-well cell repellent u-bottom plates, GreinerBio) and then centrifuged at 500 RCF, 5 min. For the TGF-β1 dose response studies, that serve as positive controls for the reporter cells, aggregates were cultured in basal chondrogenic media and different concentrations of TGF-β1 ranging from 0-10 ng/ml. For the DoE studies, aggregates were cultured in basal chondrogenic media (1 ng/ml TGF-β1) as a control or basal media supplemented with vitamins and minerals (Table 3 and Table 5). Plates were cultured for three weeks in physioxia, media was sampled and replaced three times a week with respective medium. An OT-2 (Opentrons) python coded robotic pipette, programmed at an aspiration height of 2 mm from the bottom of the wells and aspiration rate of 40 μl/s was utilized for media preparation, cell feeding, and media sampling for luciferase assay. After three weeks, cell aggregates were either fixed in neutral buffered formalin for histology or medium removed and aggregates frozen dry (−20° C.) for biochemical assays.

### Tissue Engineered Cartilage Sheets

A) Biochamber Sterilization and Assembly. Custom 3D printed biochambers that produce 1.2 cm² cartilage sheets are shown in FIG. 35. The chambers are made of an acrylonitrile butadiene styrene (ABS) seeding chamber and a 10 μm pore polyester membrane (Sterlitech). Screws, a silicone washer and ABS frits hold everything securely and prevent any leaking in between the different pieces. Furthermore, they keep the chamber elevated to allow medium to reach the membrane from the top and bottom for efficient media exchange. The chambers are contained within Nalgene containers modified to have a 0.2 μm sterile filter on the top to allow gas exchange. The Nalgene containers along with screws, silicone washer, polyester membrane and nuts were autoclaved and sterile filters fitted to the containers in a biosafety cabinet. ABS pieces were placed in a sealable container for sterilization by immersion in a 10% bleach solution, water rinse, followed by a 10% sodium thiosulfate treatment to neutralize any remaining chlorine, sterile water and isopropanol wash before drying in the biosafety cabinet. Biochambers were assembled as shown in FIG. 35a inside a biosafety hood using sterile surgical gloves and autoclaved surgical tools to handle biochamber parts. Once assembled, the polyester membrane was coated with fibronectin (50 μg/cm², Corning, in PBS) and allowed to dry in a biosafety cabinet for 1 hr.

Generation and Culture of Tissue Engineered Cartilage Sheets COL2A1-Gluc cells or uninfected primary rabbit chondrocytes were seeded at 5×10⁶ cells/cm² in ABS biochambers with a 1.2 cm² seeding area in basal chondrogenic media alone or in basal media with condition 25 at 1× and 1/15× (Table 5). Media were added in the Nalgene container outside of the biochamber making sure it did not reach the top of the biochamber and combine with the cell suspension inside the biochamber in order to allow cells to adhere to the membrane. After 1 day, medium was added to the top of the biochamber so that media exchange occurs with the inside of the whole biochamber. These were cultured in physioxia on a shaker (10 RPM) for 3-weeks with media changes three times a week. During media replacement, samples from COL2A1-Gluc biochambers were assessed for luciferase. After three weeks, cartilage sheets were collected, four (4 mm) biopsy punches were taken for mechanical assessment, and collagen cross-linking analysis, remaining pieces of the sheets were frozen (−80° C.) for biochemical assessment or stored in formalin for histology.

Luciferase Assay. Cell culture medium sampled from the seeded 96-wells (20 μL/well) was assessed using a stabilized Gaussia Luciferase buffer at a final concentration of 0.09 M MES, 0.15M Ascorbic Acid, and 4.2 μM Coelenterazine in white 96-well plates. Luminescence was measured in a plate reader (25° C., relative light units, En Vision plate reader). An OT-2 (Opentrons) python coded robotic pipette was utilized for luciferase buffer addition to white plates (GreinerBio).

Immunohistochemistry/Immunofluorescence. At the end of three-week culture, cell aggregates were fixed in 10% Neutral Buffered Formalin overnight, embedded in paraffin wax and sectioned (7 μm sections). Sections were deparaffinized and hydrated, followed by treatment with pronase (1 mg/ml, Sigma P5147, in PBS with 5 mM CaCl₂)) for epitope retrieval and incubation with primary anti-Collagen Type II (DSHB II-II6B3) followed by a biotinylated secondary and Streptavidin-HRP (BD Biosciences). II-II6B3 was deposited to the DSHB by Linsenmayer, T.F. (DSHB Hybridoma Product II-II6B3). Sections were stained with a chromogen-based substrate kit (Vector labs, VIP substrate vector kit). Engineered cartilage sheet sections were also treated with pronase and primary anti-Collagen Type II (DSHB II-II6B3) followed by VectaFluor R.T.U Antibody Kit DyLight® 488 (Vector Labs DI-2788) following manufacturer's protocol. All sections were imaged at 10× magnification.

Biochemical Assays. Frozen cell aggregates, or pieces of engineered cartilage were thawed in PBS, and enzymatically digested with Papain (25 μg/ml, Sigma, P4762, in 2 mM cysteine; 50 mM sodium phosphate; 2 mM EDTA at a pH 6.5, 100 μl) at 65° C. overnight. During digestion, plates were covered with a qPCR adhesive sealing film (USA Scientific), a silicone sheet, and steel plates clamped to the plate to prevent evaporation. After digestion half of the digest was transferred to another plate and frozen for hydroxyproline assessment. For the remaining half of the digest, papain was inactivated with 0.1M NaOH (50 μl) followed by neutralization (100 mM Na2HPO4, 0.1 N HCL, pH 1.82, 50 μl). To assess DNA, samples of the digests (20 μl) were combined with buffered Hoechst dye (#33258, 667 ng/ml, phosphate buffer pH 8, 100 μl) and fluorescence measured at an excitation of 365 nm and emission of 460 nm. For GAG assessment, samples of aggregate digest (5 μl) were combined with a 1,9-Dimethyl-methylene blue solution (195 μl) and absorbance was measured at 595 nm and 525 nm. Absorbance readings were corrected by subtracting 595 nm reading from 525 nm. Total micrograms of DNA and GAG were calculated using a Calf thymus DNA standard (Sigma) and Chondroitin Sulfate standard (Seikagaku Corp.), respectively.

For hydroxyproline (HP), the frozen digest was thawed and incubated overnight at 105° C. with 6M HCL (200 μl). Plates were covered as described above to prevent evaporation. Samples were subsequently dried at 70° C. overnight with a hydroxyproline standard (Sigma). Copper sulfate (0.15M, 10 μl) and sodium hydroxide (2.5M, 10 μl) were added to each well and incubated at 50° C. for 5 minutes, followed by hydrogen peroxide (6%, 10 μl) for 10 minutes. Sulfuric acid (1.5 M, 40 μl) and Ehrlich's reagent (20 μl) were added and samples further incubated at 70° C. for 15 minutes before reading absorbance at 505 nm. Total micrograms of hydroxyproline were calculated using the standard. Total collagen was calculated by the following formula (μg of HP×7.6=μg Total Collagen).

Collagen Cross-Link Analysis. After harvest, samples were frozen at −80° C. until use. Samples were lyophilized, and dry weights obtained. Proteoglycans were extracted using 4M guanidine hydrochloride (GuHCl) in 50 mM Tris buffer pH 7.4. The residue was exhaustively rinsed using MilliQ water to remove residual GuHCl, lyophilized and weighed. The cross-linked collagen network was depolymerized using equal volumes of pepsin (0.5 mg/mL in 0.5M acetic acid). Equivalent aliquots of dry weights were loaded on 6% polyacrylamide gels. Pepsin-extracted type II collagen from adult rabbit articular cartilage was used as a control. After electrophoresis, collagen chains were stained using Coomassie Blue. For Western blots, following SDS-PAGE the separated collagen chains were transferred, by electrophoresis, onto a polyvinyl difluoride (PVDF) membrane and probed with monoclonal antibody (mAb) 10F2 to identify the C-telopeptide of α1(II) collagen chains cross-linked to α1(II) chains. Another blot was probed with polyclonal antibody (pAb) 5890 to identify the N-telopeptide of α1(XI) chains cross-linked to α1(II) chains. This blot was then stripped and probed with mAb 1C10 to identify α1(II) chains. As described before, this determines if a heteropolymer of type II and type XI collagen had formed. Mechanical Testing

A) Compression Testing. Biopsy punches (4 mm) were thawed in Tyrode's solution (Sigma) with protease inhibitors (Sigma, P8465) and equilibrated to room temperature. Using a TA.XTPlus connect Texture analyzer a trigger force of 0.1 gram determined the height of the tissue, then 5-20% strain was applied in 5% increments with a 20-minute hold to reach equilibrium. From these results, the equilibrium force was calculated, and a stress vs strain curve generated. Young's modulus in compression was determined from the slope of these curves.

B) Tensile Testing. Biopsy punches (4 mm) were thawed in Tyrode's solution (Sigma) with protease inhibitors and equilibrated to room temperature. As previously described, a custom dog-bone punch was made from biopsy punches and punches taken from the 4 mm punch. Custom holders were made from laminate projector sheets and dog-bone punches attached using cyanoacrylate glue (Ultra Gel Control, Loctite), tissue was continuously bathed in PBS during this process. Using a TA.XTPlus connect Texture analyzer with a trigger force of 0.1 gram, tissues were stretched to failure, the equilibrium tensile force was calculated and a stress vs strain curve generated. Young's modulus in tension was determined from the slope of these curves.

Design of Experiment Response Surface Design. Design-Expert 12 (StatEase) was used to generate a surface response model to assess the effect of 15 factors: linoleic acid, cobalt, copper, chromium, iodine, manganese, molybdenum, thyroxine, vitamin A, vitamin B12, vitamin B7, vitamin D, vitamin E, vitamin K, and zinc. Table 3 shows minimum and maximum concentrations input into Design-Expert. The response surface I-optimal blocked design generated 240 total conditions to assess the response.

Design of Experiments Analysis. At the end of this experiment, responses (luminescence, metabolic activity and aggregate area) from the screen of 240 conditions as well as results from the one factor at a time approach, were analyzed (Design-Expert, StatEase). Analysis of the results indicated a quadratic model as the best fit. After transformation of the data to fit a quadratic model, Analysis of Variance (ANOVA) was used to identify the positive and negative effects on chondrogenesis as well as fit statistics for the model. The optimization module of the software was used to generate five predicted optimal combinations of factors (Table 5). Two sets of parameters were used to generate predicted conditions. For condition 25 from Table 5, all vitamins and minerals were targeted at 75% serum max except for linolenic acid, vitamin A, copper and vitamin D which are set at their predicted optima. For the other predicted conditions vitamins and minerals were set between 0.01% of serum max and serum max except for vitamin A, E and linolenic acid which were at their approximate optima. All conditions were selected to maximize luminescence for week two and week three as well as aggregate area for week three. Condition 25 also had a target of 0.2, for Resazurin (metabolic activity), the average measurement for chondrocyte aggregates, at week three.

Statistical Analysis. Statistical analysis for all experiments except for the Design of Experiments screen (analysis described above) were performed using GraphPad Prism 9 and One-way or two-way ANOVA. All data passed tests for normality. In all figures * indicates p-value<0.05, ** indicates p-value<0.01, and *** indicates p-value<0.001.

### Example 5. Biomaterial Composition and Stiffness as Decisive Properties of 3D Bioprinted Constructs for Type II Collagen Stimulation

Gelatin methacrylate (GelMA) and hyaluronic acid methacrylate (HAMA) are frequently used biomaterials for 3D bioprinting, with individual well-established material characteristics. To identify an ideal combination of GelMA and HAMA for chondrogenesis, a novel, primary human chondrocyte COL2A1-Gaussia luciferase reporter system (HuCol2gLuc) was developed. With this non-destructive, high-throughput temporal assay, Gaussia luciferase is secreted from the cells and used as a proxy for measuring type II collagen production. GelMA:HAMA ratios were screened using the reporter system before proceeding to 3D bioprinting. This method is efficient, saving on time and materials, resulting in a streamlined process of biomaterial optimization. The screen revealed that the addition of HAMA to GelMA improved chondrogenesis over GelMA (15%) alone. Storage moduli were measured using dynamic mechanical analysis of the same GelMA:HAMA ratios and established an initial threshold for chondrogenesis of ˜30 kPa. To determine if biomaterial storage moduli impact cell mobility human primary chondrocytes transduced with green fluorescent protein (GFP) were 3D bioprinted in either 1:1 or 2:1 ratios with storage moduli of 32 kPa and 57.9 kPa, respectively. This study found that reduced cell mobility, in the stiffer biomaterial had higher type II collagen expression, than the softer material with more cell mobility. Finally, after 3D bioprinting with HuCol2gLuc cells this study successfully identified an optimal combination (2:1) of GelMA:HAMA and photo-crosslinking time (38 s) for chondrogenesis.

### INTRODUCTION

Articular cartilage is a highly specialized connective tissue that functions as a frictionless surface for movement and assists in the distribution of loads. Articular cartilage has limited healing capabilities, therefore defects in cartilage continue to degenerate over time, often resulting in osteoarthritis. Surgical intervention is typically required to treat articular cartilage defects, however results are highly variable. Current treatments improve short term outcomes, but fully functional restoration of articular cartilage is hard to achieve.

Tissue engineering is an interdisciplinary field that aims to develop structural and functional alternatives for native tissue. The ideal tissue engineered cartilage construct can mimic native extracellular matrix (ECM), encapsulate cells, and not only fill and maintain the defect space while the new tissue grows, but also enable integration with the surrounding native tissue. 3D bioprinting has the potential to achieve an ideal tissue engineered construct through simultaneous extrusion of both living cells and biomaterials. Advantages of 3D bioprinting include the ability to print patient specific bioactive scaffolds and recapitulation of native tissue zonal architecture. Post-fabrication cell-seeding is not required, and tissue is therefore not hindered by limited cell penetration. Biomaterials must be both biocompatible and printable.

This study used two well characterized and frequently used biomaterials, gelatin methacrylate (GelMA) and hyaluronic acid methacrylate (HAMA). Gelatin, a hydrolysis product of collagen, contains cell adhesion arginine-glycine-aspartic acid (RGD) sites, and target degradation sequences for matrix metalloproteinases (MMP) that enable remodeling. Hyaluronic acid is an integral glycosaminoglycan (GAG) of cartilage ECM, and is found abundantly in synovial fluid. It has also been shown to provide cells with biochemical cues to regulate morphology and proliferation. Methacrylation of these materials enables photo-crosslinking to increase stability and stiffness. Lithium phenyl-2,4,6-trimethylbenzoylphosphinate (LAP) is frequently used as a photoinitiator because it can crosslink at 405 nm, thereby reducing DNA damage by UV light. The positive attributes of these materials have been established through material science characterization.

Chondrocyte response to biomaterials and bioprinting is the focus of this biology-driven approach. To identify biomaterial combinations that improve chondrogenesis, this study developed an innovative COL2A1-Gaussia luciferase primary human chondrocyte reporter system (HuCol2gLuc) for temporal analysis of type II collagen production. This high-throughput assay has previously been used and characterized in ATDC5 cells. Gaussia luciferase is secreted from the cells upon type II collagen promoter stimulation making this a non-destructive 3D culture assay and allowing for temporal analysis of type II collagen expression. This data when combined with traditional end-point assays establishes a more in-depth characterization of the chondrogenic response in biomaterials.

Cell migration is considered a hallmark of tissue-remodeling and repair. Articular chondrocytes are typically considered to be immobile due to the dense ECM. Previous studies have demonstrated that articular chondrocytes can migrate in response to various stimuli in vitro. These studies have mostly relied on 2D culture and only a few have shown chondrocyte mobility in 3D culture. Chondrocyte mobility has been observed in a 3D “dot” of 4% type I collagen in response to nitric oxide, and in a mixture of type I collagen and hyaluronic acid. A cartilage defect wound healing model using a disc of collagen type I and II containing chondrocytes with a 5 mm defect, showed migration into the defect area over 4-weeks. To the best of our knowledge, no papers have been published on cell mobility in 3D bioprinted cartilage constructs which can provide more insight as to ideal material qualities and the 4th dimension in bioprinting approaches.

This study aimed to identify ideal combinations of GelMA and HAMA to improve chondrogenesis using this HuCol2gLuc reporter system and Design of Experiment (DoE) software (Design-Expert, Stat-Ease). The DoE is a well-established technique for planning experiments using the minimum number of test groups to generate the maximum amount of information. Together these methods streamlined this optimization process before proceeding to 3D bioprinting. An optimal biomaterial composition of GelMA and HAMA was, based on biomaterial storage modulus and chondrocyte mobility and derived by this HuCol2gLuc phenotypic reporter system.

### Materials and Methods

Human chondrocyte isolation. Human articular chondrocytes were isolated from discarded surgical tissue collected from two patients during total joint replacement surgery with informed consent under IRB approved protocols (Baylor College of Medicine, H-36683, H-36374). Discarded tissue <3 h post-operation on ice in saline) was briefly stored in defined chondrogenic media (˜4 h, room temperature [RT]). Visually intact cartilage tissue was dissected from the femoral head using sterile technique (˜3 cm×1 cm) and diced into pieces <1 mm³ in DMEM-LG (Hyclone). Diced tissue was centrifuged (1 min, 100 RCF, RT), supernatant removed, and digested in hyaluronidase enzyme solution (40 mL, 660 Units/mL in DMEM-LG/F12, Sigma,) for 30 minutes (37° C.) on a nutating rocker and then centrifuged again. After the supernatant was removed, collagenase type II was added (40 mL, 583 Units/mL in DMEM/F12 with 10% FBS, Worthington Biochemical Corp CLS2) for overnight digestion (15 h). Remaining fragments were removed (70 μm Nitex filter) and the cell suspension diluted 1:1 with DMEM/F12 before centrifugation (10 min, 700 RCF, RT). Cells were resuspended in growth media (DMEM/F12 supplemented with 10% FBS (mesenchymal stromal cell selected and 1% penicillin-streptomycin). Live cells were cryopreserved or transduced as described below.

Engineering of type II collagen promoter-driven reporter human chondrocytes (HuCol2gLuc) and GFP chondrocytes (HuChon-GFP). As previously described, lentiviral plasmids (HPRM22364-LvPG02, EX-EGFP-Lv105, psPAX2 and pMD2.G) were grown in E-Coli (GCI-L3, GeneCopoeia, Inc.) and column purified (Qiagen Maxiprep) before co-transfection into HEK-293Ta cells (GeneCopoeia, Inc.). Col2gLuc or GFP psuedolentiviral particles were collected from supernatant and concentrated by centrifugation (10,000 RCF, 4° C., overnight). Freshly isolated human chondrocytes were seeded (6,100 cells/cm²) in growth media and allowed to adhere overnight to an 10 cm cell culture dish. Chondrocytes were then incubated with Col2gLuc or GFP pseudolentiviral particles (multiplicity of infection (MOI) ˜25) in Opti-MEM (Gibco) containing polybrene (4 μg/mL) at 4° C. for 15 min before placing at 37° C. overnight (5% CO₂, 5% O₂, 17 h). Following overnight incubation, pseudolentiviral particle containing media was removed and replaced with growth media. Chondrocytes were grown to 70-90% confluence before trypsinization and seeding onto synoviocyte matrix coated flasks. At ˜90% confluence, cells were trypsinized, neutralized and cryopreserved in FBS (95%) with DMSO (5%).

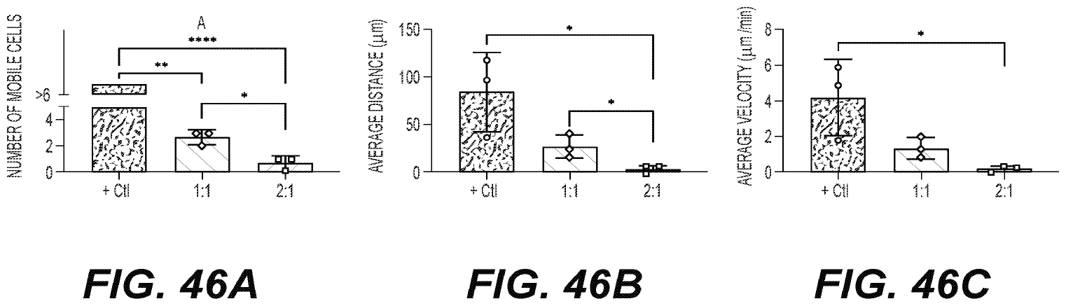

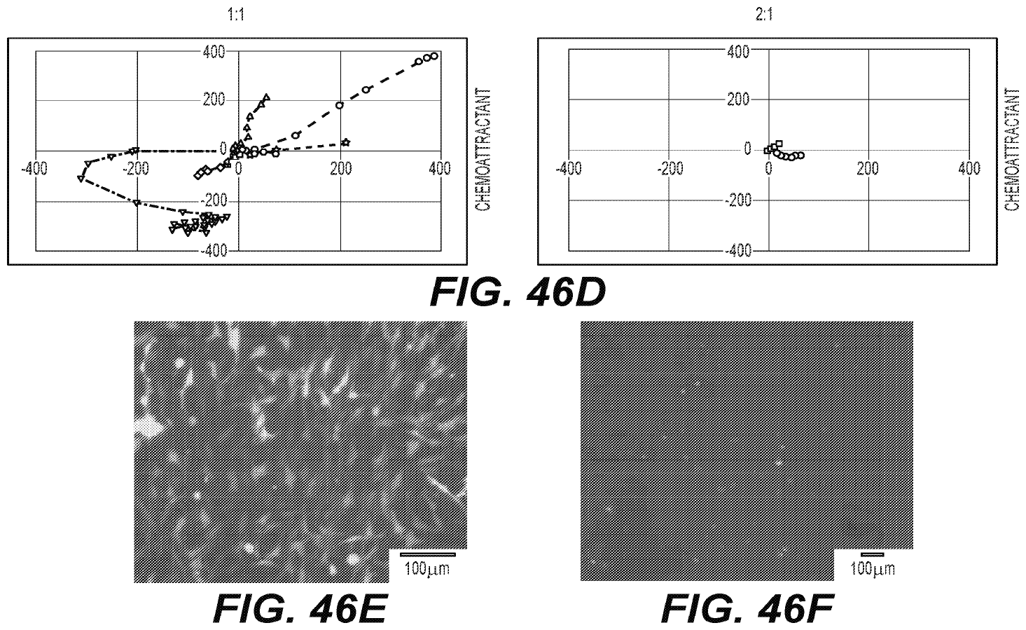

HuChon-GFP Cell Characterization and HuCol2gLuc TGFβ1 Dose Response. HuChon-GFP infected chondrocytes were assessed by microscopy (Zoe™ imager, Bio-Rad) as shown in the results (FIG. 46E). HuCol2gLuc infected chondrocytes were assessed through dose response to TGFβ1 to establish these cells as chondrogenic reporters. Reporter chondrocytes were thawed from frozen stocks, seeded onto synoviocyte derived matrix coated flasks and expanded to confluence. At confluence, cells were trypsinized (0.25% Trypsin/EDTA, 5 min, 37° C.), neutralized with growth media, then centrifuged (5 min, 500 RCF, RT). Supernatant was removed and cells resuspended in defined chondrogenic media (DMEM-HG 93.24% (Lonza), 1% 10⁻⁵M dexamethasone (Sigma), 1% ITS+premix (Becton-Dickinson), 1% Glutamax (Hyclone), 1% 100 mM Sodium Pyruvate (Hyclone), 1% MEM Non-Essential Amino Acids (Hyclone), 0.26% 50 mM L-Ascorbic Acid Phosphate (Wako), 0.5% Fungizone (Life Technologies)). Cells were plated in non-adherent 96-well sterile plates (Greiner Bio), TGFβ1 added (0-100 ng/mL, Peprotech) and centrifuged into aggregates (5 min, 500 RCF, RT). Cell aggregates were incubated for 3 weeks with media exchange every 2-3 days (37° C., 5% CO₂ and 5% O₂). Conditioned media, containing the secreted Gaussia luciferase, was assessed for luminescence (section 2.9). On day 22, cell aggregates went through either histological (section 2.12) or biochemical assessment (section 2.13).

HuCol2gLuc qPCR Assessment. HuCol2gLuc infected chondrocytes were cultured in a monolayer on 12-well cell culture plate (Corning) for qPCR gene assessment. Cells were seeded (6,100 cells/cm²) in growth media and allowed to adhere overnight. Then growth media was replaced with chondrogenic media supplemented with 0-10 ng/mL TGFβ1. By day 5, luciferase assessment (section 2.9) of culture media showed a dose response in luminescence values. Cells were extracted for RNA analysis with lysis buffer (Ambion PureLink RNA Mini kit) and the lysate frozen on dry ice and stored (−80° C., 1 week). Total RNA was isolated from lysates using column purification with on-column DNA digest. RNA purity and integrity was assessed by RNA ScreenTape (Agilent Technologies). cDNA was synthesized from 400 ng RNA using a cDNA synthesis master mix (Maxima H Minus, Thermo Scientific). qPCR was performed (QuantStudio 7 flex, Applied Biosystems) for gene expression of Hypoxanthine Phosphoribosyltransferase 1 (HRPT, reference gene, forward primer: 5′ ATTGACACTGGCAAAACAATGC 3′ (SEQ ID NO: 3), reverse primer: 5′ TCCAACACTTCGTGGGGTCC 3′ (SEQ ID NO: 4)), Gaussia luciferase (gLuc, 5′ ACGCTGCCACACCTACGA 3′ (SEQ ID NO: 5), reverse primer: 5′ CCTTGAACCCAGGAATCTCAG 3′ (SEQ ID NO: 6)) and type II collagen (COL2A1, 5′ TGGAGACTACTGGATTGACCCCAACCAA 3′ (SEQ ID NO: 7), reverse primer: 5′ TCTCGCCAGTCTCCATGTTGCAGA 3′ (SEQ ID NO: 8)). Primers, SYBR green (Applied Biosystems, Thermofisher Scientific) and cDNA were mixed and run using cycling parameters: 95° C. for 20s then 45 cycles of 95° C. 10s, 60° C. 20s, 72° C. 19s, followed by melt curve analysis. CT values were normalized to HRPT expression and then gLuc vs COL2A1 relative gene expression was plotted with 95% confidence bands (GraphPad Prism).

Preparation of Gelatin Methacrylate (GelMA) and Hyaluronic Acid Methacrylate (HAMA) hydrogels. GelMA (45-55% methacrylated, Cellink) and HAMA (30% methacrylated, molecular weight of 63 kDa via multi-angle laser light scattering, Sigma) stocks were made in Tyrode's (Sigma) containing 0.05% lithium phenyl-2,4,6-trimethylbenzoylphosphinate (LAP, Cellink). GelMA stock (15% w/v) was prepared with prewarmed Tyrode's (˜40° C.) on a hotplate stirrer. HAMA stock (2% w/v) was prepared in Tyrode's at room temperature. All stocks were stored in the dark at 4° C. until use. To prepare biomaterial combinations, hydrogel components were mixed at 40° C. using volume ratios (Table 7). Biomaterials were then cooled to ˜37° C., the cell suspension added and mixed by vortexing. The final cell concentration for all luminescence assays was 2 million cells/mL.

Design of Experiment Trial Design An optimal combined design (Design-Expert Version 13, Stat-Ease) was implemented with two mixture components: GelMA vol % and HAMA vol %, both 0-100%. Crosslinking time was a numeric factor, set as hard to change, ranging between 15 and 60 seconds. A quadratic-by-quadratic model with point exchange generated 18 total groups (Table 7). Groups were used for dynamic mechanical analysis as described below (section 2.8) by casting disc constructs (8 mm diameter×1 mm height) in silicone molds. Following casting, the constructs were crosslinked as described below (section 2.7). and then stored in Tyrode's in the dark at 4° C. overnight. Storage moduli were analyzed using a linear regression model with no transformation to determine significant factors. DoE generated groups were also used in a screen with HuCol2gLuc reporter cells, and the luminescence assay as described below (section 2.9). Analysis was completed using a linear regression model after a log base 10 transformation.

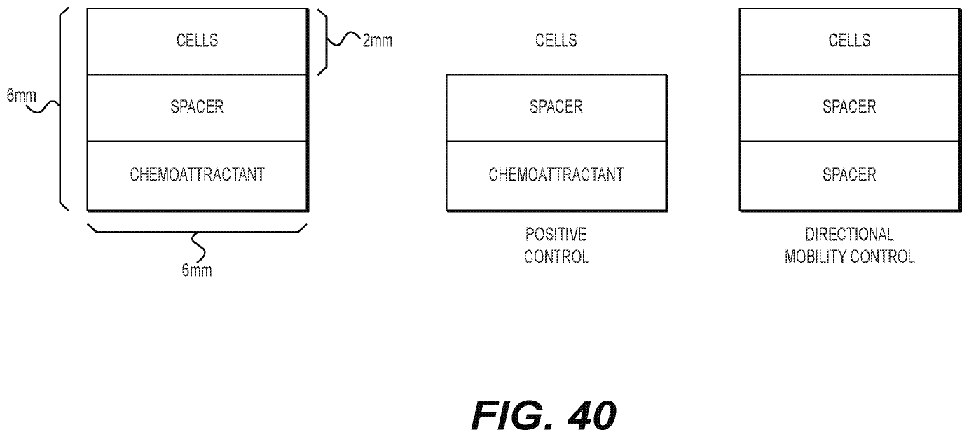

3D Bioprinting. A BioAssemblyBot pneumatic extrusion 3D bioprinter (Advanced Solutions) was used to fabricate all 3D bioprinted constructs. Biomaterials were printed using disposable, UV-blocking amber cartridges (Nordson), SmoothFlow tapered tips (25G, Nordson) with the print settings shown in Table 8. Tissue Structure Information Modeling (TSIM, Advanced Solutions) software was used to create 3D models. After printing, the constructs were photo-crosslinked (Luck Laser, 405 nm, 300 mW). The laser was focused to 8 mm beam diameter and oriented 3.5 cm above the construct. Single layer prints were used for both cell viability and mobility to obtain clearer images, and more readily quantify cell numbers. For cell viability, 2 mm×6 mm rectangular cuboids were printed in a single layer (˜0.3 mm height). For mobility, 6 mm×6 mm rectangular cuboids were printed in a single layer (˜0.3 mm height, in three 2 mm×6 mm sections, FIG. 40). Cylindrical constructs (8 mm diameter×1 mm height) were printed containing HuCol2gLuc cells for the luminescence assay and final mechanical characterization (the same dimensions used for DoE screen mechanical characterization).

Mechanical Characterization. Before testing, discs were allowed to equilibrate to room temperature and the width and height measured with calipers (Netzsch). Unconfined uniaxial compression testing was performed (DMA 242E Artemis, Netzsch). Strain (10%, 100 μm) was tested at frequencies of 0.1, 1 and 5 Hz. All tests were completed at isothermal (room) temperature and discs were maintained in Tyrode's for the duration of the test. Storage modulus (E′), loss modulus (E″) and Tan delta were measured for 30 minutes, and final values were calculated from the average values between 5 and 10 minutes, during the curve plateau.

Luciferase Assessment. Luciferase assessment was performed as a proxy to estimate type II collagen expression in both the pipetted material DoE screen, and the 3D bioprinted discs. Constructs were cultured for 22 days in defined chondrogenic media with 1 ng/ml TGFβ1 (section 2.3), with feeding every Monday, Wednesday, and Friday with sampling every Wednesday and Friday to maintain a consistent timeframe (˜48 h). For this assay, conditioned cell culture media (20 μl/well) was transferred into a white 96-well plate (Greiner Bio-One) and mixed with a stabilized luciferase assay reaction mix (50 μl) for final concentrations of 0.09 M MES, 0.15M Ascorbic Acid, and 4.2 μM Coelenterazine. Luminescence was read on a plate reader (Biotek Synergy H1 Hybrid Reader or PerkinElmer En Vision 2104 Multilabel Reader).

Cell Viability. Live/Dead staining was used to evaluate the viability of human chondrocytes (1 million cells/mL final concentration) on days 0, 1 and 7. Uninfected human chondrocytes from the same donor, at the same passage, as the HuCol2gLuc reporter cells and HuChon-GFP cells were used. GelMA (15% w/v):HAMA (2% w/v) volume ratios of 1:1, 2:1 and 3:1 were either 3D bioprinted, or pipetted (control) onto a BSA (3.15 mg/cm², Alfa Aesar) coated 24-well plate (Corning) and cultured in growth media. At each time point, media was removed and the staining solution of calcein-AM (2 μM, Invitrogen) and ethidium homodimer-1 (4 μM, Invitrogen) in sterile PBS was added to each well. Samples were incubated for 25 minutes (37° C., 5% CO₂). Imaging was performed using a Pico Imager (Molecular Devices). Images were processed and analyzed using ImageJ/Fiji with the Stardist plugin, with >100 cells quantified for each sample.

Cell Mobility. HuChon-GFP cells were used to assess cell mobility. Three 2 mm×6 mm sections were bioprinted forming one construct (FIG. 40, final 6 mm×6 mm). One section contained HuChon-GFP cells (1 million cells/mL final concentration) in GelMA (15% w/v):HAMA (2% w/v) mixtures at volume ratios of 1:1 or 2:1. The second section was comprised only of biomaterial to act as a spacer between the cells and chemoattractant, printed with the same biomaterial volume ratio the cells were in. The final section contained fibroblast growth factor 2 (100 ng/mL, basic FGF, Peprotech) in the 2:1 volume ratio. The chemoattractant was only added to the 2:1 ratio to eliminate any impact on diffusion rate by the material ratio. As a positive control for 2D cell mobility, cells were seeded directly to the 12-well plate (Corning). A separate control for directional mobility had all 3 bioprinted sections, except an extra spacer was bioprinted in place of where the chemoattractant was in the other groups. Three replicates were used for each group, and all constructs were cultured in growth media (section 2.1). Time lapse imaging was performed with a Pico Imager (Molecular Devices) at 4× magnification; images were captured every 20 minutes for 20 hours starting immediately after printing. Images were analyzed with ImageJ/Fiji (Version 2.3.0/1.53q) and the Manual Tracking plugin. For each well, all mobile cells were tracked in each well, except for the positive control which had too many mobile cells to track, 7 randomly chosen cells were chosen. Tracked results included distance, velocity, and location (X, Y coordinates).

Biochemical Assays. Two thirds of the 8 mm×1 mm bioprinted discs were frozen (−20° C.) until the time of the assay. DNA, GAG and hydroxyproline (HDP) content were measured in samples from day 0 (immediately after printing) and day 22. Samples were digested overnight (65° C.) in papain (0.025 mg/mL papain in 50 mM sodium phosphate, 2 mM EDTA, 2 mM cysteine). DNA content of the digest was measured using Hoechst dye (Ex365, Em460 nm, SpectraMax iD5, Molecular Devices) with calf thymus DNA (Sigma) as a standard. GAG content of the digest was measured using the dimethylmethylene blue (DMMB) colorimetric assay. DMMB working solution (3.2 mg/mL DMMB in 200 proof ethanol, 40 mM glycine, 40 mM NaCl at pH 1.5) was added to each sample and absorbance was read at 525 nm with correction at 595 nm (SpectraMax iD5, Molecular Devices) with chondroitin sulfate (Seikagaku Biobusiness) as a standard. For HDP, papain digested samples were acid hydrolyzed overnight (10:1 vol/vol, 6M HCL, 110° C.) and then dried overnight (70° C.). Copper sulfate (0.15M) and NaOH (2.5M) were added to each well and incubated (50° C., 5 minutes). Samples were oxidized through incubation with hydrogen peroxide (6% H₂O₂, 50° C., 10 minutes). Then sulfuric acid was added (1.5M H₂SO₄) before reaction with Ehrlich's reagent (10% w/v 4-dimethylamino benzaldehyde in 60% isopropanol, 26% perchloric acid, 14% MQ2 water). Samples were incubated again (70° C., 16 minutes). Absorbance was read at 505 nm (SpectraMax iD5, Molecular Devices) and total content was calculated using a hydroxyproline standard curve generated using hydroxyproline (Sigma), Samples of materials without cells were used to subtract background generated by gelatin. Data was then normalized to day 0.

Histology. At the end of the 22-day culture period, a third of the 8 mm×1 mm disc was fixed in 10% Neutral Buffered Formalin (Epredia) for ˜2 days. Samples were dehydrated and embedded in paraffin wax. Rehydrated sections were stained with Safranin-O (Electron Microscopy Sciences) then counterstained with Fast Green (VWR, Alfa Aesar) and hematoxylin (Electron Microscopy Sciences) for evaluation of sulfated GAG content. Immunohistochemistry was performed to evaluate type II collagen content. Pronase (1 mg/mL for 10 minutes at RT, Sigma) was used for antigen retrieval and tissue sections were blocked with BSA (3% w/v, Cohn Fraction V Alfa Aesar). The type II collagen primary antibody (1:200 in 1% BSA, DSHB, II-II6B3) was incubated for 2 hours at RT and the secondary antibody (1:200 in 1% BSA, Biotinylated horse anti-mouse, Vector Labs) was incubated for 1 hour at RT. Sections were then incubated for 30 minutes at RT with HRP-conjugated streptavidin (1:5000 in 1% BSA, Invitrogen), and staining was developed using Vector VIP Peroxidase Substrate Kit (VWR). All sections were then counter stained with Fast Green. Images were taken using a Keyence BZ-X810 microscope.

Statistical Analysis. Design of Experiment analysis was completed as described earlier (section 2.5) using Design-Expert (Version 13, StatEase). All other experiments were performed with 3-6 replicates (n=3-6). Quantitative results are shown as mean±/−standard deviation and statistical analyses were done using GraphPad Prism 9.0 or Design-Expert (Version 13, Stat-Ease). HuCol2gLuc screen data was analyzed using ANOVA and Tukey's multiple comparison. Cell viability data was analyzed with a 2-way ANOVA and Sidak's multiple comparison for analysis of printed vs pipetted controls and Tukey's multiple comparison for comparing printed conditions over time. DNA, GAG, HDP and final storage modulus were analyzed with a 2-way ANOVA with Sidak's multiple comparison. The HuCol2gLuc disc 3D bioprint and mobility were analyzed with an unpaired t-test. A value of p<0.05 was considered statistically significant.

### Results

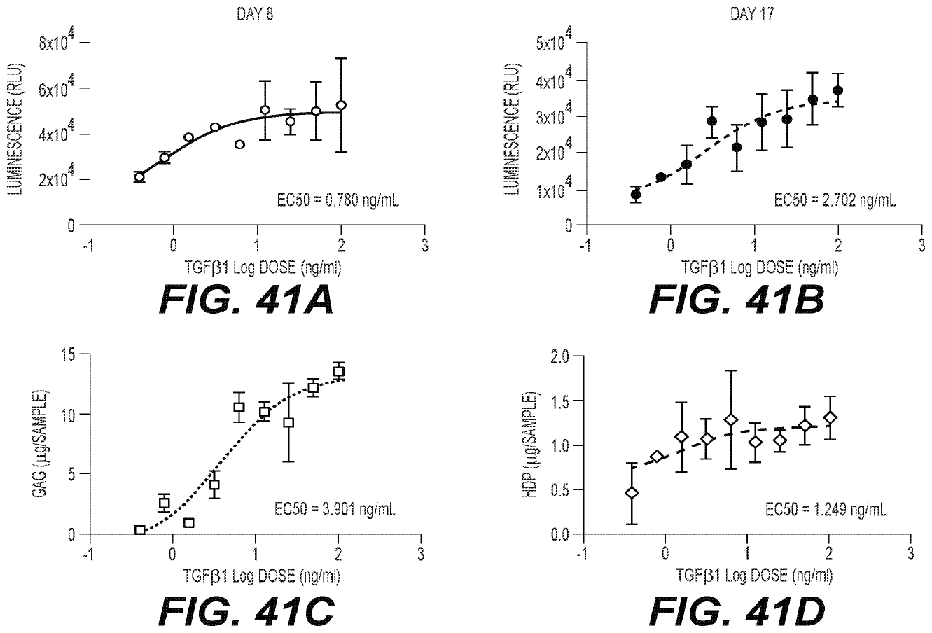

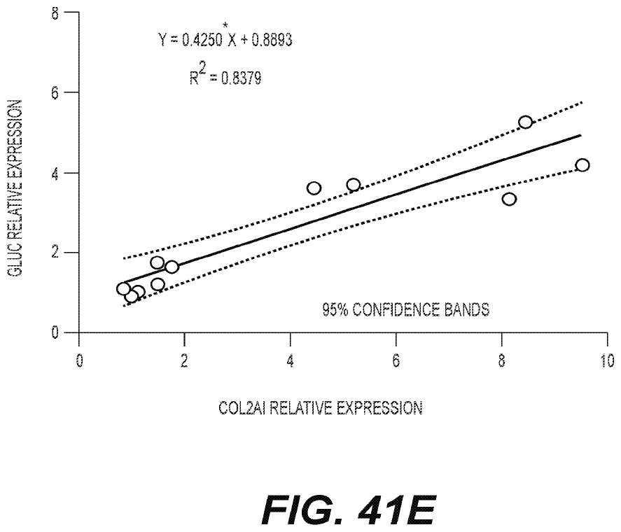

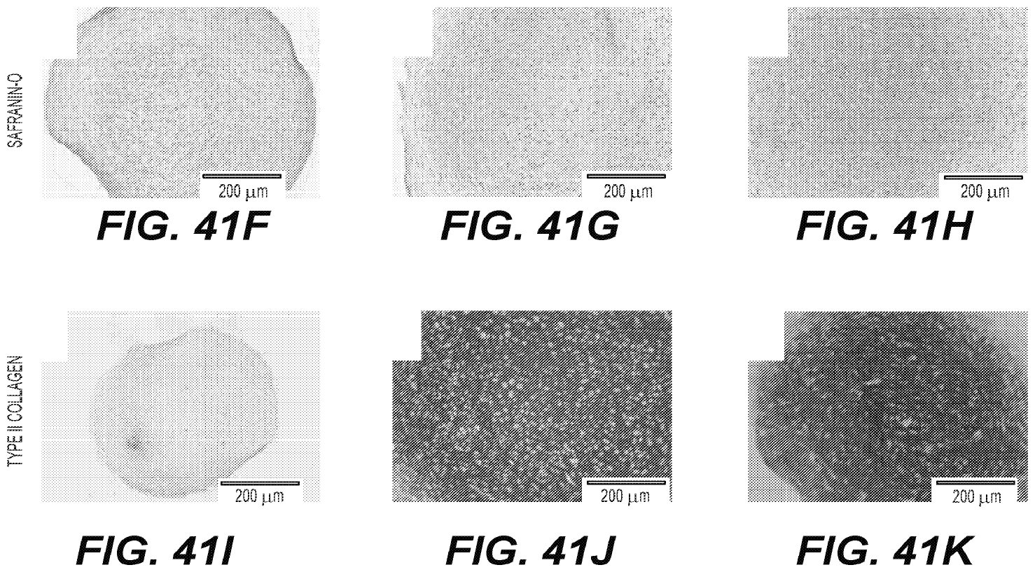

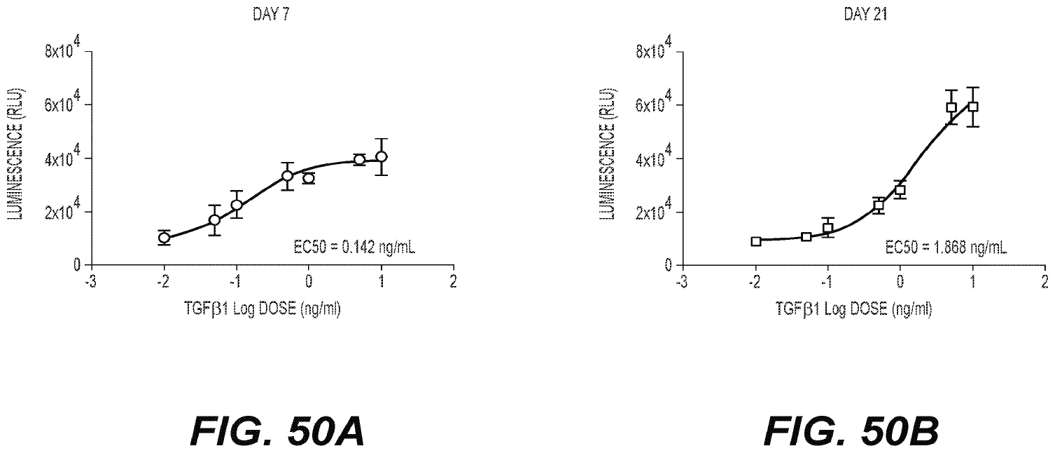

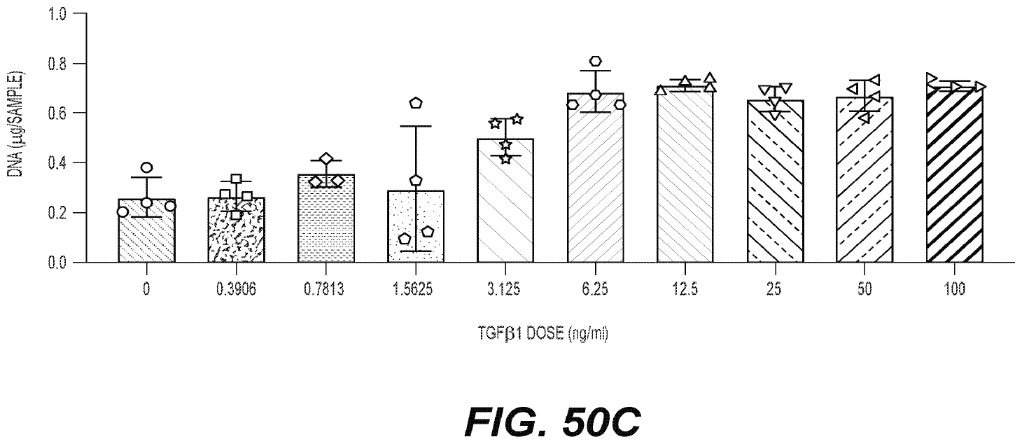

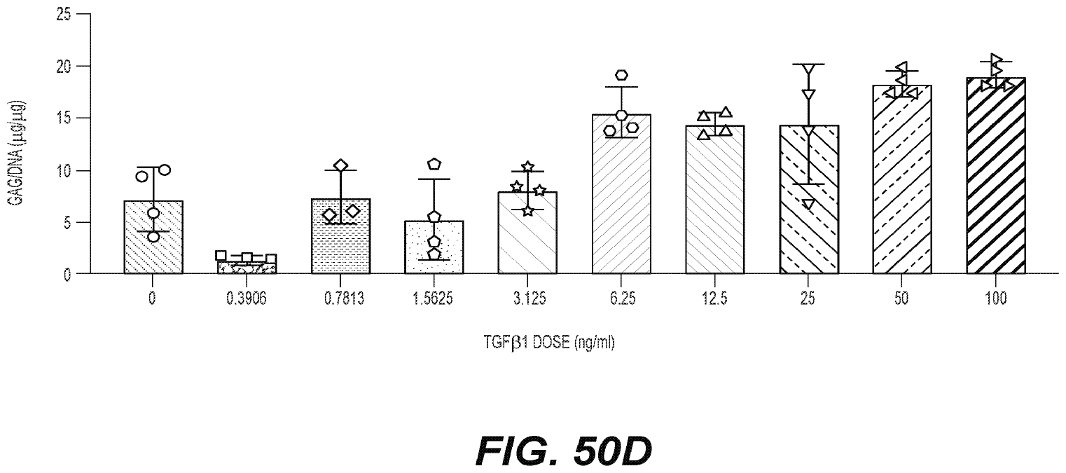

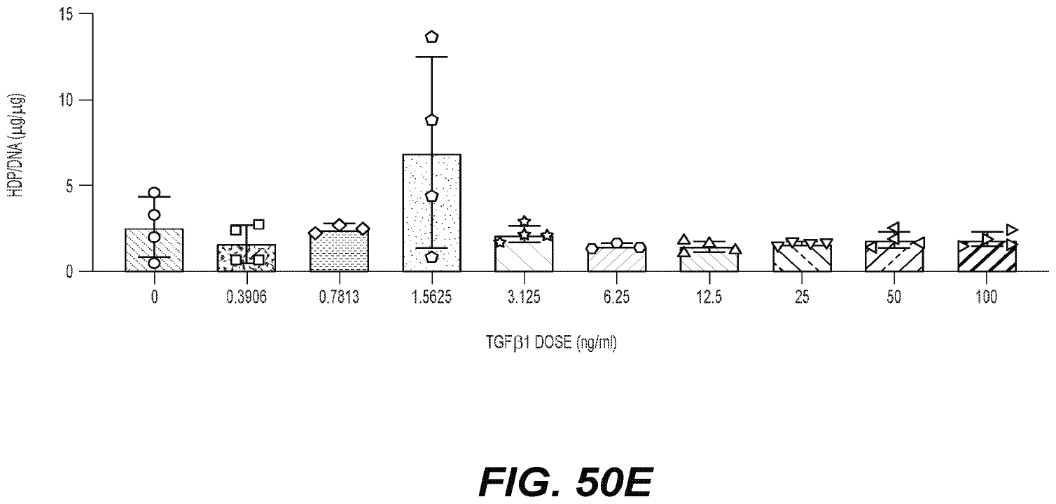

Stimulation of type II collagen by TGFβ1 in primary human chondrocytes. TGFβ1 dose response was used to characterize the engineered primary human chondrocytes transduced with a type II collagen Gaussia luciferase construct (HuCol2gLuc) derived from two donors. Dose dependent increases in luminescence are shown for day 8 (FIG. 41A) and day 17 (FIG. 41B). The excitatory concentration producing a half-maximal response (EC50) was calculated to be 0.780 ng/mL for day 8 and 2.702 ng/mL for day 17. Dose response curves for donor 2 had similar EC50 values of 0.142 ng/mL for day 7 (FIG. 50A) and 1.868 ng/mL for day 21 (FIG. 50B). Biochemical and histological analysis was performed on HuCol2gLuc reporter cells to characterize their ability to produce extracellular matrix proteins. There was a TGFβ1 dose dependent increase of DNA (FIG. 50C), GAG (FIG. 41C), and GAG/DNA (FIG. 50D). Which was also supported by Safranin-O staining (FIG. 41F-41H). HDP (FIG. 41D) and HDP/DNA (FIG. 50E) expression was consistent across groups. However, histology for type II collagen showed a TGFβ1 dose dependent increase in staining intensity (FIG. 41I-41K). This data is consistent with the dose dependent increase in luminescence, indicating the HuCol2gLuc reporter cells reflect type II collagen expression. This is further supported by qPCR analysis for COL2A1 and gLuc. As COL2A1 gene expression increased so did gLuc (FIG. 41E). Based on the maximal luminescence signal, donor 2 HuCol2gLuc cells were used for all subsequent experiments.

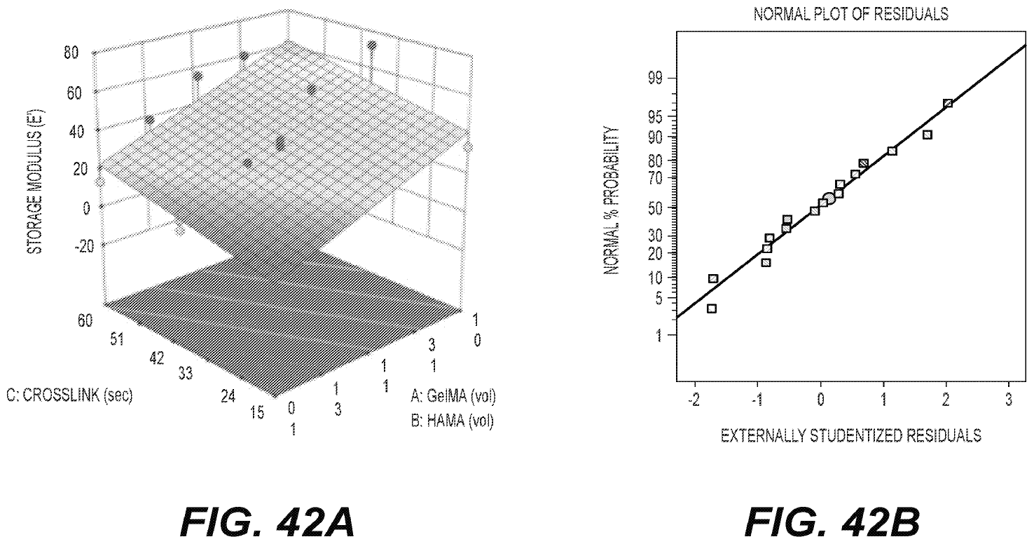

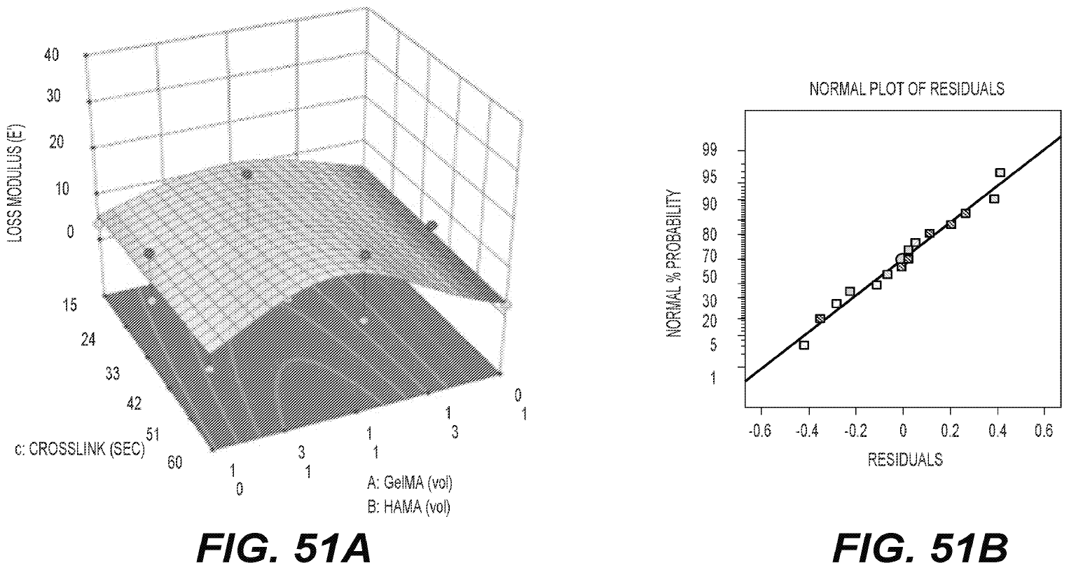

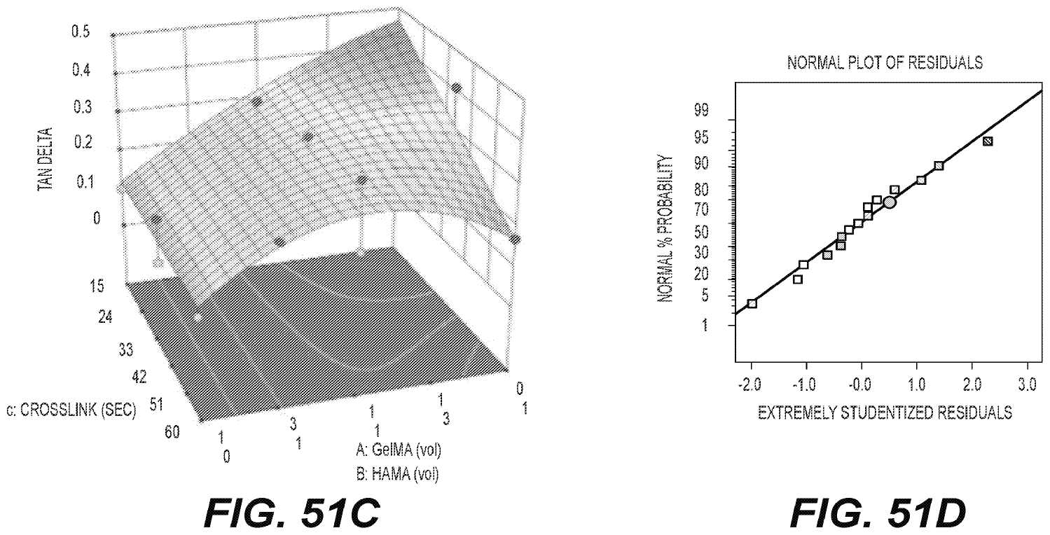

Initial mechanical characterization of GelMA:HAMA constructs. Dynamic mechanical analysis (DMA) was used to measure the moduli (storage modulus (E′), loss modulus (E″) and tan delta) of 18 different biomaterial ratios (GelMA:HAMA) after varying photocrosslinking times that were generated by the DoE software (Table 1). Storage moduli increased as the amount of GelMA increased and as crosslinking time increased (FIG. 42A). Three GelMA:HAMA ratios were too soft to be tested: 0:1 15s, 1:1 15s, and 1:3 60s. HAMA alone (0:1, at 38s and 60s) had the lowest storage moduli tested resulting in 6.9 kPa and 11.9 kPa, respectively. GelMa:HAMA 2:1 38s and 3:1 60s had the highest storage moduli tested at 57.9 kPa and 60.6 kPa, respectively. Residual data was normally distributed (FIG. 42B). It was found that the mixture of materials had a significant impact on the storage modulus (p=0.0006, ANOVA), but crosslinking time did not (p=0.0714, ANOVA). The opposite trend was observed for tan delta (FIG. 51C) in which shorter crosslinking time, and increased amount of HAMA increased tan delta. However, neither crosslinking time (p=0.2589) nor material mixture (p=0.1151) had a statistically significant impact. There were no trends observed for the loss moduli (FIG. 51A), and neither crosslinking time (p=0.9677) nor material mixture (p=0.3208) had a significant impact.

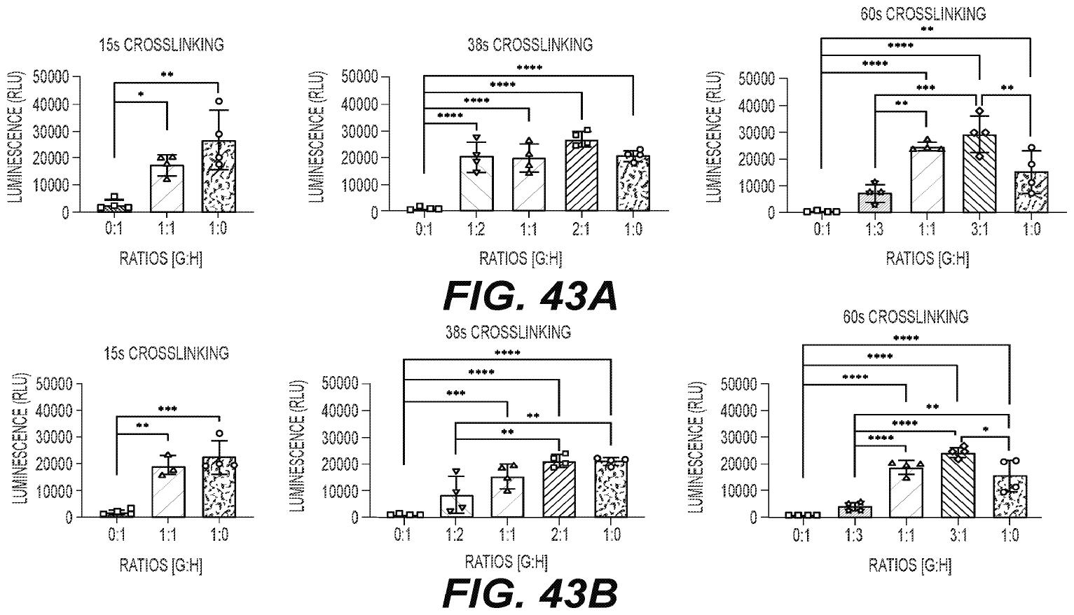

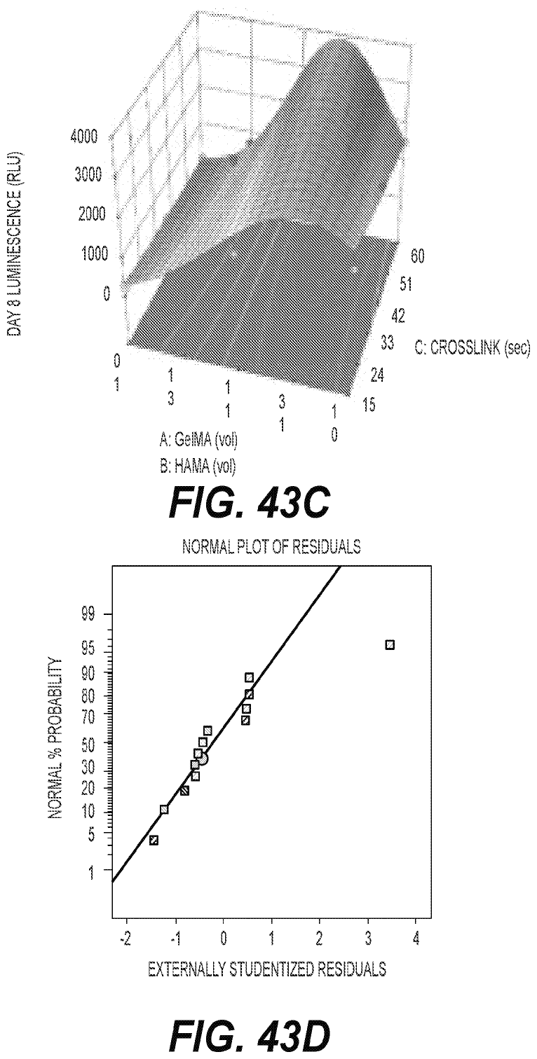

HuCol2gLuc Screen of GelMA:HAMA material combinations. HuCol2gLuc reporter cells were used to identify combinations of GelMA:HAMA that stimulated type II collagen expression. The combinations of GelMA:HAMA tested were generated by the DoE software (Table 7). These were the same 18 groups that underwent DMA assessment. Luminescence results for days 8 (FIG. 43A) and 22 (FIG. 43B) are shown with similar trends. HAMA alone (0:1) at all crosslinking times had significantly lower luminescence compared to all groups except 1:3 60s on day 8, and both 1:2 38s and 1:3 60s on day 22 (FIGS. 43A and 43B). At the shortest crosslinking time (15s) luminescence was highest in GelMA alone (FIGS. 43A and 43B). After 38s crosslinking the 2:1 ratio had the highest luminescence (FIG. 43A). Group 3:1 60s had significantly higher luminescence compared to GelMA alone on both days (FIGS. 43A and 43B). Response surface analysis shows a clear peak in chondrogenic stimulation around the 3:1 ratio, at all crosslinking times (FIG. 43C). The normality plot (FIG. 43D) demonstrates the fit of the data and therefore the validity of the analysis used.

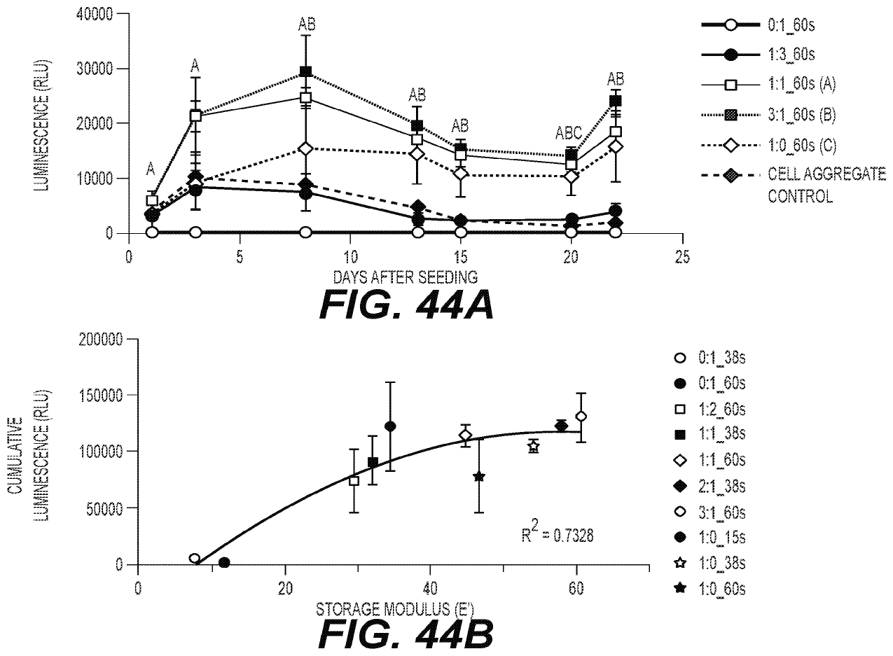

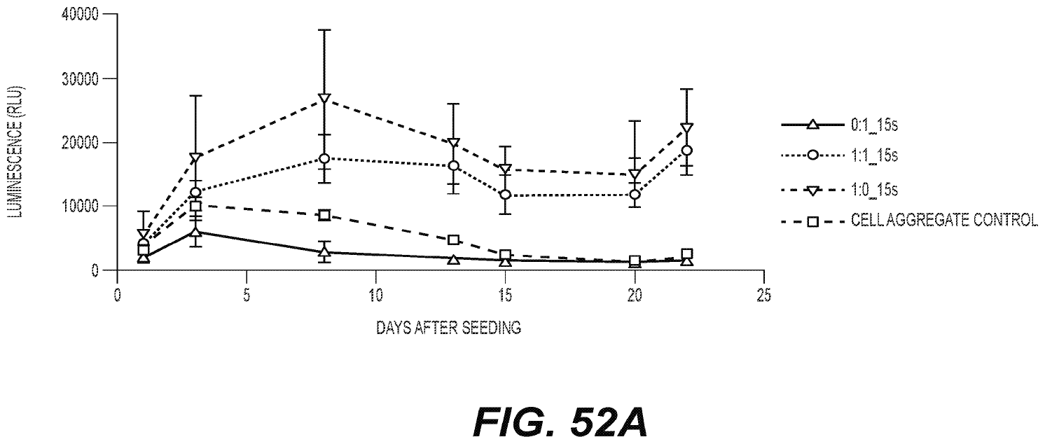

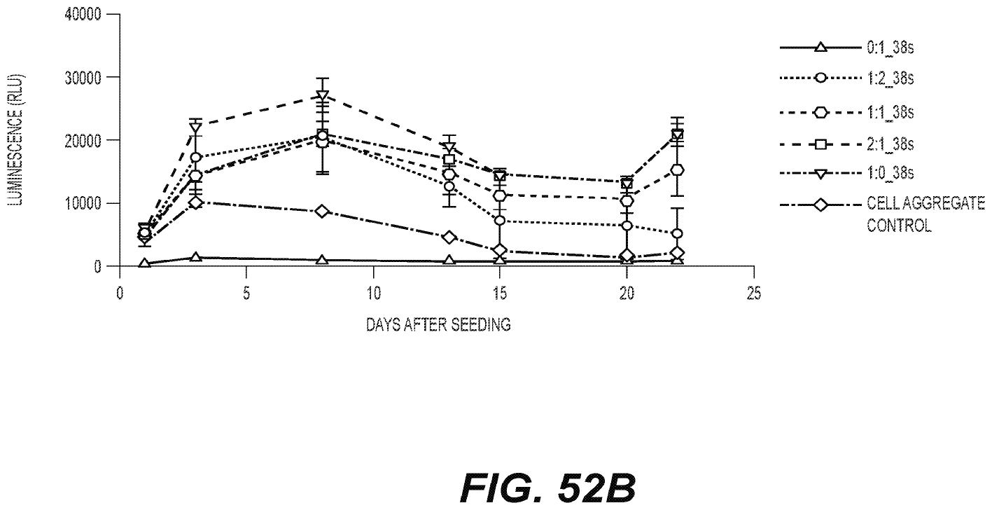

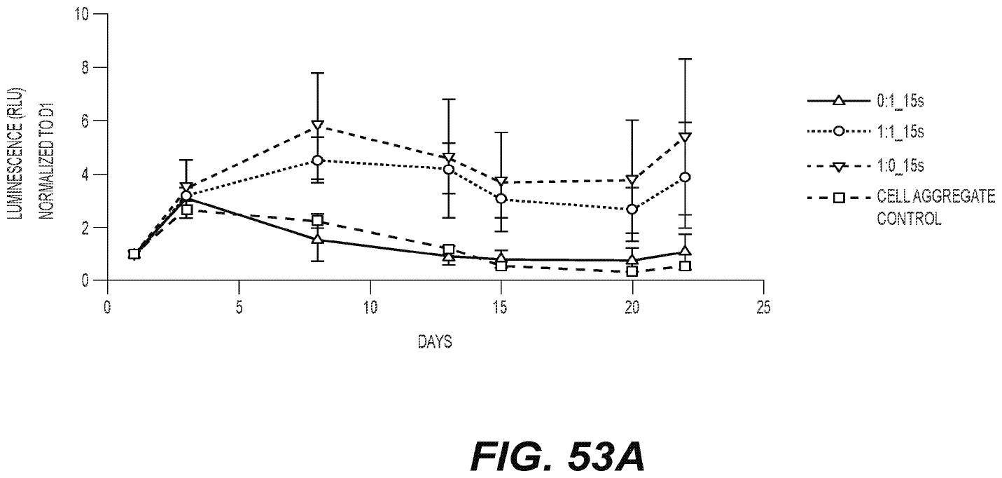

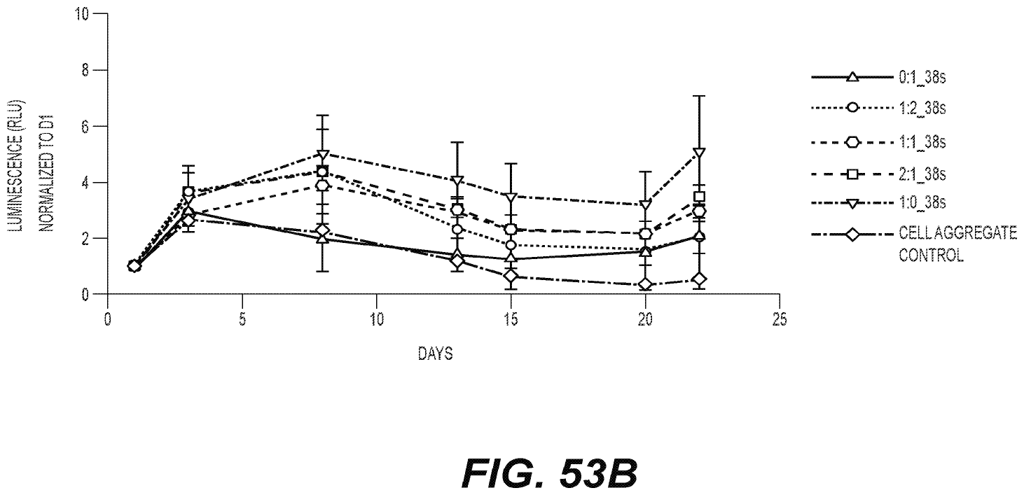

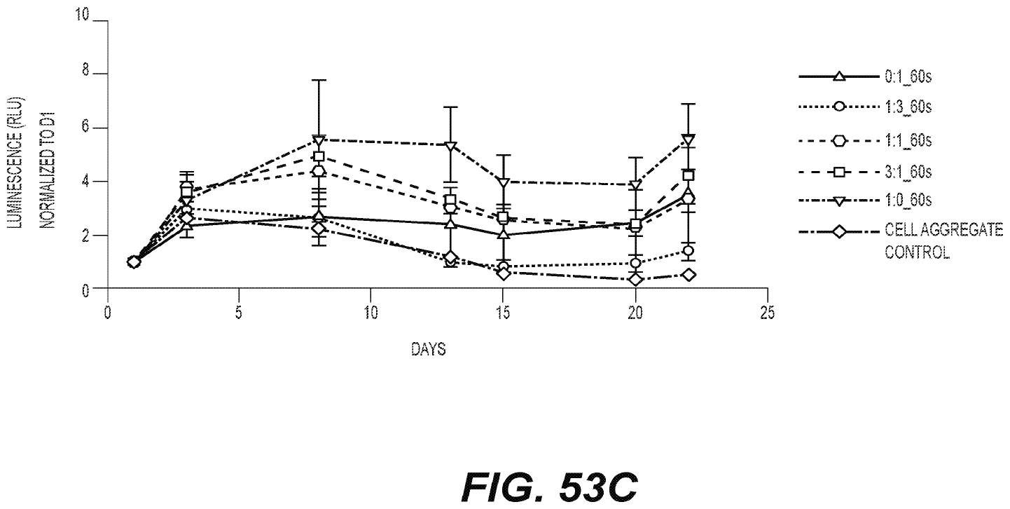

To determine if biomaterials improved luminescence compared to traditional 3D cell aggregates, HuCol2gLuc cells were seeded as aggregate cultures and luminescence assessed over 22 days. Temporal analysis of chondrogenic response showed an initial peak in luminescence on day 8 with an uptick at day 22 (FIGS. 44A, 52A and 52B). All groups except 1:3 60s and HAMA alone after all crosslinking times had higher luminescence as compared to the cell aggregate control (FIGS. 544A, 52A and 52B). However, when luminescence data is normalized to day 1 this trend changes (FIG. 53). Groups 1:3 60s, and HAMA alone at 15s and 38s were similar to the cell aggregate control, while HAMA alone at 60s had higher luminescence (FIG. 53). To analyze the impact of storage modulus on luminescence, cumulative luminescence vs. storage modulus was plotted (FIG. 44B). The softest materials tested, HAMA alone (0:1 38s and 60s) had the lowest level of luminescence. Above ˜30 kPa, there is a similar cumulative luminescence value for all groups (FIG. 44B).

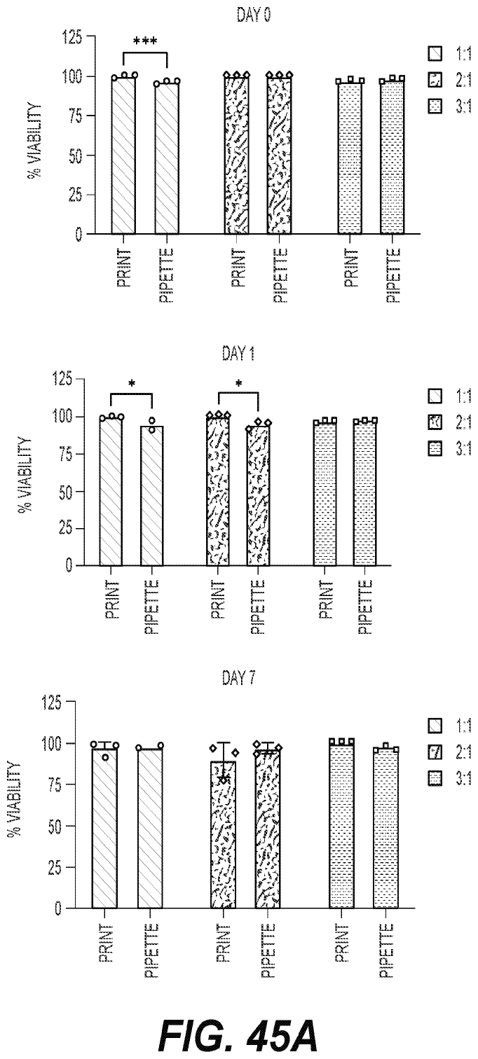

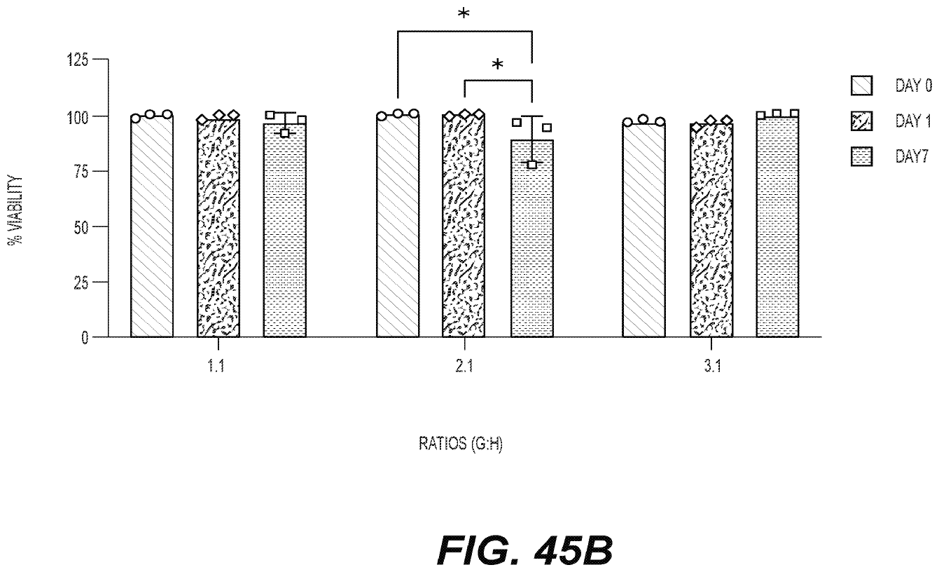

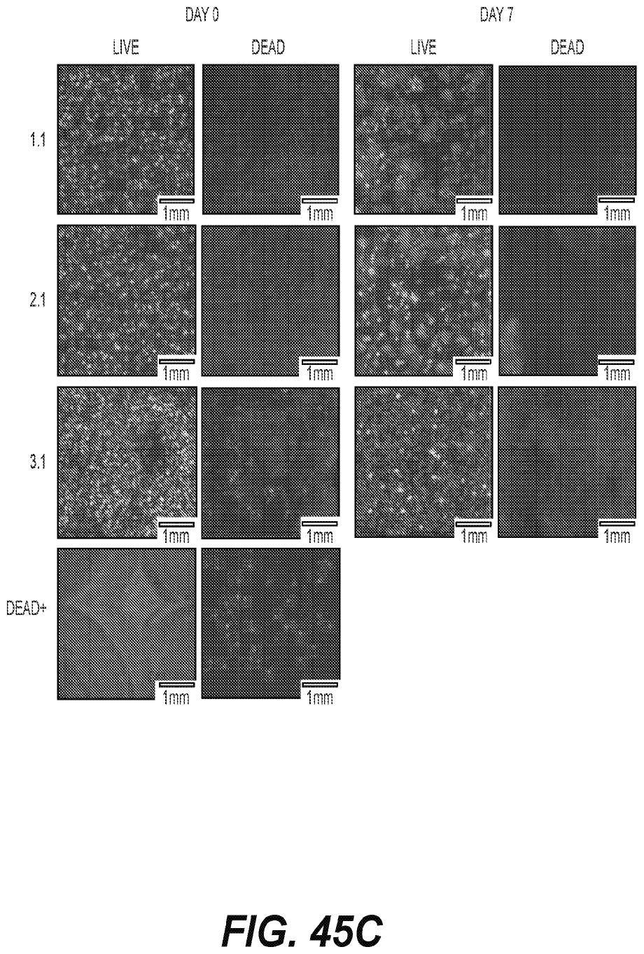

Human chondrocyte viability in 3D bioprinted constructs. GelMA:HAMA ratios of 1:1, 2:1 and 3:1 after 38s crosslinking were chosen for subsequent 3D bioprinting assays. To ensure the materials and 3D bioprinting process were biocompatible, cell viability was quantified on days 0, 1 and 7 (FIG. 45). As a control, the same biomaterial combinations were pipetted directly onto the plate. High cell viability was observed for all groups on all days, at 90% or higher (FIG. 45). On day 0 and day 1, viability was higher in the printed group at 1:1 (99%) than the pipetted (93-96%), this was also true for 2:1 on day 1 (100% for printed and ˜96% for pipetted) (FIG. 45A). However, by day 7 all groups had similarly high percent viability (above 90%). To determine if cell viability changed over time, bioprinted material ratios were compared across all three time points (FIG. 45B). While the 1:1 and 3:1 groups remained consistent, the 2:1 group had a significant decrease by day 7 (˜90%) as compared to day 0 (100%) and day 1 (99%). Representative images of all bioprinted groups on day 0 and day 7 are shown (FIG. 45C).

Human chondrocyte mobility within 3D bioprinted constructs. To determine the effect of biomaterial storage moduli on cell mobility, ratios of GelMA:HAMA ratios of 1:1 and 2:1 after 38s crosslinking were 3D bioprinted with HuChon-GFP cells encapsulated in the biomaterials. These ratios were chosen since 2:1 had a storage modulus of 57.9 kPa, and 1:1 was roughly half at 32.0 kPa. Expanded HuChon-GFP cells were fluorescent (FIG. 46E) and were readily visible within 3D bioprinted biomaterials (FIG. 46F). Timelapse images over 20 hours showed that human primary chondrocytes are mobile in 2D in vitro culture, as demonstrated by the positive control (FIG. 46A-46C). In the biomaterials cells were minimally mobile, the 1:1 ratio had significantly more mobile cells (˜2.5) compared to 2:1 (˜1) (FIG. 46A). Mobile cells also moved further (˜26 μm, FIG. 46B) and faster (˜1 μm/min, FIG. 46C) in the 1:1 ratio. Directionality of cell movement was also assessed and the 1:1 group had more variability in the movement (FIG. 46D). Overall, the softer material (1:1) had a higher level of cellular mobility as compared to the stiffer (2:1) material (FIG. 46).

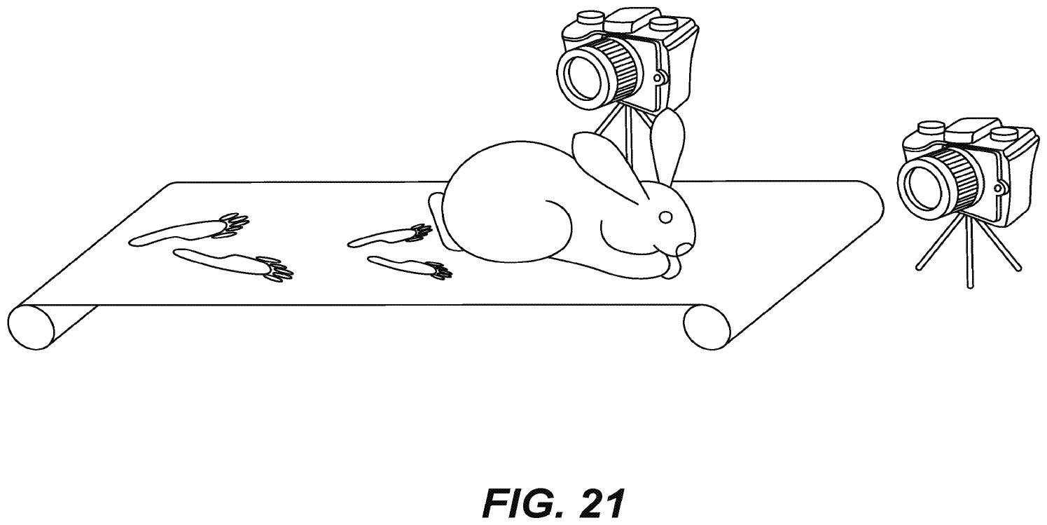

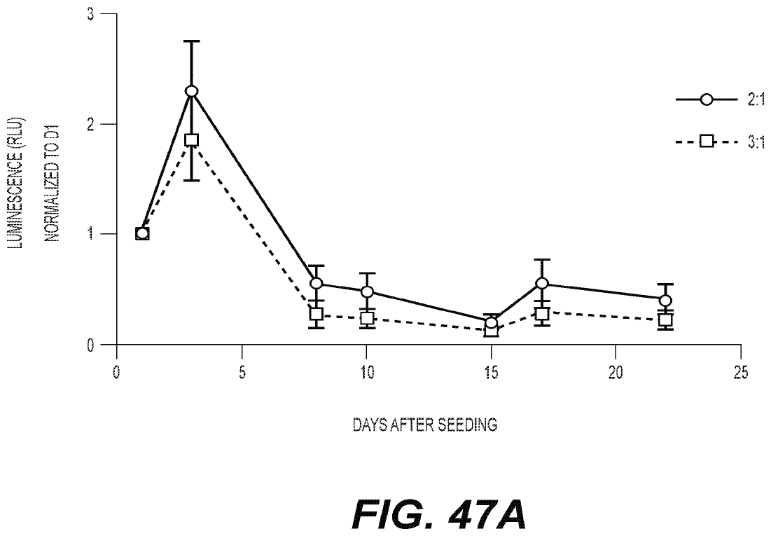

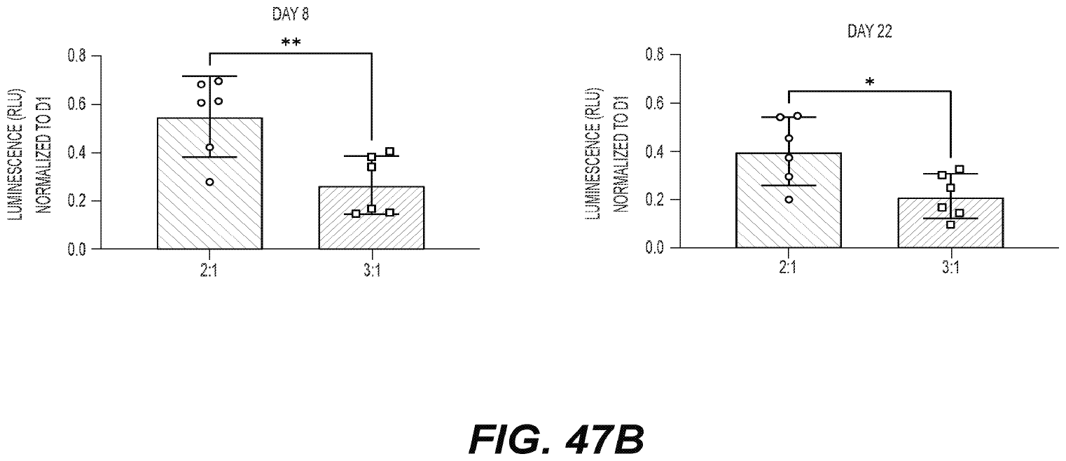

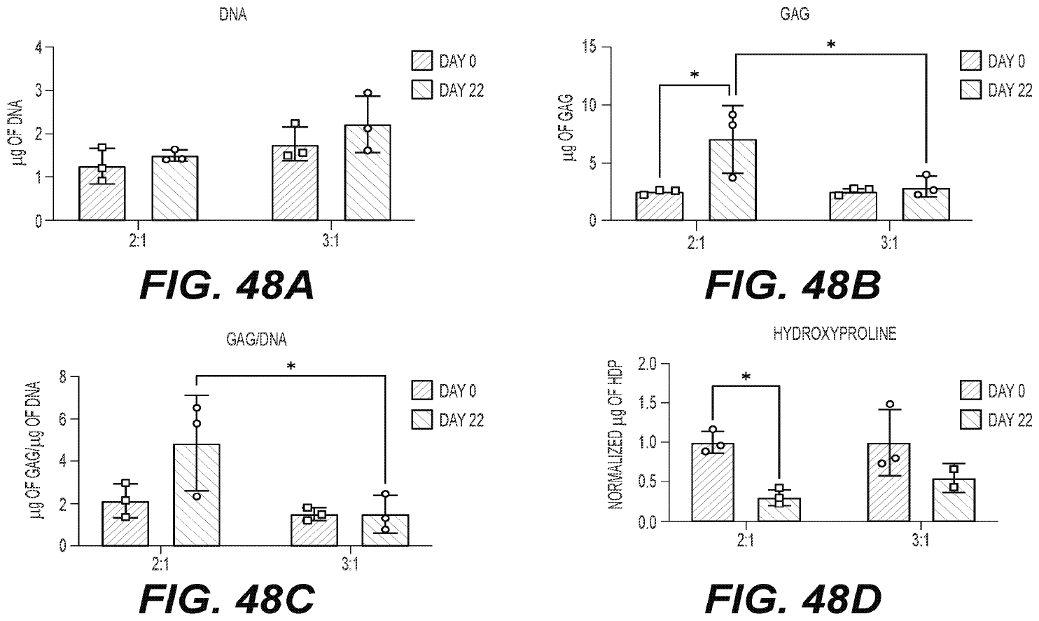

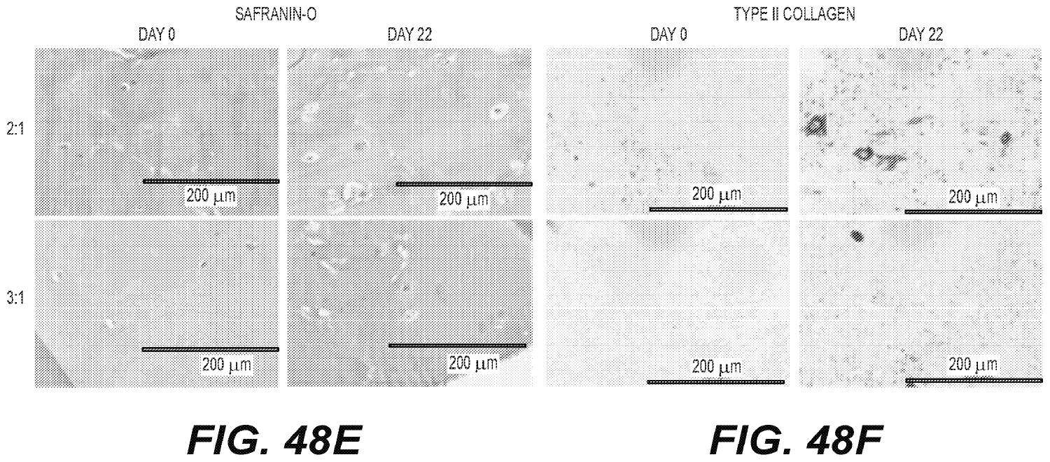

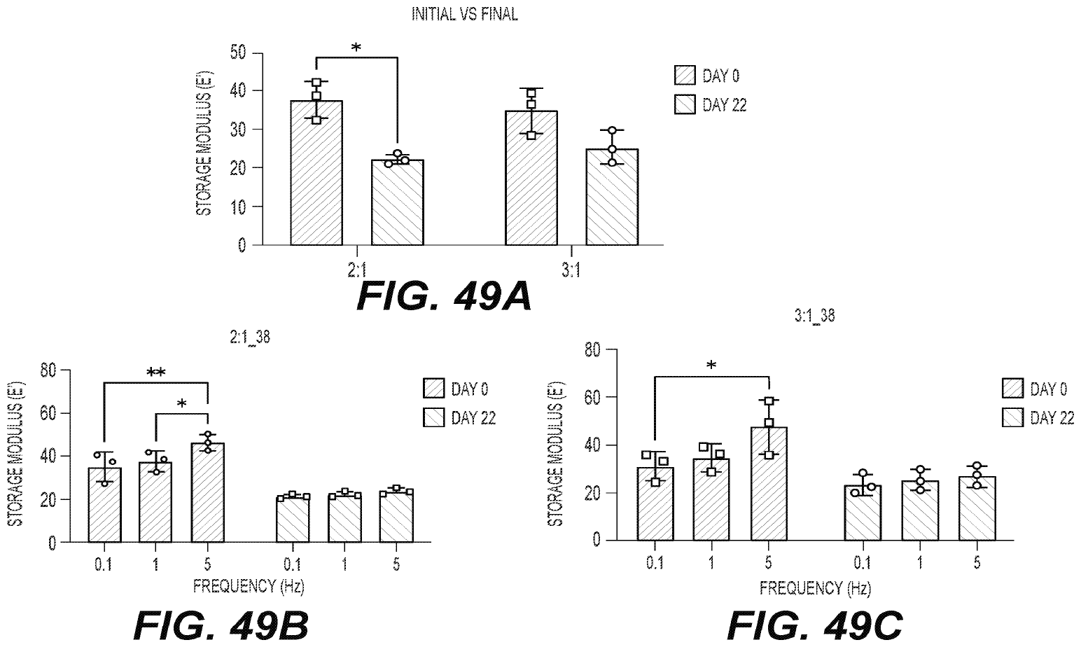

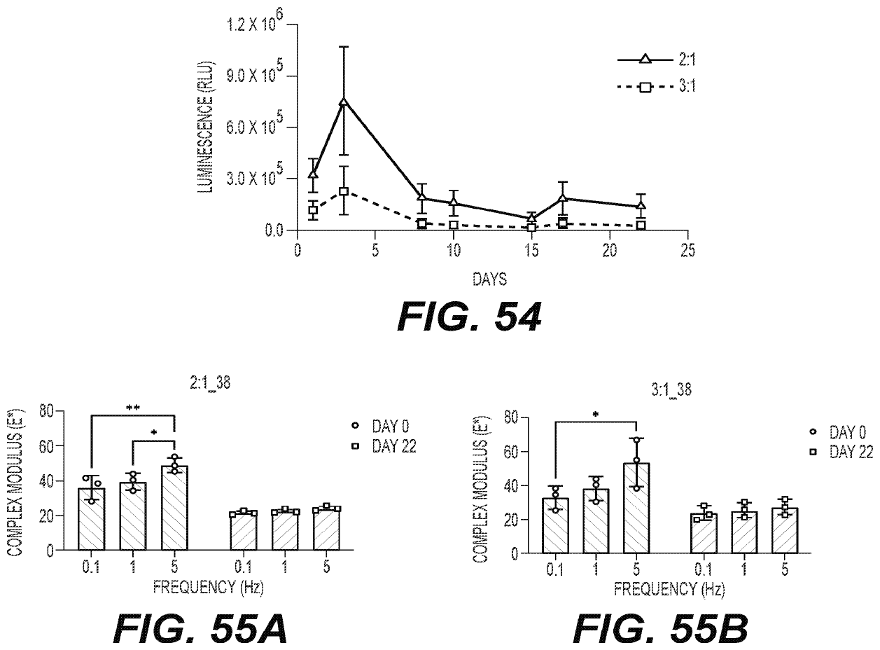

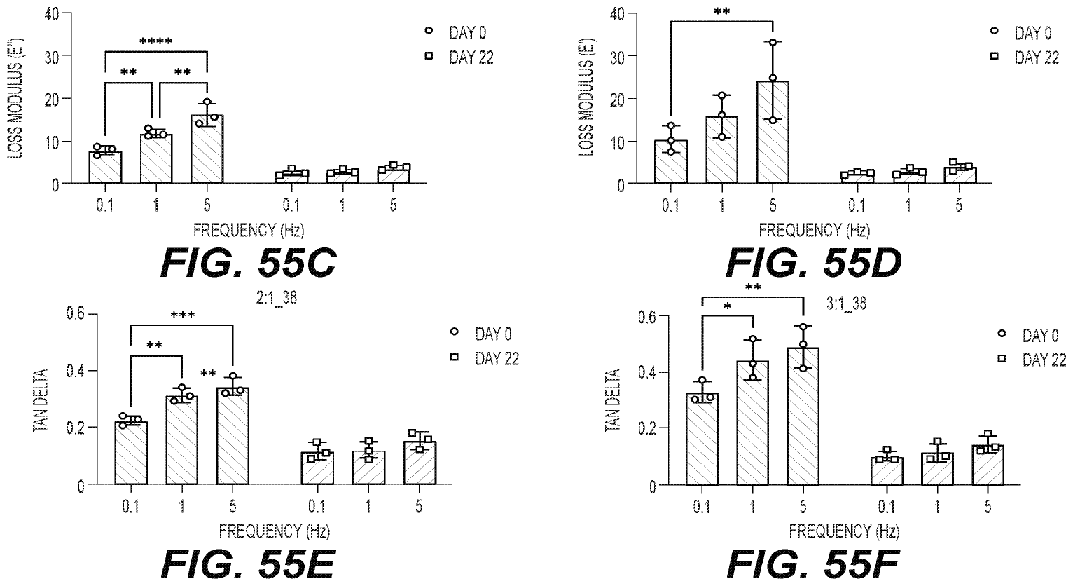

3D bioprinted discs containing HuCol2gLuc. Based on the luminescence and stiffness results generated from the HuCol2gLuc DoE screen the 2:1 and 3:1 38s groups were chosen for 3D bioprinting. HuCol2gLuc reporter cells were encapsulated in GelMA:HAMA ratios and bioprinted into discs then photo-crosslinked for 38s. To assess type II collagen expression, luminescence was measured over 22 days. Data was normalized to day 1 due to an initial difference in the raw luminescence data between the two groups (FIG. 54). Luminescence peaked on day 3 (FIG. 47A, 54). The 2:1 ratio had significantly higher luminescence at day 8 (˜0.5 RLU) and day 22 (˜0.4 RLU) when normalized to day 1 as compared to the 3:1 (Day 8 ˜0.3 and day 22 ˜0.2, FIG. 47B). Biochemical analyses were used to quantify the amount of DNA, GAG and HDP in the bioprinted constructs. DNA content was similar (1.3-2.3 μg) between both groups at both time points (FIG. 48A). However, there was a significant increase in GAG production in the 2:1 group on day 22 (˜7 μg) as compared to day 0 (˜2.5 μg), while there was no increase in the 3:1 group (FIG. 48B). The 2:1 group also had significantly more GAG expression as compared to the 3:1 group on day 22 (˜3 μg, FIG. 48B). These trends were also observed in the ratio of GAG/DNA (FIG. 48C). Histology on day 22, for both GAG (FIG. 48E) and type II collagen (FIG. 48F) shows only minor staining, predominantly pericellular. The minimal staining is consistent with amount of staining observed in the cell aggregates at a similar TGFβ1 dose (FIGS. 2F and 21). There was a notable decrease in HDP of both groups from day 0 to day 22 (FIG. 48D). This correlated with a decrease in storage modulus from day 0 to day 22 in both groups (FIG. 49A), with a statistically significant decrease in the 2:1 group (˜37 to 22 kPa). The storage modulus on day 0 increased with increasing frequency for both groups, but this increase was not observed on day 22 (FIGS. 49B and 49C). This trend was also observed in the complex modulus (FIGS. 55A and 55B), loss modulus (FIGS. 55C and 55D) and tan delta (FIGS. 55E and 55F).

For the first time, the advantages of using human primary phenotypic reporter cells for biomaterial optimization was demonstrated. HuCol2gLuc reporter cells were developed as a high-throughput assay to measure type II collagen expression. Type II collagen was used as a proxy for chondrogenesis since it is both the main component of articular cartilage, and it is difficult to achieve native levels of expression in vitro. By using a secreted Gaussia luciferase, the assay is conducted on the culture medium, allowing for a non-destructive temporal assessment of type II collagen expression over 22 days. This approach was successfully used to measure type II collagen expression within biomaterials, and not just cell aggregates. This study was further streamlined by using a Design of experiments (DoE) approach. The DoE software generates test conditions using statistical modeling to limit the number of conditions tested, while still having a good overview of the design space. A mixture design approach is an established and efficient method to investigate how biomaterial properties are affected by altering the composition and crosslinking times.

GelMA and HAMA were chosen as the biomaterials for this study since their individual material characteristics are well established and widely used in chondrogenesis. Several studies have combined the two for 3D bioprinting of cartilage to investigate numerous concepts including sterilization techniques, cell density and patterning, cell types, and printing techniques. Whilst they are not new materials, to the best of our knowledge, this study is the first to determine the optimal ratio of GelMA and HAMA for type II collagen expression in 3D bioprinting cartilage.

Articular cartilage functionality is dictated by its mechanical properties therefore, it is unsurprising that assessment of these properties in biomaterial constructs is a significant area of research. However, mechanical testing of constructs is most often performed prior to encapsulation of cells and without 3D bioprinting. A few studies have compared mechanical properties from day 0 to the end point to demonstrate how matrix deposition increases mechanical stiffness. Those studies present initial stiffness as a baseline to improve upon, instead of a necessary characteristic of the biomaterials. One study has determined the minimal initial stiffness required for chondrogenesis to be 7-33 kPa. Another determined ˜30 kPa Young's modulus to be ideal for chondrocyte redifferentiation. Due to the variety and inconsistencies in mechanical testing however, it is difficult to make comparisons between studies, and as such it has yet to be determined conclusively what initial storage modulus is required for chondrogenesis. Our results showed that a storage modulus threshold of ˜30 kPa needs to be reached to promote chondrogenesis. Storage moduli was significantly affected by the mixture of GelMA:HAMA, while no significant effects were seen for tan delta and loss moduli. Other mixtures which do impact tan delta and loss moduli can enable investigation of those parameters and their effect on chondrogenesis. Other moduli such as Young's and aggregate could offer further insight into the role of hydrogel mechanical properties on chondrogenesis. Importantly, the HuCol2gLuc reporter cells provide a rapid and easy method to identify correlations between type II collagen expression and biomaterial mechanical properties.

Using HuCol2gLuc reporter cells this study assessed type II collagen expression via luminescence in DoE generated material ratios. Previous studies showed poor cell spreading and adhesion in HAMA, this can be an explanation for the low type II collagen expression in the predominantly HAMA groups. The addition of HAMA to GelMA improved type II collagen expression over GelMA alone at the same photocrosslinking times. This is due to hyaluronic acid providing biochemical cues and being a GAG present in native cartilage tissue in combination with the cell adhesion sites found in gelatin. Simply by combining two biomaterials, type II collagen expression was improved. However, GelMA alone after 15s photocrosslinking had similar luminescence to the 2:1 38s and 3:1 60s, despite a storage modulus of ˜35 kPa as compared to ˜60 kPa in the 2:1 38s and 3:1 60s mixtures. This demonstrates a complex interplay between biomaterial composition, storage moduli and photocrosslinking on chondrogenesis. This has important implications for the field as there are a significant number of biomaterials currently available, along with different crosslinking methods, whose use in combination has not been studied. Using the high-throughput method described in this study to screen material combinations can streamline the process and help save on time and materials.

Cell mobility was used to further characterize the cell response to the biomaterials. Native chondrocytes are generally considered to be immobile due to the dense ECM, yet cell mobility is a hallmark of tissue remodeling and repair. It can be expected that during remodeling of the 3D bioprinted materials, mobility can increase. Chondrocyte mobility has been shown in vitro both in 2D and 3D culture. To our knowledge, this is the first study measuring cell mobility within a 3D bioprinted cartilage construct. This study assessed cell mobility for the first 20 hours after 3D bioprinting in either 2:1 or 1:1 ratio of GelMA:HAMA after 38s crosslinking. These ratios were chosen to further understand the role of storage moduli in cell mobility as the storage moduli of 1:1 was about half that of 2:1. These results showed the material with the higher storage modulus had significantly less cell mobility. The 2:1 ratio was also one of the ratios with the highest type II collagen expression. This indicates that chondrocytes favor an environment that restricts cell movement for chondrogenesis, because it is a closer mimic to native cartilage. It should be noted however, that only a very small fraction of the total cells in the constructs, regardless of the material ratios, demonstrated mobility. Cell mobility is assessed at different time points to determine if mobility further increases during the remodeling process and see if this has any impact on chondrogenesis.

GelMA:HAMA ratios from the screen that did not stimulate a chondrogenic response were eliminated before proceeding to the more laborious process of 3D bioprinting. Disc constructs were 3D bioprinted with the HuCol2gLuc reporter cells. GelMA:HAMA ratios of 2:1 after 38s photocrosslinking was chosen because it had one of the highest storage moduli tested and was among the highest luminescence in the screen. While 3:1 after 38s was chosen because there was a trend of luminescence increasing in the GelMA only group as crosslinking time decreased. Since 3:1 has a higher ratio of GelMA than 2:1 it was thought that the luminescence can increase if the crosslinking time was decreased from 60s to 38s. This also maintained a consistent crosslinking time between groups. Luminescence data generated from the constructs was supported by DNA, GAG and hydroxyproline biochemical data, along with safranin-O staining and immunohistochemistry for type II collagen. Both groups had a cell viability of above 90% at all time points, which is above the 70% threshold required to have a successful implant. This study identified the 2:1 ratio as having the best chondrogenic response.

Strikingly, despite good luminescence for both groups, storage moduli decreased by day 22 as compared to day 0. This trend was also observed with the complex moduli, loss moduli and tan delta. All moduli on day 0 increased with increased frequency, however by day 22 they stayed consistent across all frequencies. On day 0 the materials were more viscoelastic, but the constructs lost this property by day 22. This observation can be due to an imbalance of ECM production and material degradation i.e., the chondrocytes degraded the material faster than they produced ECM. There are several factors that likely contributed to these results. 1) The slower rate of ECM production can be due to a decreased chondrogenic capacity of the cells. While this study showed via luminescence response, deposition of GAG and HDP along with type II collagen and safranin-O staining during aggregate culture that the HuCol2gLuc reporter cells maintain their chondrogenic capacity, it should be noted that, the chondrocytes used were sourced from elderly patients undergoing a total joint replacement. However, the type of cells used in this study can be either a cell source or the target population for treatment of osteoarthritis, therefore it is important to assess biomaterial effect on these cells. 2) Cell density can be an explanation for the decrease in storage moduli. In this study, the final cell density was 2 million cells/mL. This density was chosen for the DoE screen to achieve a total of 50,000 cells/well, the same number used to seed cell aggregates, and was maintained for consistency in the subsequent 3D bioprinting. While this is a lower cell density than often reported in the literature, it is still within range for 3D bioprinting with human articular. 3) The low TGFβ1 concentration (1 ng/mL) used in the chondrogenic media. Low TGFβ1 was chosen for this study so any stimulatory effects of the biomaterials can be evident and can be attributed to the biomaterials, not masked by maximal stimulus produced by higher levels of TGFβ1. As such ECM production rate was reduced. 4) The initial threshold for chondrogenesis might be higher than what was determined here. The results herein showed that an initial storage modulus of ˜30 kPa was necessary for chondrogenesis but ˜60 kPa was the highest storage modulus determined, only double the threshold determined herein. Material degradation and ECM production can be reduced or prevented by the initial storage modulus.

This study successfully identified an optimal combination of GelMA and HAMA for chondrogenesis using HuCol2gLuc phenotypic reporter cell type II collagen expression, biomaterial storage moduli and cell mobility. The HuCol2gLuc reporter cells provided new insight on the storage modulus threshold required for chondrogenesis by correlating type II collagen expression with storage modulus of the same biomaterials. It was also determined that there is less chondrocyte mobility in the stiffer biomaterials. Together these results indicate that a biomaterial with a higher storage modulus and less cell mobility, improves chondrogenesis.

TABLE 7
Design of Experiment generated groups.

| | GelMA | HAMA | |
|---|---|---|---|
| Number | (vol) | (vol) | Crosslink(sec) |
| 1 | 0 | 1 | 15 |
| 2 | 1 | 1 | 15 |
| 3 | 1 | 0 | 15 |
| 4 | 0 | 1 | 38 |
| 5 | 0 | 1 | 38 |
| 6 | 1 | 1 | 38 |
| 7 | 1 | 0 | 38 |
| 8 | 1 | 1 | 38 |
| 9 | 1 | 0 | 38 |
| 10 | 1 | 2 | 38 |
| 11 | 1 | 1 | 38 |
| 12 | 2 | 1 | 38 |
| 13 | 0 | 1 | 60 |
| 14 | 1 | 1 | 60 |
| 15 | 1 | 1 | 60 |
| 16 | 1 | 0 | 60 |
| 17 | 1 | 3 | 60 |
| 18 | 3 | 1 | 60 |

TABLE 8
3D Bioprinting Settings

| GelMA | | | | Ink |
|---|---|---|---|---|
| (15%):HAMA (2%) | Pressure | Acceleration | Speed | Temp |
| (vol:vol) | (psi) | (mm/sec²) | (mm/sec) | (° C.) |
| 1:1 | 15-13 | 200 | 5 | 25 |
| 2:1 | 21-18 | 200 | 7 | 28 |
| 3:1 | 22-17 | 200 | 7 | 28 |

All printed with 25TT (25-gauge Taper Tip needle, Nordson), stage temp of 35° C., line width and height of 0.2 mm.

TABLE 9
Dynamic mechanical analysis data for DoE generated groups

| | | Storage | | | Loss | | | | | | Complex | | |
|---|---|---|---|---|---|---|---|---|---|---|---|---|---|
| | Group | Modulus | | | Modulus | | | | | | Modulus | | |
| | (GelMA: | (E′, kPa) | | | (E′′, kPa) | | | Tan Delta | | | (E*, kPa) | | |
| | HAMA | 0.1 | 1 | 5 | 0.1 | 1 | 5 | 0.1 | 1 | 5 | 0.1 | 1 | 5 |
| Number | Crosslinking) | Hz | Hz | Hz | Hz | Hz | Hz | Hz | Hz | Hz | Hz | Hz | Hz |
| 1 | 0:1_15 | ND | ND | ND | ND | ND | ND | ND | ND | ND | ND | ND | ND |
| 2 | 1:1 15 | ND | ND | ND | ND | ND | ND | ND | ND | ND | ND | ND | ND |
| 3 | 1:0_15 | 33.93 | 34.48 | 36.58 | 2.51 | 3.58 | 6.07 | 0.07 | 0.1 | 0.17 | 34.02 | 34.67 | 37.09 |
| 4 | 0:1_38 | 5.63 | 6.86 | 8.15 | 0.87 | 1.62 | 2.47 | 0.15 | 0.24 | 0.3 | 5.7 | 7.05 | 8.52 |
| 5 | 0:1_38 | 9.62 | 11.86 | 17.05 | 2.27 | 4.87 | 8.46 | 0.24 | 0.41 | 0.5 | 9.88 | 12.82 | 19.04 |
| 6 | 1:1_38 | 26.28 | 26.57 | 28.4 | 3.1 | 4.01 | 5.7 | 0.12 | 0.15 | 0.2 | 26.48 | 26.86 | 28.97 |
| 7 | 1:0_38 | 72.59 | 72.7 | 81.27 | 8.18 | 12.43 | 15.37 | 0.11 | 0.17 | 0.19 | 73.05 | 73.75 | 82.72 |
| 8 | 1:1_38 | 34.14 | 36.32 | 45.08 | 6.59 | 10.36 | 16.77 | 0.19 | 0.28 | 0.37 | 34.77 | 37.77 | 48.09 |
| 9 | 1:0_38 | 34.7 | 35.39 | 36.41 | 1.98 | 2.16 | 2.78 | 0.06 | 0.06 | 0.08 | 34.75 | 35.46 | 36.51 |
| 10 | 1:2_38 | 29.57 | 29.94 | 37.09 | 6.19 | 9.55 | 13.86 | 0.21 | 0.32 | 0.37 | 30.21 | 31.42 | 39.59 |
| 11 | 1:1_38 | 32 | 33.28 | 43.68 | 6.43 | 10.95 | 18.1 | 0.2 | 0.33 | 0.41 | 32.64 | 35.04 | 47.28 |
| 12 | 2:1_38 | 51.84 | 57.9 | 74.83 | 15.24 | 24.73 | 34.21 | 0.29 | 0.43 | 0.46 | 54.03 | 62.96 | 82.29 |
| 13 | 0:1_60 | 11.77 | 13.75 | 15.26 | 1.42 | 2.32 | 3.43 | 0.12 | 0.17 | 0.22 | 11.85 | 13.95 | 15.64 |
| 14 | 1:1_60 | 52.8 | 55.76 | 70.71 | 13.15 | 20.17 | 28.73 | 0.25 | 0.36 | 0.41 | 54.42 | 59.3 | 76.32 |
| 15 | 1:1_60 | 33.62 | 33.88 | 36.99 | 4.57 | 6.55 | 9.49 | 0.14 | 0.19 | 0.26 | 33.92 | 34.5 | 38.18 |
| 16 | 1:0_60 | 46.64 | 47.03 | 49.61 | 3.46 | 4.29 | 6.61 | 0.07 | 0.09 | 0.13 | 46.76 | 47.22 | 50.05 |
| 17 | 1:3_60 | ND | ND | ND | ND | ND | ND | ND | ND | ND | ND | ND | ND |
| 18 | 3:1_60 | 60.08 | 60.64 | 69.79 | 10.35 | 14.87 | 23.31 | 0.17 | 0.24 | 0.33 | 60.96 | 62.46 | 73.6 |

### Example 6. Optimizing Bioink Composition for Human Chondrocyte Expression of Lubricin

The surface zone of articular cartilage is the first area impacted by cartilage defects, commonly resulting in osteoarthritis. Chondrocytes in the surface zone of articular cartilage synthesize and secrete lubricin, a proteoglycan that functions as a lubricant protecting the deeper layers from shear stress. 3D bioprinting is a tissue engineering technique that uses cells encapsulated in biomaterials to fabricate 3D constructs. Gelatin methacrylate (GelMA) is a frequently used biomaterial for 3D bioprinting cartilage. Oxidized methacrylated alginate (OMA) is a chemically modified alginate designed for its tunable degradation rate and mechanical properties. To determine an optimal combination of GelMA and OMA for lubricin expression, we used thisnovel high-throughput human articular chondrocyte reporter system. Primary human chondrocytes were transduced with PRG4 (lubricin) promoter-driven Gaussia luciferase, allowing for temporal assessment of lubricin expression. A lubricin expression driven Design of Experiment screen and subsequent validation identified 14% GelMA/2% OMA for further study. Therefore, 14% GelMA/2% OMA, 14% GelMA and 16% GelMA were 3D bioprinted. The combination of lubricin protein expression and shape retention over the 22 days in culture, determined the 14% GelMA/2% OMA to be the optimal formulation for lubricin secretion.

### Introduction

Osteoarthritis (OA) is the most common form of arthritis, negatively impacting millions of individuals each year. OA is characterized by a loss of cartilage. Defects occur in the superficial layer of articular cartilage, and continue to degrade down to the sub-chondral bone. This makes the surface zone of articular cartilage the first area impacted by cartilage defects. The surface zone of articular cartilage functions to protect the deeper layers from shear stress. This layer is in contact with the synovial fluid of the joint, and both the collagen fibers and chondrocytes are oriented parallel to the articulating surface. There are more chondrocytes in this zone vs. deeper zones, and they primarily synthesize and secrete lubricin. Lubricin is a proteoglycan, derived from the proteoglycan 4 (PRG4) gene, which functions as a boundary lubricant. It has been shown to decrease the coefficient of friction and prevent synovial cell and protein adhesion to the cartilage surface. Lubricin is essential for fully functional articular cartilage, and mutations to the PRG4 gene result in camptodactyly-arthropathy-coxa vara-pericarditis syndrome, a disease resulting in poly-articular OA. Treatment with recombinant lubricin, or lubricin mimetics have been shown to reduce the damaging effects of surgically induced OA in rats and OA in ovariectomized rats. Lubricin is critical for functional joint tissue.

Surgeries like matrix induced autologous chondrocyte implantation are resurfacing methods commonly used to treat cartilage defects. However, these methods have drawbacks including a lack of donor tissue, inflammation, degradation and biocompatibility. 3D bioprinting is a tissue engineering technique involving the simultaneous extrusion of biomaterials and living cells. Since the cells are encapsulated within the biomaterials, there is no need for post-fabrication cell seeding and therefore there is no cell penetration limitation. 3D bioprinting has the ability to create patient specific bioactive scaffolds to treat tissue defects, and/or to resurface the joint. Biomaterials can be optimized for stimulating lubricin expression, effectively recreating that protection lost upon injury to the tissue. Patient specific cells can be encapsulated within biomaterials to further improve biocompatibility. Overall 3D bioprinting can be used to create a scaffold specifically shaped for the defect site, without the drawback of traditional therapies.

To 3D bioprint the surface zone for articular cartilage defects, biomaterials should promote lubricin expression. For an ideal construct, biomaterials also need to mimic the extracellular matrix (ECM), fill the defect space and maintain that space while integrating with the surrounding tissue. For this study, we chose to use gelatin methacrylate (GelMA) in combination with oxidized methacrylated alginate (OMA). GelMA is a well characterized and frequently used biomaterial for 3D bioprinting cartilage. OMA is chemically modified alginate developed for its tunable degradation rate. It has been shown to be ideal for 3D bioprinting due to its shear-thinning ability after calcium crosslinking. Once shear stress is removed, OMA recovers its mechanical properties rapidly. The oxidizing of alginate prior to methacrylation alters the uronate residue conformations, making it more vulnerable to hydrolysis and creating tunable degradation rates. The methacrylation of both biomaterials enables photocrosslinking by visible light when combined with the photoinitiator lithium phenyl-2,4,6-trimethylbenzoylphosphinate (LAP) for further stability and enhanced mechanical properties.

This study developed a novel reporter system with a PRG4 promoter-driven Gaussia luciferase (HuPRG4gLuc) in primary human chondrocytes. Upon PRG4 stimulation, Gaussia luciferase is secreted from the cells, making this a non-destructive assay. This is the first study using the HuPRG4gLuc cells. A similar reporter system was used to analyze type II collagen expression. The type II collagen reporter cells have been used for micronutrient optimization in chondrogenic media for a murine chondrogenic cell line (ATDC5s) and for biomaterial optimization with human articular chondrocytes. This study expand upon the previous biomaterial optimization focusing on new biomaterials and how they impact lubricin expression of human articular chondrocytes.

To streamline this biomaterial optimization, this study also used a Design of Experiment (DoE) approach. DoE software (Design-Expert, Stat-Ease) uses statistical modeling to generate a set of combinations, while also limiting the number of groups tested. Utilizing this method effectively decreases the amount of time and materials used to determine optimal groups for 3D bioprinting. Using biomaterial combinations generated by the DoE screen with the HuPRG4gLuc cells this study developed a streamlined approach for identifying biomaterial combinations that increase lubricin expression. This systematic approach identified an optimal combination of GelMA and OMA based on lubricin promoter-driven luminescence, biochemical and mechanical data.

### Materials and Methods

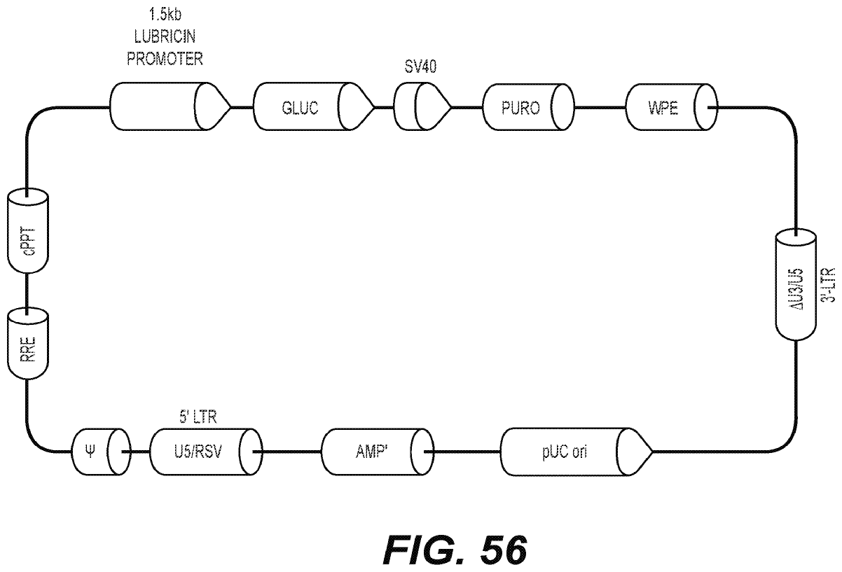

Engineering of lubricin promoter-driven reporter human chondrocytes (HuPRG4gLuc). Human articular chondrocytes (64 year old, non-diabetic female) were isolated as previously described. Briefly, macroscopically normal cartilage tissue was dissected from discarded surgical tissue from a patient undergoing total joint replacement. Cartilage was diced <1 mm³ and sequentially digested in hyaluronidase, followed by collagenase. Cells were resuspended (95% FBS, 5% DMSO), frozen, and stored in liquid nitrogen. Lentiviral vector plasmids psPAX2 (plasmid #12260; Addgene), pMD2G (plasmid #12259), and PRG4-gLuc (9,394 Bp, Genecopoeia, FIG. 56) were purified from transformed competent E. coli (GCI-L3; Genecopoeia) using a commercial kit (ZymoPURE II Plasmid Maxiprep Kit; Zymo Research Corp). HEK293Ta cells (Genecopoeia) were transfected with the purified genes using calcium phosphate nanoparticles. Lentiviral particles were collected from the media and concentrated via ultracentrifugation (30,000 RCF, 8 h, 4° C.). Human articular chondrocytes were thawed from stocks of uncultured cells, seeded, and grown prior to lentiviral infection. Chondrocytes were incubated with pseudolentiviral particles (MOI 2.3) at 4° C. for 20 minutes and then moved to a cell culture incubator (humidified 37° C. atmospheric oxygen, 5% CO₂) for 11 hours. The cells were grown to ˜90% confluence and then passaged onto synoviocyte matrix coated flasks and isolated with puromycin (2 μg/mL) for 7 days. The remaining cells were grown to ˜90% confluence prior to being trypsinized, neutralized, and cryopreserved with FBS (95%) and DMSO (5%).

HuPRG4gLuc reporter cell aggregate culture and TGFβ1 dose response. HuPRG4gLuc reporter chondrocytes were thawed from frozen stocks and seeded onto synoviocyte derived matrix coated flasks to expand to confluence (˜5 days, 37° C., 5% CO₂ and 5% O₂). Upon confluence, cells were trypsinized (0.25% Trypsin/EDTA, 5 min, 37° C.), neutralized with growth media (DMEM/F12 (Gibco) supplemented with 10% FBS (mesenchymal stromal cell selected and 1% penicillin-streptomycin (Hyclone)), then centrifuged (5 min, 500 RCF, room temperature [RT]). Cells were then resuspended in defined chondrogenic media (DMEM-HG 93.24% (Lonza), 1% 10⁻⁵ M dexamethasone (Sigma), 1% ITS+premix (Becton-Dickinson), 1% Glutamax (Hyclone), 1% 100 mM Sodium Pyruvate (Hyclone), 1% MEM Non-Essential Amino Acids (Hyclone), 0.26% 50 mM L-Ascorbic Acid Phosphate (Wako), 0.5% Fungizone (Life Technologies)), and seeded (50,000 cells/well) onto a non-adherent 96-well plate (Greiner Bio), then centrifuged to form cell aggregates (5 min, 500 RCF, RT). Cell aggregates were cultured for 3 weeks in defined chondrogenic media containing TGFβ1 (0-40 ng/mL, Peprotech), and fed 3 times a week (37° C., 5% CO₂ and 5% O₂). Media containing the secreted Gaussia luciferase was sampled twice a week for luminescence (section 2.3). On day 22, cell aggregates were divided between histological analysis (section 2.4), biochemical assessment (section 2.5) or qPCR gene expression analysis (section 2.6).

Luciferase assessment. As previously described, during the 22-day culture period, luminescence was assessed twice a week. Culture media (20 μl/well) was combined with a stabilized luciferase reaction mix (50 μl/well) on a white, 96-well plate (Greiner Bio-One). The final concentration of each component of the stabilized reaction mix was 0.09 M MES, 0.15M ascorbic acid, and 4.2 μM coelenterazine. Luminescence was read on a plate reader (PerkinElmer EnVision 2104 Multilabel Reader.

Histology. Samples were fixed in 10% neutral buffered formalin (Epredia) for ˜2 days, dehydrated and then embedded in paraffin wax. To assess sulfated GAG content, rehydrated samples were stained with safranin-O (Electron Microscopy Sciences) and counterstained with fast green (VWE, Alfa Aesar) and Weigert's iron hematoxylin (IronElectron Microscopy Sciences). Immunohistochemistry was performed for type II collagen content, as previously described and for lubricin. Briefly for type II collagen, antigen retrieval was done with Pronase (1 mg/mL for 10 minutes at RT, Sigma) followed by blocking with BSA (3% w/v, Cohn Fraction V, Alfa Aesar). Primary antibody (1:200 in 1% BSA, DSHB, II-II6B3) incubation was for 2 hours at RT and secondary antibody (1:2000 in 1% BSA, Biotinylated horse anti-mouse, Vector Labs) incubation was for 1 hour at RT. Finally, HRP-conjugated streptavidin (1:5000 in 1% BSA, Invitrogen) was incubated for 30 minutes at RT and Vector VIP Peroxidase Substrate Kit (VWR) was used to develop staining. For lubricin, antigen retrieval was performed with hyaluronidase (10 mg/mL in 20 mM sodium acetate, Sigma) incubation at 37° C. for 30 minutes. Samples were blocked with 3% BSA. Primary lubricin antibody (1:400, 1% BSA, Millipore MABT401) was incubated for 90 minutes at RT, and secondary antibody was incubated for 30 minutes at RT. HRP-streptavidin incubation and stain development was the same as for type II collagen. All sections were counter stained with Fast Green (VWR) and imaged (Keyence BZ-X810 microscope).

Biochemical assays. As previously described, frozen samples were digested in papain (0.025 mg/mL papain in 50 mM sodium phosphate, 2 mM EDTA, 2 mM cysteine) overnight. Hoechst dye was used to quantify the DNA content of the digest, with calf thymus DNA (Sigma) as the standard. Samples were read on a plate reader (Ex365, Em460 nm, SpectraMax iD5, Molecular Devices). Dimethylmethylene blue (5 ml of 3.2 mg/mL DMMB dissolved in 200 proof ethanol added to 1L of 40 mM glycine, 40 mM NaCl at pH 1.5) colorimetric assay was used to quantify GAG content of the digest with absorbance readings at 525 nm and 595 nm (correction). Hydroxyproline (HDP) content was measured for total collagen quantification. Papain digested samples were acid hydrolyzed overnight (10:1 vol/vol, 6M HCl, 110° C.) and then dried overnight (70° C.). Copper sulfate (0.15M) and NaOH (2.5M) were added and incubated (50° C., 5 minutes). Oxidation occurred through 10-minute incubation with hydrogen peroxide (6% H₂O₂, 50° C.). Sulfuric acid was added (1.5M H₂SO₄) prior to reaction with Ehrlich's reagent (10% w/v 4-dimethylamino benzaldehyde in 60% isopropanol, 26% perchloric acid, 14% MΩ water) for a final incubation (70° C., 16 minutes). Absorbance was read at 505 nm (SpectraMax iD5, Molecular Devices) with hydroxyproline standard curve generated using hydroxyproline (Sigma). For the biomaterials, samples without cells were used to subtract the background generated by gelatin.

HuPRG4gLuc reporter cells qPCR assessment. HuPRG4gLuc reporter chondrocytes were seeded in cell aggregate culture (section 2.3) and cultured in defined chondrogenic media supplemented with 0-1 ng/mL TGFβ1. On day 22, cell aggregates were frozen (−80° C.) in RNA lysis buffer for subsequent RNA extraction (Invitrogen RNA Purelink minikit). mRNA was isolated using column purification with on-column DNase treatment. RNA ScreenTape (Agilent Technologies) was used to assess RNA purity and integrity. cDNA was synthesized (Maxima H cDNA kit) and qPCR was performed (QuantStudio 7 flex, Applied Biosystems). Primers used for gene expression analysis were: Hypoxanthine Phosphoribosyltransferase 1 (HRPT, reference gene, forward primer: 5′ ATTGACACTGGCAAAACAATGC 3′ (SEQ ID NO: 9), reverse primer: 5′ TCCAACACTTCGTGGGGTCC 3′ (SEQ ID NO: 10)), Gaussia luciferase (gLuc, forward primer: 5′ ACGCTGCCACACCTACGA 3′ (SEQ ID NO: 11), reverse primer: 5′ CCTTGAACCCAGGAATCTCAG 3′ (SEQ ID NO: 12)) and lubricin (PRG4, forward primer: 5′ TTGCTCCTCTCTGTTTTCGT 3′ (SEQ ID NO: 13), reverse primer: 5′ ATACCCTTCCCCACATCTCCC 3′ (SEQ ID NO: 14)). A mixture of the primers, SYBR green (Applied Biosystems, Thermofisher Scientific) and cDNA was run with cycling parameters: 95° C. for 20s then 45 cycles of 95° C. 10s, 60° C. 20s, 72° C. 19s, followed by melt curve analysis. CT values were normalized to HRPT expression and gLuc vs PRG4 relative gene expression was plotted with 95% confidence bands (GraphPad Prism).

Design of experiment (DoE) screen design. Stat-Ease, Design-Expert (Version 13) was used to generate testing conditions for screening combinations of GelMA and OMA at different photocrosslinking times. A mixture model was used in an optimal combined design generating a total of 60 conditions. Constraints were set so the final percentage of GelMA in the mixture was between 0-12%, OMA was 0-2%, with the rest being PBS between 86-98%. The sum of the final percentages of GelMA and OMA was set to be between 2-14%, and the sum of GelMA, OMA and PBS would always equal 100%. Crosslinking time was the numeric factor, ranging between 15-60s. To determine if the addition of calcium prior to cell encapsulation had an impact on lubricin expression, calcium inclusion was a categoric factor, resulting in groups with or without calcium (final concentration 1.8 mM CaCl₂)). DoE generated testing conditions were combined with HuPRG4gLuc cells (section 2.9) and lubricin promoter-driven luciferase expression was assessed (section 2.3). Luminescence data was input into the DoE software where it suggested a square root transformation to fit the model. ANOVA analysis was used to identify if biomaterial mixture, calcium chloride addition or crosslinking time had statistically significant impact on lubricin driven luminescence.

Oxidized methacrylated alginate (OMA) synthesis and characterization. Oxidized alginate was prepared by reacting sodium alginate (LF120M, 1% aqueous solution viscosity=251 mPa·s, FMC Biopolymer) with sodium periodate (Sigma). Briefly, sodium alginate (10 g) was dissolved in ultrapure deionized water (diH₂O, 900 mL) overnight. Sodium periodate (0.54 g) was dissolved in 100 mL diH₂O, added to alginate solution under vigorous stirring to achieve 5% theoretical alginate oxidation, and allowed to react in the dark at room temperature for 24 hours. Methacrylation (20% theoretical) was performed to obtain OMA (5OX30MA) by reacting oxidized alginate with 2-aminoethyl methacrylate hydrochloride (AEMA, Polysciences). To synthesize OMA, 2-morpholinoethansulfonic acid (19.52 g, Sigma) and NaCl (17.53 g, Fisher Scientific) were directly added to the oxidized alginate solution and the pH was adjusted to 6.5. N-hydroxysuccinimide (NHS, 1.764 g, Fisher Scientific) and 1-ethyl-3-(3-dimethylaminopropyl)-carbodiimide hydrochloride (EDC, 5.832 g, Oakwood Chemical) were added to the solution under stirring to activate 30% of the carboxylic acids of the oxidized alginate. After 5 minutes, AEMA (2.532 g, molar ratio of NHS: EDC: AEMA=1:2:1) was added to the solution and the reaction was maintained in the dark at room temperature for 24 hours. The reaction mixture was precipitated into excess acetone, dried in a fume hood, and rehydrated to a 1% w/v solution in diH₂O for further purification. The OMA was purified by dialysis against diH₂O using a dialysis membrane (MWCO 3500 Da, Spectrum Laboratories) for 3 days, treated with activated carbon (5 g/L, 100 mesh, Oakwood Chemicals) for 30 minutes, filtered (0.22 μm filter) and lyophilized. To determine the levels of alginate methacrylation, the OMA was dissolved in deuterium oxide (2% w/v), and ¹H-NMR spectrum was recorded on an NMR spectrometer (600 MHz, Bruker) using 3-(trimethylsilyl) propionic acid-d4-sodium salt (0.05% w/v) as an internal standard.

Preparation of GelMA and OMA stocks and combinations. GelMA (58% methacrylated, 167 kDa, Rousselot) and OMA were reconstituted in PBS containing 0.05% lithium phenyl-2,4,6-trimethylbenzoylphosphinate (LAP, Cellink). Stocks were made by combining the material with PBS/LAP in 1.6 ml microtubes on a tube warmer at 50° C. and shaken (800 rpm) until fully dissolved. To prepare biomaterial combinations, calculated amounts of each stock were added to 1.6 ml microtubes on the tube warmer at 50° C., with a quick vortex to fully combine. PBS containing 0.05% LAP was added if further dilutions were necessary. All stocks and combinations were made less than 24 hours prior to use and stored in the dark at 4° C. To encapsulate cells, biomaterial combinations were warmed on the tube warmer to 37° C., cells added and vortexed to mix. The cell-biomaterial mixtures were either pipetted onto 96 well, white plates with a clear bottom for the DoE screen (section 2.7) and subsequent validation or added to barrels for 3D bioprinting (section 2.10).

3D Bioprinting. All 3D bioprinting was performed using a BioAssemblyBot pneumatic extrusion 3D bioprinter (Advanced Solutions) and constructs designed using the Tissue Structure Information Modeling (TSIM) software. Biomaterials were printed using 3 mL, UV blocking amber barrels (Nordson), 25G SmoothFlow tapered tips (Nordson) and print settings shown in Table 10. The hot tool attachment was adapted for use with 3 mL barrels to warm biomaterials. After printing, constructs were photo-crosslinked (Luck Laser, 405 nm, 300 mW) with the light focused to 8 mm beam diameter and set 3.5 cm above the construct. Cylindrical constructs (8 mm diameter×1 mm height) were bioprinted containing HuPRG4gLuc cells for luminescence assay, mechanical characterization, histology, and biochemical assay. To quantify cell viability, single layer rectangular cuboids (2 mm×6 mm×0.3 mm) were bioprinted containing HuPRG4gLuc cells.

TABLE 10
3D Bioprinting Settings

| | Pressure | Acceleration | Speed | Ink Temp |
|---|---|---|---|---|
| | (psi) | (mm/sec²) | (mm/sec) | (° C.) |
| 14% GelMA | 16-14 | 100 | 9 | 24 |
| 14% GelMA, 2% OMA | 33-18 | 100 | 5 | 25 |
| 16% GelMA | 15-12 | 100 | 7 | 25 |

All printed with 25TT (25-gauge Taper Tip needle, Nordson), stage temp of 35° C., line width and height of 0.2 mm.

Cell Viability. Cell viability was assessed using live/dead staining on days 0, 1 and 7. Groups 14% GelMA, 16% GelMA or 14% GelMA, 2% OMA containing HuPRG4gLuc cells (1 million cells/mL final concentration) were 3D bioprinted as previously described (section 2.10) or pipetted (control) in triplicate for each time point. As previously described, at each time point the staining solution (calcein-AM (2 μM, Invitrogen) and ethidium homodimer-1 (4 μM, Invitrogen) in sterile PBS) was added to each well, incubated for 25 minutes (37° C., 5% CO₂), removed and replaced with PBS. Images were taken using a Pico Imager (Molecular Devices) and then processed and analyzed using ImageJ/Fiji with the Stardist plugin, with >100 cells were quantified for each sample.

Mechanical characterization. Dynamic mechanical analysis (DMA) compression. Unconfined uniaxial compression testing was performed (DMA 242E Artemis, Netzsch) with a set strain of 10% (100 μm) and frequencies of 0.1, 1 and 5 Hz at room temperature. Disc width and height were measured with calipers (Netzsch). Each disc was also imaged, and surface area quantified using ImageJ/Fiji. Discs were maintained in PBS for the duration of the test. The storage modulus (E′), loss modulus (E″) and tan delta were measured.

Lap-shear. Static coefficient of friction and kinetic coefficient of friction were determined through a lap shear test on disc constructs. Day 0 discs were cast in an 8 mm diameter, 1 mm high silicone mold, while day 22 constructs were 3D bioprinted discs of the same dimensions containing HuPRG4gLuc cells and cultured for 22 days. Constructs were frozen (−80° C.) until use. Lap shear testing was preformed using a TA.XTplusC texture analyzer (Stable Micro Systems). The samples were adhered to a microscope slide (VWR) and placed securely into the top tensile clamp. A second microscope slide was secured into the bottom tensile clamp. The sample was fully submerged in PBS for the duration of the test. The sample was aligned until it was touching the second microscope slide and then compressed by ˜200 μm, creating a normal force of 1.62N. Normal force was determined by a force sensitive resistor (DF9-40, Yosoo Health Gear). The static force was determined by the peaks of the graph generated from a shear sine wave test, while the kinetic force was determined from the slope. The coefficient of friction was calculated from the force generated on the graphs, divided by the normal force.

Swelling and degradation. 3D bioprinted discs (8 mm diameter×1 mm height) were frozen (−80° C.) in pre-weighed 1.6 mL microtubes (Wₜ), lyophilized and initial dry weights (Wᵢ) measured. Chondrogenic media (700 μL, section 2.2) was added to each tube, fully submerging the lyophilized discs, and then incubated (37° C., 5% CO₂) for set time points. Media was changed weekly. On days 1, 11 and 22 all media was removed, and swollen (Wₛ) weights were measured. After weighing, samples were frozen (−80° C.) and lyophilized again, then weighed (W_{d}). To calculate the swelling ratio (Q) the swollen weight was divided by the initial weight (Q=(Wₛ−Wₜ)/W_{d}−Wₜ). The percent mass loss was calculated by (((Wᵢ−Wₜ)−(W_{d}−Wₜ))/(Wᵢ−Wₜ))×100.

Lubricin (PRG4) ELISA. To quantify secreted lubricin, cell culture medium was collected from the TGFβ1 dose response on day 16 and from the 3D bioprinted disc constructs on days 1, 10 and 22. Cell culture medium was frozen at −20° C. until use. Enzyme-Linked Immunosorbent Assay (ELISA) kit (DuoSet ELISA Ancillary Reagent Kit and Human Lubricin/PRG4 kit, R&D systems) was used following manufacturer's protocol. Lubricin content was calculated based on the standard curve.

Statistical Analysis. For the DoE screen, design of experiment analysis was completed as previously described (section 2.7). For all other experiments, statistical analysis was completed using GraphPad Prism (Version 9.0). All experiments had 3-9 replicates (n=3-9) and results are shown +/−standard deviation. A p value of <0.05 was considered statistically significant.

### Results

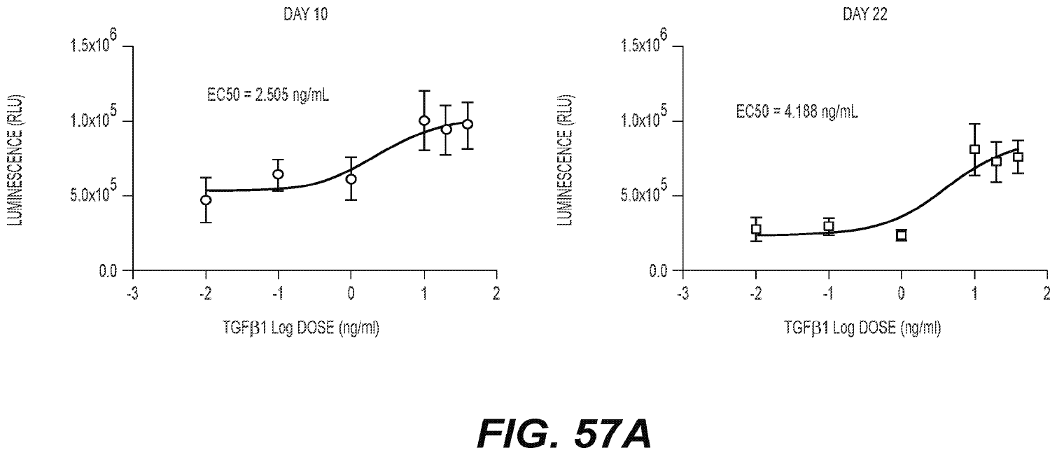

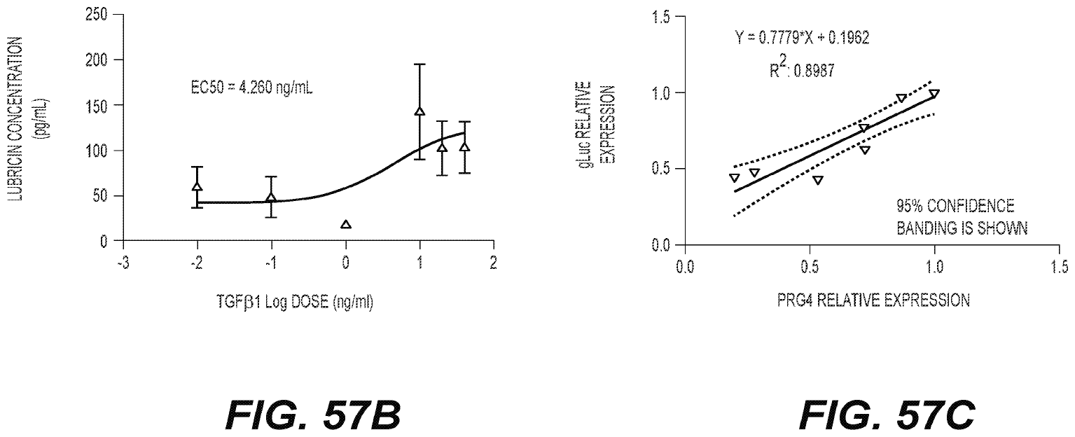

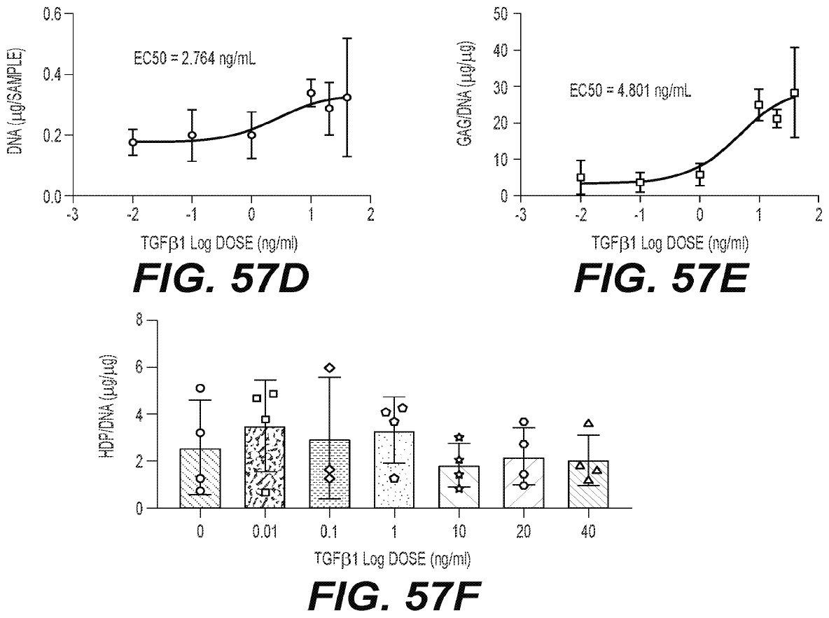

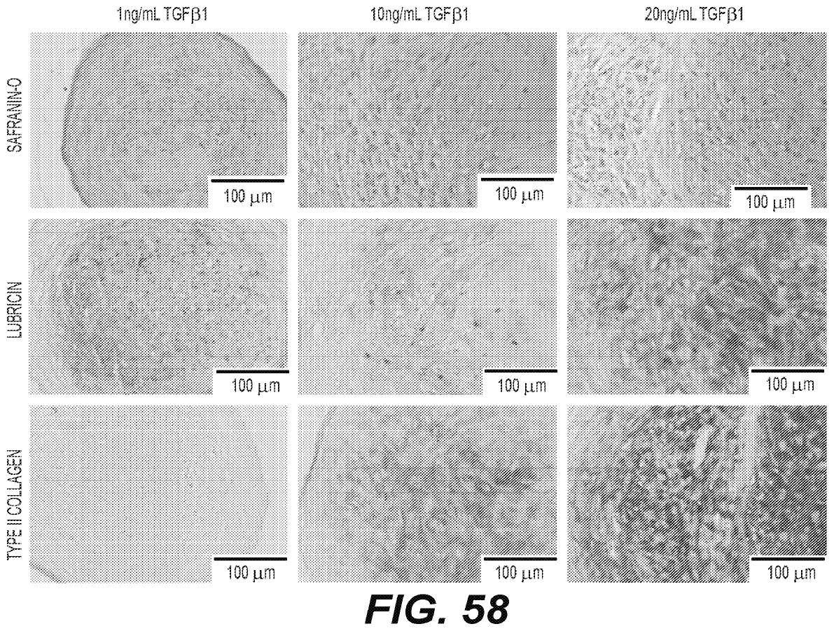

Stimulation of lubricin by TGFβ1 in human primary articular chondrocytes. To characterize human primary articular chondrocytes engineered with a PRG4 promotor driven Gaussia luciferase (HuPRG4gLuc), cell aggregates were cultured with varying doses of TGFβ1, a known inducer of lubricin. Luminescence, a proxy for lubricin, increased in a TGFβ1 dose dependent manner, as shown by the response curves for days 10 and 22 in FIG. 57A. The excitatory concentration producing a half-maximal response (EC50) was 2.5 ng/ml on day 10 and 4.2 ng/mL on day 22 (FIG. 57A). Lubricin secretion also had a TGFβ1 dose dependent increase, with an EC50 of 4.3 ng/ml (FIG. 57B), very similar to the luminescence EC50. This is further supported by qPCR gene expression analysis which correlated PRG4 expression with gLuc gene expression (FIG. 57C). There was a TGFβ1 dose dependent increase of both DNA (EC50=2.8 ng/mL, FIG. 57D) and GAG/DNA (EC50=4.8 ng/mL, FIG. 57E), which is supported by Safranin-O staining (FIG. 58). While hydroxyproline (HDP) per DNA (FIG. 57F) was consistent across groups, immunohistochemical staining for type II collagen showed an increase in staining intensity correlating with an increase in TGFβ1 (FIG. 58). Lubricin immunostaining also showed an increase in staining intensity as TGFβ1 concentration increased (FIG. 58). Together these results support using luminescence as a proxy for lubricin expression and confirmed that the engineered cells retained chondrogenic capacity.

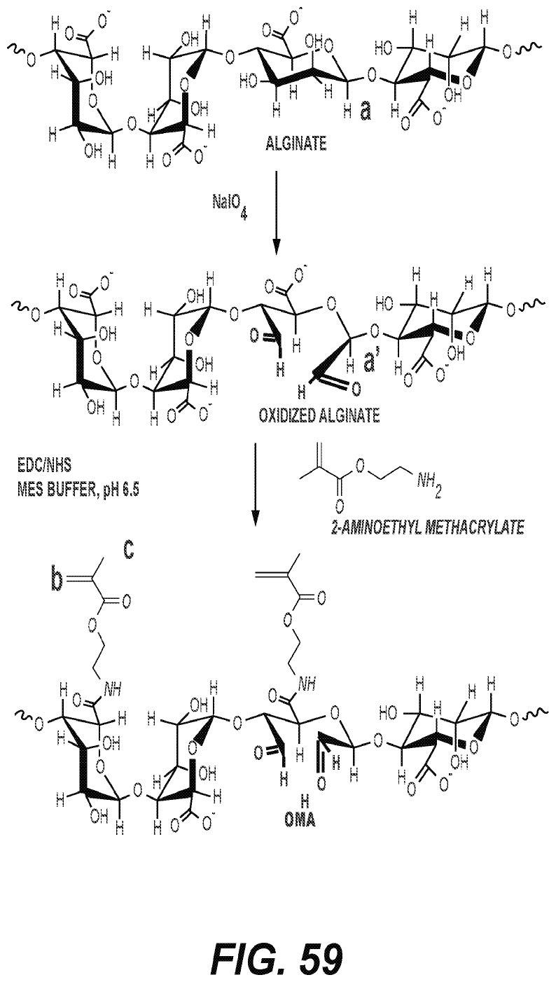

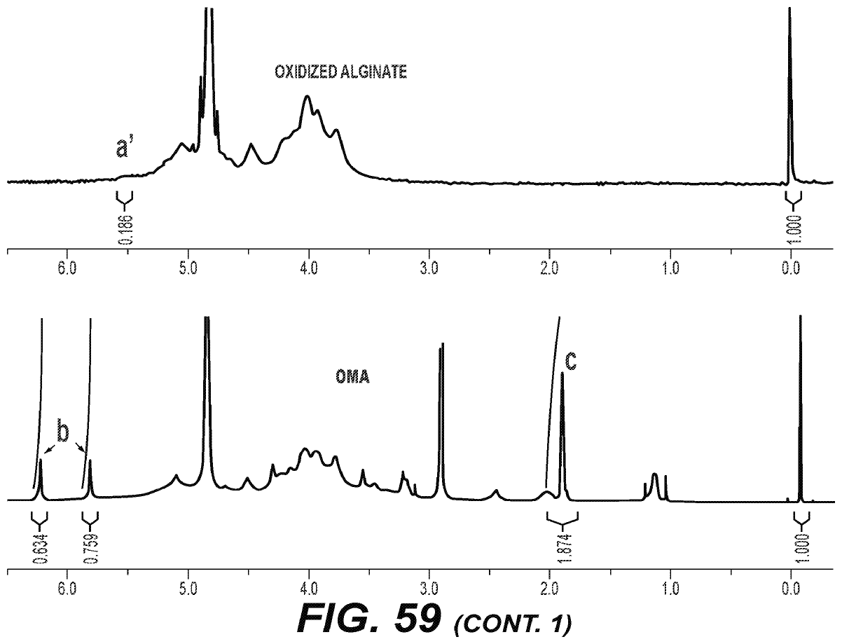

Characterization of OMA. To prepare the biodegradable and photocrosslinkable OMA, sodium alginate was oxidized using sodium periodate in aqueous solution at room temperature for 24 hours, and then methacryloyl groups were introduced onto the oxidized alginate main chains as shown in FIG. 59. The actual oxidation and methacrylation degrees of alginate were calculated from 1H-NMR spectra, which are shown in FIG. 59. In the 1H-NMR spectrum of the oxidized alginate, as a result of the oxidation, the new proton (FIG. 59, a′) on carbon 1 was formed at 5.5 ppm. The 1H-NMR spectrum of the OMA exhibited newly formed proton peaks of vinyl methylene and methyl by the reaction with AEMA at 6.2 and 5.6, and 1.9 ppm, respectively. The actual oxidation of the hydroxyl groups on carbons 2 and 3 of the repeating units of the sodium alginate was 4.8% (5% theoretical) and the actual methacrylation of the repeating units of the oxidized alginate was 18.3% (30% theoretical).

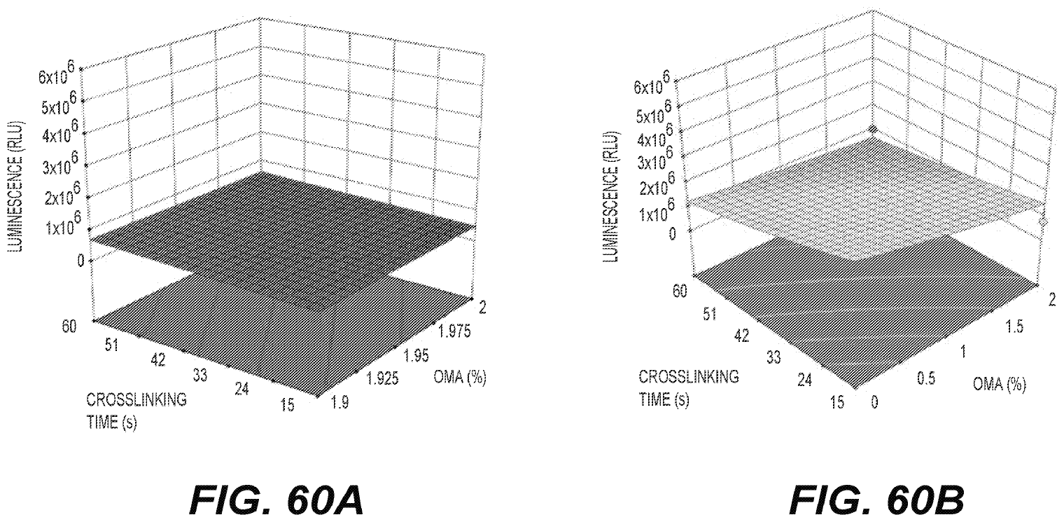

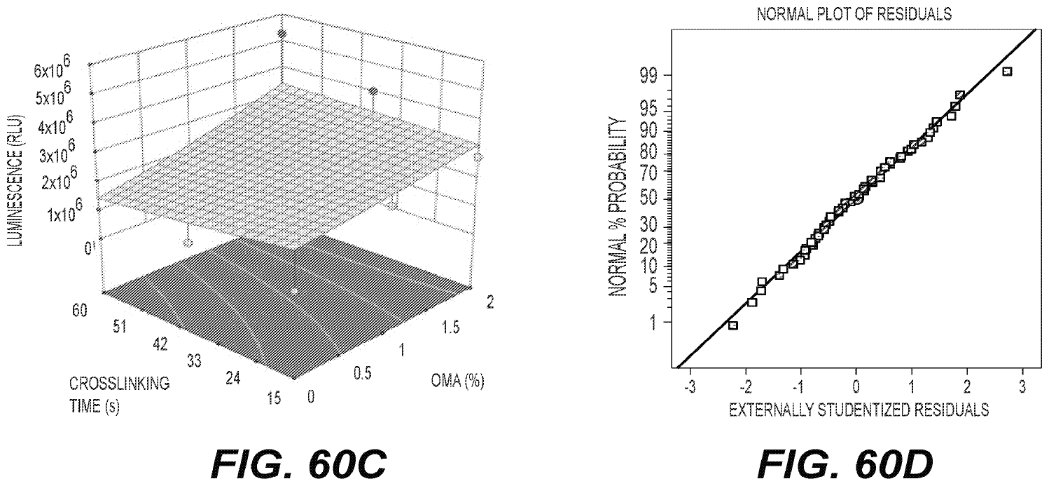

Design of Experiment (DoE) screen of GelMA and OMA combinations for lubricin expression. To identify optimal combinations of OMA and GelMA for lubricin expression, HuPRG4gLuc cells were mixed with biomaterial combinations generated by the DoE. Sixty different combinations, at different crosslinking times, with or without the addition of calcium chloride were tested. At all timepoints the mixture of the biomaterials had a significant impact on luminescence (p<0.001, ANOVA), while crosslinking time and calcium chloride were not significant factors. Day 22 luminescence is shown on 3D surface plots in FIG. 60. Groups that contained only OMA had the lowest luminescence (FIG. 60A). Increasing GelMA to 6%, increased luminescence as compared to OMA alone (FIG. 60B), with the shortest crosslinking time (15s) and no OMA having the highest luminescence. Luminescence further increased, by increasing GelMA concentration to 12% (FIG. 60C). In the groups that had 12% GelMA (FIG. 60C), luminescence further increased as the final percentage of OMA increased, with the highest luminescence expression in 12% GelMA/2% OMA. Data was normally distributed as shown in FIG. 60D.

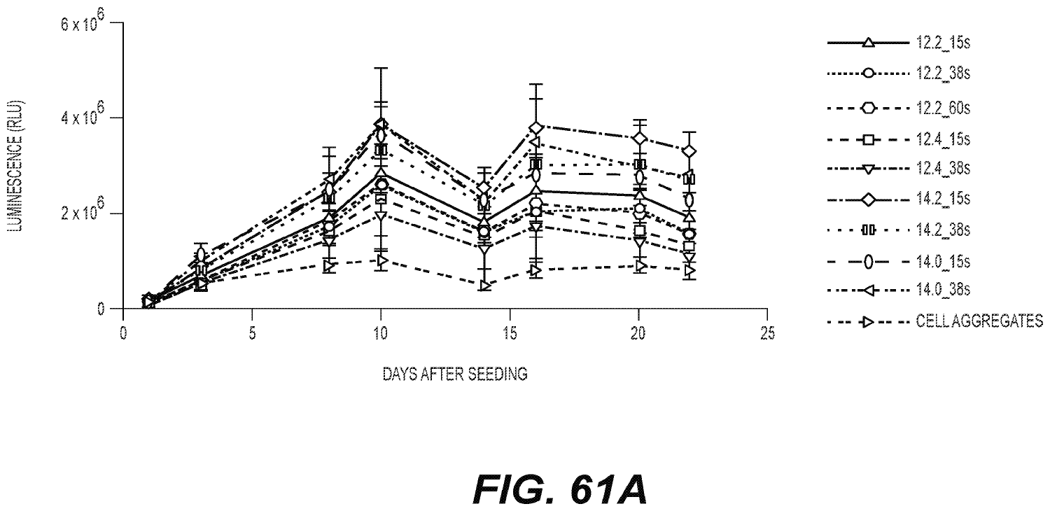

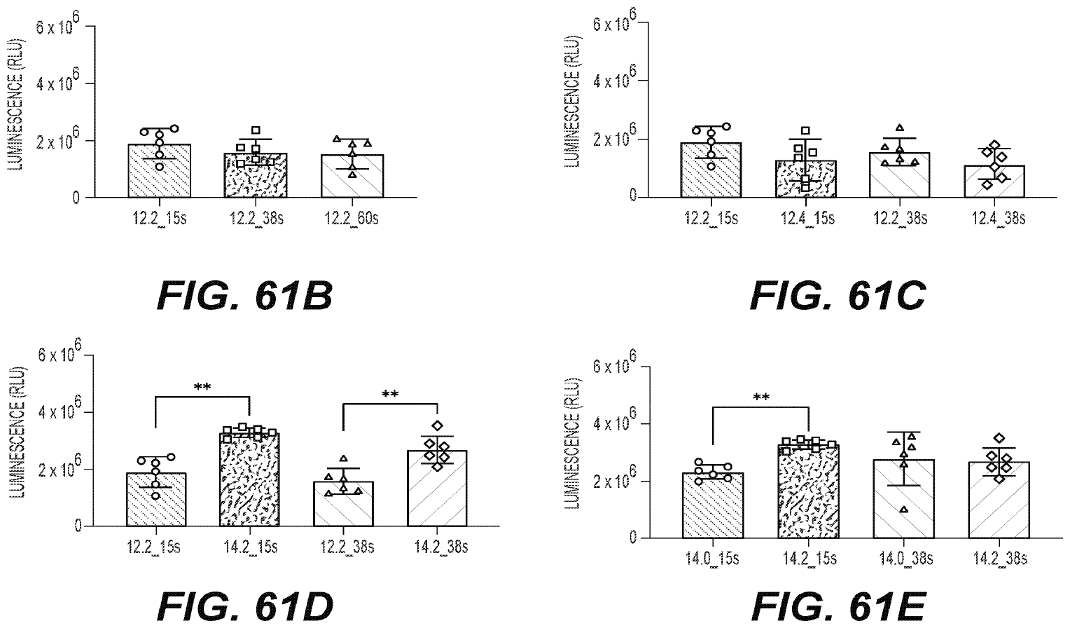

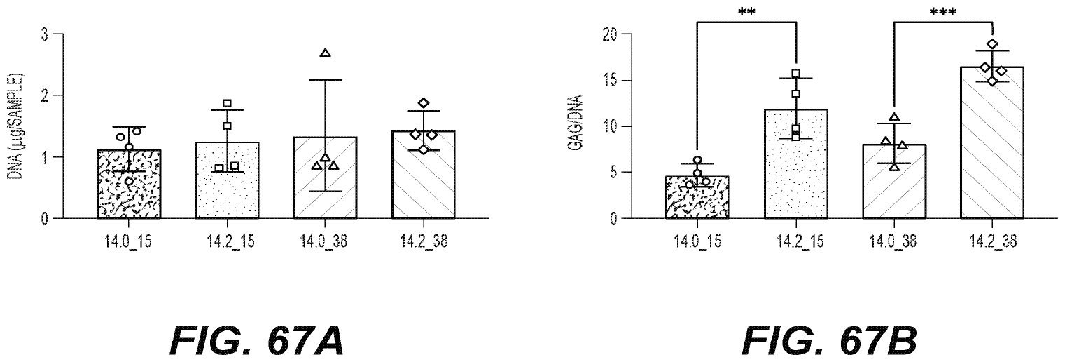

Validation of the DoE screen. Validation of the DoE screen results was performed using the HuPRG4gLuc cells cultured in combinations of GelMA and OMA. All selected biomaterial groups had higher luminescence than the cell aggregate control (FIG. 61A), indicating the mixing of chondrocytes in biomaterials increased lubricin expression. Day 22 luminescence data is shown in FIG. 6B-E. To investigate the lack of effect of crosslinking time seen in the screen, the optimal group from the screen (12% GelMA/2% OMA) was retested at the three crosslinking times (15s, 38s and 60s). Crosslinking did not have a significant effect on luminescence (FIG. 61B). Since the highest final percentage of both GelMA and OMA had the highest luminescence in the screen, both were further increased, and luminescence was assessed. Increasing the final OMA percentage to 4% did not positively impact luminescence at either crosslinking time (FIG. 61C). Increasing the final GelMA percentage to 14%, while keeping OMA consistent at 2% did significantly increase luminescence as compared to 12% GelMA/2% OMA at both crosslinking times (FIG. 61D). To determine if this increase in luminescence was solely due to increasing the GelMA percentage, 14% GelMA alone was compared to 14% GelMA/2% OMA at both crosslinking times (FIG. 61E). The group containing 2% OMA had significantly higher luminescence as compared to 14% GelMA alone at 15s crosslinking, but not 38s (FIG. 61E). The 14% GelMA/2% OMA group after 15s crosslinking was consistently the group with the highest luminescence starting on day 10 (FIG. 61A). DNA content stayed consistent across all 14% GelMA groups (FIG. 67A), but GAG/DNA was significantly higher in 14% GelMA/2% OMA group at both crosslinking times (FIG. 67B). Based on the luminescence, DNA and GAG data, the 14% GelMA/2% OMA at 15s was determined the optimal for lubricin expression.

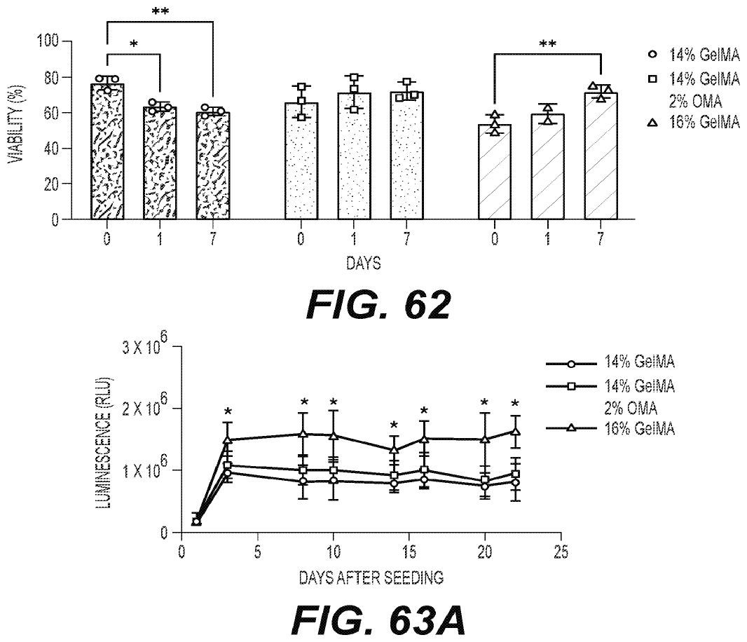

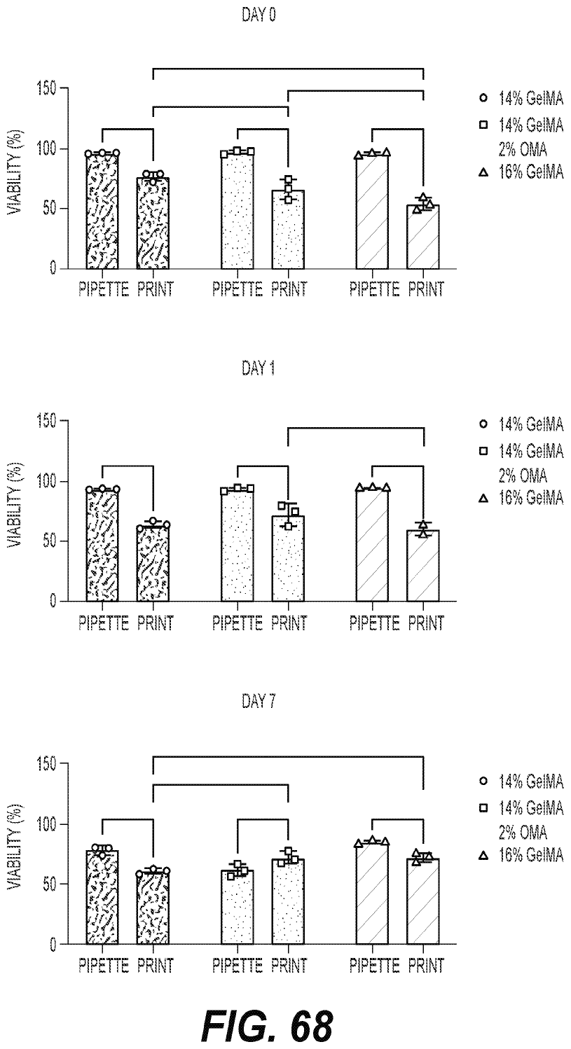

Cell viability. Cell viability was assessed in groups 14% GelMA/2% OMA, 14% GelMA and 16% GelMA to ensure the bioprinting process and biomaterials were biocompatible (FIG. 62). Groups were either 3D bioprinted or pipetted and viability was quantified on days 0, 1 and 7. On days 0 and 1 cell viability was significantly decreased in all 3D bioprinted groups, as compared to their respective pipetted controls (FIG. 68). By day 7, the 14% GelMA and 16% GelMA printed groups still had significantly lower cell viability as compared to their pipetted controls, but the 14% GelMA/2% OMA printed group was significantly higher than the pipetted (FIG. 68). The 14% GelMA printed group cell viability significantly decreased over 7 days from 77% to 61% (FIG. 62). The 14% GelMA/2% OMA group stayed consistent across all 7 days at around 72% viability (FIG. 62). Finally, on day 0, the 16% GelMA printed group had the lowest viability at only 54%, but it significantly increased to 72% by day 7 (FIG. 62).

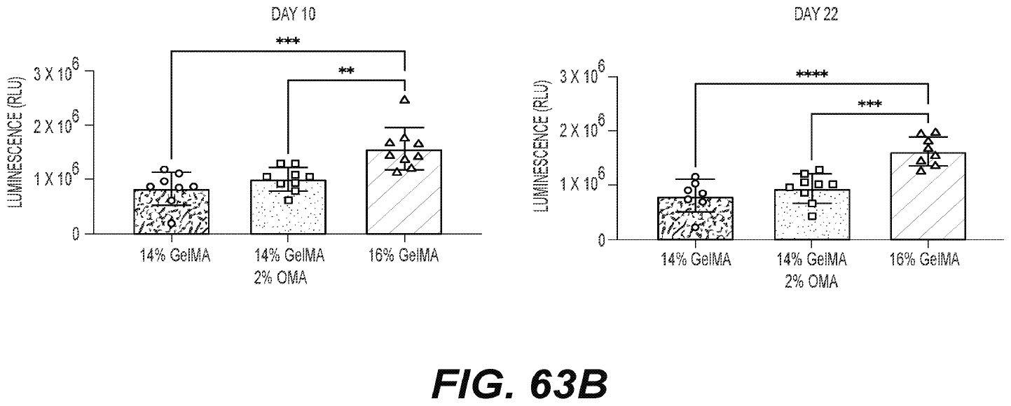

3D bioprinted disc construct containing HuPRG4gLuc cells. The 14% GelMA/2% OMA after 15s crosslinking had the highest luminescence in the validation, and improved GAG deposition, therefore it was used for 3D bioprinting disc constructs containing HuPRG4gLuc cells. As controls 14% GelMA and 16% GelMA with 15s crosslinking were also printed into discs. Luminescence was assessed over 22 days. The 16% GelMA group had significantly higher luminescence starting on day 3 as compared to the other two groups (FIG. 63A). Day 10 and Day 22 have the same trend with 16% GelMA having significantly higher luminescence, and no difference between the 14% GelMA/2% OMA and 14% GelMA groups (FIG. 63B).

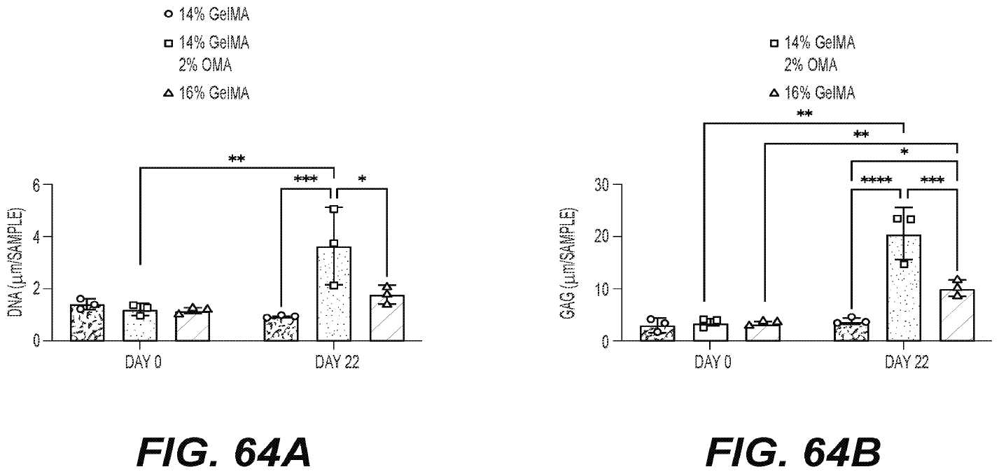

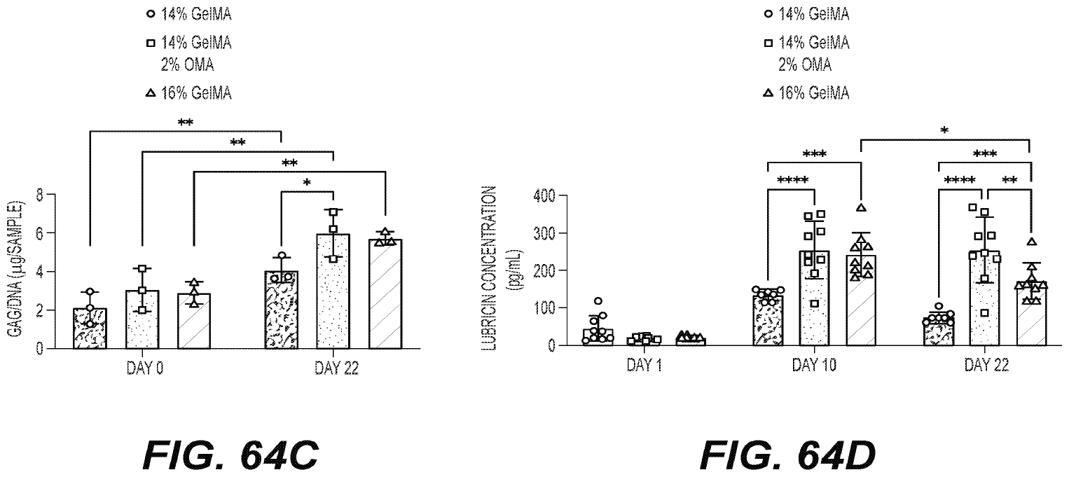

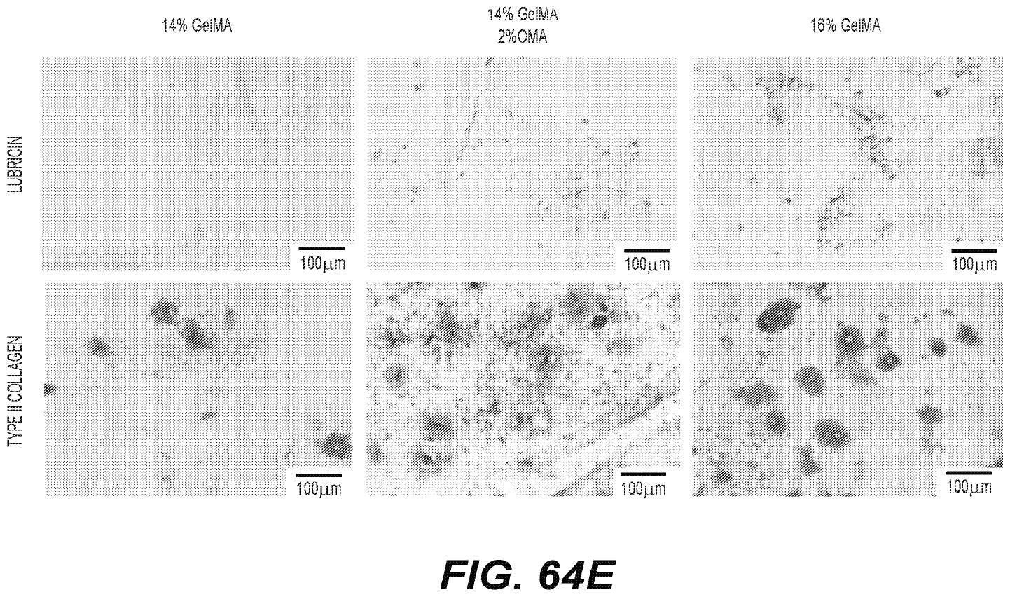

DNA and GAG content were quantified from constructs on day 0 and day 22. DNA content stayed consistent in both 14% and 16% GelMA, but the 14% GelMA/2% OMA had a significant increase from day 0 to day 22, as well as having significantly more DNA than both the 14% and 16% GelMA groups on day 22 (FIG. 64A). The 14% GelMA/2% OMA and 16% GelMA had a significant increase in GAG from day 0 to day 22, while 14% GelMA did not (FIG. 64B). The 14% GelMA/2% OMA on day 22 had significantly more GAG than both other groups (FIG. 64B). GAG/DNA significantly increased for all three groups by day 22 and the 14% GelMA/2% OMA had significantly more GAG/DNA than the 14% GelMA group (FIG. 64C), which was consistent with the GAG/DNA from the validation (FIG. 67).

Secreted lubricin content was quantified by ELISA from culture media on days 1, 10 and 22. Lubricin concentration increased from day 1 to day 10, reflecting the luminescence results (FIG. 64D). On day 10, 14% GelMA/2% OMA and 16% GelMA had significantly more lubricin than the 14% GelMA group (FIG. 64D). On day 22, the 14% GelMA/2% OMA and 16% GelMA groups still had significantly more lubricin than the 14% GelMA group, but 14% GelMA/2% OMA also had significantly more than 16% GelMA (FIG. 64D). The 16% GelMA group had a significant decrease in secreted lubricin content from day 10 to day 22 (FIG. 64D).

Lubricin content retained in the biomaterials and type II collagen content was assessed by immunohistochemistry (FIG. 64E). The 16% GelMA group had more lubricin staining than either of the other groups, while 14% GelMA had very minimal staining (FIG. 64E). The 14% GelMA group also had less type II collagen staining (FIG. 64E). The type II collagen staining in 16% GelMA was darker, but more pericellular as compared to the 14% GelMA/2% OMA group where it is lighter but more spread out into the material (FIG. 64E).

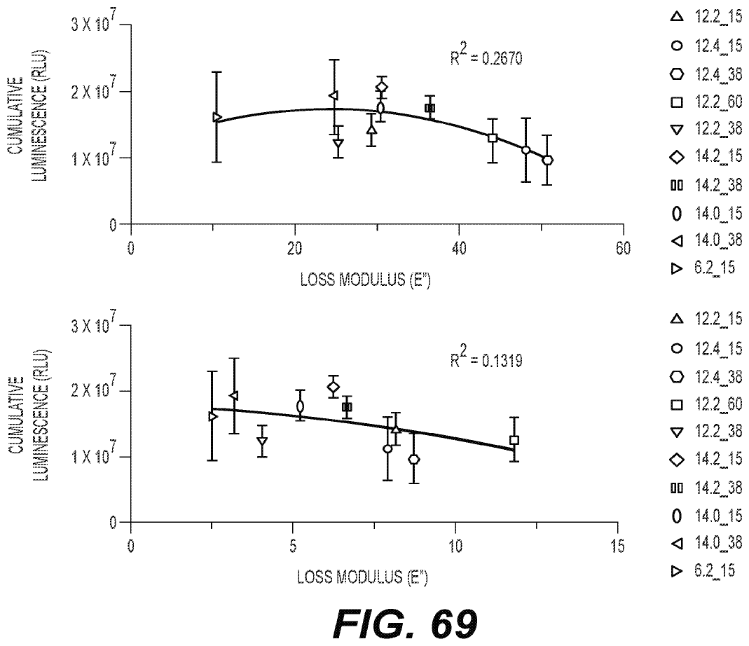

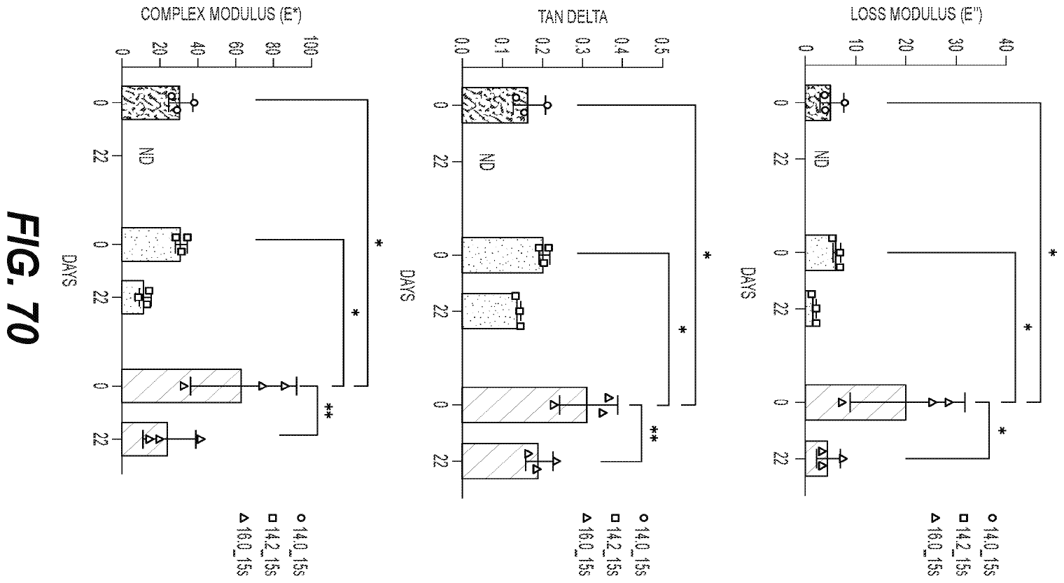

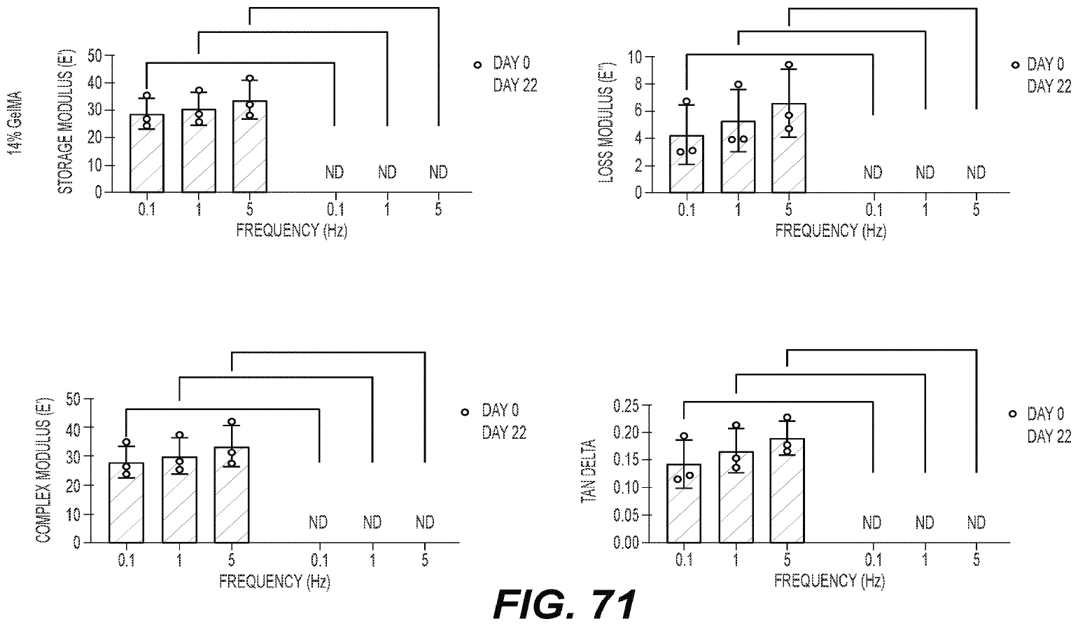

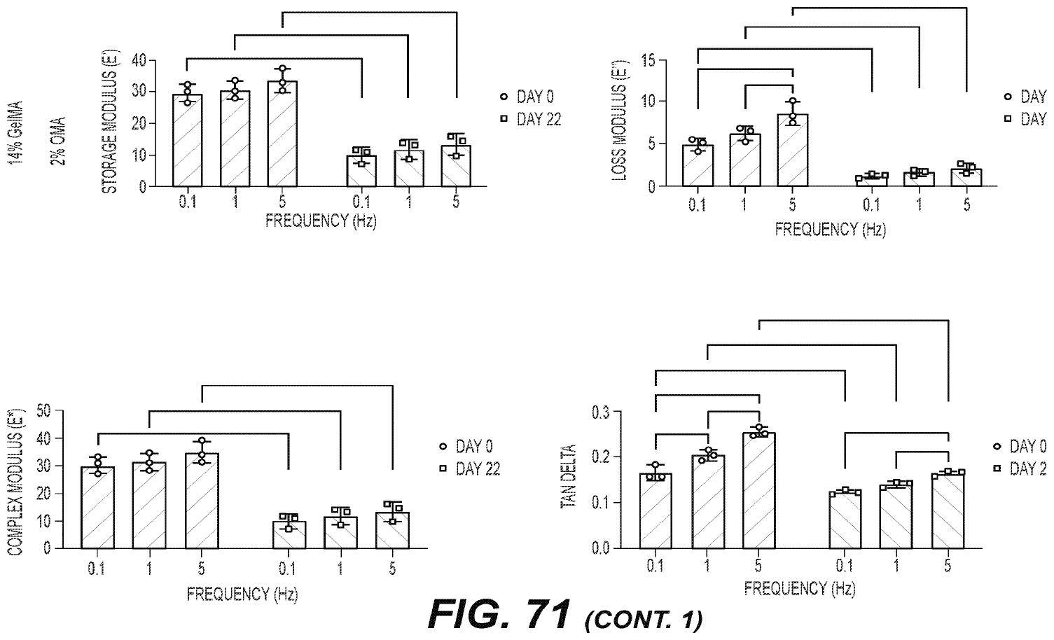

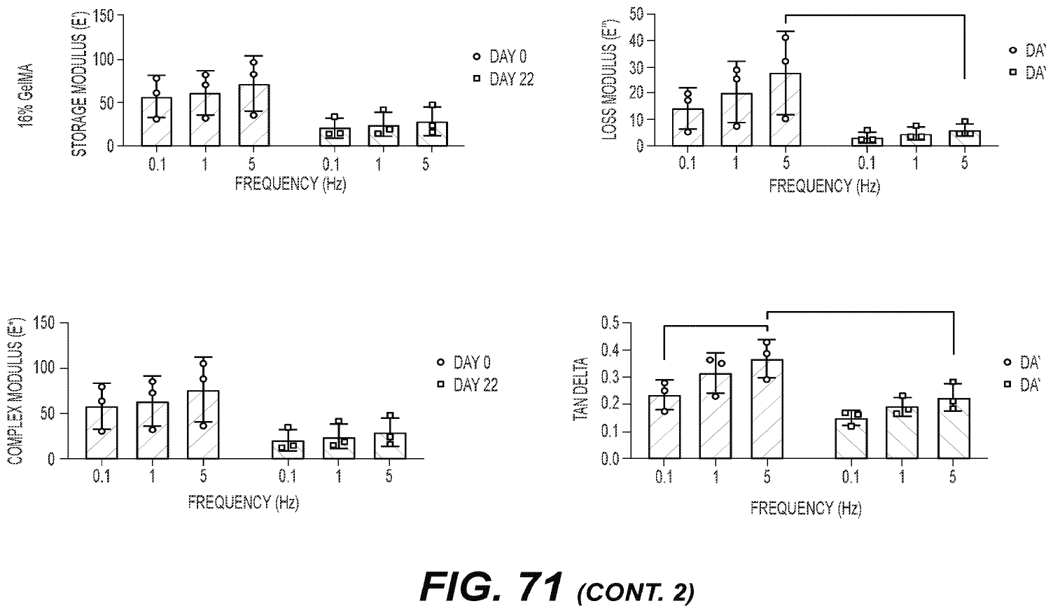

Mechanical characterization of disc constructs. To determine if hydrogel mechanical properties had an impact on lubricin expression, discs were cast and tested on a dynamic mechanic analyzer (DMA). The moduli generated were plotted vs cumulative luminescence from the validation. There was no trend for either storage modulus or loss modulus (FIG. 69) with the R² values being 0.2670 and 0.1319, respectively. This data indicates these mechanical properties (E′ 10-50 kPa) of the biomaterials tested play a minimal role in lubricin expression.

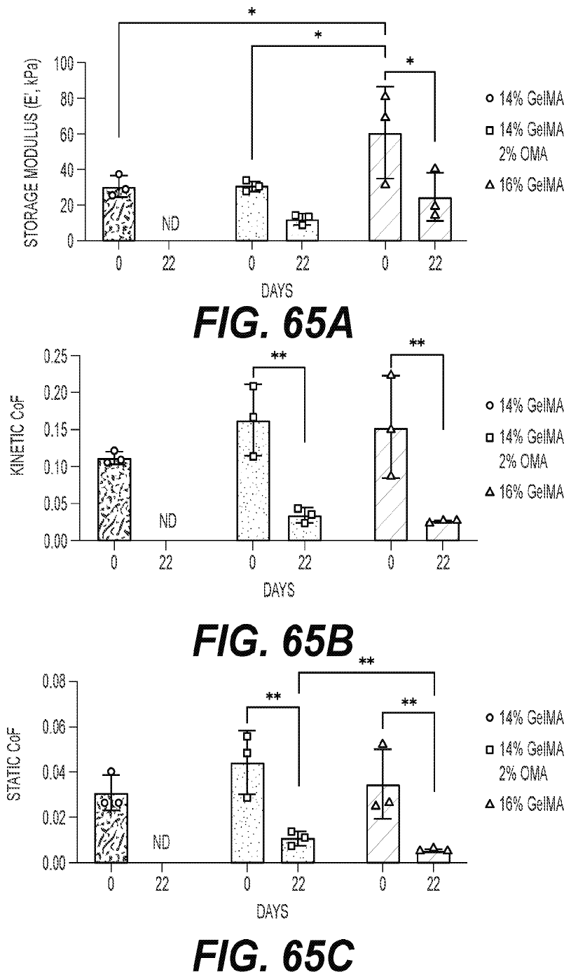

DMA analysis was also carried out to characterize 3D bioprinted discs containing HuPRG4gLuc cells on day 0 and 22 for biomaterials after 15s crosslinking. On day 0, both 14% GelMA and 14% GelMA/2% OMA had a storage modulus of ˜30 kPa (FIG. 65A). The storage modulus for 16% GelMA storage modulus was significantly higher than the other groups at ˜60 kPa (FIG. 65A). By day 22, the 14% GelMA group had contracted too much to be reliably tested. The 16% GelMA storage modulus significantly decreased by day 22 to ˜24 kPa (FIG. 65A). While the 14% GelMA/2% OMA storage modulus decreased to ˜12 kPa, but this change was not statistically significant (FIG. 65A). These trends are consistent for the loss modulus, tan delta, and complex modulus.

Lap shear testing was completed on both day 0 and day 22 constructs to determine the coefficient of friction. For both the kinetic (FIG. 65B) and static (FIG. 65C) coefficient of friction on day 0, there was no significant difference between the groups. As with the DMA testing, 14% GelMA was too small and thin by day 22 to be reliably tested. By day 22, both the 14% GelMA/2% OMA and 16% GelMA groups had a significant decrease in the static (FIG. 65C) and kinetic (FIG. 65B) coefficient of friction to ˜0.03. There was a significant difference in the static coefficient of friction on day 22 between the 14% GelMA/2% OMA (0.01) and 16% GelMA groups (0.005, FIG. 65C).

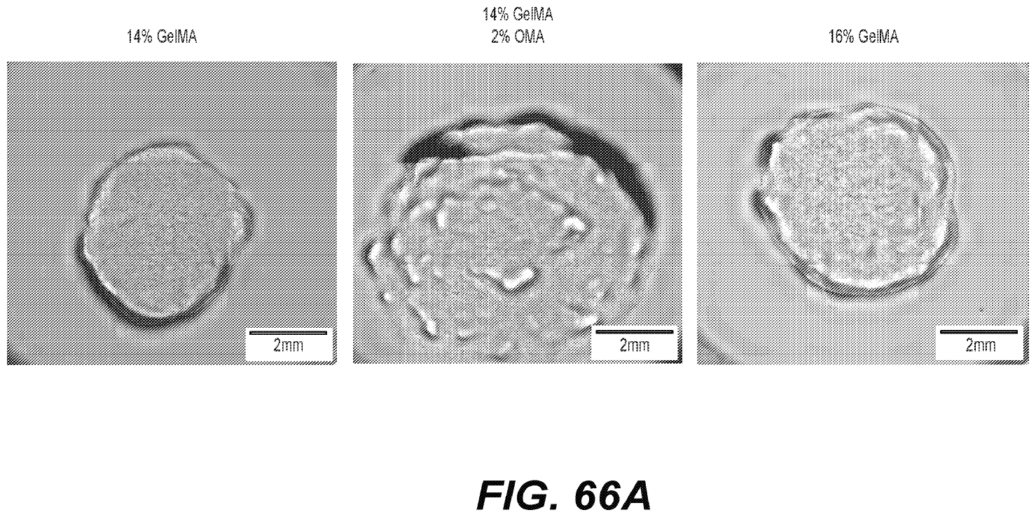

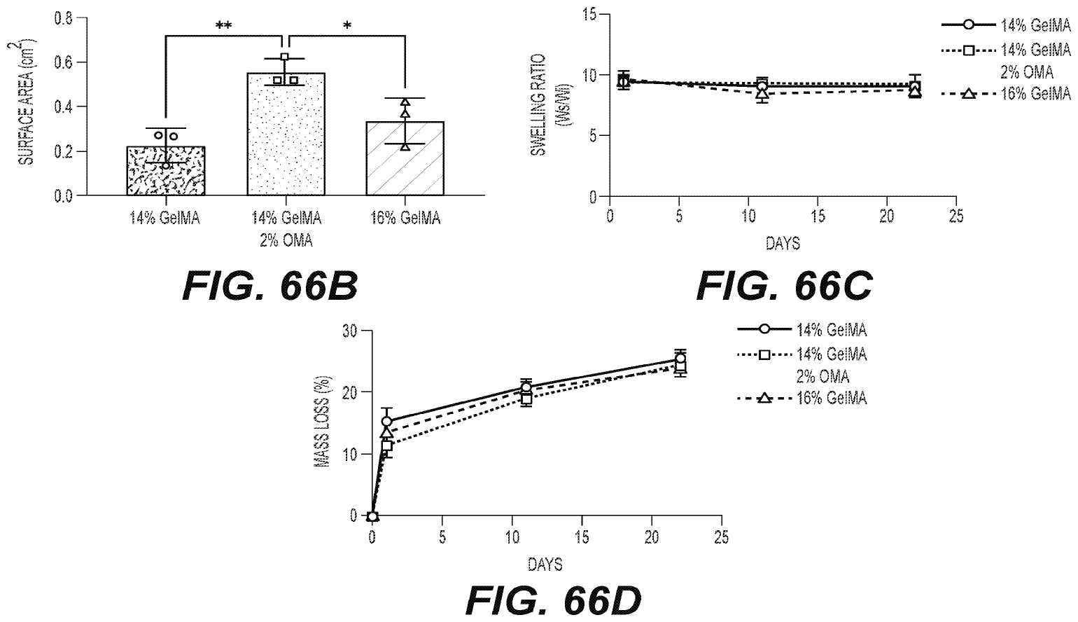

Shape fidelity of the bioprinted constructs and degradation of bioinks. Over the course of the 22 days in culture, the 14% and 16% GelMA groups noticeably decreased in size, while the 14% GelMA/2% OMA retained its shape. To quantify the size, images were taken on day 22 (FIG. 66A) and the surface area was measured. Both 14% and 16% GelMA had a significantly smaller surface area as compared to the 14% GelMA/2% OMA group (FIG. 66B). To determine the swelling ratio and mass loss discs were 3D printing without cells and incubated with chondrogenic media. The swelling ratio stayed consistent for all groups over the 22 days (FIG. 66C). All constructs loss about ˜12% of their mass on day 1 and ˜25% by day 22 (FIG. 66D). No group lost significantly more than either other group at any time point, when no cells were present.

To study potential treatments for OA, this study has developed a high-throughput reporter cell system for lubricin expression. Human primary articular chondrocytes were successfully transduced with a lubricin (PRG4) promoter-driven Gaussia luciferase (HuPRG4gLuc) while retaining chondrogenic capacity. Lubricin expression correlated well with luciferase expression both at the gene and protein level. Gaussia luciferase is secreted from the cells allowing for non-destructive, temporal analysis of lubricin expression. This study expands upon a previous biomaterial optimization now focusing on the surface zone of articular cartilage. Lubricin is an essential proteoglycan for articular cartilage function and treatment with lubricin mimetics have been shown to reduce the damage of PTOA in rats. The integrated use of DoE with a rapid and easy reporter system allowed us to identify an optimal biomaterial composition for 3D bioprinting, stimulating lubricin expression while maintaining the printed shape. To the best of our knowledge, this is the first study to assess lubricin expression in 3D bioprinted cartilage constructs. Use of this technology include identification of other lubricin stimulating conditions, and utilization of those compositions for 3D bioprinted resurfacing of the joint.

In this study, gelatin methacrylate (GelMA) and oxidized methacrylate alginate (OMA) was used. GelMA is a commonly used biomaterial for 3D bioprinting cartilage, used in about 35% of cartilage 3D bioprinting papers since 2012. While it has advantageous properties, this study sought to optimize its lubricin promoting capacity by mixing with other biomaterials. Alginate is another frequently used biomaterial, but has the distinct drawback of not being naturally degradable in humans. One method to accelerate the degradation rate of alginate is oxidation, making it more vulnerable to hydrolysis. To generate a useful biomaterial with tunable degradation rates, oxidized alginate was methacrylated adding a site for photocrosslinking. Previously, OMA containing human bone marrow-derived mesenchymal stem cells has been 3D bioprinted in complex geometries with high resolution and high cell viability. This study is the first-time mixtures of GelMA and OMA were 3D bioprinted with primary human articular chondrocytes. Previously, we hypothesized that an imbalance in ECM production and material degradation rate contributed to a decrease in material storage modulus overtime. Combining GelMA and OMA indicates that this can improve the stability of the construct, while still providing the biochemical cues necessary for lubricin expression.

To identify combinations of GelMA and OMA for lubricin expression an initial screen was implemented using groups generated by the Design of Experiment (DoE). This method uses statistical modeling to reduce the number of conditions tested, while still giving a good overview of the design space. The results of this screen showed the highest final percentage of GelMA and OMA, 12% and 2% respectively, had the highest lubricin expression. To investigate whether maximal stimulation had been achieved, the final percentages were further increased. It was found that increasing the final percentage of OMA from 2% to 4% (with 12% GelMA) was not beneficial for lubricin expression, however increasing GelMA from 12% to 14% was. In the screen 12% GelMA/2% OMA had higher lubricin expression as compared to 12% GelMA alone, and in the validation 14% GelMA/2% OMA had higher lubricin expression as compared to 14% GelMA alone in the final week. This indicates that while increasing GelMA increased lubricin expression, the addition of OMA further improved expression.

Based on luminescence and GAG/DNA results of the validation, the 14% GelMA/2% OMA after 15s crosslinking was chosen for subsequent bioprinting, while 14% GelMA and 16% GelMA (also at 15s crosslinking) were 3D bioprinted as controls. Luminescence data showed that raising the final GelMA percentage increased the lubricin expression, since the 16% GelMA group had the highest luminescence. However, 14% GelMA/2% OMA secreted more lubricin from the construct as shown by ELISA, while the 16% GelMA group retained more within the construct as shown by immunohistochemistry. This is consistent with lap-shear data that showed the 16% GelMA group had a significantly lower static coefficient of friction as compared to 14% GelMA/2% OMA. Theoretically, more lubricin was still present in the construct effectively reducing the coefficient of friction. The lubricin immunohistochemical staining was rather sparce overall, and this can be due to lubricin also being secreted. It can also be due to the cell density within the construct. The cell density was kept consistent from the screen through the bioprinting, originally chosen to compare between the cell aggregate control and the biomaterials. However, the surface zone of articular cartilage has a higher cell density than the deeper zones, and mimicking that in vitro can increase lubricin expression. It should also be noted that the chondrocytes used in this study were a mixed population, not exclusively surface zone chondrocytes. It has been shown that even with TGFβ stimulation, middle and deep zone chondrocytes had barely any lubricin expression. Incorporating solely surface zone chondrocytes can result in higher lubricin expression.

One of the requirements for an ideal tissue engineered articular cartilage construct is that it needs to not only fill the defect space but maintain that space while new tissue forms. Meaning that the 3D bioprinted construct must retain its shape while forming cartilage tissue. 3D bioprinted constructs with articular chondrocytes encapsulated had a noticeable decrease in size in both the 14% and 16% GelMA groups, while the 14% GelMA/2% OMA group retained its shape. Both the 14% and 16% GelMA groups had a significantly smaller surface area by day 22 as compared to 14% GelMA/2% OMA. The retention of area by 14% GelMA/2% OMA can be due to the higher GAG content and the increased distribution of type II collagen in the construct. Increased ECM can be replacing degrading biomaterial. Alternatively, this stabilizing effect is likely because of the presence of OMA and its controllable degradation rate. There is a chance that by adding 2% OMA to 16% GelMA to see more lubricin secretion and construct stabilization over the 22 days; however, higher final percentages of biomaterials increase the difficultly in handling and runs the risk of further reducing cell viability. Finding the balance between biomaterial degradation and tissue formation can continue to be a challenge of tissue engineering, but biomaterials like OMA can facilitate finding that equilibrium. Overall, this study has demonstrated the utility of extracellular matrix-driven secreted reporter in optimizing bioinks beyond the usual printability and cell viability metrics.

This study successfully determined an optimal combination of GelMA and OMA for surface zone articular cartilage 3D bioprinting focused on lubricin expression using this novel HuPRG4gLuc reporter cell system. Using the DoE in tandem with the HuPRG4gLuc cells created a more streamlined and systematic approach for testing biomaterials for 3D bioprinting. The 16% GelMA group had the highest luminescence, it also retained more lubricin within the construct as compared to 14% GelMA/2% OMA. The 14% GelMA/2% OMA group had higher lubricin secretion, was easier to print with and had better shape stability over the 22 days in culture. Together these results indicate the 14% GelMA/2% OMA after 15s crosslinking as the optimal combination tested for lubricin expression and 3D bioprinting of surface zone articular cartilage.

### Example 7. Screen of Natural Products to Stimulate Lubricin Production in Human Cartilage

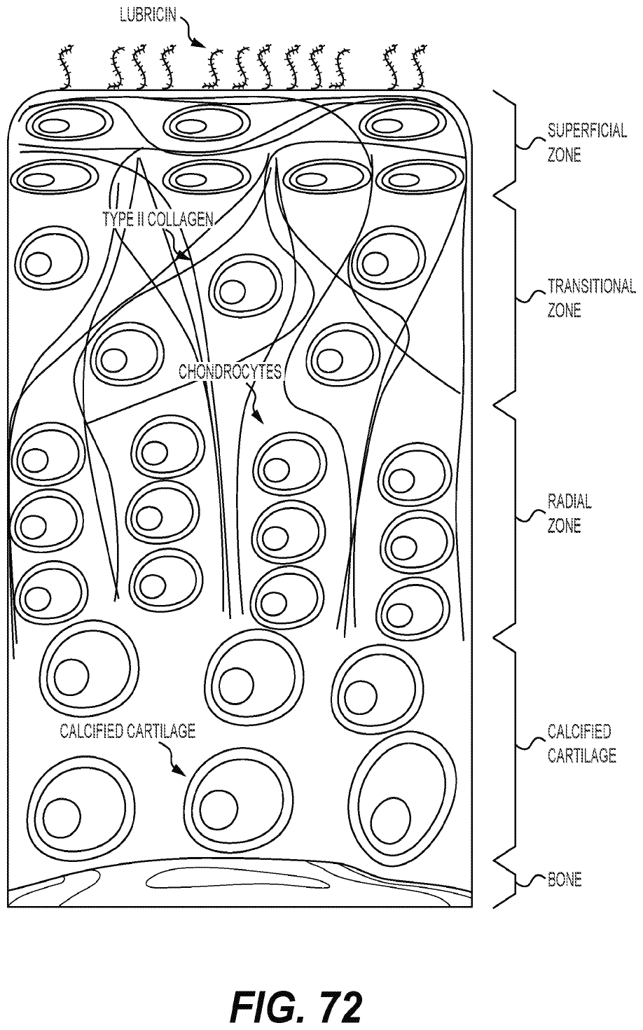

Lubricin is a potential target for disease modifying treatment of osteoarthritis Chondrocytes in the surface zone synthesize lubricin (PRG4; FIG. 72), a proteoglycan that functions as a boundary lubricant and anti-inflammatory molecule. A natural product library is a good source of bioactive molecules. There are currently no disease modifying drugs to treat osteoarthritis in the clinic.

3D culture more accurately represents articular chondrogenesis than 2D culture. In vivo effects are more accurately represented by primary human articular chondrocytes than they are by immortalized human cells or animal cells. Secreted Gaussia luciferase is a quick, stable, cheap and automation friendly method to assess 3D cartilage cultures. Cell expansion, while retaining chondrogenic capacity, can be achieved through culture on synoviocyte derived extracellular matrix. Transforming growth factor beta 1 (TGFβ1) is a known stimulator of chondrogenesis and, specifically, of lubricin.

### Methods

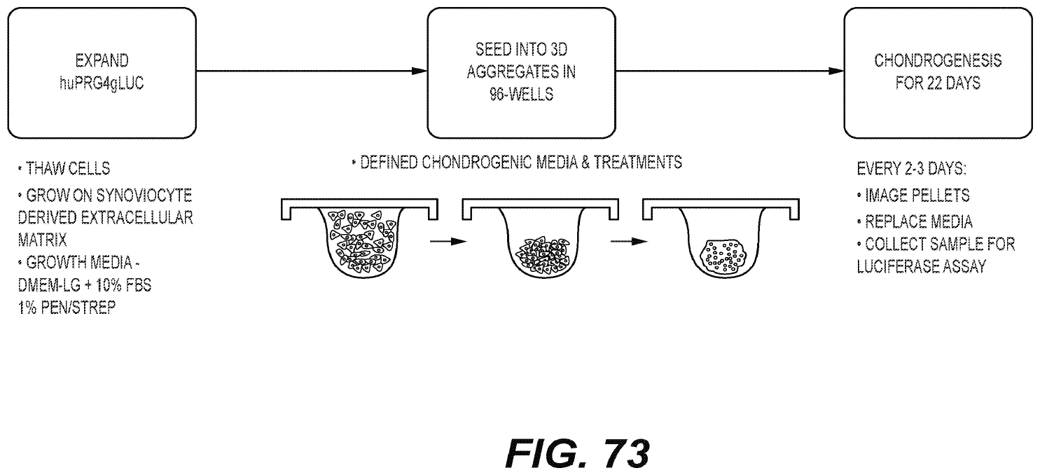

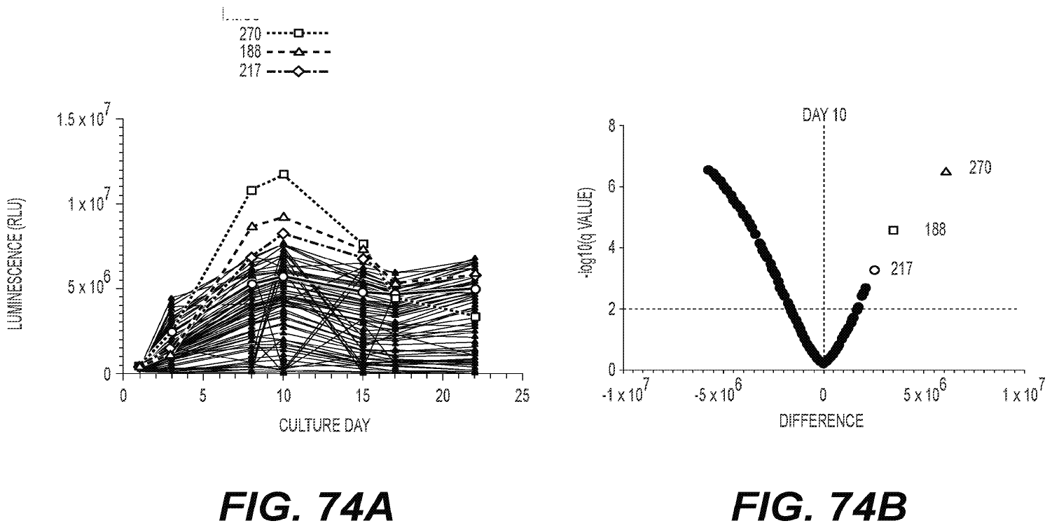

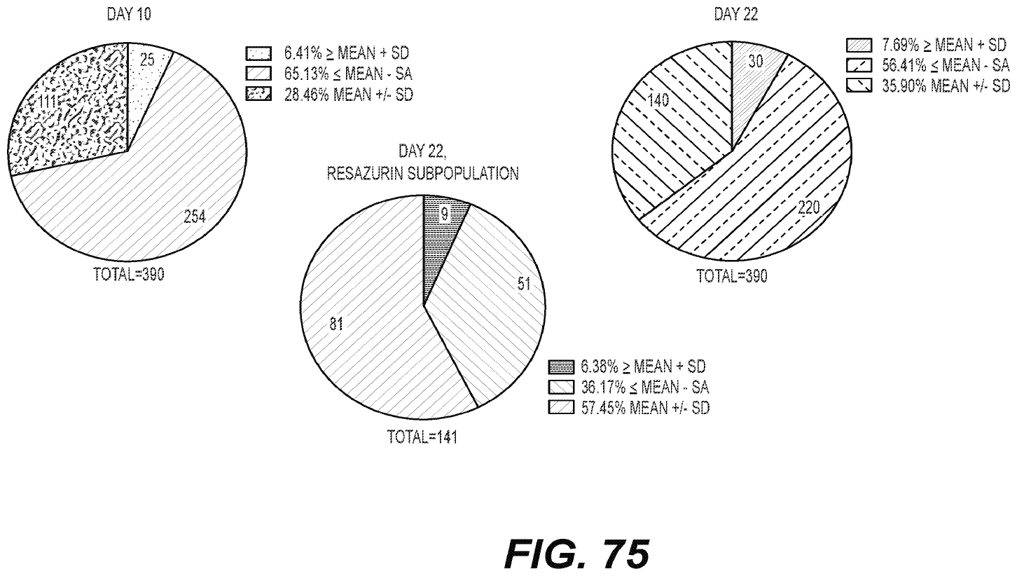

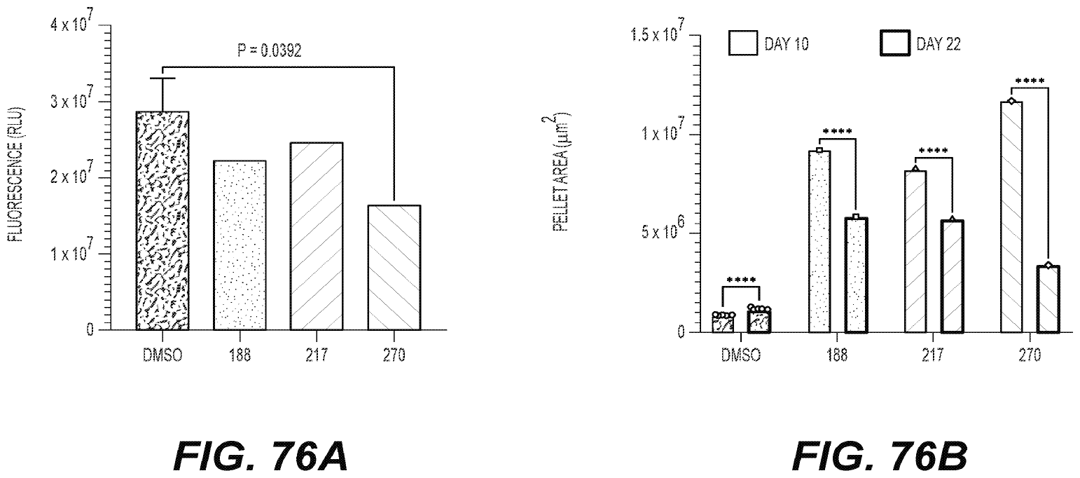

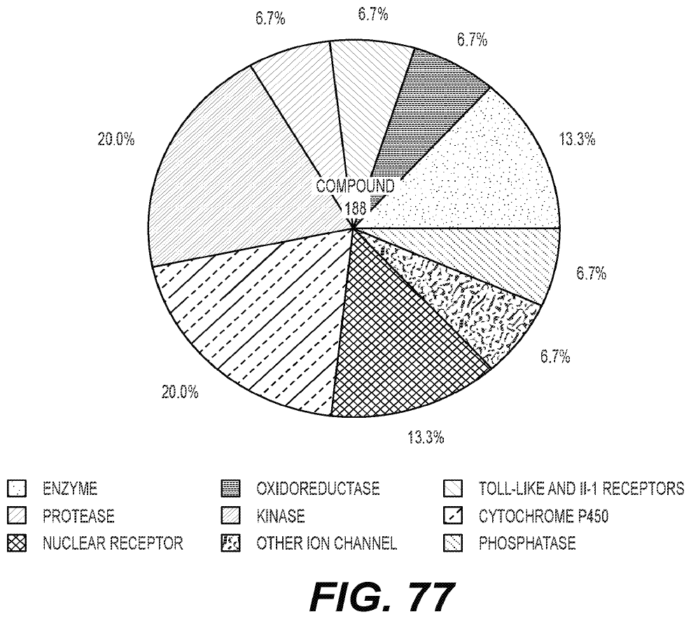

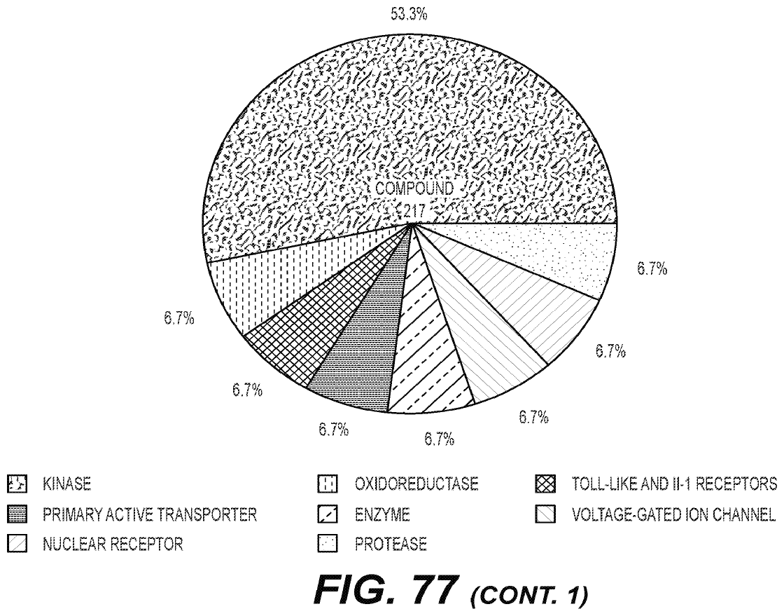

Characterization of reporter cells through TGFβ1 dose response:
Human primary chondrocytes transduced with a lubricin (PRG4) promotor driven Gaussia luciferase were expanded on synoviocyte matrix then cultured as cell aggregates in defined chondrogenic media [4,6,7] with TGFβ1 (0-40 ng/ml, FIG. 73)
Culture media from day 16 was used for human lubricin ELISA
On day 22, cell aggregates were used for qPCR (HPRT reference gene [7]) or for histology
All cultures were performed under physioxia (5% 02) [4]

Lubricin natural product screen:
Having characterized the cells as both chondrogenic and with good correlation of lubricin expression with Gaussia luciferase expression, we used those cells to screen a natural product library (390 compounds at 5 μM in well, Natural Product Library V, NCI).
Cells were treated from seeding in defined chondrogenic media containing 10 ng/ml TGFβ1 using an automated pipetting device (OT-2, Opentrons)
Aggregates were sampled and fed (OT-2, Opentrons) and imaged (M,W,F; PicoXpress), with luciferase assays (W, F)
At the end of the 22-day screen, aggregates were assayed for metabolic activity (resazurin assay) then formalin fixed and embedded for histology
Sections were stained for glycosaminoglycan (safranin-O), lubricin and type II collagen.

Human primary chondrocytes were successfully transduced with a lubricin (PRG4) promotor-driven Gaussia luciferase while still retaining their chondrogenic capacity (GAG) and type II Collagen. Lubricin expression correlated well with luciferase both in terms of gene expression and lubricin protein (ELISA) as well as histology. A natural product library was successfully screened using a robotic pipetting device. Most of the compounds tested inhibited lubricin production, but a fraction (˜6%) stimulated lubricin production. Three compounds highlighted (188, 217, 270) showed decreased metabolic activity at day 22. While all three highlighted compounds had significantly larger aggregates, those aggregates significantly decreased in size from day 10 to 22. No predicted targets were found for compound 270 while compounds 188 and 217 both had several common target classes. Human primary chondrocytes were successfully transduced with a lubricin (PRG4) promotor-driven Gaussia luciferase while still retaining their chondrogenic capacity (GAG) and type II Collagen Lubricin expression correlated well with luciferase both in terms of gene expression and lubricin protein (ELISA) as well as histology. A natural product library was successfully screened using a robotic pipetting device. Most of the compounds tested inhibited lubricin production, but a fraction (˜6%) stimulated lubricin production. Three compounds highlighted (188, 217, 270) showed decreased metabolic activity at day 22. While all three highlighted compounds had significantly larger aggregates, those aggregates significantly decreased in size from day 10 to 22. No predicted targets were found for compound 270 while compounds 188 and 217 both had several common target classes.

SEQUENCES

5′-caccGGTCTGCTTCTTGTAAAAACC-3′ (SEQ ID NO: 1)
5′-aaacGGTTTTTACAAGAAGCAGACC-3′ (SEQ ID NO: 2)
5′ ATTGACACTGGCAAAACAATGC 3′ (SEQ ID NO: 3)
5′ TCCAACACTTCGTGGGGTCC 3′ (SEQ ID NO: 4)
5′ ACGCTGCCACACCTACGA 3′ (SEQ ID NO: 5)
5′ CCTTGAACCCAGGAATCTCAG 3′ (SEQ ID NO: 6)
5′ TGGAGACTACTGGATTGACCCCAACCAA 3′ (SEQ ID NO: 7)
5′ TCTCGCCAGTCTCCATGTTGCAGA 3′ (SEQ ID NO: 8)
5′ ATTGACACTGGCAAAACAATGC 3′ (SEQ ID NO: 9)
5′ TCCAACACTTCGTGGGGTCC 3′ (SEQ ID NO: 10)
5′ ACGCTGCCACACCTACGA 3′ (SEQ ID NO: 11)
5′ CCTTGAACCCAGGAATCTCAG 3′ (SEQ ID NO: 12)
5′ TTGCTCCTCTCTGTTTTCGT 3′ (SEQ ID NO: 13)
5′ ATACCCTTCCCCACATCTCCC 3′ (SEQ ID NO: 14)
5′ GTGGTGGTCGGGGCCTT 3′ (SEQ ID NO: 15)
5′ CGGAGCAGGCAGGACAC 3′ (SEQ ID NO: 16)

1. A three-dimensional culture system for producing cartilage, said culture system comprising a chondrocyte that is genetically engineered to express a reporter protein; wherein the expression of a reporter protein is driven by a promoter, wherein the promoter is selected from a type 2 collagen promoter, a PRG4 promoter, a COL2A1 promoter, and an ACAN promoter, wherein the culture system is at about 2% to 8% oxygen.

2. The three-dimensional culture system of claim 1, wherein the culture system is at about 5% oxygen.

3. The three-dimensional culture system of claim 1, wherein the reporter protein is a bioluminescent protein.

4. (canceled)

5. A method of producing cartilage comprising using the three-dimensional culture system of claim 1.

6. A three-dimensional culture system for selecting an agent for promoting cartilage regeneration, said system comprising a chondrocyte that is genetically engineered to express a promoter, wherein the promoter is selected from a type 2 collagen promoter, a PRG4 promoter, a COL2A1 promoter, and an ACAN promoter; wherein the promoter drives the expression of a reporter protein, wherein the system is at about 2% to 8% oxygen.

7. The three-dimensional culture system of claim 6, wherein the culture system is at about 5% oxygen.

8. The three-dimensional culture system of claim 6, wherein the reporter protein is a bioluminescent protein.

9. (canceled)

10. The three-dimensional culture system of claim 6, further comprising IL-1β and/or TGF-β.

11. The three-dimensional culture system of claim 6, wherein agent is a therapeutic agent, a natural product, a mineral, or a biomaterial.

12. (canceled)

13. A method of selecting a therapeutic agent for promoting cartilage regeneration, comprising
a. contacting the chondrocyte of the three-dimensional culture system of claim 6 with the therapeutic agent;
b. obtaining a sample of the culture medium; and
c. determining a level of the reporter protein in the sample of step b;
wherein increased level of the reporter protein in the sample relative to a reference control indicates that the therapeutic agent promotes cartilage regeneration.

14. The method of claim 13, wherein the sample of the culture medium is obtained on day 5, day 15, day 20, or later after contacting the chondrocyte with the therapeutic agent.

15. (canceled)

16. (canceled)

17. The method of claim 13, wherein step a) further comprises contacting the chondrocyte with IL-1β and/or TGF-β from day 15 after contacting the chondrocyte with the therapeutic agent.

18. The method of claim 13, wherein the therapeutic agent promoting cartilage regeneration increases an expression level of a dopamine receptor in the chondrocyte.

19. The method of claim 18, wherein the dopamine receptor comprises dopamine receptor type 4.

20. A method of treating a disorder of cartilage in a subject in need, comprising administering to the subject a therapeutically effective amount of a therapeutic agent, wherein the therapeutic agent is a dopamine receptor agonist.

21. The method of claim 20, wherein the therapeutic agent increases an expression level of a dopamine receptor.

22. The method of claim 20, wherein the dopamine receptor comprises dopamine receptor type 4.

23. The method of claim 20, wherein the disorder is an articular cartilage defect.

24. The method of claim 20, wherein the disorder is osteoarthritis.

25. The method of claim 20, wherein the therapeutic agent comprises 6-Hydroxy-2-methoxyaporphine, (-)-Apoglaziovine, Pentoxifylline, 1,10-Dihydroxy-2-methoxyaporphine, Deserpidine, Vincristine sulfate, Promoline, or Calcium folinate, or a derivative thereof.

26. The method of claim 20, wherein the therapeutic agent is Promoline.

27. A three-dimensional bioprinting implant comprising one or more biomaterials and cells, wherein the biomaterials are selected from gelatin methacrylate (GelMA), hyaluronic acid methacrylate (HAMA), and oxidized methacrylated alginate (OMA).

28. The three-dimensional bioprinting implant of claim 27, wherein the implant comprises GelMA and HAMA.

29. The three-dimensional bioprinting implant of claim 28, wherein the ratio of GelMA 15% v/v and HAMA 2% v/v is about 2:1.

30. The three-dimensional bioprinting implant of claim 27, the implant comprises GelMA and oxidized methacrylated alginate (OMA).

31. The three-dimensional bioprinting implant of claim 27, wherein the cells are chondrocytes.
